SEQUENCE LISTING (186 sequences)

SEQ ID NO: 1 - protein, 21 aa, Artificial Sequence
  source: /note=“Description of Artificial Sequence: Synthetic peptide”
MALPVTALLLPLALLLHAARP

SEQ ID NO: 2 - protein, 45 aa, Artificial Sequence
  source: /note=“Description of Artificial Sequence: Synthetic polypeptide”
TTTPAPRPPTPAPTIASQPLSLRPEACRPAAGGAVHTRGLDFACD

SEQ ID NO: 3-4 - skipped (2 entries)

SEQ ID NO: 5 - protein, 10 aa, Artificial Sequence
  source: /note=“Description of Artificial Sequence: Synthetic peptide”
GGGGSGGGGS

SEQ ID NO: 6 - protein, 24 aa, Artificial Sequence
  source: /note=“Description of Artificial Sequence: Synthetic peptide”
IYIWAPLAGTCGVLLLSLVITLYC

SEQ ID NO: 7 - protein, 42 aa, Artificial Sequence
  source: /note=“Description of Artificial Sequence: Synthetic polypeptide”
KRGRKKLLYIFKQPFMRPVQTTQEEDGCSCRFPEEEEGGCEL

SEQ ID NO: 8 - DNA, 30 bp, Artificial Sequence
  source: /note=“Description of Artificial Sequence: Synthetic oligonucleotide”
ggtggcggaggttctggaggtggaggttcc

SEQ ID NO: 9 - protein, 112 aa, Artificial Sequence
  source: /note=“Description of Artificial Sequence: Synthetic polypeptide”
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 1184 bp, Artificial Sequence
  source: /note=“Description of Artificial Sequence: Synthetic polynucleotide”
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 12 - DNA, 63 bp, Artificial Sequence
  source: /note=“Description of Artificial Sequence: Synthetic oligonucleotide”
atggccctgcctgtgacagccctgctgctgcctctggctctgctgctgcatgccgctagaccc

SEQ ID NO: 13 - DNA, 135 bp, Artificial Sequence
  source: /note=“Description of Artificial Sequence: Synthetic polynucleotide”
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 14 - DNA, 123 bp, Artificial Sequence
  source: /note=“Description of Artificial Sequence: Synthetic polynucleotide”
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 15 - protein, 40 aa, Artificial Sequence
  source: /note=“Description of Artificial Sequence: Synthetic polypeptide”
  SITE (1)..(40): /note=“This sequence may encompass 1-10 ′Gly Gly Gly Ser′ repeating units”
  source: /note=“See specification as filed for detailed description of substitutions and preferred embodiments”
GGGSGGGSGGGSGGGSGGGSGGGSGGGSGGGSGGGSGGGS

SEQ ID NO: 16 - protein, 48 aa, Artificial Sequence
  source: /note=“Description of Artificial Sequence: Synthetic polypeptide”
QRRKYRSNKGESPVEPAEPCRYSCPREEEGSTIPIQEDYRKPEPACSP

SEQ ID NO: 17 - DNA, 72 bp, Artificial Sequence
  source: /note=“Description of Artificial Sequence: Synthetic oligonucleotide”
atctacatctgggcgcccttggccgggacttgtggggtccttctcctgtcactggttatcaccctttactgc

SEQ ID NO: 18 - DNA, 126 bp, Artificial Sequence
  source: /note=“Description of Artificial Sequence: Synthetic polynucleotide”
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 19 - skipped

SEQ ID NO: 20 - DNA, 336 bp, Artificial Sequence
  source: /note=“Description of Artificial Sequence: Synthetic polynucleotide”
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 21 - DNA, 336 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 22 - DNA, 813 bp, Artificial Sequence
  source: /note=“Description of Artificial Sequence: Synthetic polynucleotide”
atggccctgcccgtcaccgctctgctgctgccccttgctctgcttcttcatgcagcaaggccggacatccagatgacccaaaccacctcatccctctctgcctctcttggagacagggtgaccatttcttgtcgcgccagccaggacatcagcaagtatctgaactggtatcagcagaagccggacggaaccgtgaagctcctgatctaccatacctctcgcctgcatagcggcgtgccctcacgcttctctggaagcggatcaggaaccgattattctctcactatttcaaatcttgagcaggaagatattgccacctatttctgccagcagggtaataccctgccctacaccttcggaggagggaccaagctcgaaatcaccggtggaggaggcagcggcggtggagggtctggtggaggtggttctgaggtgaagctgcaagaatcaggccctggacttgtggccccttcacagtccctgagcgtgacttgcaccgtgtccggagtctccctgcccgactacggagtgtcatggatcagacaacctccacggaaaggactggaatggctcggtgtcatctggggtagcgaaactacttactacaattcagccctcaaaagcaggctgactattatcaaggacaacagcaagtcccaagtctttcttaagatgaactcactccagactgacgacaccgcaatctactattgtgctaagcactactactacggaggatcctacgctatggattactggggacaaggtacttccgtcactgtctcttcacaccatcatcaccatcaccatcac

SEQ ID NO: 23-24 - skipped (2 entries)

SEQ ID NO: 25 - protein, 5 aa, Artificial Sequence
  source: /note=“Description of Artificial Sequence: Synthetic peptide”
  source: /note=“See specification as filed for detailed description of substitutions and preferred embodiments”
GGGGS

SEQ ID NO: 26 - protein, 30 aa, Artificial Sequence
  source: /note=“Description of Artificial Sequence: Synthetic polypeptide”
  SITE (1)..(30): /note=“This sequence may encompass 1-6 ′Gly Gly Gly Gly Ser′ repeating units”
GGGGSGGGGSGGGGSGGGGSGGGGSGGGGS

SEQ ID NO: 27 - protein, 20 aa, Artificial Sequence
  source: /note=“Description of Artificial Sequence: Synthetic peptide”
GGGGSGGGGSGGGGSGGGGS

SEQ ID NO: 28 - protein, 15 aa, Artificial Sequence
  source: /note=“Description of Artificial Sequence: Synthetic peptide”
GGGGSGGGGSGGGGS

SEQ ID NO: 29 - protein, 4 aa, Artificial Sequence
  source: /note=“Description of Artificial Sequence: Synthetic peptide”
GGGS

SEQ ID NO: 30 - RNA, 5000 nt, Artificial Sequence
  source: /note=“Description of Artificial Sequence: Synthetic polynucleotide”
  misc_feature (1)..(5000): /note=“This sequence may encompass 50-5000 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 31 - RNA, 100 nt, Artificial Sequence
  source: /note=“Description of Artificial Sequence: Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 32 - DNA, 5000 bp, Artificial Sequence
  source: /note=“Description of Artificial Sequence: Synthetic polynucleotide”
  misc_feature (1)..(5000): /note=“This sequence may encompass 50-5000 nucleotides”
tttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttt

SEQ ID NO: 33 - RNA, 5000 nt, Artificial Sequence; identical to SEQ ID NO: 30
  source: /note=“Description of Artificial Sequence: Synthetic polynucleotide”
  misc_feature (1)..(5000): /note=“This sequence may encompass 100-5000 nucleotides”

SEQ ID NO: 34 - RNA, 400 nt, Artificial Sequence
  source: /note=“Description of Artificial Sequence: Synthetic polynucleotide”
  misc_feature (1)..(400): /note=“This sequence may encompass 100-400 nucleotides”
  source: /note=“See specification as filed for detailed description of substitutions and preferred embodiments”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 35 - RNA, 2000 nt, Artificial Sequence
  source: /note=“Description of Artificial Sequence: Synthetic polynucleotide”
  misc_feature (1)..(2000): /note=“This sequence may encompass 50-2000 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 36 - protein, 230 aa, Artificial Sequence
  source: /note=“Description of Artificial Sequence: Synthetic polypeptide”
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 37 - DNA, 690 bp, Artificial Sequence
  source: /note=“Description of Artificial Sequence: Synthetic polynucleotide”
gagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatg

SEQ ID NO: 38 - protein, 282 aa, Artificial Sequence
  source: /note=“Description of Artificial Sequence: Synthetic polypeptide”
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 39 - protein, 242 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 40 - protein, 242 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 41 - protein, 242 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 42 - protein, 242 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 43 - protein, 247 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 44 - protein, 247 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 45 - protein, 247 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 46 - protein, 247 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 47 - protein, 247 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 48 - protein, 247 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 49 - protein, 242 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 50 - protein, 242 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 51 - protein, 242 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 52 - DNA, 813 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactactcttcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagccaccaccatcatcaccatcaccat

SEQ ID NO: 53 - DNA, 813 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagccaccaccatcatcaccatcaccat

SEQ ID NO: 54 - DNA, 813 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactattcatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaacatcaccaccatcatcaccatcac

SEQ ID NO: 55 - DNA, 813 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactatcaatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaacatcaccaccatcatcaccatcac

SEQ ID NO: 56 - DNA, 828 bp, Artificial Sequence
  source: /note=“Description of Artificial Sequence: Synthetic polynucleotide”
atggccctcccagtgaccgctctgctgctgcctctcgcacttcttctccatgccgctcggcctgagatcgtcatgacccaaagccccgctaccctgtccctgtcacccggcgagagggcaaccctttcatgcagggccagccaggacatttctaagtacctcaactggtatcagcagaagccagggcaggctcctcgcctgctgatctaccacaccagccgcctccacagcggtatccccgccagattttccgggagcgggtctggaaccgactacaccctcaccatctcttctctgcagcccgaggatttcgccgtctatttctgccagcaggggaatactctgccgtacaccttcggtcaaggtaccaagctggaaatcaagggaggcggaggatcaggcggtggcggaagcggaggaggtggctccggaggaggaggttcccaagtgcagcttcaagaatcaggacccggacttgtgaagccatcagaaaccctctccctgacttgtaccgtgtccggtgtgagcctccccgactacggagtctcttggattcgccagcctccggggaagggtcttgaatggattggggtgatttggggatcagagactacttactactcttcatcacttaagtcacgggtcaccatcagcaaagataatagcaagaaccaagtgtcacttaagctgtcatctgtgaccgccgctgacaccgccgtgtactattgtgccaaacattactattacggagggtcttatgctatggactactggggacaggggaccctggtgactgtctctagccatcaccatcaccaccatcatcac

SEQ ID NO: 57 - DNA, 828 bp, Artificial Sequence
  source: /note=“Description of Artificial Sequence: Synthetic polynucleotide”
atggccctcccagtgaccgctctgctgctgcctctcgcacttcttctccatgccgctcggcctgagatcgtcatgacccaaagccccgctaccctgtccctgtcacccggcgagagggcaaccctttcatgcagggccagccaggacatttctaagtacctcaactggtatcagcagaagccagggcaggctcctcgcctgctgatctaccacaccagccgcctccacagcggtatccccgccagattttccgggagcgggtctggaaccgactacaccctcaccatctcttctctgcagcccgaggatttcgccgtctatttctgccagcaggggaatactctgccgtacaccttcggtcaaggtaccaagctggaaatcaagggaggcggaggatcaggcggtggcggaagcggaggaggtggctccggaggaggaggttcccaagtgcagcttcaagaatcaggacccggacttgtgaagccatcagaaaccctctccctgacttgtaccgtgtccggtgtgagcctccccgactacggagtctcttggattcgccagcctccggggaagggtcttgaatggattggggtgatttggggatcagagactacttactaccagtcatcacttaagtcacgggtcaccatcagcaaagataatagcaagaaccaagtgtcacttaagctgtcatctgtgaccgccgctgacaccgccgtgtactattgtgccaaacattactattacggagggtcttatgctatggactactggggacaggggaccctggtgactgtctctagccatcaccatcaccaccatcatcac

SEQ ID NO: 58 - DNA, 828 bp, Artificial Sequence
  source: /note=“Description of Artificial Sequence: Synthetic polynucleotide”
atggcactgcctgtcactgccctcctgctgcctctggccctccttctgcatgccgccaggccccaagtccagctgcaagagtcaggacccggactggtgaagccgtctgagactctctcactgacttgtaccgtcagcggcgtgtccctccccgactacggagtgtcatggatccgccaacctcccgggaaagggcttgaatggattggtgtcatctggggttctgaaaccacctactactcatcttccctgaagtccagggtgaccatcagcaaggataattccaagaaccaggtcagccttaagctgtcatctgtgaccgctgctgacaccgccgtgtattactgcgccaagcactactattacggaggaagctacgctatggactattggggacagggcactctcgtgactgtgagcagcggcggtggagggtctggaggtggaggatccggtggtggtgggtcaggcggaggagggagcgagattgtgatgactcagtcaccagccaccctttctctttcacccggcgagagagcaaccctgagctgtagagccagccaggacatttctaagtacctcaactggtatcagcaaaaaccggggcaggcccctcgcctcctgatctaccatacctcacgccttcactctggtatccccgctcggtttagcggatcaggatctggtaccgactacactctgaccatttccagcctgcagccagaagatttcgcagtgtatttctgccagcagggcaatacccttccttacaccttcggtcagggaaccaagctcgaaatcaagcaccatcaccatcatcaccaccat

SEQ ID NO: 59 - DNA, 828 bp, Artificial Sequence
  source: /note=“Description of Artificial Sequence: Synthetic polynucleotide”
atggcactgcctgtcactgccctcctgctgcctctggccctccttctgcatgccgccaggccccaagtccagctgcaagagtcaggacccggactggtgaagccgtctgagactctctcactgacttgtaccgtcagcggcgtgtccctccccgactacggagtgtcatggatccgccaacctcccgggaaagggcttgaatggattggtgtcatctggggttctgaaaccacctactaccagtcttccctgaagtccagggtgaccatcagcaaggataattccaagaaccaggtcagccttaagctgtcatctgtgaccgctgctgacaccgccgtgtattactgcgccaagcactactattacggaggaagctacgctatggactattggggacagggcactctcgtgactgtgagcagcggcggtggagggtctggaggtggaggatccggtggtggtgggtcaggcggaggagggagcgagattgtgatgactcagtcaccagccaccctttctctttcacccggcgagagagcaaccctgagctgtagagccagccaggacatttctaagtacctcaactggtatcagcaaaaaccggggcaggcccctcgcctcctgatctaccatacctcacgccttcactctggtatccccgctcggtttagcggatcaggatctggtaccgactacactctgaccatttccagcctgcagccagaagatttcgcagtgtatttctgccagcagggcaatacccttccttacaccttcggtcagggaaccaagctcgaaatcaagcaccatcaccatcatcatcaccac

SEQ ID NO: 60 - DNA, 828 bp, Artificial Sequence
  source: /note=“Description of Artificial Sequence: Synthetic polynucleotide”
atggccctcccagtgaccgctctgctgctgcctctcgcacttcttctccatgccgctcggcctgagatcgtcatgacccaaagccccgctaccctgtccctgtcacccggcgagagggcaaccctttcatgcagggccagccaggacatttctaagtacctcaactggtatcagcagaagccagggcaggctcctcgcctgctgatctaccacaccagccgcctccacagcggtatccccgccagattttccgggagcgggtctggaaccgactacaccctcaccatctcttctctgcagcccgaggatttcgccgtctatttctgccagcaggggaatactctgccgtacaccttcggtcaaggtaccaagctggaaatcaagggaggcggaggatcaggcggtggcggaagcggaggaggtggctccggaggaggaggttcccaagtgcagcttcaagaatcaggacccggacttgtgaagccatcagaaaccctctccctgacttgtaccgtgtccggtgtgagcctccccgactacggagtctcttggattcgccagcctccggggaagggtcttgaatggattggggtgatttggggatcagagactacttactacaattcatcacttaagtcacgggtcaccatcagcaaagataatagcaagaaccaagtgtcacttaagctgtcatctgtgaccgccgctgacaccgccgtgtactattgtgccaaacattactattacggagggtcttatgctatggactactggggacaggggaccctggtgactgtctctagccatcaccatcaccaccatcatcac

SEQ ID NO: 61 - DNA, 828 bp, Artificial Sequence
  source: /note=“Description of Artificial Sequence: Synthetic polynucleotide”
atggcactgcctgtcactgccctcctgctgcctctggccctccttctgcatgccgccaggccccaagtccagctgcaagagtcaggacccggactggtgaagccgtctgagactctctcactgacttgtaccgtcagcggcgtgtccctccccgactacggagtgtcatggatccgccaacctcccgggaaagggcttgaatggattggtgtcatctggggttctgaaaccacctactacaactcttccctgaagtccagggtgaccatcagcaaggataattccaagaaccaggtcagccttaagctgtcatctgtgaccgctgctgacaccgccgtgtattactgcgccaagcactactattacggaggaagctacgctatggactattggggacagggcactctcgtgactgtgagcagcggcggtggagggtctggaggtggaggatccggtggtggtgggtcaggcggaggagggagcgagattgtgatgactcagtcaccagccaccctttctctttcacccggcgagagagcaaccctgagctgtagagccagccaggacatttctaagtacctcaactggtatcagcaaaaaccggggcaggcccctcgcctcctgatctaccatacctcacgccttcactctggtatccccgctcggtttagcggatcaggatctggtaccgactacactctgaccatttccagcctgcagccagaagatttcgcagtgtatttctgccagcagggcaatacccttccttacaccttcggtcagggaaccaagctcgaaatcaagcaccatcaccatcatcaccaccat

SEQ ID NO: 62 - DNA, 813 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactacaattcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagccaccaccatcatcaccatcaccat

SEQ ID NO: 63 - DNA, 813 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactataactcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaacatcaccaccatcatcaccatcac

SEQ ID NO: 64 - protein, 271 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSHHHHHHHH

SEQ ID NO: 65 - protein, 271 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSHHHHHHHH

SEQ ID NO: 66 - protein, 271 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKHHHHHHHH

SEQ ID NO: 67 - protein, 271 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKHHHHHHHH

SEQ ID NO: 68 - protein, 276 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSHHHHHHHH

SEQ ID NO: 69 - protein, 276 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSHHHHHHHH

SEQ ID NO: 70 - protein, 276 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKHHHHHHHH

SEQ ID NO: 71 - protein, 276 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKHHHHHHHH

SEQ ID NO: 72 - protein, 276 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSHHHHHHHH

SEQ ID NO: 73 - protein, 276 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKHHHHHHHH

SEQ ID NO: 74 - protein, 271 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSHHHHHHHH

SEQ ID NO: 75 - protein, 271 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKHHHHHHHH

SEQ ID NO: 76 - protein, 271 aa, Artificial Sequence
  source: /note=“Description of Artificial Sequence: Synthetic polypeptide”
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSHHHHHHHH

SEQ ID NO: 77 - protein, 486 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - protein, 486 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 486 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 486 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 491 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - protein, 491 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - protein, 491 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - protein, 491 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - protein, 491 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 491 aa, Artificial Sequence; identical to SEQ ID NO: 85
  source: /note=“Description of Artificial Sequence: Synthetic polypeptide”

SEQ ID NO: 87 - protein, 491 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 486 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 486 aa, Artificial Sequence
  source: /note=“Description of Artificial Sequence: Synthetic polypeptide”
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - DNA, 1458 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactactcttcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 91 - DNA, 1458 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 92 - DNA, 1458 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactattcatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 93 - DNA, 1458 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactatcaatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 94 - DNA, 1473 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagcggcggaggcgggagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactactcttcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 95 - DNA, 1473 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagcggaggcggagggagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 96 - DNA, 1473 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactattcatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccggaggtggcggaagcgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 97 - DNA, 1473 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactatcaatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccggaggcggtgggtcagaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 98 - DNA, 1473 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagcggaggcggtgggagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactacaactcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 99 - DNA, 1473 bp, Artificial Sequence; identical to SEQ ID NO: 98
  source: /note=“Description of Artificial Sequence: Synthetic polynucleotide”

SEQ ID NO: 100 - DNA, 1473 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactataactcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccggaggtggcggaagcgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 101 - DNA, 1458 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactacaactcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 102 - DNA, 1458 bp, Artificial Sequence
  source: /note=“Description of Artificial Sequence: Synthetic polynucleotide”
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 103 - DNA, 847 bp, Artificial Sequence
  source: /note=“Description of Artificial Sequence: Synthetic polynucleotide”
aggtggcccgaaagtcccaaggcccaggcatctagtgttcctactgcacagccccaggcagaaggcagcctagccaaagctactactgcacctgccactacgcgcaatactggccgtggcggggaggagaagaaaaaggagaaagagaaagaagaacaggaagagagggagaccaagacccctgaatgtccatcccatacccagccgctgggcgtctatctcttgactcccgcagtacaggacttgtggcttagagataaggccacctttacatgtttcgtcgtgggctctgacctgaaggatgcccatttgacttgggaggttgccggaaaggtacccacagggggggttgaggaagggttgctggagcgccattccaatggctctcagagccagcactcaagactcacccttccgagatccctgtggaacgccgggacctctgtcacatgtactctaaatcatcctagcctgcccccacagcgtctgatggcccttagagagccagccgcccaggcaccagttaagcttagcctgaatctgctcgccagtagtgatcccccagaggccgccagctggctcttatgcgaagtgtccggctttagcccgcccaacatcttgctcatgtggctggaggaccagcgagaagtgaacaccagcggcttcgctccagcccggcccccaccccagccgggttctaccacattctgggcctggagtgtcttaagggtcccagcaccacctagcccccagccagccacatacacctgtgttgtgtcccatgaagatagcaggaccctgctaaatgcttctaggagtctggaggtttcctacgtgactgaccatt

SEQ ID NO: 104 - protein, 18 aa, Artificial Sequence
  source: /note=“Description of Artificial Sequence: Synthetic peptide”
GSTSGSGKPGSGEGSTKG

SEQ ID NO: 105 - protein, 394 aa, Artificial Sequence
  source: /note=“Description of Artificial Sequence: Synthetic polypeptide”
MALPVTALLLPLALLLHAARPPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 373 aa, Artificial Sequence
  source: /note=“Description of Artificial Sequence: Synthetic polypeptide”
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - DNA, 1182 bp, Artificial Sequence
  source: /note=“Description of Artificial Sequence: Synthetic polynucleotide”
atggccctccctgtcactgccctgcttctccccctcgcactcctgctccacgccgctagaccacccggatggtttctggactctccggatcgcccgtggaatcccccaaccttctcaccggcactcttggttgtgactgagggcgataatgcgaccttcacgtgctcgttctccaacacctccgaatcattcgtgctgaactggtaccgcatgagcccgtcaaaccagaccgacaagctcgccgcgtttccggaagatcggtcgcaaccgggacaggattgtcggttccgcgtgactcaactgccgaatggcagagacttccacatgagcgtggtccgcgctaggcgaaacgactccgggacctacctgtgcggagccatctcgctggcgcctaaggcccaaatcaaagagagcttgagggccgaactgagagtgaccgagcgcagagctgaggtgccaactgcacatccatccccatcgcctcggcctgcggggcagtttcagaccctggtcacgaccactccggcgccgcgcccaccgactccggccccaactatcgcgagccagcccctgtcgctgaggccggaagcatgccgccctgccgccggaggtgctgtgcatacccggggattggacttcgcatgcgacatctacatttgggctcctctcgccggaacttgtggcgtgctccttctgtccctggtcatcaccctgtactgcaagcggggtcggaaaaagcttctgtacattttcaagcagcccttcatgaggcccgtgcaaaccacccaggaggaggacggttgctcctgccggttccccgaagaggaagaaggaggttgcgagctgcgcgtgaagttctcccggagcgccgacgcccccgcctataagcagggccagaaccagctgtacaacgaactgaacctgggacggcgggaagagtacgatgtgctggacaagcggcgcggccgggaccccgaaatgggcgggaagcctagaagaaagaaccctcaggaaggcctgtataacgagctgcagaaggacaagatggccgaggcctactccgaaattgggatgaagggagagcggcggaggggaaaggggcacgacggcctgtaccaaggactgtccaccgccaccaaggacacatacgatgccctgcacatgcaggcccttccccctcgc

SEQ ID NO: 108 - skipped

SEQ ID NO: 109 - DNA, 521 bp, Unknown
  source: /note=“Description of Unknown: PGK Promoter sequence”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccgcctcgtccttcgcagcggccccccgggtgttcccatcgccgcttctaggcccactgcgacgcttgcctgcacttcttacacgctctgggtcccagccgcggcgacgcaaagggccttggtgcgggtctcgtcggcgcagggacgcgtttgggtcccgacggaaccttttccgcgttggggttggggcaccataagct

SEQ ID NO: 110 - DNA, 118 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtg

SEQ ID NO: 111 - DNA, 221 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacg

SEQ ID NO: 112 - DNA, 324 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccg

SEQ ID NO: 113 - DNA, 422 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccgcctcgtccttcgcagcggccccccgggtgttcccatcgccgcttctaggcccactgcgacgcttgcctgcacttcttacacgctctgggtcccagccg

SEQ ID NO: 114 - protein, 132 aa, Artificial Sequence
  source: /note=“Description of Artificial Sequence: Synthetic polypeptide”
DVPDYASLGGPSSPKKKRKVSRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETSY

SEQ ID NO: 115 - protein, 108 aa, Artificial Sequence
  source: /note=“Description of Artificial Sequence: Synthetic polypeptide”
VQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETS

SEQ ID NO: 116 - protein, 93 aa, Artificial Sequence [sequence omitted]

SEQ ID NO: 117 - protein, 95 aa, Artificial Sequence
  source: /note=“Description of Artificial Sequence: Synthetic polypeptide”
ILWHEMWHEGLIEASRLYFGERNVKGMFEVLEPLHAMMERGPQTLKETSFNQAYGRDLMEAQEWCRKYMKSGNVKDLTQAWDLYYHVFRRISKTS

SEQ ID NO: 118 - protein, 95 aa, Artificial Sequence
  source: /note=“Description of Artificial Sequence: Synthetic polypeptide”
ILWHEMWHEGLLEASRLYFGERNVKGMFEVLEPLHAMMERGPQTLKETSFNQAYGRDLMEAQEWCRKYMKSGNVKDLTQAWDLYYHVFRRISKTS

SEQ ID NO: 119 - protein, 95 aa, Artificial Sequence
  source: /note=“Description of Artificial Sequence: Synthetic polypeptide”
ILWHEMWHEGLEEASRLYFGERNVKGMFEVLEPLHAMMERGPQTLKETSFNQAYGRDLMEAQEWCRKYMKSGNVKDLLQAWDLYYHVFRRISKTS

SEQ ID NO: 120 - protein, 95 aa, Artificial Sequence
  source: /note=“Description of Artificial Sequence: Synthetic polypeptide”
  MOD_RES (12)..(12): Any amino acid
  MOD_RES (78)..(78): Any amino acid
ILWHEMWHEGLXEASRLYFGERNVKGMFEVLEPLHAMMERGPQTLKETSFNQAYGRDLMEAQEWCRKYMKSGNVKDLXQAWDLYYHVFRRISKTS

SEQ ID NO: 121 - protein, 95 aa, Artificial Sequence
  source: /note=“Description of Artificial Sequence: Synthetic polypeptide”
ILWHEMWHEGLIEASRLYFGERNVKGMFEVLEPLHAMMERGPQTLKETSFNQAYGRDLMEAQEWCRKYMKSGNVKDLLQAWDLYYHVFRRISKTS

SEQ ID NO: 122 - protein, 95 aa, Artificial Sequence
  source: /note=“Description of Artificial Sequence: Synthetic polypeptide”
ILWHEMWHEGLLEASRLYFGERNVKGMFEVLEPLHAMMERGPQTLKETSFNQAYGRDLMEAQEWCRKYMKSGNVKDLLQAWDLYYHVFRRISKTS

SEQ ID NO: 123 - DNA, 100 bp, Artificial Sequence
  source: /note=“Description of Artificial Sequence: Synthetic polynucleotide”
tttttttttttttttttttttttttttttttttttttttttttttttttttttttttttttttttttttttttttttttttttttttttttttttttttt

SEQ ID NO: 124 - protein, 244 aa, Artificial Sequence
  source: /note=“Description of Artificial Sequence: Synthetic polypeptide”
QVQLLESGAELVRPGSSVKISCKASGYAFSSYWMNWVKQRPGQGLEWIGQIYPGDGDTNYNGKFKGQATLTADKSSSTAYMQLSGLTSEDSAVYSCARKTISSVVDFYFDYWGQGTTVTGGGSGGGSGGGSGGGSELVLTQSPKFMSTSVGDRVSVTCKASQNVGTNVAWYQQKPGQSPKPLIYSATYRNSGVPDRFTGSGSGTDFTLTITNVQSKDLADYFCQYNRYPYTSFFFTKLEIKRRS

SEQ ID NO: 125 - protein, 246 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSE

SEQ ID NO: 126 - protein, 245 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 127 - DNA, 63 bp, Artificial Sequence
  source: /note=“Description of Artificial Sequence: Synthetic oligonucleotide”
atggccctccctgtcaccgccctgctgcttccgctggctcttctgctccacgccgctcggccc

SEQ ID NO: 128 - DNA, 63 bp, Artificial Sequence
  source: /note=“Description of Artificial Sequence: Synthetic oligonucleotide”
atggccttaccagtgaccgccttgctcctgccgctggccttgctgctccacgccgccaggccg

SEQ ID NO: 129 - DNA, 135 bp, Artificial Sequence
  source: /note=“Description of Artificial Sequence: Synthetic polynucleotide”
accactaccccagcaccgaggccacccaccccggctcctaccatcgcctcccagcctctgtccctgcgtccggaggcatgtagacccgcagctggtggggccgtgcatacccggggtcttgacttcgcctgcgat

SEQ ID NO: 130 - DNA, 72 bp, Artificial Sequence
  source: /note=“Description of Artificial Sequence: Synthetic oligonucleotide”
atctacatttgggcccctctggctggtacttgcggggtcctgctgctttcactcgtgatcactctttactgt

SEQ ID NO: 131 - DNA, 126 bp, Artificial Sequence
  source: /note=“Description of Artificial Sequence: Synthetic polynucleotide”
aagcgcggtcggaagaagctgctgtacatctttaagcaacccttcatgaggcctgtgcagactactcaagaggaggacggctgttcatgccggttcccagaggaggaggaaggcggctgcgaactg

SEQ ID NO: 132 - DNA, 336 bp, Artificial Sequence
  source: /note=“Description of Artificial Sequence: Synthetic polynucleotide”
cgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 133 - protein, 5 aa, Artificial Sequence
  source: /note=“Description of Artificial Sequence: Synthetic peptide”
DYGVS

SEQ ID NO: 134 - protein, 16 aa, Artificial Sequence
  source: /note=“Description of Artificial Sequence: Synthetic peptide”
VIWGSETTYYNSALKS

SEQ ID NO: 135 - protein, 16 aa, Artificial Sequence
  source: /note=“Description of Artificial Sequence: Synthetic peptide”
VIWGSETTYYSSSLKS

SEQ ID NO: 136 - protein, 16 aa, Artificial Sequence
  source: /note=“Description of Artificial Sequence: Synthetic peptide”
VIWGSETTYYQSSLKS

SEQ ID NO: 137 - protein, 16 aa, Artificial Sequence
  source: /note=“Description of Artificial Sequence: Synthetic peptide”
VIWGSETTYYNSSLKS

SEQ ID NO: 138 - protein, 12 aa, Artificial Sequence
  source: /note=“Description of Artificial Sequence: Synthetic peptide”
HYYYGGSYAMDY

SEQ ID NO: 139 - protein, 11 aa, Artificial Sequence
  source: /note=“Description of Artificial Sequence: Synthetic peptide”
RASQDISKYLN

SEQ ID NO: 140 - protein, 7 aa, Artificial Sequence
  source: /note=“Description of Artificial Sequence: Synthetic peptide”
HTSRLHS

SEQ ID NO: 141 - protein, 9 aa, Artificial Sequence
  source: /note=“Description of Artificial Sequence: Synthetic peptide”
QQGNTLPYT

SEQ ID NO: 142 - DNA, 1467 bp, Artificial Sequence
  source: /note=“Description of Artificial Sequence: Synthetic polynucleotide”
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 143 - protein, 489 aa, Artificial Sequence
  source: /note=“Description of Artificial Sequence: Synthetic polypeptide”
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 144 - protein, 266 aa, Artificial Sequence
  source: /note=“Description of Artificial Sequence: Synthetic polypeptide”
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVS

SEQ ID NO: 145 - DNA, 1383 bp, Artificial Sequence
  source: /note=“Description of Artificial Sequence: Synthetic polynucleotide”
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 146 - protein, 461 aa, Artificial Sequence
  source: /note=“Description of Artificial Sequence: Synthetic polypeptide”
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - protein, 267 aa, Artificial Sequence
  source: /note=“Description of Artificial Sequence: Synthetic polypeptide”
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 148 - protein, 465 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 149 - protein, 120 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 150 - protein, 107 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 151 - protein, 120 aa, Artificial Sequence
  source: /note=“Description of Artificial Sequence: Synthetic polypeptide”
QVQLQESGPGLVKPSETLSLTCTVSGVSLPDYGVSWIRQPPGKCLEWIGVIWGSETTYYQSSLKSRVTISKDNSKNQVSLKLSSVTAADTAVYYCAKHYYYGGSYAMDYWGQGTLVTVSS

SEQ ID NO: 152 - protein, 107 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGCGTKLEIK

SEQ ID NO: 153 - protein, 486 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 154 - DNA, 1461 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 155 - DNA, 1458 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 156 - DNA, 1398 bp, Artificial Sequence
  source: /note=“Description of Artificial Sequence: Synthetic polynucleotide”
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 157 - DNA, 360 bp, Artificial Sequence
  source: /note=“Description of Artificial Sequence: Synthetic polynucleotide”
caggtccagctgcaggaatcaggaccagggctggtgaaacctagcgaaactctgagtctgacttgtaccgtctccggggtgtctctgccagactacggcgtgagctggatcagacagccccctggcaagtgcctggagtggatcggcgtgatctggggctccgagaccacatactatcagagctccctgaagtctcgggtgaccatctccaaggacaactctaagaatcaggtgagcctgaagctgtctagcgtgaccgccgccgatacagccgtgtactattgtgccaagcactactattacggcggctcctatgccatggattactggggccagggcaccctggtgacagtgtcctct

SEQ ID NO: 158 - DNA, 360 bp, Artificial Sequence
  source: /note=“Description of Artificial Sequence: Synthetic polynucleotide”
caggtccagctgcaggaatccggcccaggactggtgaagcctagcgagaccctgtccctgacctgcacagtgagcggcgtgtccctgcccgattacggcgtgagctggatcagacagccccctggcaagtgtctggagtggatcggcgtgatctggggctctgagaccacatactatcagtcctctctgaagagcagggtgaccatctctaaggacaacagcaagaatcaggtgtccctgaagctgagctccgtgaccgcagcagatacagccgtgtactattgcgccaagcactactattacggcggctcctatgctatggattattgggggcagggcactctggtcactgtctcatca

SEQ ID NO: 159 - DNA, 360 bp, Artificial Sequence
  source: /note=“Description of Artificial Sequence: Synthetic polynucleotide”
caggtgcagctgcaggaatctggacccggactggtgaaacctagtgaaactctgtctctgacttgtaccgtctcaggggtctcactgccagactacggcgtgtcctggatcagacagccccctggcaagtgcctggagtggatcggcgtgatctggggctctgagaccacatactatcagagctccctgaagagccgggtgaccatctccaaggacaactctaagaatcaggtgtccctgaagctgtctagcgtgaccgccgccgatacagccgtgtactattgtgccaagcactactattacggcggcagctatgccatggattactggggccagggcaccctggtgacagtgtcctct

SEQ ID NO: 160 - DNA, 360 bp, Artificial Sequence
  source: /note=“Description of Artificial Sequence: Synthetic polynucleotide”
caggtccagctgcaggaaagcggcccaggactggtgaagcctagcgagaccctgtccctgacctgcacagtgagcggcgtgtccctgcctgattacggcgtgtcctggatcagacagccccctggcaagtgtctggagtggatcggcgtgatctggggctccgagaccacatactatcagtcctctctgaagtctagggtgacaatctctaaggacaacagcaagaatcaggtgagcctgaagctgagctccgtgaccgcagcagatacagccgtgtactattgtgccaagcactactattacggcggctcttatgctatggattattgggggcagggcactctggtcactgtctcaagc

SEQ ID NO: 161 - DNA, 360 bp, Artificial Sequence
  source: /note=“Description of Artificial Sequence: Synthetic polynucleotide”
caggtgcagctgcaggagagcggcccaggactggtgaagccttccgagacactgtctctgacctgtacagtgagcggcgtgtccctgcccgactacggcgtgtcctggatcagacagccacctggcaagggactggagtggatcggcgtgatctggggcagcgagaccacatactatcagagctccctgaagtccagggtgaccatcagcaaggacaactccaagaatcaggtgagcctgaagctgtctagcgtgaccgccgccgatacagccgtgtactattgcgccaagcactactattacggcggctcctatgccatggattactggggccagggcaccctggtcacagtgtcctct

SEQ ID NO: 162 - DNA, 360 bp, Artificial Sequence
  source: /note=“Description of Artificial Sequence: Synthetic polynucleotide”
caggtgcagctgcaggagtctggcccaggactggtgaagccttctgagaccctgagcctgacctgcacagtgtccggcgtgtctctgcccgattacggcgtgtcctggatcagacagccacctggcaagggactggagtggatcggcgtgatctggggctctgagaccacatactatcagtctagcctgaagagccgggtgacaatctccaaggacaactctaagaatcaggtgtccctgaagctgtcctctgtgaccgccgccgatacagccgtgtactattgtgccaagcactactattacggcggcagctatgccatggactactggggccagggcaccctggtgacagtgagctcc

SEQ ID NO: 163 - DNA, 360 bp, Artificial Sequence
  source: /note=“Description of Artificial Sequence: Synthetic polynucleotide”
caggtgcagctgcaggagtctggcccaggactggtgaagccttctgagaccctgagcctgacctgcacagtgagcggcgtgtccctgcccgattacggcgtgtcctggatcagacagccacctggcaagggactggagtggatcggcgtgatctggggcagcgagaccacatactatcagtcctctctgaagtccagggtgacaatctccaaggacaactctaagaatcaggtgagcctgaagctgagctccgtgaccgcagcagatacagccgtgtactattgcgccaagcactactattacggcggctcctatgccatggactactggggccagggcaccctggtcacagtgtctagc

SEQ ID NO: 164 - DNA, 360 bp, Artificial Sequence
  source: /note=“Description of Artificial Sequence: Synthetic polynucleotide”
caggtgcagctgcaggagtccggcccaggactggtgaagccttccgagacactgtctctgacctgtacagtgtccggcgtgtctctgcccgactacggcgtgagctggatcagacagccacctggcaagggactggagtggatcggcgtgatctggggctctgagaccacatactatcagtcctctctgaagagccgggtgaccatcagcaaggacaactccaagaatcaggtgtccctgaagctgagctccgtgaccgcagcagatacagccgtgtactattgcgccaagcactactattacggcggcagctatgccatggattactggggccagggcaccctggtgacagtgtctagc

SEQ ID NO: 165 - DNA, 321 bp, Artificial Sequence
  source: /note=“Description of Artificial Sequence: Synthetic polynucleotide”
gagatcgtgatgacccagagcccagccacactgagcctgtccccaggagagagggccacactgtcttgtagagccagccaggatatctccaagtatctgaactggtaccagcagaagcctggacaggcaccaaggctgctgatctaccacacctctagactgcacagcggcatccctgccaggttttctggcagcggctccggcacagactataccctgacaatctctagcctgcagccagaggatttcgccgtgtacttttgtcagcagggcaatactctgccatacacctttggatgcggaactaaactggaaatcaag

SEQ ID NO: 166 - DNA, 321 bp, Artificial Sequence
  source: /note=“Description of Artificial Sequence: Synthetic polynucleotide”
gaaattgtgatgacccagtcccccgctactctgtctctgtcccccggagaacgggctactctgtcttgtcgcgcttcccaggatattagcaagtacctgaactggtatcagcagaagccaggacaggcaccaaggctgctgatctaccacacctctcgcctgcacagcggcatccctgcacggttctctggcagcggctccggcacagactacaccctgacaatcagctccctgcagcctgaggatttcgccgtgtacttttgccagcagggcaataccctgccatatacatttggctgtggcaccaagctggagatcaag

SEQ ID NO: 167 - DNA, 321 bp, Artificial Sequence
  source: /note=“Description of Artificial Sequence: Synthetic polynucleotide”
gagatcgtgatgacccagtccccagccacactgagcctgtccccaggagagagggccaccctgtcttgtagagccagccaggatatctccaagtatctgaactggtaccagcagaagcctggacaggcaccaaggctgctgatctaccacacctctagactgcacagcggcatccctgccaggttttctggcagcggctccggcacagactataccctgacaatctctagcctgcagccagaggatttcgccgtgtacttttgtcagcagggaaatactctgccatacacctttggatgcggaactaaactggaaatcaag

SEQ ID NO: 168 - DNA, 321 bp, Artificial Sequence
  source: /note=“Description of Artificial Sequence: Synthetic polynucleotide”
gagattgtgatgacccagtcccccgccaccctgagtctgagccccggagaacgagctaccctgagttgccgagcttcccaggacatttccaagtacctgaactggtatcagcagaagccaggacaggcaccaaggctgctgatctaccacacctctcgcctgcacagcggcatcccagcacggttctctggcagcggctccggcacagactacaccctgacaatcagctccctgcagcctgaggatttcgccgtgtacttttgccagcagggcaataccctgccatatacatttggctgtggcaccaagctggagatcaag

SEQ ID NO: 169 - DNA, 321 bp, Artificial Sequence
  source: /note=“Description of Artificial Sequence: Synthetic polynucleotide”
gagatcgtgatgacccagtctccagccacactgtctctgagcccaggagagagggccaccctgtcttgccgcgccagccaggatatctccaagtatctgaactggtaccagcagaagccaggacaggcaccaaggctgctgatctaccacacctctcgcctgcacagcggcatcccagcacggttctccggctctggcagcggcacagactacaccctgacaatctcctctctgcagcccgaggatttcgccgtgtatttttgccagcagggcaataccctgccttacacatttggccagggcaccaagctggagatcaag

SEQ ID NO: 170 - DNA, 321 bp, Artificial Sequence
  source: /note=“Description of Artificial Sequence: Synthetic polynucleotide”
gagatcgtgatgacccagagcccagccacactgagcctgtccccaggagagagggccaccctgagctgcagagcctcccaggatatctctaagtatctgaactggtaccagcagaagcctggacaggcaccaaggctgctgatctaccacaccagcagactgcactccggcatccctgcaaggttctctggcagcggctccggcacagactacaccctgacaatctctagcctgcagcctgaggatttcgccgtgtatttttgtcagcagggcaataccctgccatacacatttggccagggcaccaagctggagatcaag

SEQ ID NO: 171 - DNA, 321 bp, Artificial Sequence
  source: /note=“Description of Artificial Sequence: Synthetic polynucleotide”
gagatcgtgatgacccagagcccagccacactgtctctgagcccaggagagagggccaccctgagctgtcgcgcctcccaggatatctctaagtatctgaactggtaccagcagaagccaggacaggcaccaaggctgctgatctaccacaccagccgcctgcactccggcatcccagcacggttctccggctctggcagcggcacagactacaccctgacaatctcctctctgcagcccgaggatttcgccgtgtatttttgccagcagggcaataccctgccttacacatttggccagggcaccaagctggagatcaag

SEQ ID NO: 172 - DNA, 321 bp, Artificial Sequence
  source: /note=“Description of Artificial Sequence: Synthetic polynucleotide”
gagatcgtgatgacccagtctccagccacactgagcctgtccccaggagagagggccaccctgtcttgcagagccagccaggatatctccaagtatctgaactggtaccagcagaagcctggacaggcaccaaggctgctgatctaccacacctctagactgcacagcggcatcccagcaaggttctctggcagcggctccggcacagactacaccctgacaatctctagcctgcagcctgaggatttcgccgtgtatttttgccagcagggcaataccctgccatacacatttggccagggcaccaagctggagatcaag

SEQ ID NO: 173 - DNA, 801 bp, Artificial Sequence
  source: /note=“Description of Artificial Sequence: Synthetic polynucleotide”
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 174 - DNA, 1401 bp, Artificial Sequence
  source: /note=“Description of Artificial Sequence: Synthetic polynucleotide”
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 175 - protein, 467 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 176 - DNA, 735 bp, Artificial Sequence
  source: /note=“Description of Artificial Sequence: Synthetic polynucleotide”
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 177 - protein, 245 aa, Artificial Sequence; identical to SEQ ID NO: 126
  source: /note=“Description of Artificial Sequence: Synthetic polypeptide”

SEQ ID NO: 178 - DNA, 801 bp, Artificial Sequence
  source: /note=“Description of Artificial Sequence: Synthetic polynucleotide”
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 179 - DNA, 1317 bp, Artificial Sequence
  source: /note=“Description of Artificial Sequence: Synthetic polynucleotide”
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 180 - protein, 439 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 181 - DNA, 735 bp, Artificial Sequence
  source: /note=“Description of Artificial Sequence: Synthetic polynucleotide”
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 182 - protein, 245 aa, Artificial Sequence; identical to SEQ ID NO: 126
  source: /note=“Description of Artificial Sequence: Synthetic polypeptide”

SEQ ID NO: 183 - DNA, 1395 bp, Artificial Sequence
  source: /note=“Description of Artificial Sequence: Synthetic polynucleotide”
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 184 - protein, 464 aa, Artificial Sequence
  source: /note=“Description of Artificial Sequence: Synthetic polypeptide”
QVQLLESGAELVRPGSSVKISCKASGYAFSSYWMNWVKQRPGQGLEWIGQIYPGDGDTNYNGKFKGQATLTADKSSSTAYMQLSGLTSEDSAVYSCARKTISSVVDFYFDYWGQGTTVTGGGSGGGSGGGSGGGSELVLTQSPKFMSTSVGDRVSVTCKASQNVGTNVAWYQQKPGQSPKPLIYSATYRNSGVPDRFTGSGSGTDFTLTITNVQSKDLADYFCQYNRYPYTSFFFTKLEIKRRSKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 185 - protein, 439 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRL

SEQ ID NO: 186 - protein, 819 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

(19) United States
(12) Patent Application Publication
(10) Pub. No.: US 2023/0111593 A1
(43) Pub. Date: Apr. 13, 2023

(54) METHOD OF PREDICTING RESPONSE TO CHIMERIC ANTIGEN RECEPTOR THERAPY

(71) Applicants: NOVARTIS AG, Basel (CH); THE TRUSTEES OF THE UNIVERSITY OF PENNSYLVANIA, Philadelphia, PA (US)

(72) Inventors: Stephen SCHUSTER, Springfield, PA (US); Yubing TONG, Springfield, PA (US); Jayaram K. UDUPA, Philadelphia, PA (US); Drew A. TORIGIAN, Philadelphia, PA (US)

(21) Appl. No.: 17/799,171

(22) PCT Filed: Feb. 12, 2021
(86) PCT No.: PCT/US2021/018029

§ 371 Date: Aug. 11, 2022

Related U.S. Application Data
(60) Provisional application No. 62/976,877, filed on Feb. 14, 2020.

(51) Int. Cl. G16H 50/50; A61K 35/17; A61K 38/17; A61P 35/00; G06V 10/82; G06V 10/764; G06T 7/00

(52) U.S. Cl. CPC G16H 50/50; A61K 35/17; A61K 38/1774; A61P 35/00; G06V 10/82; G06V 10/764; G06T 7/0012; G06T 2207/20084; G06T 2207/30096; G06V 2201/03

(57) ABSTRACT

This disclosure provides methods and systems for determining a lesion-level treatment response to a chimeric antigen receptor (CAR) therapy, e.g., a CAR CD19 therapy, and uses of said methods and systems for evaluating the responsiveness of a subject to a CAR CD19 therapy, and for treating a subject with a CAR CD19 therapy.

### CROSS-REFERENCE TO RELATED APPLICATIONS

This application claims priority to U.S. Ser. No. 62/976,877 filed on Feb. 14, 2020. The entire contents of the aforesaid application are incorporated herein by reference.

### SEQUENCE LISTING

The instant application contains a Sequence Listing which has been submitted electronically in ASCII format and is hereby incorporated by reference in its entirety. Said ASCII copy, created on Feb. 11, 2021, is named N2067-7168WO_SL.txt and is 326,807 bytes in size.

### FIELD OF THE INVENTION

The present invention relates generally to methods of predicting response to Chimeric Antigen Receptor (CAR) therapy.

### BACKGROUND OF THE INVENTION

Adoptive cell transfer (ACT) therapy with autologous T cells, especially with T cells transduced with Chimeric Antigen Receptors (CAR T-cell therapy), has shown promise in several hematologic cancer trials. However, the efficacy or CAR T-cell therapy can be improved. Efforts to identify biomarkers for predicting response to CAR T-cell therapy to improve efficacy, has focused on laboratory/pathology-based analysis instead of medical image-based analysis. There is a need for improved methods for predicting and/or evaluating a response to CAR T-cell therapy.

### SUMMARY OF THE INVENTION

The present disclosure pertains, at least in part, to a method, e.g., a computer-implemented method, for determining, e.g., predicting, a lesion-level treatment response to a chimeric antigen receptor (CAR) therapy, e.g., a therapy comprising an immune effector cell or population of immune effector cells that expresses a CAR that binds to CD19 (also referred to herein as a “CAR19 therapy” or a “CD19 CAR therapy”). The method further comprises a rule-based reasoning methodology for patient-level response prediction. Also provided herein is a system and a non-transitory computer-readable medium for determining a lesion-level response. Furthermore, the disclosure provides a method for treating a subject having, or at risk of having a lymphoma (e.g., a lymphoma described herein, e.g., DLBCL or FL), by administering a CAR19 therapy to the subject responsive to a determination, e.g., prediction, of a lesion-level treatment response to the CAR19 therapy. In addition, provided herein is a method of evaluating the responsiveness of a subject having, or at risk of having a lymphoma, to a CAR19 therapy using a lesion-level treatment response prediction method disclosed herein. CAR19 therapies, and methods of making and using the same, are further disclosed.

In an aspect, the disclosure provides a computer-implemented method for determining, e.g., predicting, a lesion-level treatment response to a therapy comprising an immune effector cell or population of immune effector cells that expresses a Chimeric Antigen Receptor (CAR) that binds to CD19 (a “CAR19 therapy”), said method comprising:

acquiring, e.g., receiving, an image of a lesion of a subject, e.g., a subject having or at risk of having a lymphoma (“acquired image”); and

processing the image with a neural network (“processed image”),

wherein the neural network outputs a classification result indicating the lesion-level treatment response to the CAR19 therapy.

In another aspect, provided herein is a system for determining, e.g., predicting, a lesion-level treatment response to a therapy comprising an immune effector cell or population of immune effector cells that expresses a Chimeric Antigen Receptor (CAR) that binds to CD19 (a “CAR19 therapy”), said system comprising:

a processor; and

a storage device storing instructions that, when executed by the processor, cause the processor to:

acquire, e.g., receive, an image of a lesion of a subject, e.g., a subject having, or at risk of having, a lymphoma (“acquired image”); and

process the image with a neural network (“processed image”),

wherein the neural network outputs a classification result indicating the lesion-level treatment response to the CAR19 therapy.

In yet another aspect, the disclosure provides a non-transitory computer-readable medium for determining, e.g., predicting, a lesion-level treatment response to a therapy comprising an immune effector cell or population of immune effector cells that expresses a Chimeric Antigen Receptor (CAR) that binds to CD19 (a “CAR19 therapy”), said medium comprising instructions that, when executed by a processor, cause the processor to:

acquire, e.g., receive, an image of a lesion of the subject, e.g., a subject having, or at risk of having, a lymphoma (“acquired image”); and

process the image with a neural network (“processed image”),

wherein the neural network outputs a classification result indicating the lesion-level treatment response to the CAR19 therapy.

In an aspect, provided herein is a method for treating a subject having, or at risk of having, lymphoma, comprising:

responsive to a determination, e.g., prediction, of a lesion-level treatment response to a therapy comprising an immune effector cell or population of immune effector cells that expresses a Chimeric Antigen Receptor (CAR) that binds to CD19 (a “CAR19 therapy”),

administering the CAR19 therapy to the subject, thereby treating the subject,

wherein said determination, e.g., prediction, comprises:

acquiring, e.g., receiving, an image of a lesion of the subject (“acquired image”); and

processing the image with a neural network (“processed image”), wherein the neural network outputs a classification result indicating the lesion-level treatment response to the CAR19 therapy.

In another aspect, the disclosure provides a method for evaluating, or predicting the responsiveness of, a subject having or at risk of having a lymphoma to a CAR19 therapy, said method comprising:

determining, e.g., predicting, of a lesion-level treatment response to a therapy comprising an immune effector cell or population of immune effector cells that expresses a Chimeric Antigen Receptor (CAR) that binds to CD19 (a “CAR19 therapy”), with a neural network, wherein said determining comprises:

acquiring, e.g., receiving, an image of a lesion of the subject (“acquired image”); and

processing the image with the neural network (“processed image”), wherein the neural network outputs a classification result indicating the lesion-level treatment response to the CAR19 therapy; and

thereby evaluating the subject, or predicting the responsiveness of the subject, to the CAR19 therapy.

In some embodiments of any of the methods, systems, mediums, method for treating, or method for evaluating a subject provided herein, the CAR19 therapy is a therapy comprising immune effector cells expressing an anti-CD19 binding domain, a transmembrane domain, and an intracellular signaling domain comprising a stimulatory domain.

In some embodiments of any of the methods, systems, mediums, method for treating, or method for evaluating a subject provided herein, the lymphoma, e.g., relapsed and/or refractory lymphoma, is chosen from diffuse large B-cell lymphoma (DLBCL) or follicular lymphoma (FL). In some embodiments, the lymphoma is DLBCL, e.g., relapsed and/or refractory DLBCL. In some embodiments, the lymphoma is FL, e.g., relapsed and/or refractory FL.

In some embodiments of any of the methods, systems, mediums, method for treating, or method for evaluating a subject provided herein, the lesion-level treatment response is indicative of responsiveness to the CAR19 therapy. In some embodiments, the lesion-level treatment response is evaluated using one, two, three or more (all) of the following parameters: 1) a change in lesion size; 2) a change in metabolic activity; 3) a change in lesion morphology; or 4) a change in lesion density. In some embodiments, the lesion-level treatment response is evaluated using a change in lesion size. In some embodiments, the lesion-level treatment response is evaluated using a change in metabolic activity. In some embodiments, the lesion-level treatment response is evaluated using a change in lesion morphology. In some embodiments, the lesion-level treatment response is evaluated using a change in lesion density.

In some embodiments, a decrease in one or more of 1-4 is indicative of a positive response to the CAR19 therapy.

In some embodiments, an increase or lack of a detectable change in one or more of 1-4 is indicative of a negative response to the CAR19 therapy.

In some embodiments of any of the methods, systems, mediums, method for treating, or method for evaluating a subject provided herein, the prediction of the lesion-level treatment response is followed by a rule-based reasoning method, to thereby determine a patient-level response prediction. In some embodiments, the patient-level response prediction comprises an All rule and/or a Majority Rule. In some embodiments, the patient-level response prediction comprises an All rule. In some embodiments, the patient-level response prediction comprises a Majority rule.

Those skilled in the art will recognize or be able to ascertain using no more than routine experimentation, many equivalents to the specific embodiments of the invention described herein. Such equivalents are intended to be encompassed by the following enumerated embodiments.

### Enumerated Embodiments

1. A computer-implemented method for determining, e.g., predicting, a lesion-level treatment response to a therapy comprising an immune effector cell or population of immune effector cells that expresses a Chimeric Antigen Receptor (CAR) that binds to CD19 (a “CAR19 therapy”), said method comprising:

acquiring, e.g., receiving, an image of a lesion of a subject, e.g., a subject having or at risk of having a lymphoma (“acquired image”); and

processing the image with a neural network (“processed image”),

wherein the neural network outputs a classification result indicating the lesion-level treatment response to the CAR19 therapy.
2. A system for determining, e.g., predicting, a lesion-level treatment response to a therapy comprising an immune effector cell or population of immune effector cells that expresses a Chimeric Antigen Receptor (CAR) that binds to CD19 (a “CAR19 therapy”), said system comprising:

a processor; and

a storage device storing instructions that, when executed by the processor, cause the processor to:
acquire, e.g., receive, an image of a lesion of a subject, e.g., a subject having, or at risk of having, a lymphoma (“acquired image”); and

process the image with a neural network (“processed image”),

wherein the neural network outputs a classification result indicating the lesion-level treatment response to the CAR19 therapy.
3. A non-transitory computer-readable medium for determining, e.g., predicting, a lesion-level treatment response to a therapy comprising an immune effector cell or population of immune effector cells that expresses a Chimeric Antigen Receptor (CAR) that binds to CD19 (a “CAR19 therapy”), said medium comprising instructions that, when executed by a processor, cause the processor to:

acquire, e.g., receive, an image of a lesion of the subject, e.g., a subject having, or at risk of having, a lymphoma (“acquired image”); and

process the image with a neural network (“processed image”),

wherein the neural network outputs a classification result indicating the lesion-level treatment response to the CAR19 therapy.
4. A method for treating a subject having, or at risk of having, lymphoma, comprising:

responsive to a determination, e.g., prediction, of a lesion-level treatment response to a therapy comprising an immune effector cell or population of immune effector cells that expresses a Chimeric Antigen Receptor (CAR) that binds to CD19 (a “CAR19 therapy”),

administering the CAR19 therapy to the subject, thereby treating the subject, wherein said determination, e.g., prediction, comprises:

acquiring, e.g., receiving, an image of a lesion of the subject (“acquired image”); and

processing the image with a neural network (“processed image”), wherein the neural network outputs a classification result indicating the lesion-level treatment response to the CAR19 therapy.

5. A method for evaluating, or predicting the responsiveness of, a subject having or at risk of having a lymphoma to a CAR19 therapy, said method comprising:

determining, e.g., predicting, of a lesion-level treatment response to a therapy comprising an immune effector cell or population of immune effector cells that expresses a Chimeric Antigen Receptor (CAR) that binds to CD19 (a “CAR19 therapy”), with a neural network, wherein said determining comprises:

acquiring, e.g., receiving, an image of a lesion of the subject (“acquired image”); and

processing the image with the neural network (“processed image”), wherein the neural network outputs a classification result indicating the lesion-level treatment response to the CAR19 therapy; and

thereby evaluating the subject, or predicting the responsiveness of the subject, to the CAR19 therapy.

6. The method, system or medium of any one of embodiments 1-5, wherein the CAR19 therapy is a therapy comprising immune effector cells expressing an anti-CD19 binding domain, a transmembrane domain, and an intracellular signaling domain comprising a stimulatory domain.
7. The method, system or medium of any one of embodiments 1-6, wherein the lymphoma is chosen from diffuse large B-cell lymphoma (DLBCL) or follicular lymphoma.
8. The method, system or medium of any of the preceding embodiments, wherein the acquired image of the lesion comprises at least one, two, or three images.
9. The method, system or medium of any of the preceding embodiments, wherein the acquired image is a pre-treatment image, e.g., prior to a CAR19 therapy.
10. The method, system or medium of any of the preceding embodiments, wherein the acquired image is a post-treatment image, e.g., after a CAR19 therapy and/or a different therapy (e.g., chemotherapy or radiotherapy).
11. The method, system or medium of any of the preceding embodiments, wherein the acquired image comprises a plurality of images, e.g., at least two images of different views of the same lesion in the subject.
12. The method, system or medium of any of the preceding embodiments, wherein the acquired image comprises a sagittal view, a coronal view, a transverse view, a longitudinal view, or a combination thereof, of the same lesion.
13. The method, system or medium of any of the preceding embodiments, wherein the acquired image is obtained by at least one imaging modality chosen from computed tomography (CT) (e.g., diagnostic CT (dCT), low dose CT (lCT)), positron emission tomography (PET), magnetic resonance imaging (MRI), single-photon emission computerized tomography (SPECT), PET lCT, PET/MRI, SPECT lCT, lCT/PET, or a combination thereof.
14. The method, system or medium of any of the preceding embodiments, wherein the acquired image is one of a single volume of interest (VOI)-restricted image slice passing through a mid-portion of a lesion, three contiguous VOI-restricted image slices passing through a mid-portion of a lesion, a single whole image slice passing through a mid portion of a lesion, three contiguous whole image slices passing through a mid-portion of a lesion, or a single VOI-slice and a single whole slice passing through a mid portion of a lesion and combined into two channels of one input sample, or a combination thereof.
15. The method, system or medium of any of the preceding embodiments, wherein the acquired image is processed by a neural network having been trained on dCT image data.
16. The method, system or medium of any of the preceding embodiments, wherein the acquired image is processed by a neural network having been trained on lCT image data.
17. The method, system or medium of any of the preceding embodiments, wherein the acquired image is processed by a neural network having been trained on PET image data.
18. The method, system or medium of any one of embodiments 13b-13d, wherein the acquired image is processed by a neural network having been additionally trained, separately on lCT image data or PET image data.
19. The method, system or medium of embodiment 18, wherein the acquired image is processed by the neural network having been additionally trained, separately on lCT image data and PET image data.
20. The method, system or medium of any of the preceding embodiments, wherein the acquired image comprises an image of at least one, two, three, four, five, ten, twenty, fifty, one hundred, five hundred or one thousand lesions in the subject.
21. The method, system or medium of any of the preceding embodiments, wherein the lesion of the subject is a nodal or extra-nodal lesion, or a combination thereof.
22. The method, system or medium of any of the preceding embodiments, wherein the lesion of the subject is detected in the lymph nodes.
23. The method, system or medium of any of the preceding embodiments, wherein the lesion-level treatment response is indicative of responsiveness to the CAR19 therapy.
24. The method, system or medium of any of the preceding embodiments, wherein the lesion-level treatment response is evaluated using one or more of the following parameters: 1) a change in lesion size; 2) a change in lesion metabolic activity; 3) a change in lesion morphology; 4) a change in lesion intensity (e.g., lesion attenuation (on CT), lesion signal intensity (on MRI, whether T1-weighted, T2-weighted, or diffusion-weighted), or lesion contrast enhancement (on CT or MRI)); 5) a change in lesion morphology (e.g., a lesion size, a lesion volume, or a lesion shape); 6) a change in lesion radiotracer uptake (e.g., FDG uptake on PET imaging or DOTATE uptake on PET imaging); 7) a change in lesion texture (e.g., on CT, MRI, PET, or SPECT); and/or 8) a change in non-lesion tissue properties (e.g., on CT, MRI, PET, or SPECT in terms of tissue morphology, radiotracer activity, intensity, or texture).
25. The method, system or medium of embodiment 24, wherein a decrease in one, two, three or more (all) of 1-8 is indicative of a positive response to the CAR19 therapy.
26. The method, system or medium of embodiment 24 or 25, wherein an increase or lack of detectable change in one, two, three or more (all) of 1-8 is indicative of a negative response to the CAR19 therapy.
27. The method, system or medium of embodiment 25 or 26, wherein the positive or negative response is classified as a binary classification.
28. The method, system or medium of embodiment 27, wherein the positive response corresponds to a first classification result of the binary classification.
29. The method, system or medium of embodiment 27 or 28, wherein the negative response corresponds to a second classification result of the binary classification.
30. The method, system or medium of any one of embodiments 25-29, further comprising determining the percentage of lesions with a first classification result relative to the total number of lesions with a processed image “processed lesions”).
31. The method, system or medium of embodiment 30, wherein a percentage of 50% or greater is indicative of a positive response to the CAR19 therapy.
32. The method, system or medium of embodiment 30, wherein a percentage of less than 50% is indicative of a negative treatment response to the CAR19 therapy.
33. The method, system or medium of any of the preceding embodiments, wherein the prediction of the lesion-level treatment response is followed by a rule-based reasoning method, to thereby determine a patient-level response prediction.
34. The method, system or medium of embodiment 33, wherein the patient-level response prediction comprises at least one rule.
35. The method, system or medium of embodiment 33 or 34, wherein the patient-level response prediction comprises an All rule or a Majority Rule.
36. The method, system or medium of embodiment 35, wherein a responder in the All rule of the patient-level response prediction is a subject in whom all evaluated lesions have responded, or are predicted to respond to the CAR19 therapy.
37. The method, system or medium of embodiment 35, wherein a non-responder in the All rule of the patient-level response prediction is a subject in whom at least one evaluated lesion has not responded, or is predicted not to respond to the CAR19 therapy.
38. The method, system or medium of any one of embodiments 35-37, wherein a responder in the Majority rule of the patient-level response prediction is a subject in whom a majority of the evaluated lesions (e.g., based on a % threshold, e.g., at least or greater than 60% (e.g., 60%, 65%, 70%, 75%, 80%, 85%, 90%, 95% or greater) of all evaluated lesions from the same subject) have responded, or are predicted to respond to the CAR19 therapy.
39. The method, system or medium of any one of embodiment 35-38, wherein a non-responder in the Majority rule of the patient-level response prediction is a subject in whom a majority of the evaluated lesions have not responded (e.g., based on a % threshold, e.g., at least or greater than 60% (e.g., 60%, 65%, 70%, 75%, 80%, 85%, 90%, 95% or greater) of all evaluated lesions from the same subject) have not responded, or are predicted not to respond to the CAR19 therapy.
40. The method, system or medium of any of embodiments 35-39, wherein the subject is classified as a responder or a non-responder in the patient-level response prediction.
41. The method, system or medium of embodiment 40, wherein the responder according to the patient-level response prediction is a subject who has, or has shown a clinical response, e.g., a detectable clinical response, e.g., a complete response or a partial response.
42. The method, system or medium of embodiment 40, wherein the non-responder according to the patient-level response prediction is a subject who does not have, or has not shown a clinical response, e.g., does not have a detectable clinical response, e.g., has progressive disease.
43. The method, system or medium of any of embodiments 33-41, wherein a responder according to the patient-level response prediction corresponds to a subject having a low score in a clinical stratification tool, e.g., International Prognostic Index (IPI) for DLBCL or follicular lymphoma international prognostic index (FLIPI) for follicular lymphoma.
44. The method, system or medium of embodiment 43, wherein the responder according to the patient-level response prediction has an IPI score of ≤2.
45. The method, system or medium of embodiment 43, wherein the responder according to the patient-level response prediction has a FLIPI score of ≤2.
46. The method, system or medium of any of embodiments 33-40 or 42, wherein a non-responder according to the patient-level response prediction corresponds to a subject having a high score in a clinical stratification tool, e.g., International Prognostic Index (IPI) for DLBCL or follicular lymphoma international prognostic index (FLIPI) for follicular lymphoma.
47. The method, system or medium of embodiment 46, wherein the non-responder according to the patient-level response prediction has an IPI score of >2.
48. The method, system or medium of embodiment 46, wherein the non-responder according to the patient-level response prediction has a FLIPI score of >2.
49. The method, system or medium of any of the preceding embodiments, further comprising comparing results to a clinical stratification tool, e.g., International Prognostic Index (IPI) for DLBCL or follicular lymphoma international prognostic index (FLIPI) for follicular lymphoma.
50. The method, system or medium of any of the preceding embodiments, wherein the neural network comprises a plurality of layers.
51. The method, system or medium of embodiment 50, wherein the plurality of layers comprises at least two of: an input layer, a convolutional layer, a fully connected layer, a pooling layer, a classification layer, and an output layer.
52. The method, system or medium of any of the preceding embodiments, wherein the neural network is based on a pre-trained convolutional neural network (CNN).
53. The method, system or medium of embodiment 52, wherein the pre-trained convolutional neural network is one of VGGNet, AlexNet, ResNet, SqueezeNet, or GoogLeNet.
54. The method, system or medium of embodiment 39, wherein the VGGNet is VGG-16 or VGG-19.
55. The method, system or medium of embodiment 53 or 54, wherein the ResNet is one of ResNeXt or DenseNet.
56. The method, system or medium of embodiment 52, wherein the neural network is trained according to:

loading a pre-trained neural network;

modifying at least one layer of the pre-trained neural network, wherein the modifying includes replacing the at least one layer with at least one of a fully connected layer, a softmax layer, or a binary classification output layer, thereby creating a modified neural network;

loading a set of labeled pre-treatment digital images; and

re-training the modified neural network using the set of labeled pre-treatment images.

57. The method, system or medium of embodiment 56, wherein the neural network is trained according to:

loading the pre-trained neural network comprising an input layer, a first convolutional layer with ReLU (rectified linear unit), a first max pooling layer, a second convolutional layer with ReLU, a second max pooling layer, a third convolutional layer with ReLU, a fourth convolutional layer with ReLU, a third max pooling layer, a first fully connected layer with ReLU and dropout, a second fully connected layer with ReLU and dropout, a third fully connected layer, a softmax layer, and an output layer; and

replacing the third fully connected layer, the softmax layer, and the output layer with, respectively, a new fully connected layer, a new softmax layer, and a binary classification output layer, thereby forming the modified neural network.

58. The method, system or medium of embodiment 56, wherein replacing the at least one layer comprises:

replacing three layers of the pre-trained neural network with a fully connected layer, a softmax layer, and a binary classification output layer.

59. The method, system or medium of any one of embodiments 56-58, wherein parameters of the modified neural network are optimized using Stochastic Gradient Descent (SGD), Stochastic Gradient Descent with momentum, Simulated Annealing (SA), or Conjugate Gradient (CG).
60. The method, system or medium of any one of embodiments 56-59, wherein the set of labeled pre-treatment digital images of images is augmented before the re-training, the augmentation comprising adding augmented images to the set of labeled pre-treatment digital images by performing at least one of: at least one of a random flip along a vertical axis, a random translation of up to 10 pixels horizontally and vertically, an affine operation, or a combination thereof, on one or more of each image in the set of labeled pre-treatment digital images.
60. The method, system or medium of any one of embodiments 56-60, wherein the set of pre-treatment digital images is labeled according to:

placing a volume of interest (VOI) around each individual lesion in the set of pre-treatment digital images; and

categorizing each lesion in the set of images based on a post-treatment response of the lesion.

61. The method, system or medium of embodiment 60, wherein the categorizing includes assigning either a first value or a second value to each lesion.
62. The method, system or medium of embodiment 61, wherein the first value corresponds to a lesion with no response to the CAR19 therapy and the second value corresponds to a full positive response or partial positive response to the CAR19 therapy.
63. The method, system or medium of any one of embodiments 56-62, wherein the set of labeled pre-treatment digital images is generated from an imaging modality chosen from computed tomography (CT) (e.g., diagnostic CT (dCT), low dose CT (lCT)), positron emission tomography (PET), magnetic resonance imaging (MRI), single-photon emission computerized tomography (SPECT), PET lCT, PET/MRI, SPECT lCT, lCT/PET, or a combination thereof.
64. The method, system or medium of any one of embodiments 56-63, before the modified neural network is re-trained, the set of labeled pre-treatment digital images is pre-processed by performing mean subtracting and down sampling using bilinear interpolation on each image of the set of labeled pre-treatment digital images.
65. The method, system or medium of any of the preceding embodiments, wherein the neural network is trained with a batch size of at least 1, 2, 3, 4, 5, 6, 7, 8, 10, 16, 20, 30, 32, 64, 128, 256, 512, or 1024.
66. The method, system or medium of embodiment 65, wherein the neural network is trained with a batch size between 5 and 30.
67. The method, system or medium of embodiment 66, wherein the neural network is trained with a batch size of 5, 10, 20 or 30.
68. The method, system or medium of any of the preceding embodiments, wherein the neural network is trained with an epoch number of at least 1, 2, 3, 4, 5, 8, 9, 10, 16, 20, 32, 40, 64, 80, 100, 128, 200, 256, 512, or 1024.
69. The method, system or medium of embodiment 68, wherein the neural network is trained with an epoch number between 40 and 200.
70. The method, system or medium of embodiment 69, wherein the neural network is trained with an epoch number of 40, 80, 100 or 200.
71. The method, system or medium of any of the preceding embodiments, wherein the neural network is trained with a learning rate between 0.0 and 1.0.
72. The method, system or medium of embodiment 71, wherein the neural network is trained with a learning rate between 10⁻⁵ and 10⁻³.
73. The method, system or medium of embodiment 72, wherein the neural network is trained with a learning rate of 10⁻⁴.
74. The method, system or medium of embodiments 56-73, wherein the set of pre-treatment digital images is divided into a divided set if images comprising: a training set, a validation set, and a testing set.
75. The method, system or medium of embodiment 74, wherein the divided set of images is divided according to a ratio of 6:2:2.
76. The method, system or medium of embodiments 74 or 75, wherein each image in the divided set of images is resized.
77. The method, system or medium of any one of embodiments 74-76, wherein the testing set is independent of the validation set and the training set.
78. The method, system or medium of any one of embodiments 74-77, wherein for each of the training set, validation set, and testing set, metrics of accuracy (ACC), sensitivity (SEN), specificity (SPE), and area under the curve (AUC) are calculated, and a mean and a standard deviation (SD) is calculated for each metric.
79. The method, system or medium of embodiment 78, wherein one or more receiving operating curves (ROC) is generated.
80. The method, system or medium of embodiment 79, wherein one or more of the ROC, metrics, means, and standard deviations is displayed in a graphical user interface for an operator to finetune the neural network.
81. The method, system or medium of any of the preceding embodiments, wherein a T-test with two sides is performed to verify predictive results between different scenarios, parameters, or imaging modalities.
82. The method, system or medium of any of the preceding embodiments, wherein the modified neural network is re-trained anew utilizing transfer learning.
83. The method, system or medium of embodiment 82, wherein the modified neural network is re-trained anew for each of a dCT image data set, an lCT image data set, and a PET image data set.
84. The method, system or medium of embodiment 83 or 84, wherein for each re-training, the modified neural network receives, at an input of the modified neural network, an input of: a single volume of interest (VOI)-restricted image slice passing through a mid-portion of a lesion, three contiguous VOI-restricted image slices passing through a mid-portion of a lesion, a single whole image slice passing through a mid portion of a lesion, three contiguous whole image slices passing through a mid-portion of a lesion, or a single VOI-slice and a single whole slice combined into two channels of one input sample, or a combination thereof.
85. The method, system or medium of any one of embodiments 82-84, wherein modified neural network is re-trained anew for each of a single volume of interest (VOI)-restricted image slice passing through a mid-portion of a lesion, three contiguous VOI-restricted image slices passing through a mid-portion of a lesion, a single whole image slice passing through a mid portion of a lesion, three contiguous whole image slices passing through a mid-portion of a lesion and a single VOI-slice and a single whole slice combined into two channels of one input sample.
86. The method, system or medium of embodiments any of the preceding embodiments, wherein the modified neural network is re-trained utilizing incremental transfer learning.
87. The method, system or medium of embodiment 86, wherein the incremental transfer learning comprises:

loading an additional set of labeled pre-treatment images; and

performing at least a second re-training by re-training the modified neural network using the additional set of labeled pre-treatment images.

88. The method, system or medium of embodiment 87, wherein the additional set of labeled pre-treatment images comprises images generated from one of: computed tomography (CT) (e.g., diagnostic CT (dCT), low dose CT (lCT)), positron emission tomography (PET), magnetic resonance imaging (MRI), single-photon emission computerized tomography (SPECT), PET lCT, PET/MRI, SPECT lCT, lCT/PET, or a combination thereof.
89. The method, system or medium of embodiments 87 or 88, wherein the incremental learning comprises at least a third re-training by re-training the modified neural network using a second additional set of labeled pre-treatment images.
90. The method, system or medium of embodiment 89, wherein the additional second set of labeled pre-treatment images comprises images generated from one of computed tomography (CT) (e.g., diagnostic CT (dCT), low dose CT (lCT)), positron emission tomography (PET), magnetic resonance imaging (MRI), single-photon emission computerized tomography (SPECT), PET lCT, PET/MRI, SPECT lCT, lCT/PET, or a combination thereof.
91. The method, system or medium of any one of embodiments 86-90, wherein the incremental transfer learning comprises:

for each re-training, the modified neural network receives, at an input of the modified neural network, an input of: a single volume of interest (VOI)-restricted image slice passing through a mid-portion of a lesion, three contiguous VOI-restricted image slices passing through a mid-portion of a lesion, a single whole image slice passing through a mid portion of a lesion, three contiguous whole image slices passing through a mid-portion of a lesion, or a single VOI-slice and a single whole slice combined into two channels of one input sample, or a combination thereof.

92. The method, system or medium of embodiment 91, wherein modified neural network is re-trained anew for each of a single whole image slice passing through mid-portions of lesions and three contiguous whole image slices passing through mid-portions of lesions.
93. The method, system or medium of any one of embodiments 86-92, wherein the modified neural network is first re-trained utilizing transfer learning with a dCT image data set, and then re-trained separately for each of an lCT image data set and a PET image data set.
94. The method, system or medium of any of the preceding embodiments, wherein if the subject is classified as a responder according to a patient-level response prediction, the CAR19 therapy is initiated or continued.
95. The method, system or medium of embodiment 94, wherein the responder according to the patient-level response prediction corresponds to a subject having a low score in a clinical stratification tool, e.g., International Prognostic Index (IPI) for DLBCL or follicular lymphoma international prognostic index (FLIPI) for follicular lymphoma.
96. The method, system or medium of embodiment 94 or 95, wherein a responder is a subject who has a clinical response, e.g., a detectable clinical response, e.g., a complete response or a partial response.
97. The method, system or medium of any one of embodiments 94-96, wherein the responder has an IPI score of ≤2.
98. The method, system or medium of any one of embodiments 94-97, wherein the responder has a FLIPI score of ≤2.
99. The method, system or medium of any one of embodiments 1-98, wherein if the subject is classified as a non-responder according to a patient-level response prediction, the CAR19 therapy is altered, e.g., not initiated, modified (e.g., altered dose or timing), or discontinued.
100. The method, system or medium of embodiment 99, wherein a non-responder according to the patient-level response prediction corresponds to a subject having a high score in a clinical stratification tool, e.g., International Prognostic Index (IPI) for DLBCL or follicular lymphoma international prognostic index (FLIPI) for follicular lymphoma.
101. The method, system or medium of embodiment 99 or 100, wherein the non-responder is a subject who does not have a clinical response, e.g., does not have a detectable clinical response, e.g., has progressive disease.
102. The method, system or medium of any one of embodiments 99-101, wherein the non-responder has an IPI score of >2.
103. The method, system or medium of any one of embodiments 99-102, wherein the non-responder has a FLIPI score of >2.
104. The method of any one of any one of embodiments 94-103, wherein, the method further comprises comparing the results of the patient-level prediction with a clinical stratification tool, e.g., IPI or FLIPI.
105. The method, system or medium of any of the preceding embodiments, wherein the CAR comprises an antibody molecule which includes an anti-CD19 binding domain, a transmembrane domain, and an intracellular signaling domain comprising a stimulatory domain, and wherein said anti-CD19 binding domain comprises one or more of light chain complementary determining region 1 (LC CDR1), light chain complementary determining region 2 (LC CDR2), and light chain complementary determining region 3 (LC CDR3) of any anti-CD19 light chain binding domain amino acid sequence listed in Table 3B, and one or more of heavy chain complementary determining region 1 (HC CDR1), heavy chain complementary determining region 2 (HC CDR2), and heavy chain complementary determining region 3 (HC CDR3) of any anti-CD19 heavy chain binding domain amino acid sequence listed in Table 3A.
106. The method, system or medium of any of the preceding embodiments, wherein the CAR is a CD19 CAR, e.g., a CAR comprising an scFv amino acid sequence listed in Table 3, e.g., amino acid sequence of SEQ ID NO: 39-51 or a CAR comprising the amino acid sequence of SEQ ID NO: 77-89.
107. The method, system or medium of embodiments 105 or 106, wherein the anti-CD19 binding domain comprises the sequence of SEQ ID NO: 40, or SEQ ID NO:51.
108. The method, system or medium of any of the preceding embodiments, wherein the CAR comprises a polypeptide having the sequence of SEQ ID NO:78, or SEQ ID NO: 89.
109. The method, system or medium of any of the preceding embodiments, wherein the CAR19 therapy is a therapy comprising one or more of CTL-019, CTL-119, and CAR 2A.
110. The method, system or medium of any of embodiments 1 to 104, wherein the CAR is a CD19 CAR, e.g., a CAR comprising an scFv amino acid sequence listed in Table 3, e.g., amino acid sequence of SEQ ID NO: 144 or SEQ ID NO: 147, or a CAR comprising the amino acid sequence of SEQ ID NO: 143 or SEQ ID NO: 146.
111. The method, system or medium of embodiment 110, wherein the anti-CD19 binding domain comprises the amino acid sequence of SEQ ID NO: 144 or SEQ ID NO: 147.
112. The method, system or medium of embodiment 110 or 111, wherein the CAR comprises a polypeptide having the amino acid sequence of SEQ ID NO: 143 or SEQ ID NO: 146.
113. The method, system or medium of any of the preceding embodiments, wherein the subject is evaluated prior to receiving the CAR19 therapy.
114. The method, system or medium of any of the preceding embodiments, wherein the response to the CAR19 therapy is evaluated prior to the initiation of the therapy.
115. The method of embodiment 5, wherein the evaluating step or predicting step occurs prior to treatment with the CAR19 therapy.
116. The method of embodiment 5, wherein the evaluating step or predicting step occurs before, after or during the CAR19 therapy, e.g., at least two time points before, after and/or during the CAR19 therapy.
117. The method, system or medium of any of the preceding embodiments, wherein the subject is undergoing the CAR19 therapy.
118. The method, system or medium of any of the preceding embodiments, wherein the subject has received the CAR19 therapy.

### BRIEF DESCRIPTION OF THE DRAWINGS

FIG. 1 is a schematic showing the subject inclusion and exclusion criteria for the study described in Example 1.

FIG. 2 shows the deep learning-based architecture for lesion-level treatment response prediction.

FIG. 3 shows the strategy of transfer learning and incremental learning on lesion-level prediction.

FIG. 4 shows receiver operator characteristic (ROC) curves for diagnostic performance of lesion-level treatment response prediction using transfer learning for 4 scenarios (1 VOI, 1 slice, 3 VOIs, 3 slices) on 3 imaging modalities (diagnostic computed tomography (dCT), low-dose computed tomography (lCT), and positron emission tomography (PET) images). AUC=area under the curve, TP=true positive fraction, FP=false positive fraction.

FIG. 5 shows receiver operator characteristic (ROC) curves for diagnostic performance of lesion-level treatment response prediction using transfer learning for 1 VOI plus 1 slice and 1 slice scenarios on diagnostic computed tomography (dCT), low-dose computed tomography (lCT), and positron emission tomography (PET) images. AUC=area under the curve, TP=true positive fraction, FP=false positive fraction.

FIG. 6 shows receiver operator characteristic (ROC) curves for diagnostic performance of lesion-level treatment response prediction using incremental learning vs. transfer learning for 1 slice and 3 slice scenarios on diagnostic computed tomography (dCT), low-dose computed tomography (lCT), and positron emission tomography (PET) images. AUC=area under the curve, TP=true positive fraction, FP=false positive fraction.

FIG. 7 shows training/validation curves from one of the 10 repeat experiments with E=80 and B=5.

FIG. 8 is a block diagram of a distributed computer system 800 including a computer system 802.

FIG. 9 is flowchart depicting the transfer learning method 900.

FIG. 10 is flowchart depicting the incremental learning method 1000.

### DETAILED DESCRIPTION

Described herein, inter alia, is a method for determining, e.g., predicting, a lesion-level treatment response to a therapy comprising an immune effector cell or population of immune effector cells that expresses a Chimeric Antigen Receptor (CAR) that binds to CD19 (a “CAR19 therapy”), based, e.g., on deep-learning (DL) image analysis. The method further comprises a rule-based reasoning methodology for patient-level response prediction. The medical image-based approach disclosed herein to determine personalized response prediction to CAR T-cell therapies has several unique advantages over conventional methods including, but not limited to: (1) the use of pre-existing diagnostic imaging data sets previously acquired for clinical purposes; (2) the lack of invasiveness; (3) the extraction of regional phenotypic information from disease and extra-disease sites throughout the body that may be heterogeneous; and (4) production-mode efficiency.

Also disclosed herein, is a system and a non-transitory computer-readable medium for determining a lesion-level response to a CAR therapy. In addition, the disclosure provides a method of treating a subject having, or at risk of having a lymphoma (e.g. DLBCL or FL), wherein responsive to a determination, e.g., prediction, of a lesion-level treatment response to a therapy comprising an immune effector cell or population of immune effector cells that expresses a Chimeric Antigen Receptor (CAR) that binds to CD19 (a “CAR19 therapy”), administering the CAR19 therapy to the subject, thereby treating the subject. Also disclosed herein is a method of evaluating a subject having, or at risk of having a lymphoma, comprising determining, e.g., predicting, of a lesion-level treatment response to a therapy comprising a Chimeric Antigen Receptor 19 (CAR19) immune effector cell (“CAR19 therapy”), with a neural network. In an embodiment, the determination comprises: acquiring, e.g., receiving, an image of a lesion of the subject (“acquired image”); and/or processing the image with the neural network (“processed image”). In an embodiment, the neural network outputs a classification result indicating the lesion-level treatment response to the CAR19 therapy. CAR19 therapies, and methods of making and using the same are also disclosed.

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The phrase “lesion-level treatment response” as used herein, refers to a prediction and/or evaluation of responsiveness of a lesion to a therapy. In an embodiment, the lesion is in a subject or in a sample from the subject. In an embodiment, a lesion-level treatment response comprises an output from a neural network, e.g., as described herein. In an embodiment, a lesion-level treatment response is indicative of responsiveness to a therapy, e.g., a CAR19 therapy. In an embodiment, the lesion-level treatment response is evaluated using one, two, three or all of the following parameters: 1) a change in lesion size; 2) a change in metabolic activity; 3) a change in lesion morphology; or 4) a change in lesion density.

The phrase “classification result” as used herein, refers to an output from a neural network. In an embodiment, a classification result is a binary classification result.

The phrase “patient-level response” as used herein, refers to a prediction of responsiveness of a subject (e.g., patient) to a therapy. In an embodiment, the therapy is a CAR19 therapy. In an embodiment, the patient-level response comprises a rule-based reasoning method which, e.g., is based on a lesion-level treatment response. In an embodiment, the patient-level response comprises an All rule or a Majority rule. In an embodiment, a patient-level response prediction classifies a subject as a responder or a non-responder. In an embodiment, a responder is a subject who has, or has shown a clinical response, e.g., a detectable clinical response, e.g., a complete response or a partial response. In an embodiment, a non-responder is a subject who does not have, or has not shown a clinical response, e.g., does not have a detectable clinical response, e.g., has progressive disease.

The term “responder” as used herein, refers to a subject who meets a pre-determined threshold according to the All rule, the Majority rule, a prognostic index (e.g., a patient stratification tool) and/or a clinical response criterion. In an embodiment, a pre-determined threshold for a responder according to the All rule is a subject in whom all evaluated lesions have responded, or are predicted to respond to the therapy, e.g., CAR19 therapy. In an embodiment, a pre-determined threshold for a responder according to the Majority rule is a subject in whom a majority of the evaluated lesions (e.g., based on a % threshold, e.g., at least or greater than 60% (e.g., 60%, 65%, 70%, 75%, 80%, 85%, 90%, 95% or greater) of all evaluated lesions from the same subject) have responded, or are predicted to respond to the therapy, e.g., CAR19 therapy. In an embodiment, a pre-determined threshold for a responder according to a clinical response criterion is a subject who has, or has shown a clinical response, e.g., a detectable clinical response, e.g., a complete response or a partial response. In an embodiment, a pre-determined threshold for a responder according to the International Prognostic Index (IPI) for DLBCL; is a subject having a high score, e.g., a score of >2. In an embodiment, a pre-determined threshold for a responder according to the follicular lymphoma international prognostic index (FLIPI) is a subject having a high score, e.g., a score of >2.

The term “non-responder” as used herein, refers to a subject who meets a pre-determined threshold according to the All rule, the Majority rule, a prognostic index (e.g., a patient stratification tool) and/or a clinical response criterion. In an embodiment, a pre-determined threshold for a non-responder according to the All rule is subject in whom at least one evaluated lesion has not responded, or is predicted not to respond to the therapy, e.g., CAR19 therapy. In an embodiment, a pre-determined threshold for a non-responder according to the Majority rule a subject in whom a majority of the evaluated lesions have not responded (e.g., based on a % threshold, e.g., at least or greater than 60% (e.g., 60%, 65%, 70%, 75%, 80%, 85%, 90%, 95% or greater) of all evaluated lesions from the same subject) have not responded, or are predicted not to respond to the therapy, e.g., CAR19 therapy. In an embodiment, a pre-determined threshold for a non-responder according to clinical response criteria is a subject who does not have, or has not shown a clinical response, e.g., does not have a detectable clinical response, e.g., has progressive disease. In an embodiment, a pre-determined threshold for a non-responder according to the International Prognostic Index (IPI) for DLBCL; is a subject having a low score, e.g., a score of ≤2. In an embodiment, a pre-determined threshold for a non-responder according to the follicular lymphoma international prognostic index (FLIPI) is a subject having a low score, e.g., a score of ≤2.

The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some embodiments, the set of polypeptides are in the same polypeptide chain (e.g., comprise a chimeric fusion protein). In some embodiments, the set of polypeptides are not contiguous with each other, e.g., are in different polypeptide chains. In some embodiments, the set of polypeptides include a dimerization switch that, upon the presence of a dimerization molecule, can couple the polypeptides to one another, e.g., can couple an antigen binding domain to an intracellular signaling domain. In one embodiment, the stimulatory molecule of the CAR is the zeta chain associated with the T cell receptor complex. In one aspect, the cytoplasmic signaling domain comprises a primary signaling domain (e.g., a primary signaling domain of CD3-zeta). In one embodiment, the cytoplasmic signaling domain further comprises one or more functional signaling domains of at least one costimulatory molecule as defined below. In one embodiment, the costimulatory molecule is a costimulatory molecule described herein, e.g., 4-1BB (i.e., CD137), CD27, ICOS, and/or CD28. In one embodiment, the CAR comprises a chimeric fusion protein comprising an extracellular antigen binding domain, a transmembrane domain and an intracellular signaling domain comprising a functional signaling domain of a stimulatory molecule. In one embodiment, the CAR comprises a chimeric fusion protein comprising an extracellular antigen binding domain, a transmembrane domain and an intracellular signaling domain comprising a functional signaling domain of a co-stimulatory molecule and a functional signaling domain of a stimulatory molecule. In one embodiment, the CAR comprises a chimeric fusion protein comprising an extracellular antigen binding domain, a transmembrane domain and an intracellular signaling domain comprising two functional signaling domains of one or more co-stimulatory molecule(s) and a functional signaling domain of a stimulatory molecule. In one embodiment, the CAR comprises a chimeric fusion protein comprising an extracellular antigen binding domain, a transmembrane domain and an intracellular signaling domain comprising at least two functional signaling domains of one or more co-stimulatory molecule(s) and a functional signaling domain of a stimulatory molecule. In one embodiment, the CAR comprises an optional leader sequence at the amino-terminus (N-terminus) of the CAR fusion protein. In one embodiment, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

A CAR that comprises an antigen binding domain (e.g., a scFv, or TCR) that binds to a specific tumor antigen X, such as those described herein, is also referred to as XCAR or CARX. For example, a CAR that comprises an antigen binding domain that binds to CD19 is referred to as CD19CAR or CAR19.

The term “signaling domain” refers to the functional portion of a protein which acts by transmitting information within the cell to regulate cellular activity via defined signaling pathways by generating second messengers or functioning as effectors by responding to such messengers.

The term “antibody,” as used herein, refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)₂, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

The term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The portion of a CAR comprising an antibody or antibody fragment thereof may exist in a variety of forms where the antigen binding domain is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one embodiment, the antigen binding domain of a CAR comprises an antibody fragment. In a further embodiment, the CAR comprises an antibody fragment that comprises a scFv.

As used herein, the term “antigen binding domain” or “antibody molecule” refers to a protein, e.g., an immunoglobulin chain or fragment thereof, comprising at least one immunoglobulin variable domain sequence. The term “antigen binding domain” or “antibody molecule” encompasses antibodies and antibody fragments. In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The portion of the CAR of the invention comprising an antigen binding domain, e.g., an antibody or antibody fragment thereof, may exist in a variety of forms where the antigen binding domain is expressed as part of a contiguous polypeptide chain including, for example, a single domain antibody fragment (sdAb), a single chain antibody (scFv), a humanized antibody, or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a CAR composition of the invention comprises an antibody fragment. In a further aspect, the CAR comprises an antibody fragment that comprises a scFv.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

The term “complementarity determining region” or “CDR,” as used herein, refers to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), or a combination thereof. Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically

The term “xenogeneic” refers to any material derived from an animal of a different species.

“Derived from” as that term is used herein, indicates a relationship between a first and a second molecule. It generally refers to structural similarity between the first molecule and a second molecule and does not connote or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connote or include a limitation to a particular process of producing the intracellular signaling domain, e.g., it does not mean that, to provide the intracellular signaling domain, one must start with a CD3zeta sequence and delete unwanted sequence, or impose mutations, to arrive at the intracellular signaling domain.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR described herein can be replaced with other amino acid residues from the same side chain family and the altered CAR can be tested using the functional assays described herein.

The term “stimulation,” refers to a primary response induced by binding of a stimulatory molecule (e.g., a TCR/CD3 complex or CAR) with its cognate ligand (or tumor antigen in the case of a CAR) thereby mediating a signal transduction event, such as, but not limited to, signal transduction via the TCR/CD3 complex or signal transduction via the appropriate NK receptor or signaling domains of the CAR. Stimulation can mediate altered expression of certain molecules.

The term “stimulatory molecule,” refers to a molecule expressed by an immune cell (e.g., T cell, NK cell, B cell) that provides the cytoplasmic signaling sequence(s) that regulate activation of the immune cell in a stimulatory way for at least some aspect of the immune cell signaling pathway. In one aspect, the signal is a primary signal that is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing cytoplasmic signaling sequence that is of particular use in the invention includes, but is not limited to, those derived from CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12. In a specific CAR of the invention, the intracellular signaling domain in any one or more CARS of the invention comprises an intracellular signaling sequence, e.g., a primary signaling sequence of CD3-zeta. In a specific CAR of the invention, the primary signaling sequence of CD3-zeta is the sequence provided as SEQ ID NO:9 (mutant CD3 zeta), or the equivalent residues from a non-human species, e.g., mouse, rodent, monkey, ape and the like. In a specific CAR of the invention, the primary signaling sequence of CD3-zeta is the sequence as provided in SEQ ID NO:10 (wild-type human CD3 zeta), or the equivalent residues from a non-human species, e.g., mouse, rodent, monkey, ape and the like.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

An “intracellular signaling domain,” as the term is used herein, refers to an intracellular portion of a molecule. The intracellular signaling domain can generate a signal that promotes an immune effector function of the CAR containing cell, e.g., a CART cell. Examples of immune effector function, e.g., in a CART cell, include cytolytic activity and helper activity, including the secretion of cytokines. In embodiments, the intracellular signaling domain is the portion of a protein which transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, CD278 (“ICOS”), FcεRI, and CD66d, CD32, DAP10, and DAP12.

The term “zeta” or alternatively “zeta chain”, “CD3-zeta” or “TCR-zeta” is defined as the protein provided as GenBank Acc. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain that are sufficient to functionally transmit an initial signal necessary for T cell activation. In one aspect the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof. In one aspect, the “zeta stimulatory domain” or a “CD3-zeta stimulatory domain” is the sequence provided as SEQ ID NO:9. In one aspect, the “zeta stimulatory domain” or a “CD3-zeta stimulatory domain” is the sequence provided as SEQ ID NO:10.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to MHC class I molecule, TNF receptor proteins, Immunoglobulin-like proteins, cytokine receptors, integrins, signalling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

A costimulatory intracellular signaling domain refers to an intracellular portion of a costimulatory molecule. The intracellular signaling domain can comprise the entire intracellular portion, or the entire native intracellular signaling domain, of the molecule from which it is derived, or a functional fragment thereof.

The intracellular signaling domain can comprise the entire intracellular portion, or the entire native intracellular signaling domain, of the molecule from which it is derived, or a functional fragment thereof.

The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or the equivalent residues from a non-human species, e.g., mouse, rodent, monkey, ape and the like. In one aspect, the “4-1BB costimulatory domain” is the sequence provided as SEQ ID NO:7 or the equivalent residues from a non-human species, e.g., mouse, rodent, monkey, ape and the like.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloid-derived phagocytes.

“Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines.

The term “depletion” or “depleting”, as used interchangeably herein, refers to the decrease or reduction of the level or amount of a cell, a protein, or macromolecule in a sample after a process, e.g., a selection step, e.g., a negative selection, is performed. The depletion can be a complete or partial depletion of the cell, protein, or macromolecule. In an embodiment, the depletion is at least a 1%, 2%, 5%, 10%, 15%, 20%, 25%, 30%, 35%, 40%, 45%, 50%, 55%, 60%, 65%, 70%, 75%, 80%, 85%, 90%, 95%, or 99% decrease or reduction of the level or amount of a cell, a protein, or macromolecule, as compared to the level or amount of the cell, protein or macromolecule in the sample before the process was performed.

The term “enriched” or “enrichment”, as used interchangeably herein, refers to the increase of the level or amount of a cell, a protein, or macromolecule in a sample after a process, e.g., a selection step, e.g., a positive selection, is performed. The enrichment can be a complete or partial enrichment of the cell, protein, or macromolecule. In an embodiment, the enrichment is at least 1%, e.g., at least 1-200%, e.g., at least 1-10, 10-20, 20-30, 30-50, 50-70, 70-90, 90-110, 110-130, 130-150, 150-170, or 170-200% increase of the level or amount of a cell, a protein, or macromolecule, as compared to the level or amount of the cell, protein or macromolecule in a reference sample. In some embodiments, the enrichment is at least 5%, e.g., at least 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 99% increase of the level or amount of a cell, a protein, or macromolecule, as compared to the level or amount of the cell, protein or macromolecule in a reference sample. In some embodiments, the enrichment is at least 1.1 fold, e.g., 1.1-200 fold, e.g., 1.1-10, 10-20, 20-30, 30-50, 50-70, 70-90, or 90-100 fold increase of the level or amount of a cell, a protein, or macromolecule, as compared to the level or amount of the cell, protein or macromolecule in a reference sample. In some embodiments, the reference sample can be a same sample, e.g., the sample before the process was performed. In some embodiments, the same sample refers to the sample on which the enrichment is subsequently performed, e.g., a pre-enrichment population, e.g., a starting population. In some embodiments, the reference sample can be a different sample, e.g., a sample on which the process is not performed.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acid (DNA) or ribonucleic acid (RNA), or a combination of a DNA or RNA thereof, and polymers thereof in either single- or double-stranded form. The term “nucleic acid” includes a gene, cDNA or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The term “flexible polypeptide linker” or “linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)ₙ, where n is a positive integer equal to or greater than 1. For example, n=1, n=2, n=3, n=4, n=5, n=6, n=7, n=8, n=9 and n=10 (SEQ ID NO: 15). In one embodiment, the flexible polypeptide linkers include, but are not limited to, (Gly₄Ser)₄ (SEQ ID NO:27) or (Gly₄Ser)₃ (SEQ ID NO:28). In another embodiment, the linkers include multiple repeats of (Gly₂Ser), (GlySer) or (Gly₃Ser) (SEQ ID NO:29). Also included within the scope of the invention are linkers described in WO2012/138475, incorporated herein by reference).

As used herein, a 5′ cap (also termed an RNA cap, an RNA 7-methylguanosine cap or an RNA m⁷G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, e.g., mRNA, that has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In some embodiments of a construct for transient expression, the polyA is between 50 and 5000 (SEQ ID NO: 30), e.g., greater than 64, e.g., greater than 100, e.g., greater than 300 or 400 poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

Apheresis is the process in which whole blood is removed from an individual, separated into select components, and the remainder returned to circulation. Generally, there are two methods for the separation of blood components, centrifugal and non-centrifugal. Leukapheresis results in the active selection and removal of the patient's white blood cells.

As used herein, the terms “treat”, “treatment” and “treating” refer to the reduction or amelioration of the progression, severity and/or duration of a proliferative disorder, or the amelioration of one or more symptoms (e.g., one or more discernible symptoms) of a proliferative disorder resulting from the administration of one or more therapies (e.g., one or more therapeutic agents such as a CAR of the invention). In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human). In one embodiment, the subject is a patient.

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

In the context of the present invention, “tumor antigen” or “hyperproliferative disorder antigen” or “antigen associated with a hyperproliferative disorder” refers to antigens that are common to specific hyperproliferative disorders. In certain embodiments, the tumor antigen is derived from a cancer including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer, ovarian cancer, pancreatic cancer, and the like.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a cognate binding partner protein present in a sample, but which antibody or ligand does not substantially recognize or bind other molecules in the sample.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

### Method of Predicting Responsiveness to a CAR Therapy

In an aspect, this disclosure provides a method for determining, e.g., predicting, a lesion-level treatment response to a chimeric antigen receptor (CAR) therapy, e.g., a CAR 19 therapy, based, e.g., on deep-learning (DL) image analysis. In an embodiment, the method further comprises a rule-based reasoning methodology for patient-level response prediction.

In an aspect, the disclosure provides a system and a non-transitory computer-readable medium for determining a lesion-level response to a CAR therapy, e.g., a CAR19 therapy.

The medical image-based approach disclosed herein to determine personalized response prediction to CAR T-cell therapies has certain unique advantages including: (1) the use of pre-existing diagnostic imaging data sets previously acquired for clinical purposes; (2) the lack of invasiveness; (3) the extraction of regional phenotypic information from disease and extra-disease sites throughout the body that may be heterogeneous; and (4) production-mode efficiency.

In some embodiments, the method for determining, e.g., predicting, a lesion-level treatment response comprises: acquiring, e.g., receiving, an image of a lesion of a subject, e.g., a subject having or at risk of having a lymphoma (“acquired image”); and processing the image with a neural network (“processed image”).

In embodiments, the neural network outputs a classification result indicating the lesion-level treatment response to the CAR19 therapy.

In some embodiments, acquiring, e.g., receiving, an image of a lesion of a subject, comprises acquiring at least one, two, three, four, five, six, seven, eight, nine or ten images. In some embodiments, the acquired image comprises a plurality of images, e.g., at least two, three, four, five, six, seven, eight, nine or ten images of different views of the same lesion in the subject. In some embodiments, the acquired image comprises a sagittal view, a coronal view, a transverse view, a longitudinal view, or a combination thereof, of the same lesion.

In some embodiments, the acquired image is obtained by at least one imaging modality chosen from: computed tomography (CT) (e.g., diagnostic CT (dCT), low dose CT (lCT)), positron emission tomography (PET), magnetic resonance imaging (MRI), single-photon emission computerized tomography (SPECT), PET lCT, PET/MRI, SPECT lCT, lCT/PET, or a combination thereof. In some embodiments, the acquired image is obtained by computed tomography (CT) (e.g., diagnostic CT (dCT), low dose CT (lCT)). In some embodiments, the acquired image is obtained by positron emission tomography (PET). In some embodiments, the acquired image is obtained by magnetic resonance imaging (MRI). In some embodiments, the acquired image is obtained by single-photon emission computerized tomography (SPECT). In some embodiments, the acquired image is obtained by PET lCT. In some embodiments, the acquired image is obtained by PET/MRI. In some embodiments, the acquired image is obtained by SPECT lCT. In some embodiments, the acquired image is obtained by lCT/PET.

In some embodiments, the acquired image is a pre-treatment image, e.g., prior to a CAR19 therapy.

In other embodiments, the acquired image is a post-treatment image, e.g., after a CAR19 therapy and/or a different therapy (e.g., chemotherapy or radiotherapy).

Method of Evaluation and/or Treatment

The method of predicting a lesion-level treatment response and/or a patient-level response disclosed herein can be used in a method of evaluating, or predicting the responsiveness of a subject having, or at risk of having a lymphoma, to a CAR19 therapy. The method of predicting a lesion-level treatment response and/or a patient-level response can also be used in a method of treating a subject having, or at risk of having a lymphoma wherein the method comprises administering to the subject an effective amount of a CAR19 therapy.

In some embodiments of a method of treating a subject with a CAR19 therapy, or a method of evaluating a subject for responsiveness to a CAR19 therapy, the method comprises determining, e.g., predicting, a lesion-level treatment response to the CAR19 therapy with a neural network. In some embodiments, said determination comprises acquiring, e.g., receiving, an image of a lesion of the subject (“acquired image”); and processing the image with a neural network (“processed image”), wherein the neural network outputs a classification result indicating the lesion-level treatment response to the CAR19 therapy.

In some embodiments, the method further comprises a patient-level response prediction. In some embodiments, the patient-level response prediction comprises a rule-based reasoning method. In some embodiments, the patient-level response prediction comprises an All rule or a Majority rule.

In some embodiments, the patient-level response prediction comprises an All rule. In some embodiments, a subject is classified as a responder or non-responder according to the All rule. In some embodiments, a responder in the All rule of the patient-level response prediction is a subject in whom all evaluated lesions have responded, or are predicted to respond to the CAR19 therapy. In some embodiments, a non-responder in the All rule of the patient-level response prediction is a subject in whom at least one evaluated lesion has not responded, or is predicted not to respond to the CAR19 therapy.

In some embodiments, the patient-level response prediction comprises a Majority rule. In some embodiments, a subject is classified as a responder or non-responder according to the Majority rule. In some embodiment, a responder in the Majority rule of the patient-level response prediction is a subject in whom a majority of the evaluated lesions (e.g., based on a % threshold, e.g., at least or greater than 60% (e.g., 60%, 65%, 70%, 75%, 80%, 85%, 90%, 95% or greater) of all evaluated lesions from the same subject) have responded, or are predicted to respond to the CAR19 therapy. In some embodiments, a non-responder in the Majority rule of the patient-level response prediction is a subject in whom a majority of the evaluated lesions have not responded (e.g., based on a % threshold, e.g., at least or greater than 60% (e.g., 60%, 65%, 70%, 75%, 80%, 85%, 90%, 95% or greater) of all evaluated lesions from the same subject) have not responded, or are predicted not to respond to the CAR19 therapy.

In some embodiments, a responder according to the patient-level response prediction, e.g., based on the All rule or the Majority rule, is a subject who has, or has shown a clinical response, e.g., a detectable clinical response, e.g., a complete response or a partial response.

In some embodiments, a non-responder according to the patient-level response prediction, e.g., based on the All rule or the Majority rule, is a subject who does not have, or has not shown a clinical response, e.g., does not have a detectable clinical response, e.g., has progressive disease.

### Prognostic Tools or Patient Stratification Tools

Prognostic tools or patient stratification tools can also be used to classify patients as responders or non-responders. For example, the International Prognostic Index (IPI) for DLBCL, or the follicular lymphoma international prognostic index (FLIPI) for follicular lymphoma can be used to classify or stratify DLBCL and FL patients respectively.

International Prognostic Index (IPI) is a clinical tool developed for predicting prognosis of patients with non-Hodgkin's lymphoma, e.g., DLBCL. According to IPI, one point is assigned for each of the following risk factors: age >60 years; Stage III or IV disease; elevated serum LDH levels; ECOG/Zubrod performance status of 2, 3, or 4; or more than 1 extranodal site with disease involvement. The sum of the points correlates with risk groups as follows: 0-1 points correlates with low risk groups; 2 points correlates with low-intermediate risk groups; 3 points correlates with high-intermediate risk groups; and 4-5 points correlates with high risk groups. The IPI index is disclosed and described in detail in the article titled “A predictive model for aggressive non-Hodgkin's lymphoma” published in the New England Journal of Medicine (1993) Volume 329(14) pp. 987-94, the entire contents of which is incorporated by reference in its entirety.

Based on IPI, the follicular lymphoma international prognostic index (FLIPI) was developed as a prognostic tool for follicular lymphoma. According to FLIPI, one point is assigned for each of the following adverse prognostic factors: age greater than 60; Stage III or IV of disease; greater than 4 lymph node group involvement; serum hemoglobin of less than 12 g/dL; or elevated serum LDH levels. The sum of points allotted correlates with risk groups as follows: 0-1 points correlates with low risk groups; 2 points correlates with intermediate risk groups; and 3-5 points correlates with high risk groups. The FLIPI index is disclosed and described in detail in Solal-Celigny P et al. (2010) International Journal of Hematology, Volume 92(2) pp. 246-254, the entire contents of which is incorporated by reference in its entirety.

In some embodiments, a responder according to the patient-level response prediction, e.g., based on the All rule or the Majority rule, is a subject who has, or has shown a clinical response, e.g., a detectable clinical response, e.g., a complete response or a partial response. In some embodiments, a responder according to the patient-level response prediction corresponds to a patient having a low score in a clinical stratification tool, e.g., International Prognostic Index (IPI) for DLBCL or follicular lymphoma international prognostic index (FLIPI) for follicular lymphoma. In some embodiments, the responder according to the patient-level response prediction has an IPI score of ≤2. In some embodiments, the responder according to the patient-level response prediction has a FLIPI score of ≤2.

In some embodiments, a non-responder according to the patient-level response prediction, e.g., based on the All rule or the Majority rule, is a subject who does not have, or has not shown a clinical response, e.g., does not have a detectable clinical response, e.g., has progressive disease. In some embodiments, a non-responder according to the patient-level response prediction corresponds to a subject having a high score in a clinical stratification tool, e.g., International Prognostic Index (IPI) for DLBCL and follicular lymphoma international prognostic index (FLIPI) for follicular lymphoma. In some embodiments, the non-responder according to the patient-level response prediction has an IPI score of >2. In some embodiments, the non-responder according to the patient-level response prediction has a FLIPI score of >2.

### Computing System and Associated Learning Methods

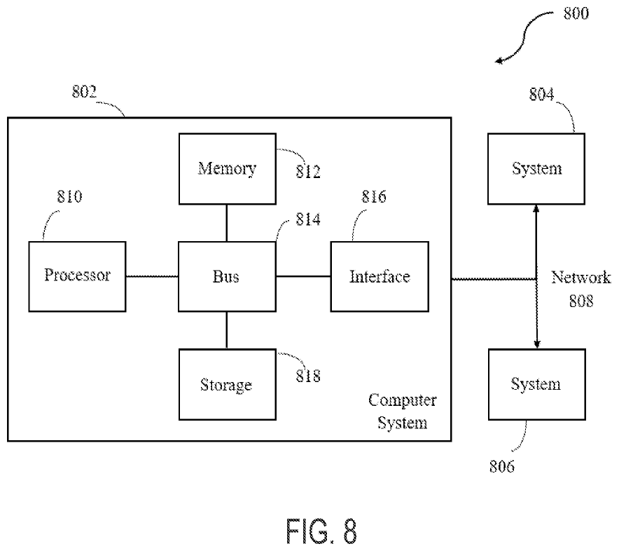

FIG. 8 depicts a block diagram of a distributed computer system 800, in which various aspects and functions discussed herein may be practiced. The distributed computer system 800 may include one or more computer systems. For example, as illustrated, the distributed computer system 800 includes three computer systems 802, 804, 806. As shown, the computer systems 802, 804 and 806 are interconnected by, and may exchange data through, a communication network 808. The network 808 may include any communication network through which computer systems may exchange data. To exchange data via the network 808, the computer systems 802, 804, and 806 and the network 808 may use various methods, protocols and standards including, among others, token ring, Ethernet, Wireless Ethernet, Bluetooth, radio signaling, infra-red signaling, TCP/IP, UDP, HTTP, FTP, SNMP, SMS, MMS, SS7, JSON, XML, REST, SOAP, CORBA HOP, RMI, DCOM and Web Services.

According to some embodiments, the functions and operations discussed for processing image data according to any method disclosed herein can be executed on computer systems 802, 804 and 806 individually and/or in combination. For example, the computer systems 802, 804, and 806 support, for example, participation in a collaborative network. In one alternative embodiment, a single computer system (e.g., 802) can process the image data. The computer systems 802, 804 and 806 may include personal computing devices such as cellular telephones, smart phones, tablets, “fablets,” etc., and may also include desktop computers, laptop computers, etc.

Various aspects and functions in accord with embodiments discussed herein may be implemented as specialized hardware or software executing in one or more computer systems including the computer system 802 shown in FIG. 8. In one embodiment, computer system 802 is a personal computing device specially configured to execute the processes and/or operations discussed herein. As depicted, the computer system 802 includes at least one processor 810 (e.g., a single core or a multi-core processor), a memory 812, a bus 814, input/output interfaces (e.g., 816) and storage 818. The processor 810, which may include one or more microprocessors or other types of controllers, can perform a series of instructions that manipulate data. As shown, the processor 810 is connected to other system components, including a memory 812, by an interconnection element (e.g., the bus 814).

The memory 812 and/or storage 818 may be used for storing programs and data during operation of the computer system 802. For example, the memory 812 may be a relatively high performance, volatile, random access memory such as a dynamic random access memory (DRAM) or static memory (SRAM). In addition, the memory 812 may include any device for storing data, such as a disk drive or other non-volatile storage device, such as flash memory, solid state, or phase-change memory (PCM). In further embodiments, the functions and operations discussed with respect to image data processing according to any method disclosed herein can be embodied in an application that is executed on the computer system 802 from the memory 812 and/or the storage 818. For example, the application can be made available through an “app store” for download and/or purchase. Once installed or made available for execution, computer system 702 can be specially configured to execute the processing an image according to any method disclosed herein.

Computer system 802 also includes one or more interfaces 816 such as input devices (e.g., camera for capturing images), output devices and combination input/output devices. The output devices may include a display for outputting a graphical user interface (GUI), which a user may interact with in order to carry out the methods disclosed herein. The interfaces 816 may receive input, provide output, or both. The storage 818 may include a computer-readable and computer-writeable nonvolatile storage medium in which instructions are stored that define a program to be executed by the processor. The storage system 818 also may include information that is recorded, on or in, the medium, and this information may be processed by the application. A medium that can be used with various embodiments may include, for example, optical disk, magnetic disk or flash memory, SSD, among others. Further, aspects and embodiments are not to a particular memory system or storage system.

In some embodiments, the computer system 802 may include an operating system that manages at least a portion of the hardware components (e.g., input/output devices, touch screens, cameras, etc.) included in computer system 802. One or more processors or controllers, such as processor 810, may execute an operating system which may be, among others, a Windows-based operating system (e.g., Windows NT, M E, XP, Vista, 7, 8, 10, or RT) available from the Microsoft Corporation, an operating system available from Apple Computer (e.g., MAC OS, including System X), one of many Linux-based operating system distributions (for example, the Enterprise Linux operating system available from Red Hat Inc.), a Solaris operating system available from Sun Microsystems, or a UNIX operating systems available from various sources. Many other operating systems may be used, including operating systems designed for personal computing devices (e.g., iOS, Android, etc.) and embodiments are not limited to any particular operating system.

The processor and operating system together define a computing platform on which applications (e.g., “apps” available from an “app store”) may be executed. Additionally, various functions for generating and manipulating images may be implemented in a non-programmed environment (for example, documents created in HTML, XML or other format that, when viewed in a window of a browser program, render aspects of a graphical-user interface or perform other functions). Further, various embodiments in accord with aspects of the present invention may be implemented as programmed or non-programmed components, or any combination thereof. Various embodiments may be implemented in part as MATLAB functions, scripts, and/or batch jobs. Thus, the invention is not limited to a specific programming language and any suitable programming language could also be used.

In one embodiment, a transfer learning program or incremental learning program implementing one or more methods disclosed herein is implemented on computer system 802 and/or one or more of computer systems 804 and 806. System 802 may be, for example, a personal computer (PC) executing the program in MATLAB 2018b under Ubuntu 16.04 OS. The memory 812 of system 802 may include 64 GB RAM. The processor 810 of system 802 may be one of four Intel Core i7 CPUs included in system 802. Additionally, bus 814 of system 802 may be connected to two Nvidia 1080Ti graphics processing unit (GPU) cards with 22 GB GPU RAM in total. The one or more of computer systems 804 and 806 may each be another PC with a respective memory of 24 GB RAM, processors including four Intel Core i7 CPUs, and one Nvidia TITAN V GPU card with 12 GB GPU RAM connected to a respective bus of the one or more of computer systems 804 and 806. Further, various embodiments of systems implementing the transfer learning program or incremental learning program may utilize a different operating system, use a different type or number of processors, a different amount of system memory, or a different GPU. In other embodiments, the program may be implemented entirely on a single computer system.

Although the computer system 802 is shown by way of example as one type of computer system upon which various functions for processing image data according to any method disclosed herein may be practiced, aspects and embodiments are not limited to being implemented on the computer system, shown in FIG. 8. Various aspects and functions may be practiced on one or more computers or similar devices having different architectures or components than that shown in FIG. 8.

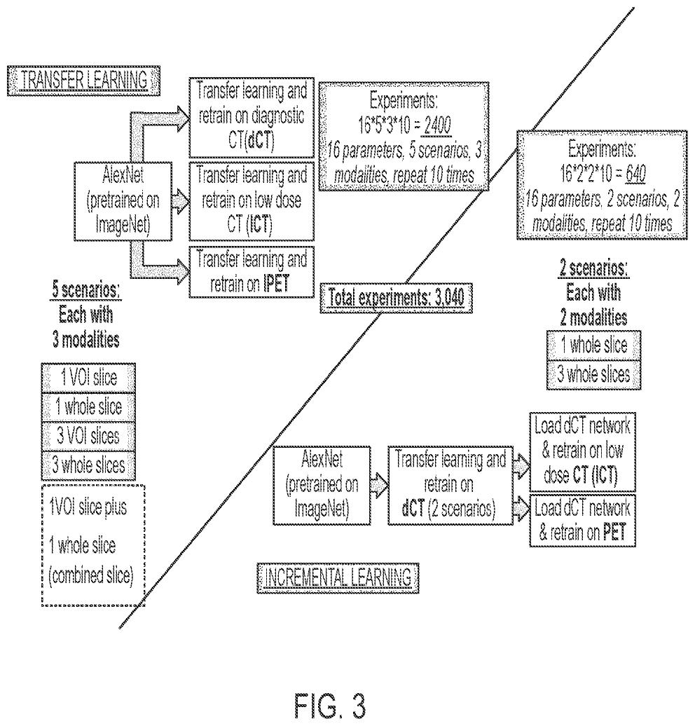

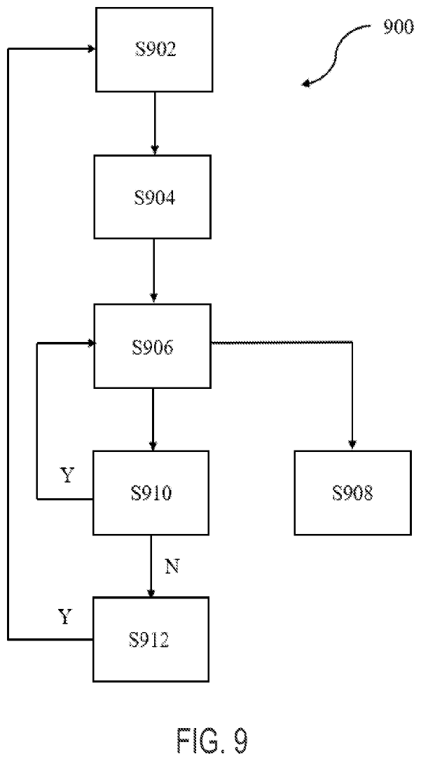

FIG. 9 is flowchart depicting the transfer learning method 900. The method 900, for example, includes embodiments described by the transfer learning shown in FIG. 3 as well as the enumerated embodiments and examples discussed herein. The method 900 may be implemented, for example, on one or more computers in distributed computer system 800. However, aspects and embodiments are not limited to being implemented on only computer system 802 or any subset of computer systems in the distribute computer system 800. Any computer system suitable for deep learning applications may be utilized, preferably where the computer system includes one or more dedicated GPUs.

In step S902, image data for a given scenario, such as the 1 VOI slice scenario, is received as described in the enumerated embodiments and examples discussed herein. The image data may include any one or more of the input scenarios shown in the transfer learning or incremental learning in FIG. 3 and described in the enumerated embodiments and examples. In other embodiments more than one scenario may be combined together to form a new scenario.

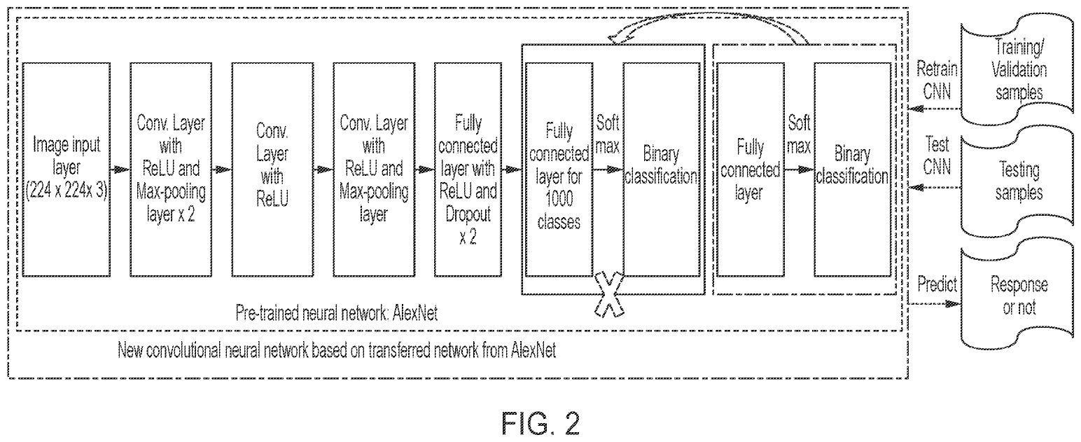

In step S904, a pre-trained neural network, or any other network, that has been modified for a new task is loaded as described herein. For example, the new convolutional neural network from FIG. 2 is loaded. Here, a batch size and epoch may be specified.

In step S906, transfer learning is performed on the modified network using the image data for a given imaging modality, such as dCT, lCT, PET, or any other medical imaging modality. In one embodiment, each iteration of step S906 uses the same network loaded in step S904.

In step S908, the trained, modified neural network from step S906 is output and stored for use with any prediction task disclosed herein.

In step S910, a determination is made if there is a remaining imaging modality to utilize for the given scenario. If so, step S906 is repeated to create an additional trained network to output and store at step S908. If not, the method proceeds to step S912.

In step S912, a determination is made if another scenario for a same or different imaging modality from that already processed by step S902 remains. If so, steps S902, S904, S906, S908, and S910 are repeated. If not, method 900 ends and each of the one or more stored neural networks in step S908 is usable for a prediction task according to any of enumerated embodiments and examples discussed herein.

In another embodiment, the image data for all scenarios to be processed may be input at step S902, and step S912 proceeds to step S904 instead of step S902 in the event another scenario remains. In various embodiments, the steps or portions of the steps may be performed in a different order to achieve the same result(s) produced at step S908.

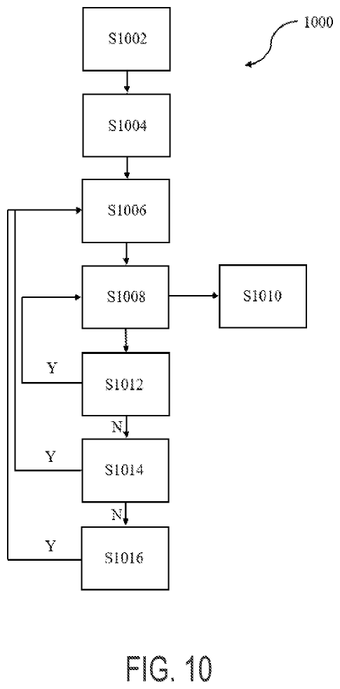

FIG. 10 is flowchart depicting the incremental learning method 1000. The method 1000, for example, includes embodiments described by the transfer learning and incremental learning in FIG. 3 as well as the enumerated embodiments and examples discussed herein. The method 1000 may be implemented, for example, on one or more computers in distributed computer system 800. However, aspects and embodiments are not limited to being implemented on only computer system 802 or any subset of computer systems in the distribute computer system 800. Any computer system suitable for deep learning applications may be utilized, preferably where the computer system includes one or more dedicated GPUs.

In step S1002, image data is received as described in the enumerated embodiments and examples discussed herein. The image data may include any one or more of the input scenarios shown in the transfer learning or incremental learning in FIG. 3 and described in the enumerated embodiments and examples. In other embodiments more than one scenario may be combined together to form a new scenario.

In step S1004, a pre-trained neural network, or any other network, that has been modified for a new task is loaded as described herein. For example, the new convolutional neural network from FIG. 2 is loaded. Here, a batch size and epoch may be specified. In one embodiment steps S1002 and S1004 are performed in the opposite order.

In step S1006, transfer learning is performed on the modified network from step S1004 using the image data for a first imaging modality, such as dCT, and a given scenario, such as the 1 VOI slice scenario. In one embodiment, each iteration of step S1006 uses the same network loaded in step S1004.

In step S1008, the network trained in step S1006 is re-trained for a second imaging modality, such as lCT. In one embodiment the first imaging modality and second imaging modality may be the same. In another embodiment the first imaging modality and second imaging modality may be different.

In step S1010, the re-trained network from step S1008 is output and stored for use with any prediction task disclosed herein.

In step S1012, a determination is made if another second imaging modality, such as PET, exists for the given scenario processed by steps S1006 and S1008. If so, step S1008 is repeated. If not, the method proceeds to step S1014.

In step S1014, a determination is made if another scenario for the first modality remains. If so, steps S1006, S1008, S1010, and S1012 are repeated to produce one or more new re-trained networks to store. If not, the method proceeds to step S1016.

In step S1016, a determination is made if another first modality remains. If so, steps S1006, S1008, S1010, S1012, and S1014 are repeated to produce one or more new re-trained networks to store. If not, method 1000 ends and each of the one or more stored neural networks in step S1010 is usable for a prediction task according to any of enumerated embodiments and examples discussed herein. In various embodiments, the steps or portions of the steps may be performed in a different order to achieve the same result(s) produced at step S1010.

### Chimeric Antigen Receptor (CAR)

The present invention provides immune effector cells (e.g., T cells, NK cells) that are engineered to contain one or more CARs that direct the immune effector cells to cancer. This is achieved through an antigen binding domain on the CAR that is specific for a cancer associated antigen. There are two classes of cancer associated antigens (tumor antigens) that can be targeted by the CARs described herein: (1) cancer associated antigens that are expressed on the surface of cancer cells; and (2) cancer associated antigens that itself is intracellular, however, a fragment of such antigen (peptide) is presented on the surface of the cancer cells by MHC (major histocompatibility complex).

Accordingly, an immune effector cell, e.g., obtained by a method described herein, can be engineered to contain a CAR that targets the CD19 antigen.

Bispecific CARs

In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope.

In certain embodiments, the antibody molecule is a multi-specific (e.g., a bispecific or a trispecific) antibody molecule. Protocols for generating bispecific or heterodimeric antibody molecules, and various configurations for bispecific antibody molecules, are described in, e.g., paragraphs 455-458 of WO2015/142675, filed Mar. 13, 2015, which is incorporated by reference in its entirety.

In one aspect, the bispecific antibody molecule is characterized by a first immunoglobulin variable domain sequence, e.g., a scFv, which has binding specificity for CD19, e.g., comprises a scFv as described herein, or comprises the light chain CDRs and/or heavy chain CDRs from a scFv described herein, and a second immunoglobulin variable domain sequence that has binding specificity for a second epitope on a different antigen.

Chimeric TCR

In one aspect, the antibodies and antibody fragments of the present invention (e.g., CD19 antibodies and fragments) can be grafted to one or more constant domain of a T cell receptor (“TCR”) chain, for example, a TCR alpha or TCR beta chain, to create a chimeric TCR. Without being bound by theory, it is believed that chimeric TCRs will signal through the TCR complex upon antigen binding. For example, an scFv as disclosed herein, can be grafted to the constant domain, e.g., at least a portion of the extracellular constant domain, the transmembrane domain and the cytoplasmic domain, of a TCR chain, for example, the TCR alpha chain and/or the TCR beta chain. As another example, an antibody fragment, for example a VL domain as described herein, can be grafted to the constant domain of a TCR alpha chain, and an antibody fragment, for example a VH domain as described herein, can be grafted to the constant domain of a TCR beta chain (or alternatively, a VL domain may be grafted to the constant domain of the TCR beta chain and a VH domain may be grafted to a TCR alpha chain). As another example, the CDRs of an antibody or antibody fragment may be grafted into a TCR alpha and/or beta chain to create a chimeric TCR. For example, the LCDRs disclosed herein may be grafted into the variable domain of a TCR alpha chain and the HCDRs disclosed herein may be grafted to the variable domain of a TCR beta chain, or vice versa. Such chimeric TCRs may be produced, e.g., by methods known in the art (For example, Willemsen R A et al, Gene Therapy 2000; 7: 1369-1377; Zhang T et al, Cancer Gene Ther 2004; 11: 487-496; Aggen et al, Gene Ther. 2012 April; 19(4):365-74).

Non-Antibody Scaffolds

In embodiments, the antigen binding domain comprises a non-antibody scaffold, e.g., a fibronectin, ankyrin, domain antibody, lipocalin, small modular immuno-pharmaceutical, maxybody, Protein A, or affilin. The non-antibody scaffold has the ability to bind to target antigen on a cell. In embodiments, the antigen binding domain is a polypeptide or fragment thereof of a naturally occurring protein expressed on a cell. In some embodiments, the antigen binding domain comprises a non-antibody scaffold. A wide variety of non-antibody scaffolds can be employed so long as the resulting polypeptide includes at least one binding region which specifically binds to the target antigen on a target cell.

Non-antibody scaffolds include: fibronectin (Novartis, MA), ankyrin (Molecular Partners AG, Zurich, Switzerland), domain antibodies (Domantis, Ltd., Cambridge, Mass., and Ablynx nv, Zwijnaarde, Belgium), lipocalin (Pieris Proteolab AG, Freising, Germany), small modular immuno-pharmaceuticals (Trubion Pharmaceuticals Inc., Seattle, Wash.), maxybodies (Avidia, Inc., Mountain View, Calif.), Protein A (Affibody AG, Sweden), and affilin (gamma-crystallin or ubiquitin) (Scil Proteins GmbH, Halle, Germany).

In an embodiment the antigen binding domain comprises the extracellular domain, or a counter-ligand binding fragment thereof, of molecule that binds a counterligand on the surface of a target cell.

The immune effector cells can comprise a recombinant DNA construct comprising sequences encoding a CAR, wherein the CAR comprises an antigen binding domain (e.g., antibody or antibody fragment, TCR or TCR fragment) that binds specifically to a tumor antigen, e.g., a tumor antigen described herein, and an intracellular signaling domain. The intracellular signaling domain can comprise a costimulatory signaling domain and/or a primary signaling domain, e.g., a zeta chain. As described elsewhere, the methods described herein can include transducing a cell, e.g., from the population of T regulatory-depleted cells, with a nucleic acid encoding a CAR, e.g., a CAR described herein.

In specific aspects, a CAR comprises a scFv domain, wherein the scFv may be preceded by an optional leader sequence such as provided in SEQ ID NO: 1, and followed by an optional hinge sequence such as provided in SEQ ID NO:2 or SEQ ID NO:36 or SEQ ID NO:38, a transmembrane region such as provided in SEQ ID NO:6, an intracellular signalling domain that includes SEQ ID NO:7 or SEQ ID NO:16 and a CD3 zeta sequence that includes SEQ ID NO:9 or SEQ ID NO:10, e.g., wherein the domains are contiguous with and in the same reading frame to form a single fusion protein.

In one aspect, an exemplary CAR constructs comprise an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), and an intracellular stimulatory domain (e.g., an intracellular stimulatory domain described herein). In one aspect, an exemplary CAR construct comprises an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), an intracellular costimulatory signaling domain (e.g., a costimulatory signaling domain described herein) and/or an intracellular primary signaling domain (e.g., a primary signaling domain described herein).

An exemplary leader sequence is provided as SEQ ID NO: 1. An exemplary hinge/spacer sequence is provided as SEQ ID NO: 2 or SEQ ID NO:36 or SEQ ID NO:38. An exemplary transmembrane domain sequence is provided as SEQ ID NO:6. An exemplary sequence of the intracellular signaling domain of the 4-1BB protein is provided as SEQ ID NO: 7. An exemplary sequence of the intracellular signaling domain of CD27 is provided as SEQ ID NO:16. An exemplary CD3zeta domain sequence is provided as SEQ ID NO: 9 or SEQ ID NO:10.

In one aspect, the immune effector cell comprises a recombinant nucleic acid construct comprising a nucleic acid molecule encoding a CAR, wherein the nucleic acid molecule comprises a nucleic acid sequence encoding an antigen binding domain, wherein the sequence is contiguous with and in the same reading frame as the nucleic acid sequence encoding an intracellular signaling domain. An exemplary intracellular signaling domain that can be used in the CAR includes, but is not limited to, one or more intracellular signaling domains of, e.g., CD3-zeta, CD28, CD27, 4-1BB, and the like. In some instances, the CAR can comprise any combination of CD3-zeta, CD28, 4-1BB, and the like.

The nucleic acid sequences coding for the desired molecules can be obtained using recombinant methods known in the art, such as, for example by screening libraries from cells expressing the nucleic acid molecule, by deriving the nucleic acid molecule from a vector known to include the same, or by isolating directly from cells and tissues containing the same, using standard techniques. Alternatively, the nucleic acid of interest can be produced synthetically, rather than cloned.

Nucleic acids encoding a CAR can be introduced into the immune effector cells using, e.g., a retroviral or lentiviral vector construct.

Nucleic acids encoding a CAR can also be introduced into the immune effector cell using, e.g., an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequence (“UTR”) (e.g., a 3′ and/or 5′ UTR described herein), a 5′ cap (e.g., a 5′ cap described herein) and/or Internal Ribosome Entry Site (IRES) (e.g., an IRES described herein), the nucleic acid to be expressed, and a polyA tail, typically 50-2000 bases in length (SEQ ID NO: 35) (e.g., described in the Examples, e.g., SEQ ID NO:35). RNA so produced can efficiently transfect different kinds of cells. In one embodiment, the template includes sequences for the CAR. In an embodiment, an RNA CAR vector is transduced into a cell, e.g., a T cell by electroporation.

### Antigen Binding Domain

In one aspect, a plurality or population of the immune effector cells include a nucleic acid encoding a CAR that comprises a target-specific binding element otherwise referred to as an antigen binding domain. The choice of binding element depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen binding domain in a CAR described herein include those associated with viral, bacterial and parasitic infections, autoimmune disease and cancer cells.

In one aspect, the portion of the CAR comprising the antigen binding domain comprises an antigen binding domain that targets a tumor antigen, e.g., a tumor antigen described herein.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment.

In an embodiment, the antigen binding domain comprises an anti-CD19 antibody, or fragment thereof, e.g., an scFv. For example, the antigen binding domain comprises a variable heavy chain and a variable light chain listed in Table 1. The linker sequence joining the variable heavy and variable light chains can be, e.g., any of the linker sequences described herein, or alternatively, can be GSTSGSGKPGSGEGSTKG (SEQ ID NO:104).

TABLE 1
Anti-CD19 antibody binding domains

| | | |
|---|---|---|
| CD19 | huscFv1 | EIVMTQSPATLSLSPGERATLSCRASQDISKYLNWYQQKPGQAPRLLIYHTSRL |
| | (SEQ ID | HSGIPARFSGSGSGTDYTLTISSLQPEDFAVYFCQQGNTLPYTFGQGTKLEIKG |
| | NO: 39) | GGGSGGGGSGGGGSQVQLQESGPGLVKPSETLSLTCTVSGVSLPDYGVSWIRQP |
| | | PGKGLEWIGVIWGSETTYYSSSLKSRVTISKDNSKNQVSLKLSSVTAADTAVYY |
| | | CAKHYYYGGSYAMDYWGQGTLVTVSS |
| CD19 | huscFv2 | Eivmtqspatlslspgeratlscrasqdiskylnwyqqkpgqaprlliyhtsrlhsgip |
| | (SEQ ID | arfsgsgsgtdytltisslqpedfavyfcqqgntlpytfgqgtkleikggggsggggsg |
| | NO: 40) | gggsqvqlqesgpglvkpsetlsltctvsgvslpdygvswirqppgkglewigviwgse |
| | | ttyyqsslksrvtiskdnsknqvslklssvtaadtavyycakhyyyggsyamdywgqgt |
| | | lvtvss |
| CD19 | huscFv3 | Qvqlqesgpglvkpsetlsltctvsgvslpdygvswirqppgkglewigviwgsettyy |
| | (SEQ ID | ssslksrvtiskdnsknqvslklssvtaadtavyycakhyyyggsyamdywgqgtlvtv |
| | NO: 41) | ssggggsggggsggggseivmtqspatlslspgeratlscrasqdiskylnwyqqkpgq |
| | | aprlliyhtsrlhsgiparfsgsgsgtdytitisslqpedfavyfcgqgntlpytfgqg |
| | | tkleik |
| CD19 | huscFv4 | Qvqlqesgpglvkpsetlsltctvsgvslpdygvswirqppgkglewigviwgsettyy |
| | (SEQ ID | qsslksrvtiskdnsknqvslklssvtaadtavyycakhyyyggsyamdywgqgtlvtv |
| | NO: 42) | ssggggsggggsggggseivmtqspatlslspgeratlscrasqdiskylnwyqqkpgq |
| | | aprlliyhtsrlhsgiparfsgsgsgtdytltisslqpedfavyfcqqgntlpytfgqg |
| | | tkleik |
| CD19 | huscFv5 | Eivmtqspatlslspgeratlscrasqdiskylnwyqqkpgqaprlliyhtsrlhsgip |
| | (SEQ ID | arfsgsgsgtdytltisslqpedfavyfcqqgntlpytfgqgtkleikggggsggggsg |
| | NO: 43) | gggsggggsqvqlqesgpglvkpsetlsltctvsgvslpdygvswirqppgkglewigv |
| | | iwgsettyyssslksrvtiskdnsknqvslklssvtaadtavyycakhyyyggsyamdy |
| | | wgqgtlvtvss |
| CD19 | huscFv6 | Eivmtqspatlslspgeratlscrasqdiskylnwyqqkpgqaprlliyhtsrlhsgip |
| | (SEQ ID | arfsgsgsgtdytitisslqpedfavyfcqqgntlpytfgqgtkleikggggsggggsg |
| | NO: 44) | gggsggggsqvqlqesgpglvkpsetlsltctvsgvslpdygvswirqppgkglewigv |
| | | iwgsettyyqsslksrvtiskdnsknqvslklssvtaadtavyycakhyyyggsyamdy |
| | | wgqgtlvtvss |
| CD19 | huscFv7 | Qvqlqesgpglvkpsetlsltctvsgvslpdygvswirqppgkglewigviwgsettyy |
| | (SEQ ID | ssslksrvtiskdnsknqvslklssvtaadtavyycakhyyyggsyamdywgqgtlvtv |
| | NO: 45) | ssggggsggggsggggsggggseivmtqspatlslspgeratlscrasqdiskylnwyq |
| | | qkpgqaprlliyhtsrlhsgiparfsgsgsgtdytltisslqpedfavyfcqqgntlpy |
| | | tfgqgtklelk |
| CD19 | huscFv8 | Qvqlqesgpglvkpsetlsltctvsgvslpdygvswirqppgkglewigviwgsettyy |
| | (SEQ ID | qsslksrvtiskdnsknqvslklssvtaadtavyycakhyyyggsyamdywgqgtlvtv |
| | NO: 46) | ssggggsggggsggggsggggseivmtqspatlslspgeratlscrasqdiskylnwyq |
| | | qkpgqaprlliyhtsrlhsgiparfsgsgsgtdytltisslqpedfavyfcqqgntlpy |
| | | tfgqgtkleik |
| CD19 | huscFv9 | Eivmtqspatlslspgeratlscrasqdiskylnwyqqkpgqaprlliyhtsrlhsgip |
| | (SEQ ID | arfsgsgsgtdytltisslqpedfavyfcqqgntlpytfgqgtkleikggggsggggsg |
| | NO: 47) | gggsggggsqvqlqesgpglvkpsetlsltctvsgvslpdygvswirqppgkglewigv |
| | | iwgsettyynsslksrvtiskdnsknqvslklssvtaadtavyycakhyyyggsyamdy |
| | | wgqgtlvtvss |
| CD19 | HuscFv10 | Qvqlqesgpglvkpsetlsltctvsgvslpdygvswirqppgkglewigviwgsettyy |
| | (SEQ ID | nsslksrvtiskdnsknqvslklssvtaadtavyycakhyyyggsyamdywgqgtlvtv |
| | NO: 48) | ssggggsggggsggggsggggseivmtqspatlslspgeratlscrasqdiskylnwyq |
| | | qkpgqaprlliyhtsrlhsgiparfsgsgsgtdytltisslqpedfavyfcqqgntlpy |
| | | tfgqgtkleik |
| CD19 | HuscFv11 | Eivmtqspatlslspgeratlscrasqdiskylnwyqqkpgqaprlliyhtsrlhsgip |
| | (SEQ ID | arfsgsgsgtdytltisslqpedfavyfcqqgntlpytfgggtkleikggggsggggsg |
| | NO: 49) | gggsqvqlqesgpglvkpsetlsltctvsgvslpdygvswirqppgkglewigviwgse |
| | | ttyynsslksrvtiskdnsknqvslklssvtaadtavyycakhyyyggsyamdywgqgt |
| | | lvtvss |
| CD19 | HuscFv12 | Qvqlqesgpglvkpsetlsltctvsgvslpdygvswirqppgkglewigviwgsettyy |
| | (SEQ ID | nsslksrvtiskdnsknqvslklssvtaadtavyycakhyyyggsyamdywgqgtlvtv |
| | NO: 50) | ssggggsggggsggggseivmtqspatlslspgeratlscrasqdiskylnwyqqkpgq |
| | | aprlliyhtsrlhsgiparfsgsgsgtdytltisslqpedfavyfcqqgntlpytfgqg |
| | | tkleik |
| CD19 | muCTL019 | Diqmtqttsslsaslgdrvtiscrasqdiskylnwyqqkpdgtvklliyhtsrlhsgvp |
| | (SEQ | srfsgsgsgtdysltisnleqediatyfcqqgntlpytfgggtkleitggggsggggsg |
| | ID NO: | gggsevklqesgpglvapsqslsvtctvsgvslpdygvswirqpprkglewlgviwgse |
| | 51) | ttyynsalksrltiikdnsksqvfikmnslqtddtaiyycakhyyyggsyamdywgqgt |
| | | svtvss |

TABLE 2
Additional murine anti-CD19 antibody binding domains and CARs

| | | |
|---|---|---|
| mCAR1 | SEQ ID | QVQLLESGAELVRPGSSVKISCKASGYAFSSYWMNWVKQRPGQGLEWIGQIYPGDG |
| scFv | NO: 124 | DTNYNGKFKGQATLTADKSSSTAYMQLSGLTSEDSAVYSCARKTISSVVDFYFDYW |
| | | GQGTTVTGGGSGGGSGGGSGGGSELVLTQSPKFMSTSVGDRVSVTCKASQNVGTNV |
| | | AWYQQKPGQSPKPLIYSATYRNSGVPDRFTGSGSGTDFTLTITNVQSKDLADYFCQ |
| | | YNRYPYTSFFFTKLEIKRRS |
| mCAR1 | SEQ ID | QVQLLESGAELVRPGSSVKISCKASGYAFSSYWMNWVKQRPGQGLEWIGQIYPGDG |
| Full - aa | NO: 184 | DTNYNGKFKGQATLTADKSSSTAYMQLSGLTSEDSAVYSCARKTISSVVDFYFDYW |
| | | GQGTTVTGGGSGGGSGGGSGGGSELVLTQSPKFMSTSVGDRVSVTCKASQNVGTNV |
| | | AWYQQKPGQSPKPLIYSATYRNSGVPDRFTGSGSGTDFTLTITNVQSKDLADYFCQ |
| | | YNRYPYTSFFFTKLEIKRRSKIEVMYPPPYLDNEKSNGTIIHVKGKHLCPSPLFPG |
| | | PSKPFWVLVVVGGVLACYSLLVTVAFIIFWVRSKRSRLLHSDYMNMTPRRPGPTRK |
| | | HYQPYAPPRDFAAYRSRVKFSRSADAPAYQQGQNQLYNELNLGRREEYDVLDKRRG |
| | | RDPEMGGKPRRKNPQEGLYNELQKDKMAEAYSEIGMKGERRRGKGHDGLYQGLSTA |
| | | TKDTYDALHMQALPPR |
| mCAR2 | SEQ ID | DIQMTQTTSSLSASLGDRVTISCRASQDISKYLNWYQQKPDGTVKLLIYHTSRLHS |
| scFv | NO: 125 | GVPSRFSGSGSGTDYSLTISNLEQEDIATYFCQQGNTLPYTFGGGTKLEITGSTSG |
| | | SGKPGSGEGSTKGEVKLQESGPGLVAPSQSLSVTCTVSGVSLPDYGVSWIRQPPRK |
| | | GLEWLGVIWGSETTYYNSALKSRLTIIKDNSKSQVFLKMNSLQTDDTAIYYCAKHY |
| | | YYGGSYAMDYWGQGTSVTVSSE |
| mCAR2 | SEQ ID | DIQMTQTTSSLSASLGDRVTISCRASQDISKYLNWYQQKPDGTVKLLIYHTSRLHS |
| CAR - aa | NO: 185 | GVPSRFSGSGSGTDYSLTISNLEQEDIATYFCQQGNTLPYTFGGGTKLEITGSTSG |
| | | SGKPGSGEGSTKGEVKLQESGPGLVAPSQSLSVTCTVSGVSLPDYGVSWIRQPPRK |
| | | GLEWLGVIWGSETTYYNSALKSRLTIIKDNSKSQVFLKMNSLQTDDTAIY |
| | | YCAKHYYYGGSYAMDYWGQGTSVTVSSESKYGPPCPPCPMFWVLVVVGGVLACYSL |
| | | LVTVAFIIFWVKRGRKKLLYIFKQPFMRPVQTTQEEDGCSCRFEEEEGGCELRVKF |
| | | SRSADAPAYQQGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHMQALPPRL |
| mCAR2 | SEQ ID | DIQMTQTT SSLSASLGDR VTISCRASQD ISKYLNWYQQ KPDGTVKLLI |
| Full - aa | NO: 186 | YHTSRLHSGV PSRFSGSGSG TDYSLTISNL EQEDIATYFC QQGNTLPYTF |
| | | GGGTKLEITG STSGSGKPGS GEGSTKGEVK LQESGPGLVA PSQSLSVTCT |
| | | VSGVSLPDYG VSWIRQPPRK GLEWLGVIWG SETTYYNSAL KSRLTIIKDN |
| | | SKSQVFLKMN SLQTDDTAIY YCAKHYYYGG SYAMDYWGQG TSVTVSSESK |
| | | YGPPCPPCPM FWVLVVVGGV LACYSLLVTV |
| | | AFIIFWVKRG RKKLLYIFKQ PFMRPVQTTQ EEDGCSCRFE EEEGGCELRV |
| | | KFSRSADAPA YQQGQNQLYN ELNLGRREEY DVLDKRRGRD PEMGGKPRRK |
| | | NPQEGLYNEL QKDKMAEAYS EIGMKGERRR GKGHDGLYQG LSTATKDTYD |
| | | ALHMQALPPR LEGGGEGRGS LLTCGDVEEN PGPRMLLLVT SLLLCELPHP |
| | | AFLLIPRKVC NGIGIGEFKD SLSINATNIK HFKNCTSISG DLHILPVAFR |
| | | GDSFTHTPPL DPQELDILKT VKEITGFLLI QAWPENRTDL HAFENLEIIR |
| | | GRTKQHGQFS LAVVSLNITS LGLRSLKEIS DGDVIISGNK NLCYANTINW |
| | | KKLFGTSGQK TKIISNRGEN SCKATGQVCH ALCSPEGCWG PEPRDCVSCR |
| | | NVSRGRECVD KCNLLEGEPR EFVENSECIQ CHPECLPQAM NITCTGRGPD |
| | | NCIQCAHYID GPHCVKTCPA GVMGENNTLV WKYADAGHVC HLCHPNCTYG |
| | | CTGPGLEGCP TNGPKIPSIA TGMVGALLLL LVVALGIGLF M |
| mCAR3 | SEQ ID | DIQMTQTTSSLSASLGDRVTISCRASQDISKYLNWYQQKPDGTVKLLIYHTSRLHS |
| scFv | NO: 126 | GVPSRFSGSGSGTDYSLTISNLEQEDIATYFCQQGNTLPYTFGGGTKLEITGSTSG |
| | | SGKPGSGEGSTKGEVKLQESGPGLVAPSQSLSVTCTVSGVSLPDYGVSWIRQPPRK |
| | | GLEWLGVIWGSETTYYNSALKSRLTIIKDNSKSQVFLKMNSLQTDDTAIYYCAKHY |
| | | YYGGSYAMDYWGQGTSVTVSS |
| mCAR3 | SEQ ID | DIQMTQTTSSLSASLGDRVTISCRASQDISKYLNWYQQKPDGTVKLLIYHTSRLHS |
| Full - aa | NO: 175 | GVPSRFSGSGSGTDYSLTISNLEQEDIATYFCQQGNTLPYTFGGGTKLEITGSTSG |
| | | SGKPGSGEGSTKGEVKLQESGPGLVAPSQSLSVTCTVSGVSLPDYGVSWIRQPPRK |
| | | GLEWLGVIWGSETTYYNSALKSRLTIIKDNSKSQVFLKMNSLQTDDTAIYYCAKHY |
| | | YYGGSYAMDYWGQGTSVTVSSAAAIEVMYPPPYLDNEKSNGTIIHVKGKHLCPSPL |
| | | FPGPSKPFWVLVVVGGVLACYSLLVTVAFIIFWVRSKRSRLLHSDYMNMTPRRPGP |
| | | TRKHYQPYAPPRDFAAYRSRVKFSRSADAPAYQQGQNQLYNELNLGRREEYDVLDK |
| | | RRGRDPEMGGKPRRKNPQEGLYNELQKDKMAEAYSEIGMKGERRRGKGHDGLYQGL |
| | | STATKDTYDALHMQALPPR |

In some embodiments, the antigen binding domain comprises a HC CDR1, a HC CDR2, and a HC CDR3 of any heavy chain binding domain amino acid sequences listed in Table 1 or Table 2. In embodiments, the antigen binding domain further comprises a LC CDR1, a LC CDR2, and a LC CDR3. In embodiments, the antigen binding domain comprises a LC CDR1, a LC CDR2, and a LC CDR3 of any light chain binding domain amino acid sequences listed in Table 1 or Table 2.

In some embodiments, the antigen binding domain comprises one, two or all of LC CDR1, LC CDR2, and LC CDR3 of any light chain binding domain amino acid sequences listed in Table 1, and one, two or all of HC CDR1, HC CDR2, and HC CDR3 of any heavy chain binding domain amino acid sequences listed in Table 1.

In some embodiments, the antigen binding domain comprises one, two or all of LC CDR1, LC CDR2, and LC CDR3 of any light chain binding domain amino acid sequences listed in Table 2, and one, two or all of HC CDR1, HC CDR2, and HC CDR3 of any heavy chain binding domain amino acid sequences listed in Table 2.

Any CD19 CAR, e.g., the CD19 antigen binding domain of any known CD19 CAR, can be used in accordance with the present disclosure. For example, LG-740; CD19 CAR described in the U.S. Pat. Nos. 8,399,645; 7,446,190; Xu et al., Leuk Lymphoma. 2013 54(2):255-260(2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122 (25):4129-39(2013); and 16th Annu Meet Am Soc Gen Cell Ther (ASGCT) (May 15-18, Salt Lake City) 2013, Abst 10.

In one embodiment, the CAR T cell that specifically binds to CD19 has the USAN designation TISAGENLECLEUCEL-T. CTL019 is made by a gene modification of T cells is mediated by stable insertion via transduction with a self-inactivating, replication deficient Lentiviral (LV) vector containing the CTL019 transgene under the control of the EF-1 alpha promoter. CTL019 can be a mixture of transgene positive and negative T cells that are delivered to the subject on the basis of percent transgene positive T cells.

In other embodiments, the CAR-expressing cells can specifically bind to human CD19, e.g., can include a CAR molecule, or an antigen binding domain (e.g., a humanized antigen binding domain) according to Table 3 of WO2014/153270, incorporated herein by reference.

In one aspect, the anti-tumor antigen binding domain is a fragment, e.g., a single chain variable fragment (scFv). In one aspect, the anti-a cancer associate antigen as described herein binding domain is a Fv, a Fab, a (Fab′)2, or a bi-functional (e.g. bi-specific) hybrid antibody (e.g., Lanzavecchia et al., Eur. J. Immunol. 17, 105 (1987)). In one aspect, the antibodies and fragments thereof of the invention binds a cancer associate antigen as described herein protein with wild-type or enhanced affinity.

In some instances, scFvs can be prepared according to a method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, which are incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)n, where n is a positive integer equal to or greater than 1 (SEQ ID NO:25). In one embodiment, the linker can be (Gly₄Ser)₄ (SEQ ID NO:27) or (Gly₄Ser)₃ (SEQ ID NO:28). Variation in the linker length may retain or enhance activity, giving rise to superior efficacy in activity studies.

In another aspect, the antigen binding domain is a T cell receptor (“TCR”), or a fragment thereof, for example, a single chain TCR (scTCR). Methods to make such TCRs are known in the art. See, e.g., Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Vα and Vβ genes from a T cell clone linked by a linker (e.g., a flexible peptide). This approach is very useful to cancer associated target that itself is intracellular, however, a fragment of such antigen (peptide) is presented on the surface of the cancer cells by MHC.

### Transmembrane Domain

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one aspect, the transmembrane domain is one that is associated with one of the other domains of the CAR. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CART.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the CAR has bound to a target. A transmembrane domain of particular use in this invention may include at least the transmembrane region(s) of e.g., the alpha, beta or zeta chain of the T-cell receptor, CD28, CD27, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIR2DS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C, or CD19.

In one embodiment, the CAR molecule comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154. In one embodiment, the transmembrane domain comprises a sequence of SEQ ID NO: 6. In one embodiment, the transmembrane domain comprises an amino acid sequence having at least one, two or three modifications (e.g., substitutions) but not more than 20, 10 or 5 modifications (e.g., substitutions) of an amino acid sequence of SEQ ID NO: 6, or a sequence with 95-99% identity to an amino acid sequence of SEQ ID NO: 6.

In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge, e.g., an IgG4 hinge, or a CD8a hinge. In one embodiment, the hinge or spacer comprises (e.g., consists of) the amino acid sequence of SEQ ID NO:2. In one aspect, the transmembrane domain comprises (e.g., consists of) a transmembrane domain of SEQ ID NO: 6.

In one aspect, the hinge or spacer comprises an IgG4 hinge. For example, in one embodiment, the hinge or spacer comprises a hinge of the amino acid sequence ESKYGPPCPPCPAPEFLGGPSVFLFPPKPKDTLMISRTPEVTCVVVDVSQEDPEVQFNW YVDGVEVHNAKTKPREEQFNSTYRVVSVLTVLHQDWLNGKEYKCKVSNKGLPSSIEK TISKAKGQPREPQVYTLPPSQEEMTKNQVSLTCLVKGFYPSDIAVEWESNGQPENNYK TTPPVLDSDGSFFLYSRLTVDKSRWQEGNVFSCSVMHEALHNHYTQKSLSLSLGKM (SEQ ID NO:36). In some embodiments, the hinge or spacer comprises a hinge encoded by a nucleotide sequence of

(SEQ ID NO: 37)
GAGAGCAAGTACGGCCCTCCCTGCCCCCCTTGCCCTGCCCCCGAGTTCCT
GGGCGGACCCAGCGTGTTCCTGTTCCCCCCCAAGCCCAAGGACACCCTGA
TGATCAGCCGGACCCCCGAGGTGACCTGTGTGGTGGTGGACGTGTCCCAG
GAGGACCCCGAGGTCCAGTTCAACTGGTACGTGGACGGCGTGGAGGTGCA
CAACGCCAAGACCAAGCCCCGGGAGGAGCAGTTCAATAGCACCTACCGGG
TGGTGTCCGTGCTGACCGTGCTGCACCAGGACTGGCTGAACGGCAAGGAA
TACAAGTGTAAGGTGTCCAACAAGGGCCTGCCCAGCAGCATCGAGAAAAC
CATCAGCAAGGCCAAGGGCCAGCCTCGGGAGCCCCAGGTGTACACCCTGC
CCCCTAGCCAAGAGGAGATGACCAAGAACCAGGTGTCCCTGACCTGCCTG
GTGAAGGGCTTCTACCCCAGCGACATCGCCGTGGAGTGGGAGAGCAACGG
CCAGCCCGAGAACAACTACAAGACCACCCCCCCTGTGCTGGACAGCGACG
GCAGCTTCTTCCTGTACAGCCGGCTGACCGTGGACAAGAGCCGGTGGCAG
GAGGGCAACGTCTTTAGCTGCTCCGTGATGCACGAGGCCCTGCACAACCA
CTACACCCAGAAGAGCCTGAGCCTGTCCCTGGGCAAGATG.

In one aspect, the hinge or spacer comprises an IgD hinge. For example, in one embodiment, the hinge or spacer comprises a hinge of the amino acid sequence RWPESPKAQASSVPTAQPQAEGSLAKATTAPATTRNTGRGGEEKKKEKEKEEQEERET KTPECPSHTQPLGVYLLTPAVQDLWLRDKATFTCFVVGSDLKDAHLTWEVAGKVPTG GVEEGLLERHSNGSQSQHSRLTLPRSLWNAGTSVTCTLNHPSLPPQRLMALREPAAQA PVKLSLNLLASSDPPEAASWLLCEVSGFSPPNILLMWLEDQREVNTSGFAPARPPPQPG STTFWAWSVLRVPAPPSPQPATYTCVVSHEDSRTLLNASRSLEVSYVTDH (SEQ ID NO:38). In some embodiments, the hinge or spacer comprises a hinge encoded by a nucleotide sequence of

(SEQ ID NO: 103)
AGGTGGCCCGAAAGTCCCAAGGCCCAGGCATCTAGTGTTCCTACTGCACA
GCCCCAGGCAGAAGGCAGCCTAGCCAAAGCTACTACTGCACCTGCCACTA
CGCGCAATACTGGCCGTGGCGGGGAGGAGAAGAAAAAGGAGAAAGAGAAA
GAAGAACAGGAAGAGAGGGAGACCAAGACCCCTGAATGTCCATCCCATAC
CCAGCCGCTGGGCGTCTATCTCTTGACTCCCGCAGTACAGGACTTGTGGC
TTAGAGATAAGGCCACCTTTACATGTTTCGTCGTGGGCTCTGACCTGAAG
GATGCCCATTTGACTTGGGAGGTTGCCGGAAAGGTACCCACAGGGGGGGT
TGAGGAAGGGTTGCTGGAGCGCCATTCCAATGGCTCTCAGAGCCAGCACT
CAAGACTCACCCTTCCGAGATCCCTGTGGAACGCCGGGACCTCTGTCACA
TGTACTCTAAATCATCCTAGCCTGCCCCCACAGCGTCTGATGGCCCTTAG
AGAGCCAGCCGCCCAGGCACCAGTTAAGCTTAGCCTGAATCTGCTCGCCA
GTAGTGATCCCCCAGAGGCCGCCAGCTGGCTCTTATGCGAAGTGTCCGGC
TTTAGCCCGCCCAACATCTTGCTCATGTGGCTGGAGGACCAGCGAGAAGT
GAACACCAGCGGCTTCGCTCCAGCCCGGCCCCCACCCCAGCCGGGTTCTA
CCACATTCTGGGCCTGGAGTGTCTTAAGGGTCCCAGCACCACCTAGCCCC
CAGCCAGCCACATACACCTGTGTTGTGTCCCATGAAGATAGCAGGACCCT
GCTAAATGCTTCTAGGAGTCTGGAGGTTTCCTACGTGACTGACCATT.

In one aspect, the transmembrane domain may be recombinant, in which case it will comprise predominantly hydrophobic residues such as leucine and valine. In one aspect a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in one aspect, the linker comprises the amino acid sequence of GGGGSGGGGS (SEQ ID NO: 5). In some embodiments, the linker is encoded by a nucleotide sequence of

(SEQ ID NO: 8)
GGTGGCGGAGGTTCTGGAGGTGGAGGTTCC.

In one aspect, the hinge or spacer comprises a KIR2DS2 hinge.

### Cytoplasmic Domain

The cytoplasmic domain or region of the CAR includes an intracellular signaling domain. An intracellular signaling domain is generally responsible for activation of at least one of the normal effector functions of the immune cell in which the CAR has been introduced.

Examples of intracellular signaling domains for use in a CAR described herein include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the invention include those of TCR zeta, FcR gamma, FcR beta, CD3 gamma, CD3 delta, CD3 epsilon, CD5, CD22, CD79a, CD79b, CD278 (also known as “ICOS”), FcεRI, DAP10, DAP12, and CD66d. In one embodiment, a CAR of the invention comprises an intracellular signaling domain, e.g., a primary signaling domain of CD3-zeta, e.g., a CD3-zeta sequence described herein.

In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

### Costimulatory Signaling Domain

The intracellular signalling domain of the CAR can comprise the CD3-zeta signaling domain by itself or it can be combined with any other desired intracellular signaling domain(s) useful in the context of a CAR of the invention. For example, the intracellular signaling domain of the CAR can comprise a CD3 zeta chain portion and a costimulatory signaling domain. The costimulatory signaling domain refers to a portion of the CAR comprising the intracellular domain of a costimulatory molecule. In one embodiment, the intracellular domain is designed to comprise the signaling domain of CD3-zeta and the signaling domain of CD28. In one aspect, the intracellular domain is designed to comprise the signaling domain of CD3-zeta and the signaling domain of ICOS.

A costimulatory molecule can be a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). Further examples of such costimulatory molecules include CDS, ICAM-1, GITR, BAFFR, HVEM (LIGHTR), SLAMF7, NKp80 (KLRF1), NKp30, NKp44, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, NKG2D, NKG2C and PAG/Cbp.

The intracellular signaling sequences within the cytoplasmic portion of the CAR may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequences. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue.

In one aspect, the intracellular signaling domain is designed to comprise the signaling domain of CD3-zeta and the signaling domain of CD28.

In one aspect, the intracellular signaling domain is designed to comprise the signaling domain of CD3-zeta and the signaling domain of 4-1BB.

In one embodiment, the signaling domain of 4-1BB is a signaling domain of SEQ ID NO: 7. In one embodiment, the 4-1BB costimulatory domain comprises a sequence of SEQ ID NO: 7. In one embodiment, the 4-1BB costimulatory domain comprises an amino acid sequence having at least one, two or three modifications (e.g., substitutions) but not more than 20, 10 or 5 modifications (e.g., substitutions) of an amino acid sequence of SEQ ID NO: 7, or a sequence with at least 95%, e.g., 95-99%, identity to an amino acid sequence of SEQ ID NO:7.

In one embodiment, the signaling domain of CD3-zeta is a signaling domain of SEQ ID NO: 9. In one embodiment, the CD3-zeta costimulatory domain comprises a sequence of SEQ ID NO: 9. In one embodiment, the CD3-zeta costimulatory domain comprises an amino acid sequence having at least one, two or three modifications (e.g., substitutions) but not more than 20, 10 or 5 modifications (e.g., substitutions) of an amino acid sequence of SEQ ID NO: 9, or a sequence with at least 95%, e.g., 95-99%, identity to an amino acid sequence of SEQ ID NO:9.

In one aspect, the intracellular signaling domain is designed to comprise the signaling domain of CD3-zeta and the signaling domain of CD27. In one aspect, the signaling domain of CD27 comprises an amino acid sequence of

(SEQ ID NO: 16)
QRRKYRSNKGESPVEPAEPCRYSCPREEEGSTIPIQEDYRKPEPACSP.

In one aspect, the signalling domain of CD27 is encoded by a nucleic acid sequence of

(SEQ ID NO: 14)
AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC.

In one embodiment, the CD27 costimulatory domain comprises a sequence of SEQ ID NO: 14. In one embodiment, the CD27 costimulatory domain comprises an amino acid sequence having at least one, two or three modifications (e.g., substitutions) but not more than 20, 10 or 5 modifications (e.g., substitutions) of an amino acid sequence of SEQ ID NO: 14, or a sequence with at least 95%, e.g., 95-99%, identity to an amino acid sequence of SEQ ID NO:14.

In one aspect, the CAR-expressing cell described herein can further comprise a second CAR, e.g., a second CAR that includes a different antigen binding domain, e.g., to the same target or a different target (e.g., a target other than a cancer associated antigen described herein or a different cancer associated antigen described herein, e.g., CD19, CD33, CLL-1, CD34, FLT3, or folate receptor beta). In one embodiment, the second CAR includes an antigen binding domain to a target expressed the same cancer cell type as the cancer associated antigen. In one embodiment, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. While not wishing to be bound by theory, placement of a costimulatory signaling domain, e.g., 4-1BB, CD28, ICOS, CD27 or OX-40, onto the first CAR, and the primary signaling domain, e.g., CD3 zeta, on the second CAR can limit the CAR activity to cells where both targets are expressed. In one embodiment, the CAR expressing cell comprises a first cancer associated antigen CAR that includes an antigen binding domain that binds a target antigen described herein, a transmembrane domain and a costimulatory domain and a second CAR that targets a different target antigen (e.g., an antigen expressed on that same cancer cell type as the first target antigen) and includes an antigen binding domain, a transmembrane domain and a primary signaling domain. In another embodiment, the CAR expressing cell comprises a first CAR that includes an antigen binding domain that binds a target antigen described herein, a transmembrane domain and a primary signaling domain and a second CAR that targets an antigen other than the first target antigen (e.g., an antigen expressed on the same cancer cell type as the first target antigen) and includes an antigen binding domain to the antigen, a transmembrane domain and a costimulatory signaling domain.

In another aspect, the disclosure features a population of CAR-expressing cells, e.g., CART cells. In some embodiments, the population of CAR-expressing cells comprises a mixture of cells expressing different CARs.

For example, in one embodiment, the population of CART cells can include a first cell expressing a CAR having an antigen binding domain to a cancer associated antigen described herein, and a second cell expressing a CAR having a different antigen binding domain, e.g., an antigen binding domain to a different a cancer associated antigen described herein, e.g., an antigen binding domain to a cancer associated antigen described herein that differs from the cancer associate antigen bound by the antigen binding domain of the CAR expressed by the first cell.

As another example, the population of CAR-expressing cells can include a first cell expressing a CAR that includes an antigen binding domain to a cancer associated antigen described herein, and a second cell expressing a CAR that includes an antigen binding domain to a target other than a cancer associate antigen as described herein. In one embodiment, the population of CAR-expressing cells includes, e.g., a first cell expressing a CAR that includes a primary intracellular signaling domain, and a second cell expressing a CAR that includes a secondary signaling domain.

In another aspect, the disclosure features a population of cells wherein at least one cell in the population expresses a CAR having an antigen binding domain to a cancer associated antigen described herein, and a second cell expressing another agent, e.g., an agent which enhances the activity of a CAR-expressing cell. For example, in one embodiment, the agent can be an agent which inhibits an inhibitory molecule. Inhibitory molecules, e.g., PD-1, can, in some embodiments, decrease the ability of a CAR-expressing cell to mount an immune effector response. Examples of inhibitory molecules include PD-1, PD-L1, PD-L2, CTLA4, TIM3, CEACAM (CEACAM-1, CEACAM-3, and/or CEACAM-5), LAGS, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF (e.g., TGF beta). In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD-1, PD-L1, CTLA4, TIM3, CEACAM (CEACAM-1, CEACAM-3, and/or CEACAM-5), LAGS, VISTA, BTLA, TIGIT, LAIR1, CD160, 2B4 and TGF beta, or a fragment of any of these, and a second polypeptide which is an intracellular signaling domain described herein (e.g., comprising a costimulatory domain (e.g., 41BB, CD27, OX40 or CD28, e.g., as described herein) and/or a primary signaling domain (e.g., a CD3 zeta signaling domain described herein). In one embodiment, the agent comprises a first polypeptide of PD-1 or a fragment thereof, and a second polypeptide of an intracellular signaling domain described herein (e.g., a CD28 signaling domain described herein and/or a CD3 zeta signaling domain described herein).

The sequences of anti-CD19 binding domains are provided herein in Table 1. Full CAR constructs can be generated using any of the antigen binding domains described in Table 1 with one or more additional CAR component provided below.

leader (amino acid sequence)(SEQ ID NO: 1)
MALPVTALLLPLALLLHAARP
leader (nucleic acid sequence)(SEQ ID NO: 12)
ATGGCCCTGCCTGTGACAGCCCTGCTGCTGCCTCTGGCTCTGCTGCTGCATG
CCGCTAGACCC
leader (nucleic acid sequence 2)(SEQ ID NO: 127)
ATGGCCCTCCCTGTCACCGCCCTGCTGCTTCCGCTGGCTCTTCTGCTCCACGCCGCT
CGGCCC
leader (nucleic acid sequence 3)(SEQ ID NO: 128)
ATGGCCTTACCAGTGACCGCCTTGCTCCTGCCGCTGGCCTTGCTGCTCCACGCCGCC
AGGCCG
CD8 hinge (amino acid sequence)(SEQ ID NO: 2)
TTTPAPRPPTPAPTIASQPLSLRPEACRPAAGGAVHTRGLDFACD
CD8 hinge (nucleic acid sequence)(SEQ ID NO: 13)
ACCACGACGCCAGCGCCGCGACCACCAACACCGGCGCCCACCATCGCGTC
GCAGCCCCTGTCCCTGCGCCCAGAGGCGTGCCGGCCAGCGGCGGGGGGCGCAGTG
CACACGAGGGGGCTGGACTTCGCCTGTGAT
CD8 hinge (nucleic acid sequence 2)(SEQ ID NO: 129)
ACCACTACCCCAGCACCGAGGCCACCCACCCCGGCTCCTACCATCGCCTCC
CAGCCTCTGTCCCTGCGTCCGGAGGCATGTAGACCCGCAGCTGGTGGGGCCGTGCA
TACCCGGGGTCTTGACTTCGCCTGCGAT
CD8 transmembrane (amino acid sequence)(SEQ ID NO: 6)
IYIWAPLAGTCGVLLLSLVITLYC
transmembrane (nucleic acid sequence)(SEQ ID NO: 17)
ATCTACATCTGGGCGCCCTTGGCCGGGACTTGTGGGGTCCTTCTCCTGTCAC
TGGTTATCACCCTTTACTGC
transmembrane (nucleic acid sequence 2)(SEQ ID NO: 130)
ATCTACATTTGGGCCCCTCTGGCTGGTACTTGCGGGGTCCTGCTGCTTTCAC
TCGTGATCACTCTTTACTGT
4-1BB Intracellular domain (amino acid sequence)(SEQ ID NO: 7)
KRGRKKLLYIFKQPFMRPVQTTQEEDGCSCRFPEEEEGGCEL
4-1BB Intracellular domain (nucleic acid sequence)(SEQ ID NO: 18)
AAACGGGGCAGAAAGAAACTCCTGTATATATTCAAACAACCATTTATGAG
ACCAGTACAAACTACTCAAGAGGAAGATGGCTGTAGCTGCCGATTTCCAGAAGAA
GAAGAAGGAGGATGTGAACTG
4-1BB Intracellular domain (nucleic acid sequence 2)(SEQ ID NO: 131)
AAGCGCGGTCGGAAGAAGCTGCTGTACATCTTTAAGCAACCCTTCATGAGG
CCTGTGCAGACTACTCAAGAGGAGGACGGCTGTTCATGCCGGTTCCCAGAGGAGG
AGGAAGGCGGCTGCGAACTG
CD3 zeta domain (amino acid sequence)(SEQ ID NO: 9)
RVKFSRSADAPAYKQGQNQLYNELNLGRREEYDVLDKRRGRDPEMGGKPRR
KNPQEGLYNELQKDKMAEAYSEIGMKGERRRGKGHDGLYQGLSTATKDTYDALHM
QALPPR
CD3 zeta (nucleic acid sequence)(SEQ ID NO: 20)
AGAGTGAAGTTCAGCAGGAGCGCAGACGCCCCCGCGTACAAGCAGGGCCA
GAACCAGCTCTATAACGAGCTCAATCTAGGACGAAGAGAGGAGTACGATGTTTTG
GACAAGAGACGTGGCCGGGACCCTGAGATGGGGGGAAAGCCGAGAAGGAAGAAC
CCTCAGGAAGGCCTGTACAATGAACTGCAGAAAGATAAGATGGCGGAGGCCTACA
GTGAGATTGGGATGAAAGGCGAGCGCCGGAGGGGCAAGGGGCACGATGGCCTTTA
CCAGGGTCTCAGTACAGCCACCAAGGACACCTACGACGCCCTTCACATGCAGGCCC
TGCCCCCTCGC
CD3 zeta (nucleic acid sequence 2)(SEQ ID NO: 132)
CGCGTGAAATTCAGCCGCAGCGCAGATGCTCCAGCCTACAAGCAGGGGCA
GAACCAGCTCTACAACGAACTCAATCTTGGTCGGAGAGAGGAGTACGACGTGCTG
GACAAGCGGAGAGGACGGGACCCAGAAATGGGCGGGAAGCCGCGCAGAAAGAAT
CCCCAAGAGGGCCTGTACAACGAGCTCCAAAAGGATAAGATGGCAGAAGCCTATA
GCGAGATTGGTATGAAAGGGGAACGCAGAAGAGGCAAAGGCCACGACGGACTGT
ACCAGGGACTCAGCACCGCCACCAAGGACACCTATGACGCTCTTCACATGCAGGC
CCTGCCGCCTCGG
CD3 zeta domain (amino acid sequence; NCBI Reference Sequence NM_000734.3)(SEQ
ID NO: 10)
RVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYDALHM
QALPPR
CD3 zeta (nucleic acid sequence; NCBI Reference Sequence NM_000734.3); (SEQ
ID NO: 21)
agagtgaagttcagcaggagcgcagacgcccccgcgtaccagcagggccagaaccagctctataacgagctcaatctag
gacgaagagaggagtacgatgttttggacaagagacgtggccgggaccctgagatggggggaaagccgagaaggaagaaccctca
ggaaggcctgtacaatgaactgcagaaagataagatggcggaggcctacagtgagattgggatgaaaggcgagcgccggaggggca
aggggcacgatggcctttaccagggtctcagtacagccaccaaggacacctacgacgcccttcacatgcaggccctgccccctcgc
IgG4 Hinge (amino acid sequence) (SEQ ID NO: 36)
ESKYGPPCPPCPAPEFLGGPSVFLFPPKPKDTLMISRTPEVTCVVVDVSQEDPEV
QFNWYVDGVEVHNAKTKPREEQFNSTYRVVSVLTVLHQDWLNGKEYKCKVSNKGLP
SSIEKTISKAKGQPREPQVYTLPPSQEEMTKNQVSLTCLVKGFYPSDIAVEWESNGQPE
NNYKTTPPVLDSDGSFFLYSRLTVDKSRWQEGNVFSCSVMHEALHNHYTQKSLSLSLG
KM
IgG4 Hinge (nucleotide sequence)(SEQ ID NO: 37)
GAGAGCAAGTACGGCCCTCCCTGCCCCCCTTGCCCTGCCCCCGAGTTCCTG
GGCGGACCCAGCGTGTTCCTGTTCCCCCCCAAGCCCAAGGACACCCTGATGATCAG
CCGGACCCCCGAGGTGACCTGTGTGGTGGTGGACGTGTCCCAGGAGGACCCCGAG
GTCCAGTTCAACTGGTACGTGGACGGCGTGGAGGTGCACAACGCCAAGACCAAGC
CCCGGGAGGAGCAGTTCAATAGCACCTACCGGGTGGTGTCCGTGCTGACCGTGCTG
CACCAGGACTGGCTGAACGGCAAGGAATACAAGTGTAAGGTGTCCAACAAGGGCC
TGCCCAGCAGCATCGAGAAAACCATCAGCAAGGCCAAGGGCCAGCCTCGGGAGCC
CCAGGTGTACACCCTGCCCCCTAGCCAAGAGGAGATGACCAAGAACCAGGTGTCC
CTGACCTGCCTGGTGAAGGGCTTCTACCCCAGCGACATCGCCGTGGAGTGGGAGA
GCAACGGCCAGCCCGAGAACAACTACAAGACCACCCCCCCTGTGCTGGACAGCGA
CGGCAGCTTCTTCCTGTACAGCCGGCTGACCGTGGACAAGAGCCGGTGGCAGGAG
GGCAACGTCTTTAGCTGCTCCGTGATGCACGAGGCCCTGCACAACCACTACACCCA
GAAGAGCCTGAGCCTGTCCCTGGGCAAGATG
EF1 alpha promoter (SEQ ID NO: 11)
CGTGAGGCTCCGGTGCCCGTCAGTGGGCAGAGCGCACATCGCCCACAGTCC
CCGAGAAGTTGGGGGGAGGGGTCGGCAATTGAACCGGTGCCTAGAGAAGGTGGCG
CGGGGTAAACTGGGAAAGTGATGTCGTGTACTGGCTCCGCCTTTTTCCCGAGGGTG
GGGGAGAACCGTATATAAGTGCAGTAGTCGCCGTGAACGTTCTTTTTCGCAACGGG
TTTGCCGCCAGAACACAGGTAAGTGCCGTGTGTGGTTCCCGCGGGCCTGGCCTCTT
TACGGGTTATGGCCCTTGCGTGCCTTGAATTACTTCCACCTGGCTGCAGTACGTGAT
TCTTGATCCCGAGCTTCGGGTTGGAAGTGGGTGGGAGAGTTCGAGGCCTTGCGCTT
AAGGAGCCCCTTCGCCTCGTGCTTGAGTTGAGGCCTGGCCTGGGCGCTGGGGCCGC
CGCGTGCGAATCTGGTGGCACCTTCGCGCCTGTCTCGCTGCTTTCGATAAGTCTCTA
GCCATTTAAAATTTTTGATGACCTGCTGCGACGCTTTTTTTCTGGCAAGATAGTCTT
GTAAATGCGGGCCAAGATCTGCACACTGGTATTTCGGTTTTTGGGGCCGCGGGCGG
CGACGGGGCCCGTGCGTCCCAGCGCACATGTTCGGCGAGGCGGGGCCTGCGAGCG
CGGCCACCGAGAATCGGACGGGGGTAGTCTCAAGCTGGCCGGCCTGCTCTGGTGC
CTGGCCTCGCGCCGCCGTGTATCGCCCCGCCCTGGGCGGCAAGGCTGGCCCGGTCG
GCACCAGTTGCGTGAGCGGAAAGATGGCCGCTTCCCGGCCCTGCTGCAGGGAGCT
CAAAATGGAGGACGCGGCGCTCGGGAGAGCGGGCGGGTGAGTCACCCACACAAA
GGAAAAGGGCCTTTCCGTCCTCAGCCGTCGCTTCATGTGACTCCACGGAGTACCGG
GCGCCGTCCAGGCACCTCGATTAGTTCTCGAGCTTTTGGAGTACGTCGTCTTTAGGT
TGGGGGGAGGGGTTTTATGCGATGGAGTTTCCCCACACTGAGTGGGTGGAGACTG
AAGTTAGGCCAGCTTGGCACTTGATGTAATTCTCCTTGGAATTTGCCCTTTTTGAGT
TTGGATCTTGGTTCATTCTCAAGCCTCAGACAGTGGTTCAAAGTTTTTTTCTTCCAT
TTCAGGTGTCGTGA.
Gly/Ser (SEQ ID NO: 25)
GGGGS
Gly/Ser (SEQ ID NO: 26): This sequence may encompass 1-6 “Gly Gly Gly Gly Ser” repeating
units
GGGGSGGGGS GGGGSGGGGS GGGGSGGGGS
Gly/Ser (SEQ ID NO: 27)
GGGGSGGGGS GGGGSGGGGS
Gly/Ser (SEQ ID NO: 28)
GGGGSGGGGS GGGGS
Gly/Ser (SEQ ID NO: 29)
GGGS
PolyA (SEQ ID NO: 30): A5000
PolyA (SEQ ID NO: 31): A100
PolyT (SEQ ID NO: 32): T5000
PolyA (SEQ ID NO: 33): A5000
PolyA (SEQ ID NO: 34): A400
PolyA (SEQ ID NO: 35): A2000
Gly/Ser (SEQ ID NO: 15): This sequence may encompass 1-10 “Gly Gly Gly Ser”
repeating units
GGGSGGGSGG GSGGGSGGGS GGGSGGGSGG GSGGGSGGGS

Exemplary CD19 CAR constructs that can be used in the methods described herein are shown in Table 3:

TABLE 3
Exemplary CD19 CAR Constructs

| | | |
|---|---|---|
| | SEQ ID | |
| Name | NO: | Sequence |

CAR1

| | | |
|---|---|---|
| CAR1 scFv | 39 | EIVMTQSPATLSLSPGERATLSCRASQDISKYLNWYQQKPGQAPRLLIYHT |
| domain | | SRLHSGIPARFSGSGSGTDYTLTISSLQPEDFAVYFCQQGNTLPYTFGQGT |
| | | KLEIKGGGGSGGGGSGGGGSQVQLQESGPGLVKPSETLSLTCTVSGVSLPD |
| | | YGVSWIRQPPGKGLEWIGVIWGSETTYYSSSLKSRVTISKDNSKNQVSLKL |
| | | SSVTAADTAVYYCAKHYYYGGSYAMDYWGQGTLVTVSS |
| 103101 | 52 | atggccctccctgtcaccgccctgctgcttccgctggctcttctgctccacgccgc |
| CARI | | tcggcccgaaattgtgatgacccagtcacccgccactcttagcctttcacccggtg |
| Soluble | | agcgcgcaaccctgtcttgcagagcctcccaagacatctcaaaataccttaattgg |
| scFv - nt | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagccaggtccaactccaagaaa |
| | | gcggaccgggtcttgtgaagccatcagaaactctttcactgacttgtactgtgagc |
| | | ggagtgtctctccccgattacggggtgtcttggatcagacagccaccggggaaggg |
| | | tctggaatggattggagtgatttggggctctgagactacttactactcttcatccc |
| | | tcaagtcacgcgtcaccatctcaaaggacaactctaagaatcaggtgtcactgaaa |
| | | ctgtcatctgtgaccgcagccgacaccgccgtgtactattgcgctaagcattacta |
| | | ttatggcgggagctacgcaatggattactggggacagggtactctggtcaccgtgt |
| | | ccagccaccaccatcatcaccatcaccat |
| 103101 | 64 | MALPVTALLLPLALLLHAARPeivmtqspatlslspgeratlscrasqdiskylnw |
| CAR1 | | yqqkpgqaprlliyhtsrlhsgiparfsgsgsgtdytitisslqpedfavyfcqqg |
| Soluble | | ntlpytfgqgtkleikggggsggggsggggsqvqlqesgpglvkpsetlsltctvs |
| scFv - aa | | gvslpdygvswirqppgkglewigviwgsettyyssslksrvtiskdnsknqvslk |
| | | lssvtaadtavyycakhyyyqqsyamdywqqqtlvtvsshhhhhhhh |
| 104875 | 90 | atggccctccctgtcaccgccctgctgcttccgctggctcttctgctccacgccgc |
| CAR 1- | | tcggcccgaaattgtgatgacccagtcacccgccactcttagcctttcacccggtg |
| Full - nt | | agcgcgcaaccctgtcttgcagagcctcccaagacatctcaaaataccttaattgg |
| | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagccaggtccaactccaagaaa |
| | | gcggaccgggtcttgtgaagccatcagaaactctttcactgacttgtactgtgagc |
| | | ggagtgtctctccccgattacggggtgtcttggatcagacagccaccggggaaggg |
| | | tctggaatggattggagtgatttggggctctgagactacttactactcttcatccc |
| | | tcaagtcacgcgtcaccatctcaaaggacaactctaagaatcaggtgtcactgaaa |
| | | ctgtcatctgtgaccgcagccgacaccgccgtgtactattgcgctaagcattacta |
| | | ttatggcgggagctacgcaatggattactggggacagggtactctggtcaccgtgt |
| | | ccagcaccactaccccagcaccgaggccacccaccccggctcctaccatcgcctcc |
| | | cagcctctgtccctgcgtccggaggcatgtagacccgcagctggtggggccgtgca |
| | | tacccggggtcttgacttcgcctgcgatatctacatttgggcccctctggctggta |
| | | cttgcggggtcctgctgctttcactcgtgatcactctttactgtaagcgcggtcgg |
| | | aagaagctgctgtacatctttaagcaacccttcatgaggcctgtgcagactactca |
| | | agaggaggacggctgttcatgccggttcccagaggaggaggaaggcggctgcgaac |
| | | tgcgcgtgaaattcagccgcagcgcagatgctccagcctacaagcaggggcagaac |
| | | cagctctacaacgaactcaatcttggtcggagagaggagtacgacgtgctggacaa |
| | | gcggagaggacgggacccagaaatgggcgggaagccgcgcagaaagaatccccaag |
| | | agggcctgtacaacgagctccaaaaggataagatggcagaagcctatagcgagatt |
| | | ggtatgaaaggggaacgcagaagaggcaaaggccacgacggactgtaccagggact |
| | | cagcaccgccaccaaggacacctatgacgctcttcacatgcaggccctgccgcctc |
| | | gg |
| 104875 | 77 | MALPVTALLLPLALLLHAARPeivmtqspatlslspgeratlscrasqdiskylnw |
| CAR 1- | | yqqkpgqaprlliyhtsrlhsgiparfsgsgsgtdytltisslqpedfavyfcqqg |
| Full - aa | | ntlpytfgqgtkleikggggsggggsggggsqvqlqesgpglvkpsetlsltctvs |
| | | gvslpdygvswirqppgkglewigviwgsettyyssslksrvtiskdnsknqvslk |
| | | lssvtaadtavyycakhyyyggsyamdywgqgtlvtvsstttpaprpptpaptias |
| | | qplslrpeacrpaaggavhtrgldfacdiyiwaplagtcgvlllslvitlyckrgr |
| | | kkllyifkqpfmrpvqttqeedgcscrfpeeeeggcelrvkfsrsadapaykqgqn |
| | | qlynelnlgrreeydvldkrrgrdpemggkprrknpqeglynelqkdkmaeaysei |
| | | gmkgerrrgkghdglyqglstatkdtydalhmqalppr |

CAR 2 (CTL119)

| | | |
|---|---|---|
| CAR2 scFv | 40 | eivmtqspatlslspgeratlscrasqdiskylnwyqqkpgqaprlliyhtsrlhs |
| domain | | giparfsgsgsgtdytltisslqpedfavyfcqqgntlpytfgqgtkleikggggs |
| | | ggggsggggsqvqlqesgpglvkpsetlsltctvsgvslpdygvswirqppgkgle |
| | | wigviwgsettyyqsslksrvtiskdnsknqvslklssvtaadtavyycakhyyyg |
| | | gsyamdywgqgtlvtvss |
| 103102 | 53 | atggccctccctgtcaccgccctgctgcttccgctggctcttctgctccacgccgc |
| CAR2- | | tcggcccgaaattgtgatgacccagtcacccgccactcttagcctttcacccggtg |
| Soluble | | agcgcgcaaccctgtcttgcagagcctcccaagacatctcaaaataccttaattgg |
| scFv - nt | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagccaggtccaactccaagaaa |
| | | gcggaccgggtcttgtgaagccatcagaaactctttcactgacttgtactgtgagc |
| | | ggagtgtctctccccgattacggggtgtcttggatcagacagccaccggggaaggg |
| | | tctggaatggattggagtgatttggggctctgagactacttactaccaatcatccc |
| | | tcaagtcacgcgtcaccatctcaaaggacaactctaagaatcaggtgtcactgaaa |
| | | ctgtcatctgtgaccgcagccgacaccgccgtgtactattgcgctaagcattacta |
| | | ttatggcgggagctacgcaatggattactggggacagggtactctggtcaccgtgt |
| | | ccagccaccaccatcatcaccatcaccat |
| 103102 | 65 | MALPVTALLLPLALLLHAARPeivmtqspatlslspgeratlscrasqdiskylnw |
| CAR2- | | yqqkpgqaprlliyhtsrlhsgiparfsgsgsgtdytltisslqpedfavyfcqqg |
| Soluble | | ntlpytfgqgtkleikggggsggggsggggsqvqlqesgpglvkpsetlsltctvs |
| scFv - aa | | gvslpdygvswirqppgkglewigviwgsettyyqsslksrvtiskdnsknqvslk |
| | | lssvtaadtavyycakhyyyqqsyamdywgqgtlvtvsshhhhhhhh |
| 104876 | 91 | atggccctccctgtcaccgccctgctgcttccgctggctcttctgctccacgccgc |
| CAR 2- | | tcggcccgaaattgtgatgacccagtcacccgccactcttagcctttcacccggtg |
| Full - nt | | agcgcgcaaccctgtcttgcagagcctcccaagacatctcaaaataccttaattgg |
| (CTL119) | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagccaggtccaactccaagaaa |
| | | gcggaccgggtcttgtgaagccatcagaaactctttcactgacttgtactgtgagc |
| | | ggagtgtctctccccgattacggggtgtcttggatcagacagccaccggggaaggg |
| | | tctggaatggattggagtgatttggggctctgagactacttactaccaatcatccc |
| | | tcaagtcacgcgtcaccatctcaaaggacaactctaagaatcaggtgtcactgaaa |
| | | ctgtcatctgtgaccgcagccgacaccgccgtgtactattgcgctaagcattacta |
| | | ttatggcgggagctacgcaatggattactggggacagggtactctggtcaccgtgt |
| | | ccagcaccactaccccagcaccgaggccacccaccccggctcctaccatcgcctcc |
| | | cagcctctgtccctgcgtccggaggcatgtagacccgcagctggtggggccgtgca |
| | | tacccggggtcttgacttcgcctgcgatatctacatttgggcccctctggctggta |
| | | cttgcggggtcctgctgctttcactcgtgatcactctttactgtaagcgcggtcgg |
| | | aagaagctgctgtacatctttaagcaacccttcatgaggcctgtgcagactactca |
| | | agaggaggacggctgttcatgccggttcccagaggaggaggaaggcggctgcgaac |
| | | tgcgcgtgaaattcagccgcagcgcagatgctccagcctacaagcaggggcagaac |
| | | cagctctacaacgaactcaatcttggtcggagagaggagtacgacgtgctggacaa |
| | | gcggagaggacgggacccagaaatgggcgggaagccgcgcagaaagaatccccaag |
| | | agggcctgtacaacgagctccaaaaggataagatggcagaagcctatagcgagatt |
| | | ggtatgaaaggggaacgcagaagaggcaaaggccacgacggactgtaccagggact |
| | | cagcaccgccaccaaggacacctatgacgctcttcacatgcaggccctgccgcctc |
| | | gg |
| 104876 | 78 | MALPVTALLLPLALLLHAARPeivmtqspatlslspgeratlscrasqdiskylnw |
| CAR 2- | | yqqkpgqaprlliyhtsrlhsgiparfsgsgsgtdytltisslqpedfavyfcqqg |
| Full - aa | | ntlpytfgqgtkleikggggsggggsggggsqvqlqesqpglvkpsetlsltctvs |
| (CTL119) | | gvslpdygvswirqppgkglewigviwgsettyyqsslksrvtiskdnsknqvslk |
| | | lssvtaadtavyycakhyyyggsyamdywgqgtlvtvsstttpaprpptpaptias |
| | | qplslrpeacrpaaggavhtrgldfacdiyiwaplagtcgvlllslvitlyckrgr |
| | | kkllyifkqpfmrpvqttqeedgcscrfpeeeeggcelrvkfsrsadapaykqgqn |
| | | qlynelnlgrreeydvldkrrgrdpemggkprrknpqeglynelqkdkmaeaysei |
| | | gmkgerrrgkghdglyqglstatkdtydalhmqalppr |

CAR 3

| | | |
|---|---|---|
| CAR3 scFv | 41 | qvqlqesgpglvkpsetlsltctvsgvslpdygvswirqppgkglewigviwgset |
| domain | | tyyssslksrvtiskdnsknqvslklssvtaadtavyycakhyyyggsyamdywgq |
| | | gtlvtvssggggsggggsggggseivmtqspatlslspgeratlscrasqdiskyl |
| | | nwyqqkpgqaprlliyhtsrlhsgiparfsgsgsgtdytltisslqpedfavyfcq |
| | | qgntlpytfgqgtkleik |
| 103104 | 54 | atggctctgcccgtgaccgcactcctcctgccactggctctgctgcttcacgccgc |
| CAR 3 - | | tcgcccacaagtccagcttcaagaatcagggcctggtctggtgaagccatctgaga |
| Soluble | | ctctgtccctcacttgcaccgtgagcggagtgtccctcccagactacggagtgagc |
| scFv - nt | | tggattagacagcctcccggaaagggactggagtggatcggagtgatttggggtag |
| | | cgaaaccacttactattcatcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccgaaatcgtgatgacccagagccctgcaaccctgtcc |
| | | ctttctcccggggaacgggctaccctttcttgtcgggcatcacaagatatctcaaa |
| | | atacctcaattggtatcaacagaagccgggacaggcccctaggcttcttatctacc |
| | | acacctctcgcctgcatagcgggattcccgcacgctttagcgggtctggaagcggg |
| | | accgactacactctgaccatctcatctctccagcccgaggacttcgccgtctactt |
| | | ctgccagcagggtaacaccctgccgtacaccttcggccagggcaccaagcttgaga |
| | | tcaaacatcaccaccatcatcaccatcac |
| 103104 | 66 | MALPVTALLLPLALLLHAARPqvqlqesgpglvkpsetlsltctvsgvslpdygvs |
| CAR 3 - | | wirqppgkglewigviwgsettyyssslksrvtiskdnsknqvslklssvtaadta |
| Soluble | | vyycakhyyyggsyamdywgqgtlvtvssggggsggggsggggseivmtqspatls |
| scFv - aa | | lspgeratlscrasqdiskylnwyqqkpgqaprlliyhtsrlhsgiparfsgsgsg |
| | | tdytltisslqpedfavyfcqqgntlpytfgqgtkleikhhhhhhhh |
| 104877 | 92 | atggctctgcccgtgaccgcactcctcctgccactggctctgctgcttcacgccgc |
| CAR 3- | | tcgcccacaagtccagcttcaagaatcagggcctggtctggtgaagccatctgaga |
| Full - nt | | ctctgtccctcacttgcaccgtgagcggagtgtccctcccagactacggagtgagc |
| | | tggattagacagcctcccggaaagggactggagtggatcggagtgatttggggtag |
| | | cgaaaccacttactattcatcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccgaaatcgtgatgacccagagccctgcaaccctgtcc |
| | | ctttctcccggggaacgggctaccctttcttgtcgggcatcacaagatatctcaaa |
| | | atacctcaattggtatcaacagaagccgggacaggcccctaggcttcttatctacc |
| | | acacctctcgcctgcatagcgggattcccgcacgctttagcgggtctggaagcggg |
| | | accgactacactctgaccatctcatctctccagcccgaggacttcgccgtctactt |
| | | ctgccagcagggtaacaccctgccgtacaccttcggccagggcaccaagcttgaga |
| | | tcaaaaccactactcccgctccaaggccacccacccctgccccgaccatcgcctct |
| | | cagccgctttccctgcgtccggaggcatgtagacccgcagctggtggggccgtgca |
| | | tacccggggtcttgacttcgcctgcgatatctacatttgggcccctctggctggta |
| | | cttgcggggtcctgctgctttcactcgtgatcactctttactgtaagcgcggtcgg |
| | | aagaagctgctgtacatctttaagcaacccttcatgaggcctgtgcagactactca |
| | | agaggaggacggctgttcatgccggttcccagaggaggaggaaggcggctgcgaac |
| | | tgcgcgtgaaattcagccgcagcgcagatgctccagcctacaagcaggggcagaac |
| | | cagctctacaacgaactcaatcttggtcggagagaggagtacgacgtgctggacaa |
| | | gcggagaggacgggacccagaaatgggcgggaagccgcgcagaaagaatccccaag |
| | | agggcctgtacaacgagctccaaaaggataagatggcagaagcctatagcgagatt |
| | | ggtatgaaaggggaacgcagaagaggcaaaggccacgacggactgtaccagggact |
| | | cagcaccgccaccaaggacacctatgacgctcttcacatgcaggccctgccgcctc |
| | | gg |
| 104877 | 79 | MALPVTALLLPLALLLHAARPqvqlqesgpglvkpsetlsltctvsgvslpdygvs |
| CAR 3- | | wirqppqkglewigviwgsettyyssslksrvtiskdnsknqvslklssvtaadta |
| Full - aa | | vyycakhyyyggsyamdywgqgtlvtvssggggsggggsggggseivmtqspatls |
| | | lspgeratlscrasqdiskylnwyqqkpgqaprlliyhtsrlhsgiparfsgsgsg |
| | | tdytltisslqpedfavyfcqqgntlpytfgqgtkleiktttpaprpptpaptias |
| | | qplslrpeacrpaaggavhtrgldfacdiyiwaplagtcgvlllslvitlyckrgr |
| | | kkllyifkqpfmrpvqttqeedgcscrfpeeeeggcelrvkfsrsadapaykqgqn |
| | | qlynelnlgrreeydvldkrrgrdpemggkprrknpqeglynelqkdkmaeaysei |
| | | gmkgerrrgkghdglyqglstatkdtydalhmqalppr |

CAR 4

| | | |
|---|---|---|
| CAR4 scFv | 42 | qvqlqesgpglvkpsetlsltctvsgvslpdygvswirqppgkglewigviwgset |
| domain | | tyyqsslksrvtiskdnsknqvslklssvtaadtavyycakhyyyggsyamdywgq |
| | | gtlvtvssggggsggggsggggseivmtqspatlslspgeratlscrasqdiskyl |
| | | nwyqqkpgqaprlliyhtsrlhsgiparfsgsgsgtdytltisslqpedfavyfcq |
| | | qgntlpytfgqgtkleik |
| 103106 | 55 | atggctctgcccgtgaccgcactcctcctgccactggctctgctgcttcacgccgc |
| CAR4- | | tcgcccacaagtccagcttcaagaatcagggcctggtctggtgaagccatctgaga |
| Soluble | | ctctgtccctcacttgcaccgtgagcggagtgtccctcccagactacggagtgagc |
| scFv - nt | | tggattagacagcctcccggaaagggactggagtggatcggagtgatttggggtag |
| | | cgaaaccacttactatcaatcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccgaaatcgtgatgacccagagccctgcaaccctgtcc |
| | | ctttctcccggggaacgggctaccctttcttgtcgggcatcacaagatatctcaaa |
| | | atacctcaattggtatcaacagaagccgggacaggcccctaggcttcttatctacc |
| | | acacctctcgcctgcatagcgggattcccgcacgctttagcgggtctggaagcggg |
| | | accgactacactctgaccatctcatctctccagcccgaggacttcgccgtctactt |
| | | ctgccagcagggtaacaccctgccgtacaccttcggccagggcaccaagcttgaga |
| | | tcaaacatcaccaccatcatcaccatcac |
| 103106 | 67 | MALPVTALLLPLALLLHAARPqvqlqesgpglvkpsetlsltctvsgvslpdygvs |
| CAR4- | | wirqppgkglewigviwgsettyyqsslksrvtiskdnsknqvslklssvtaadta |
| Soluble | | vyycakhyyyggsyamdywgqgtlvtvssggggsggggsggggseivmtqspatls |
| scFv -aa | | lspgeratlscrasqdiskylnwyqqkpgqaprlliyhtsrlhsgiparfsgsgsg |
| | | tdytltisslqpedfavyfcqqgntlpytfgqgtkleikhhhhhhhh |
| 104878 | 93 | atggctctgcccgtgaccgcactcctcctgccactggctctgctgcttcacgccgc |
| CAR 4- | | tcgcccacaagtccagcttcaagaatcagggcctggtctggtgaagccatctgaga |
| Full - nt | | ctctgtccctcacttgcaccgtgagcggagtgtccctcccagactacggagtgagc |
| | | tggattagacagcctcccggaaagggactggagtggatcggagtgatttggggtag |
| | | cgaaaccacttactatcaatcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccgaaatcgtgatgacccagagccctgcaaccctgtcc |
| | | ctttctcccggggaacgggctaccctttcttgtcgggcatcacaagatatctcaaa |
| | | atacctcaattggtatcaacagaagccgggacaggcccctaggcttcttatctacc |
| | | acacctctcgcctgcatagcgggattcccgcacgctttagcgggtctggaagcggg |
| | | accgactacactctgaccatctcatctctccagcccgaggacttcgccgtctactt |
| | | ctgccagcagggtaacaccctgccgtacaccttcggccagggcaccaagcttgaga |
| | | tcaaaaccactactcccgctccaaggccacccacccctgccccgaccatcgcctct |
| | | cagccgctttccctgcgtccggaggcatgtagacccgcagctggtggggccgtgca |
| | | tacccggggtcttgacttcgcctgcgatatctacatttgggcccctctggctggta |
| | | cttgcggggtcctgctgctttcactcgtgatcactctttactgtaagcgcggtcgg |
| | | aagaagctgctgtacatctttaagcaacccttcatgaggcctgtgcagactactca |
| | | agaggaggacggctgttcatgccggttcccagaggaggaggaaggcggctgcgaac |
| | | tgcgcgtgaaattcagccgcagcgcagatgctccagcctacaagcaggggcagaac |
| | | cagctctacaacgaactcaatcttggtcggagagaggagtacgacgtgctggacaa |
| | | gcggagaggacgggacccagaaatgggcgggaagccgcgcagaaagaatccccaag |
| | | agggcctgtacaacgagctccaaaaggataagatggcagaagcctatagcgagatt |
| | | ggtatgaaaggggaacgcagaagaggcaaaggccacgacggactgtaccagggact |
| | | cagcaccgccaccaaggacacctatgacgctcttcacatgcaggccctgccgcctc |
| | | gg |
| 104878 | 80 | MALPVTALLLPLALLLHAARPqvqlqesgpglvkpsetlsltctvsgvslpdygvs |
| CAR 4- | | wirqppgkglewigviwgsettyyqsslksrvtiskdnsknqvslklssvtaadta |
| Full - aa | | vyycakhyyyggsyamdywgqgtlvtvssggggsggggsggggseivmtqspatls |
| | | lspgeratlscrasqdiskylnwyqqkpgqaprlliyhtsrlhsgiparfsgsgsg |
| | | tdytltisslqpedfavyfcqqgntlpytfgqgtklelktttpaprpptpaptias |
| | | qplslrpeacrpaaggavhtrgldfacdiyiwaplagtcgvlllslvitlyckrgr |
| | | kkllyifkqpfmrpvqttqeedgcscrfpeeeeggcelrvkfsrsadapaykqgqn |
| | | qlynelnlgrreeydvldkrrgrdpemggkprrknpqeglynelqkdkmaeaysei |
| | | gmkgerrrgkghdglyqglstatkdtydalhmqalppr |

CAR5

| | | |
|---|---|---|
| CAR5 scFv | 43 | eivmtqspatlslspgeratlscrasqdiskylnwyqqkpgqaprlliyhtsrlhs |
| domain | | giparfsgsgsgtdytltisslqpedfavyfcqqgntlpytfgqgtkleikggggs |
| | | ggggsggggsggggsqvqlqesgpglvkpsetlsltctvsgvslpdygvswirqpp |
| | | gkglewigviwgsettyyssslksrvtiskdnsknqvslklssvtaadtavyycak |
| | | hyyyggsyamdywgqgtlvtvss |
| 99789 | 56 | atggccctcccagtgaccgctctgctgctgcctctcgcacttcttctccatgccgc |
| CAR5- | | tcggcctgagatcgtcatgacccaaagccccgctaccctgtccctgtcacccggcg |
| Soluble | | agagggcaaccctttcatgcagggccagccaggacatttctaagtacctcaactgg |
| scFv - nt | | tatcagcagaagccagggcaggctcctcgcctgctgatctaccacaccagccgcct |
| | | ccacagcggtatccccgccagattttccgggagcgggtctggaaccgactacaccc |
| | | tcaccatctcttctctgcagcccgaggatttcgccgtctatttctgccagcagggg |
| | | aatactctgccgtacaccttcggtcaaggtaccaagctggaaatcaagggaggcgg |
| | | aggatcaggcggtggcggaagcggaggaggtggctccggaggaggaggttcccaag |
| | | tgcagcttcaagaatcaggacccggacttgtgaagccatcagaaaccctctccctg |
| | | acttgtaccgtgtccggtgtgagcctccccgactacggagtctcttggattcgcca |
| | | gcctccggggaagggtcttgaatggattggggtgatttggggatcagagactactt |
| | | actactcttcatcacttaagtcacgggtcaccatcagcaaagataatagcaagaac |
| | | caagtgtcacttaagctgtcatctgtgaccgccgctgacaccgccgtgtactattg |
| | | tgccaaacattactattacggagggtcttatgctatggactactggggacagggga |
| | | ccctggtgactgtctctagccatcaccatcaccaccatcatcac |
| 99789 | 68 | MALPVTALLLPLALLLHAARPeivmtqspatlslspgeratlscrasqdiskylnw |
| CAR5- | | yqqkpgqaprlliyhtsrlhsgiparfsgsgsgtdytltisslqpedfavyfcqqg |
| Soluble | | ntlpytfgqgtkleikggggsggggsggggsggggsqvqlqesgpglvkpsetlsl |
| scFv -aa | | tctvsgvslpdygvswirqppgkglewigviwgsettyyssslksrvtiskdnskn |
| | | qvslklssvtaadtavyycakhyyyqqsyamdywqqqtlvtvsshhhhhhhh |
| 104879 | 94 | atggccctccctgtcaccgccctgctgcttccgctggctcttctgctccacgccgc |
| CAR5- | | tcggcccgaaattgtgatgacccagtcacccgccactcttagcctttcacccggtg |
| Full - nt | | agcgcgcaaccctgtcttgcagagcctcccaagacatctcaaaataccttaattgg |
| | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagcggcggaggcgggagccagg |
| | | tccaactccaagaaagcggaccgggtcttgtgaagccatcagaaactctttcactg |
| | | acttgtactgtgagcggagtgtctctccccgattacggggtgtcttggatcagaca |
| | | gccaccggggaagggtctggaatggattggagtgatttggggctctgagactactt |
| | | actactcttcatccctcaagtcacgcgtcaccatctcaaaggacaactctaagaat |
| | | caggtgtcactgaaactgtcatctgtgaccgcagccgacaccgccgtgtactattg |
| | | cgctaagcattactattatggcgggagctacgcaatggattactggggacagggta |
| | | ctctggtcaccgtgtccagcaccactaccccagcaccgaggccacccaccccggct |
| | | cctaccatcgcctcccagcctctgtccctgcgtccggaggcatgtagacccgcagc |
| | | tggtggggccgtgcatacccggggtcttgacttcgcctgcgatatctacatttggg |
| | | cccctctggctggtacttgcggggtcctgctgctttcactcgtgatcactctttac |
| | | tgtaagcgcggtcggaagaagctgctgtacatctttaagcaacccttcatgaggcc |
| | | tgtgcagactactcaagaggaggacggctgttcatgccggttcccagaggaggagg |
| | | aaggcggctgcgaactgcgcgtgaaattcagccgcagcgcagatgctccagcctac |
| | | aagcaggggcagaaccagctctacaacgaactcaatcttggtcggagagaggagta |
| | | cgacgtgctggacaagcggagaggacgggacccagaaatgggcgggaagccgcgca |
| | | gaaagaatccccaagagggcctgtacaacgagctccaaaaggataagatggcagaa |
| | | gcctatagcgagattggtatgaaaggggaacgcagaagaggcaaaggccacgacgg |
| | | actgtaccagggactcagcaccgccaccaaggacacctatgacgctcttcacatgc |
| | | aggccctgccgcctcgg |
| 104879 | 81 | MALPVTALLLPLALLLHAARPeivmtqspatlslspgeratlscrasqdiskylnw |
| CAR 5- | | yqqkpgqaprlliyhtsrlhsgiparfsgsgsgtdytltisslqpedfavyfcqqg |
| Full - aa | | ntlpytfgqgtkleikggggsggggsggggsggggsqvqlqesgpglvkpsetlsl |
| | | tctvsgvslpdygvswirqppgkglewigviwgsettyyssslksrvtiskdnskn |
| | | qvslklssvtaadtavyycakhyyyggsyamdywqqqtlvtvsstttpaprpptpa |
| | | ptiasqplslrpeacrpaaggavhtrgldfacdiyiwaplagtcgvlllslvitly |
| | | ckrgrkkllyifkqpfmrpvqttqeedgcscrfpeeeeggcelrvkfsrsadapay |
| | | kqgqnqlynelnlgrreeydvldkrrgrdpemggkprrknpqeglynelqkdkmae |
| | | ayseigmkgerrrgkghdglyqglstatkdtydalhmqalppr |

CAR 6

| | | |
|---|---|---|
| CAR6 | 44 | eivmtqspatlslspgeratlscrasqdiskylnwyqqkpgqaprlliyhtsrlhs |
| scFv | | giparfsgsgsgtdytltisslqpedfavyfcqqgntlpytfgqgtkleikggggs |
| domain | | ggggsggggsggggsqvqlqesgpglvkpsetlsltctvsgvslpdygvswirqpp |
| | | gkglewigviwgsettyyqsslksrvtiskdnsknqvslklssvtaadtavyycak |
| | | hyyyggsyamdywgqgtlvtvss |
| 99790 | 57 | atggccctcccagtgaccgctctgctgctgcctctcgcacttcttctccatgccgc |
| CAR6- | | tcggcctgagatcgtcatgacccaaagccccgctaccctgtccctgtcacccggcg |
| Soluble | | agagggcaaccctttcatgcagggccagccaggacatttctaagtacctcaactgg |
| scFv - nt | | tatcagcagaagccagggcaggctcctcgcctgctgatctaccacaccagccgcct |
| | | ccacagcggtatccccgccagattttccgggagcgggtctggaaccgactacaccc |
| | | tcaccatctcttctctgcagcccgaggatttcgccgtctatttctgccagcagggg |
| | | aatactctgccgtacaccttcggtcaaggtaccaagctggaaatcaagggaggcgg |
| | | aggatcaggcggtggcggaagcggaggaggtggctccggaggaggaggttcccaag |
| | | tgcagcttcaagaatcaggacccggacttgtgaagccatcagaaaccctctccctg |
| | | acttgtaccgtgtccggtgtgagcctccccgactacggagtctcttggattcgcca |
| | | gcctccggggaagggtcttgaatggattggggtgatttggggatcagagactactt |
| | | actaccagtcatcacttaagtcacgggtcaccatcagcaaagataatagcaagaac |
| | | caagtgtcacttaagctgtcatctgtgaccgccgctgacaccgccgtgtactattg |
| | | tgccaaacattactattacggagggtcttatgctatggactactggggacagggga |
| | | ccctggtgactgtctctagccatcaccatcaccaccatcatcac |
| 99790 | 69 | MALPVTALLLPLALLLHAARPeivmtqspatlslspgeratlscrasqdiskylnw |
| CAR6 - | | yqqkpgqaprlliyhtsrlhsgiparfsgsgsgtdytltisslqpedfavyfcqqg |
| Soluble | | ntlpytfgqgtkleikggggsggggsggggsggggsqvqlqesgpglvkpsetlsl |
| scFv - aa | | tctvsgvslpdygvswirqppgkglewigviwgsettyyqsslksrvtiskdnskn |
| | | qvslklssvtaadtavyycakhyyyggsyamdywgqgtlvtvsshhhhhhhh |
| 104880 | 95 | atggccctccctgtcaccgccctgctgcttccgctggctcttctgctccacgccgc |
| CAR6- | | tcggcccgaaattgtgatgacccagtcacccgccactcttagcctttcacccggtg |
| Full - nt | | agcgcgcaaccctgtcttgcagagcctcccaagacatctcaaaataccttaattgg |
| | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagcggaggcggagggagccagg |
| | | tccaactccaagaaagcggaccgggtcttgtgaagccatcagaaactctttcactg |
| | | acttgtactgtgagcggagtgtctctccccgattacggggtgtcttggatcagaca |
| | | gccaccggggaagggtctggaatggattggagtgatttggggctctgagactactt |
| | | actaccaatcatccctcaagtcacgcgtcaccatctcaaaggacaactctaagaat |
| | | caggtgtcactgaaactgtcatctgtgaccgcagccgacaccgccgtgtactattg |
| | | cgctaagcattactattatggcgggagctacgcaatggattactggggacagggta |
| | | ctctggtcaccgtgtccagcaccactaccccagcaccgaggccacccaccccggct |
| | | cctaccatcgcctcccagcctctgtccctgcgtccggaggcatgtagacccgcagc |
| | | tggtggggccgtgcatacccggggtcttgacttcgcctgcgatatctacatttggg |
| | | cccctctggctggtacttgcggggtcctgctgctttcactcgtgatcactctttac |
| | | tgtaagcgcggtcggaagaagctgetgtacatctttaagcaaccctteatgaggee |
| | | tgtgcagactactcaagaggaggacggctgttcatgccggttcccagaggaggagg |
| | | aaggcggctgcgaactgcgcgtgaaattcagccgcagcgcagatgctccagcctac |
| | | aagcaggggcagaaccagctctacaacgaactcaatcttggtcggagagaggagta |
| | | cgacgtgctggacaagcggagaggacgggacccagaaatgggcgggaagccgcgca |
| | | gaaagaatccccaagagggcctgtacaacgagctccaaaaggataagatggcagaa |
| | | gcctatagcgagattggtatgaaaggggaacgcagaagaggcaaaggccacgacgg |
| | | actgtaccagggactcagcaccgccaccaaggacacctatgacgctcttcacatgc |
| | | aggccctgccgcctcgg |
| 104880 | 82 | MALPVTALLLPLALLLHAARPeivmtqspatlslspgeratlscrasqdiskylnw |
| CAR6- | | yqqkpgqaprlliyhtsrlhsgiparfsgsgsgtdytltisslqpedfavyfcqqg |
| Full - aa | | ntlpytfgqgtkleikggggsggggsggggsggggsqvqlqesgpglvkpsetlsl |
| | | tctvsgvslpdygvswirqppgkglewigviwgsettyyqsslksrvtiskdnskn |
| | | qvslklssvtaadtavyycakhyyyggsyamdywgqgtlvtvsstttpaprpptpa |
| | | ptiasqplslrpeacrpaaggavhtrgldfacdiyiwaplagtcgvlllslvitly |
| | | ckrgrkkllyifkqpfmrpvqttqeedgcscrfpeeeeggcelrvkfsrsadapay |
| | | kqgqnqlynelnlgrreeydvldkrrgrdpemggkprrknpqeglynelqkdkmae |
| | | ayseigmkgerrrgkghdglyqglstatkdtydalhmqalppr |

CAR 7

| | | |
|---|---|---|
| CAR7 scFv | 45 | qvqlqesgpglvkpsetlsltctvsgvslpdygvswirqppgkglewigviwgset |
| domain | | tyyssslksrvtiskdnsknqvslklssvtaadtavyycakhyyyggsyamdywgq |
| | | gtlvtvssggggsggggsggggsggggseivmtqspatlslspgeratlscrasqd |
| | | iskylnwyqqkpgqaprlliyhtsrlhsgiparfsgsgsgtdytltisslqpedfa |
| | | vyfcqqgntlpytfgqgtkleik |
| 100796 | 58 | atggcactgcctgtcactgccctcctgctgcctctggccctccttctgcatgccgc |
| CAR7 - | | caggccccaagtccagctgcaagagtcaggacccggactggtgaagccgtctgaga |
| Soluble | | ctctctcactgacttgtaccgtcagcggcgtgtccctccccgactacggagtgtca |
| scFv - nt | | tggatccgccaacctcccgggaaagggcttgaatggattggtgtcatctggggttc |
| | | tgaaaccacctactactcatcttccctgaagtccagggtgaccatcagcaaggata |
| | | attccaagaaccaggtcagccttaagctgtcatctgtgaccgctgctgacaccgcc |
| | | gtgtattactgcgccaagcactactattacggaggaagctacgctatggactattg |
| | | gggacagggcactctcgtgactgtgagcagcggcggtggagggtctggaggtggag |
| | | gatccggtggtggtgggtcaggcggaggagggagcgagattgtgatgactcagtca |
| | | ccagccaccctttctctttcacccggcgagagagcaaccctgagctgtagagccag |
| | | ccaggacatttctaagtacctcaactggtatcagcaaaaaccggggcaggcccctc |
| | | gcctcctgatctaccatacctcacgccttcactctggtatccccgctcggtttagc |
| | | ggatcaggatctggtaccgactacactctgaccatttccagcctgcagccagaaga |
| | | tttcgcagtgtatttctgccagcagggcaatacccttccttacaccttcggtcagg |
| | | gaaccaagctcgaaatcaagcaccatcaccatcatcaccaccat |
| 100796 | 70 | MALPVTALLLPLALLLHAARPqvqlqesgpglvkpsetlsllctvsgvslpdygvs |
| CAR7 - | | wirqppgkglewigviwgsettyyssslksrvtiskdnsknqvslklssvtaadta |
| Soluble | | vyycakhyyyggsyamdywgqgtlvtvssggggsggggsggggsggggseivmtqs |
| scFv - aa | | patlslspgeratlscrasqdiskylnwyqqkpgqaprlliyhtsrlhsgiparfs |
| | | gsgsgtdytltisslqpedfavyfcqqgntlpytfqqqtkleikhhhhhhhh |
| 104881 | 96 | atggctctgcccgtgaccgcactcctcctgccactggctctgctgcttcacgccgc |
| CAR 7 | | tcgcccacaagtccagcttcaagaatcagggcctggtctggtgaagccatctgaga |
| Full - nt | | ctctgtccctcacttgcaccgtgagcggagtgtccctcccagactacggagtgagc |
| | | tggattagacagcctcccggaaagggactggagtggatcggagtgatttggggtag |
| | | cgaaaccacttactattcatcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccggaggtggcggaagcgaaatcgtgatgacccagagc |
| | | cctgcaaccctgtccctttctcccggggaacgggctaccctttcttgtcgggcatc |
| | | acaagatatctcaaaatacctcaattggtatcaacagaagccgggacaggccccta |
| | | ggcttcttatctaccacacctctcgcctgcatagcgggattcccgcacgctttagc |
| | | gggtctggaagcgggaccgactacactctgaccatctcatctctccagcccgagga |
| | | cttcgccgtctacttctgccagcagggtaacaccctgccgtacaccttcggccagg |
| | | gcaccaagcttgagatcaaaaccactactcccgctccaaggccacccacccctgcc |
| | | ccgaccatcgcctctcagccgctttccctgcgtccggaggcatgtagacccgcagc |
| | | tggtggggccgtgcatacccggggtcttgacttcgcctgcgatatctacatttggg |
| | | cccctctggctggtacttgcggggtcctgctgctttcactcgtgatcactctttac |
| | | tgtaagcgcggtcggaagaagctgctgtacatctttaagcaacccttcatgaggcc |
| | | tgtgcagactactcaagaggaggacggctgttcatgccggttcccagaggaggagg |
| | | aaggcggctgcgaactgcgcgtgaaattcagccgcagcgcagatgctccagcctac |
| | | aagcaggggcagaaccagctctacaacgaactcaatcttggtcggagagaggagta |
| | | cgacgtgctggacaagcggagaggacgggacccagaaatgggcgggaagccgcgca |
| | | gaaagaatccccaagagggcctgtacaacgagctccaaaaggataagatggcagaa |
| | | gcctatagcgagattggtatgaaaggggaacgcagaagaggcaaaggccacgacgg |
| | | actgtaccagggactcagcaccgccaccaaggacacctatgacgctcttcacatgc |
| | | aggccctgccgcctcgg |
| 104881 | 83 | MALPVTALLLPLALLLHAARPqvqlqesqpqlvkpsetlsltctvsqvslpdygvs |
| CAR 7 | | wirqppqkqlewigviwgsettyyssslksrvtiskdnsknqvslklssvtaadta |
| Full - aa | | vyycakhyyyggsyamdywqqqtlvtvssqqqqsqqqqsqqqqsqqqqseivmtqs |
| | | patlslspgeratlscrasqdiskylnwyqqkpgqaprlliyhtsrlhsgiparfs |
| | | gsgsgtdytltisslqpedfavyfcqqgntlpytfgqgtkleiktttpaprpptpa |
| | | ptiasqplslrpeacrpaaggavhtrgldfacdiyiwaplagtcgvlllslvitly |
| | | ckrgrkkllyifkqpfmrpvqttqeedgcscrfpeeeeggcelrvkfsrsadapay |
| | | kqgqnqlynelnlgrreeydvldkrrgrdpemggkprrknpqeglynelqkdkmae |
| | | ayseigmkgerrrgkghdglyqglstatkdtydalhmqalppr |

CAR 8

| | | |
|---|---|---|
| CAR8 scFv | 46 | qvqlqesgpglvkpsetlsltctvsgvslpdygvswirqppgkglewigviwgset |
| domain | | tyyqsslksrvtiskdnsknqvslklssvtaadtavyycakhyyyggsyamdywgq |
| | | gtlvtvssggggsggggsggggsggggseivmtqspatlslspgeratlscrasqd |
| | | iskylnwyqqkpgqaprlliyhtsrlhsgiparfsgsgsgtdytltisslqpedfa |
| | | vyfcqqgntlpytfgqgtkleik |
| 100798 | 59 | atggcactgcctgtcactgccctcctgctgcctctggccctccttctgcatgccgc |
| CAR8 - | | caggccccaagtccagctgcaagagtcaggacccggactggtgaagccgtctgaga |
| Soluble | | ctctctcactgacttgtaccgtcagcggcgtgtccctccccgactacggagtgtca |
| scFv - nt | | tggatccgccaacctcccgggaaagggcttgaatggattggtgtcatctggggttc |
| | | tgaaaccacctactaccagtcttccctgaagtccagggtgaccatcagcaaggata |
| | | attccaagaaccaggtcagccttaagctgtcatctgtgaccgctgctgacaccgcc |
| | | gtgtattactgcgccaagcactactattacggaggaagctacgctatggactattg |
| | | gggacagggcactctcgtgactgtgagcagcggcggtggagggtctggaggtggag |
| | | gatccggtggtggtgggtcaggcggaggagggagcgagattgtgatgactcagtca |
| | | ccagccaccctttctctttcacccggcgagagagcaaccctgagctgtagagccag |
| | | ccaggacatttctaagtacctcaactggtatcagcaaaaaccggggcaggcccctc |
| | | gcctcctgatctaccatacctcacgccttcactctggtatccccgctcggtttagc |
| | | ggatcaggatctggtaccgactacactctgaccatttccagcctgcagccagaaga |
| | | tttcgcagtgtatttctgccagcagggcaatacccttccttacaccttcggtcagg |
| | | gaaccaagctcgaaatcaagcaccatcaccatcatcatcaccac |
| 100798 | 71 | MALPVTALLLPLALLLHAARPqvqlqesgpglvkpsetlsltctvsgvslpdygvs |
| CAR8 - | | wirqppgkglewigviwgsettyyqsslksrvtiskdnsknqvslklssvtaadta |
| Soluble | | vyycakhyyyggsyamdywgqgtlvtvssggggsggggsggggsggggseivmtqs |
| scFv - aa | | patlslspgeratlscrasqdiskylnwyqqkpgqaprlliyhtsrlhsgiparfs |
| | | gsgsgtdytltisslqpedfavyfcqqgntlpytfgqgtkleikhhhhhhhh |
| 104882 | 97 | atggctctgcccgtgaccgcactcctcctgccactggctctgctgcttcacgccgc |
| CAR 8- | | tcgcccacaagtccagcttcaagaatcagggcctggtctggtgaagccatctgaga |
| Full - nt | | ctctgtccctcacttgcaccgtgagcggagtgtccctcccagactacggagtgagc |
| | | tggattagacagcctcccggaaagggactggagtggatcggagtgatttggggtag |
| | | cgaaaccacttactatcaatcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccggaggcggtgggtcagaaatcgtgatgacccagagc |
| | | cctgcaaccctgtccctttctcccggggaacgggctaccctttcttgtcgggcatc |
| | | acaagatatctcaaaatacctcaattggtatcaacagaagccgggacaggccccta |
| | | ggcttcttatctaccacacctctcgcctgcatagcgggattcccgcacgctttagc |
| | | gggtctggaagcgggaccgactacactctgaccatctcatctctccagcccgagga |
| | | cttcgccgtctacttctgccagcagggtaacaccctgccgtacaccttcggccagg |
| | | gcaccaagcttgagatcaaaaccactactcccgctccaaggccacccacccctgcc |
| | | ccgaccatcgcctctcagccgctttccctgcgtccggaggcatgtagacccgcagc |
| | | tggtggggccgtgcatacccggggtcttgacttcgcctgcgatatctacatttggg |
| | | cccctctggctggtacttgcggggtcctgctgctttcactcgtgatcactctttac |
| | | tgtaagcgcggtcggaagaagctgctgtacatctttaagcaacccttcatgaggcc |
| | | tgtgcagactactcaagaggaggacggctgttcatgccggttcccagaggaggagg |
| | | aaggcggctgcgaactgcgcgtgaaattcagccgcagcgcagatgctccagcctac |
| | | aagcaggggcagaaccagctctacaacgaactcaatcttggtcggagagaggagta |
| | | cgacgtgctggacaagcggagaggacgggacccagaaatgggcgggaagccgcgca |
| | | gaaagaatccccaagagggcctgtacaacgagctccaaaaggataagatggcagaa |
| | | gcctatagcgagattggtatgaaaggggaacgcagaagaggcaaaggccacgacgg |
| | | actgtaccagggactcagcaccgccaccaaggacacctatgacgctcttcacatgc |
| | | aggccctgccgcctcgg |
| 104882 | 84 | MALPVTALLLPLALLLHAARPqvqlqesgpglvkpsetlsltctvsgvslpdygvs |
| CAR 8- | | wirqppqkglewigviwgsettyyqsslksrvtiskdnsknqvslklssvtaadta |
| Full - aa | | vyycakhyyyggsyamdywgqgtlvtvssggggsggggsggggsggggseivmtqs |
| | | patlslspgeratlscrasqdiskylnwyqqkpgqaprlliyhtsrlhsgiparfs |
| | | gsgsgtdytltisslqpedfavyfcqqgntlpytfgqgtkleiktttpaprpptpa |
| | | ptiasqplslrpeacrpaaggavhtrgldfacdiyiwaplagtcgvlllslvitly |
| | | ckrgrkkllyifkqpfmrpvqttqeedgcscrfpeeeeggcelrvkfsrsadapay |
| | | kqgqnqlynelnlgrreeydvldkrrgrdpemggkprrknpqeglynelqkdkmae |
| | | ayseigmkgerrrgkghdglyqglstatkdtydalhmqalppr |

CAR 9

| | | |
|---|---|---|
| CAR9 scFv | 47 | eivmtqspatlslspgeratlscrasqdiskylnwyqqkpgqaprlliyhtsrlhs |
| domain | | giparfsgsgsgtdytltisslqpedfavyfcqqgntlpytfgqgtkleikggggs |
| | | ggggsggggsggggsqvqlqesgpglvkpsetlsltctvsgvslpdygvswirqpp |
| | | gkglewigviwgsettyynsslksrvtiskdnsknqvslklssvtaadtavyycak |
| | | hyyyggsyamdywgqgtlvtvss |
| 99789 | 60 | atggccctcccagtgaccgctctgctgctgcctctcgcacttcttctccatgccgc |
| CAR9 - | | tcggcctgagatcgtcatgacccaaagccccgctaccctgtccctgtcacccggcg |
| Soluble | | agagggcaaccctttcatgcagggccagccaggacatttctaagtacctcaactgg |
| scFv - nt | | tatcagcagaagccagggcaggctcctcgcctgctgatctaccacaccagccgcct |
| | | ccacagcggtatccccgccagattttccgggagcgggtctggaaccgactacaccc |
| | | tcaccatctcttctctgcagcccgaggatttcgccgtctatttctgccagcagggg |
| | | aatactctgccgtacaccttcggtcaaggtaccaagctggaaatcaagggaggcgg |
| | | aggatcaggcggtggcggaagcggaggaggtggctccggaggaggaggttcccaag |
| | | tgcagcttcaagaatcaggacccggacttgtgaagccatcagaaaccctctccctg |
| | | acttgtaccgtgtccggtgtgagcctccccgactacggagtctcttggattcgcca |
| | | gcctccggggaagggtcttgaatggattggggtgatttggggatcagagactactt |
| | | actacaattcatcacttaagtcacgggtcaccatcagcaaagataatagcaagaac |
| | | caagtgtcacttaagctgtcatctgtgaccgccgctgacaccgccgtgtactattg |
| | | tgccaaacattactattacggagggtcttatgctatggactactggggacagggga |
| | | ccctggtgactgtctctagecatcaccatcaccaccatcatcac |
| 99789 | 72 | MALPVTALLLPLALLLHAARPeivmtqspatlslspgeratlscrasqdiskylnw |
| CAR9 - | | yqqkpgqaprlliyhtsrlhsgiparfsgsgsgtdytltisslqpedfavyfcqqg |
| Soluble | | ntlpytfgqgtkleikggggsggggsggggsggggsqvqlqesgpglvkpsetlsl |
| scFv - aa | | tctvsgvslpdygvswirqppgkglewigviwgsettyynsslksrvtiskdnskn |
| | | qvslklssvtaadtavyycakhyyyggsyamdywgqgtlvtvsshhhhhhhh |
| 105974 | 98 | atggccctccctgtcaccgccctgctgcttccgctggctcttctgctccacgccgc |
| CAR 9- | | tcggcccgaaattgtgatgacccagtcacccgccactcttagcctttcacccggtg |
| Full - nt | | agcgcgcaaccctgtcttgcagagcctcccaagacatctcaaaataccttaattgg |
| | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagcggaggcggtgggagccagg |
| | | tccaactccaagaaagcggaccgggtcttgtgaagccatcagaaactctttcactg |
| | | acttgtactgtgagcggagtgtctctccccgattacggggtgtcttggatcagaca |
| | | gccaccggggaagggtctggaatggattggagtgatttggggctctgagactactt |
| | | actacaactcatccctcaagtcacgcgtcaccatctcaaaggacaactctaagaat |
| | | caggtgtcactgaaactgtcatctgtgaccgcagccgacaccgccgtgtactattg |
| | | cgctaagcattactattatggcgggagctacgcaatggattactggggacagggta |
| | | ctctggtcaccgtgtccagcaccactaccccagcaccgaggccacccaccccggct |
| | | cctaccatcgcctcccagcctctgtccctgcgtccggaggcatgtagacccgcagc |
| | | tggtggggccgtgcatacccggggtcttgacttcgcctgcgatatctacatttggg |
| | | cccctctggctggtacttgcggggtcctgctgctttcactcgtgatcactctttac |
| | | tgtaagcgcggtcggaagaagctgetgtacatctttaagcaaccctteatgaggee |
| | | tgtgcagactactcaagaggaggacggctgttcatgccggttcccagaggaggagg |
| | | aaggcggctgcgaactgcgcgtgaaattcagccgcagcgcagatgctccagcctac |
| | | aagcaggggcagaaccagctctacaacgaactcaatcttggtcggagagaggagta |
| | | cgacgtgctggacaagcggagaggacgggacccagaaatgggcgggaagccgcgca |
| | | gaaagaatccccaagagggcctgtacaacgagctccaaaaggataagatggcagaa |
| | | gcctatagcgagattggtatgaaaggggaacgcagaagaggcaaaggccacgacgg |
| | | actgtaccagggactcagcaccgccaccaaggacacctatgacgctcttcacatgc |
| | | aggccctgccgcctcgg |
| 105974 | 85 | MALPVTALLLPLALLLHAARPeivmtqspatlslspgeratlscrasqdiskylnw |
| CAR 9- | | yqqkpgqaprlliyhtsrlhsgiparfsgsgsgtdytltisslqpedfavyfcqqg |
| Full - aa | | ntlpytfgqgtkleikggggsggggsggggsggggsqvqlqesqpglvkpsetlsl |
| | | tctvsgvslpdygvswirqppgkglewigviwgsettyynsslksrvtiskdnskn |
| | | qvslklssvtaadtavyycakhyyyggsyamdywgqgtlvtvsstttpaprpptpa |
| | | ptiasqplslrpeacrpaaggavhtrgldfacdiyiwaplagtcgvlllslvitly |
| | | ckrgrkkllyifkqpfmrpvqttqeedgcscrfpeeeeggcelrvkfsrsadapay |
| | | kqgqnqlynelnlgrreeydvldkrrgrdpemggkprrknpqeglynelqkdkmae |
| | | ayseigmkgerrrgkghdglyqglstatkdtydalhmqalppr |

CAR10

| | | |
|---|---|---|
| CAR10 | 48 | qvqlqesgpglvkpsetlsltctvsgvslpdygvswirqppgkglewigviwgset |
| scFv | | tyynsslksrvtiskdnsknqvslklssvtaadtavyycakhyyyggsyamdywgq |
| domain | | gtlvtvssggggsggggsggggsggggseivmtqspatlslspgeratlscrasqd |
| | | iskylnwyqqkpgqaprlliyhtsrlhsgiparfsgsgsgtdytltisslqpedfa |
| | | vyfcqqgntlpytfgqgtkleik |
| 100796 | 61 | atggcactgcctgtcactgccctcctgctgcctctggccctccttctgcatgccgc |
| CAR10 - | | caggccccaagtccagctgcaagagtcaggacccggactggtgaagccgtctgaga |
| Soluble | | ctctctcactgacttgtaccgtcagcggcgtgtccctccccgactacggagtgtca |
| scFv - nt | | tggatccgccaacctcccgggaaagggcttgaatggattggtgtcatctggggttc |
| | | tgaaaccacctactacaactcttccctgaagtccagggtgaccatcagcaaggata |
| | | attccaagaaccaggtcagccttaagctgtcatctgtgaccgctgctgacaccgcc |
| | | gtgtattactgcgccaagcactactattacggaggaagctacgctatggactattg |
| | | gggacagggcactctcgtgactgtgagcagcggcggtggagggtctggaggtggag |
| | | gatccggtggtggtgggtcaggcggaggagggagcgagattgtgatgactcagtca |
| | | ccagccaccctttctctttcacccggcgagagagcaaccctgagctgtagagccag |
| | | ccaggacatttctaagtacctcaactggtatcagcaaaaaccggggcaggcccctc |
| | | gcctcctgatctaccatacctcacgccttcactctggtatccccgctcggtttagc |
| | | ggatcaggatctggtaccgactacactctgaccatttccagcctgcagccagaaga |
| | | tttcgcagtgtatttctgccagcagggcaatacccttccttacaccttcggtcagg |
| | | gaaccaagctcgaaatcaagcaccatcaccatcatcaccaccat |
| 100796 | 73 | MALPVTALLLPLALLLHAARPqyqlqesgpglvkpsetlsktctvsgvslpdygvs |
| CAR10 - | | wirqppgkglewigviwgsettyynsslksrvtiskdnsknqvslklssvtaadta |
| Soluble | | vyycakhyyyggsyamdywgqgtlvtvssggggsggggsggggsggggseivmtqs |
| scFv - aa | | patlslspgeratlscrasqdiskylnwyqqkpgqaprlliyhtsrlhsgiparfs |
| | | gsgsgtdytltisslqpedfavyfcqqgntlpytfgggtkleikhhhhhhhh |
| 105975 | 99 | atggccctccctgtcaccgccctgctgcttccgctggctcttctgctccacgccgc |
| CAR 10 | | tcggcccgaaattgtgatgacccagtcacccgccactcttagcctttcacccggtg |
| Full - nt | | agcgcgcaaccctgtcttgcagagcctcccaagacatctcaaaataccttaattgg |
| | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagcggaggcggtgggagccagg |
| | | tccaactccaagaaagcggaccgggtcttgtgaagccatcagaaactctttcactg |
| | | acttgtactgtgagcggagtgtctctccccgattacggggtgtcttggatcagaca |
| | | gccaccggggaagggtctggaatggattggagtgatttggggctctgagactactt |
| | | actacaactcatccctcaagtcacgcgtcaccatctcaaaggacaactctaagaat |
| | | caggtgtcactgaaactgtcatctgtgaccgcagccgacaccgccgtgtactattg |
| | | cgctaagcattactattatggcgggagctacgcaatggattactggggacagggta |
| | | ctctggtcaccgtgtccagcaccactaccccagcaccgaggccacccaccccggct |
| | | cctaccatcgcctcccagcctctgtccctgcgtccggaggcatgtagacccgcagc |
| | | tggtggggccgtgcatacccggggtcttgacttcgcctgcgatatctacatttggg |
| | | cccctctggctggtacttgcggggtcctgctgctttcactcgtgatcactctttac |
| | | tgtaagcgcggtcggaagaagctgctgtacatctttaagcaacccttcatgaggcc |
| | | tgtgcagactactcaagaggaggacggctgttcatgccggttcccagaggaggagg |
| | | aaggcggctgcgaactgcgcgtgaaattcagccgcagcgcagatgctccagcctac |
| | | aagcaggggcagaaccagctctacaacgaactcaatcttggtcggagagaggagta |
| | | cgacgtgctggacaagcggagaggacgggacccagaaatgggcgggaagccgcgca |
| | | gaaagaatccccaagagggcctgtacaacgagctccaaaaggataagatggcagaa |
| | | gcctatagcgagattggtatgaaaggggaacgcagaagaggcaaaggccacgacgg |
| | | actgtaccagggactcagcaccgccaccaaggacacctatgacgctcttcacatgc |
| | | aggccctgccgcctcgg |
| 105975 | 86 | MALPVTALLLPLALLLHAARPEIVMTQSPATLSLSPGERATLSCRASQDISKYLNW |
| CAR 10 | | YQQKPGQAPRLLIYHTSRLHSGIPARFSGSGSGTDYTLTISSLQPEDFAVYFCQQG |
| Full - aa | | NTLPYTFGQGTKLEIKGGGGSGGGGSGGGGSGGGGSQVQLQESGPGLVKPSETLSL |
| | | TCTVSGVSLPDYGVSWIRQPPGKGLEWIGVIWGSETTYYNSSLKSRVTISKDNSKN |
| | | QVSLKLSSVTAADTAVYYCAKHYYYGGSYAMDYWGQGTLVTVSSTTTPAPRPPTPA |
| | | PTIASQPLSLRPEACRPAAGGAVHTRGLDFACDIYIWAPLAGTCGVLLLSLVITLY |
| | | CKRGRKKLLYIFKQPFMRPVQTTQEEDGCSCRFPEEEEGGCELRVKFSRSADAPAY |
| | | KQGQNQLYNELNLGRREEYDVLDKRRGRDPEMGGKPRRKNPQEGLYNELQKDKMAE |
| | | AYSEIGMKGERRRGKGHDGLYQGLSTATKDTYDALHMQALPPR |

CAR11

| | | |
|---|---|---|
| CAR11 | 49 | eivmtqspatlslspgeratlscrasqdiskylnwyqqkpgqaprlliyhtsrlhs |
| scFv | | giparfsgsgsgtdytltisslqpedfavyfcqqgntlpytfgqgtkleikggggs |
| domain | | ggggsggggsqvqlqesgpglvkpsetlsltctvsgvslpdygvswirqppgkgle |
| | | wigviwgsettyynsslksrvtiskdnsknqvslklssvtaadtavyycakhyyyg |
| | | gsyamdywgqgtlvtvss |
| 103101 | 62 | Atggccctccctgtcaccgccctgctgcttccgctggctcttctgctccacgccgc |
| CAR11 - | | tcggcccgaaattgtgatgacccagtcacccgccactcttagcctttcacccggtg |
| Soluble | | agcgcgcaaccctgtcttgcagagcctcccaagacatctcaaaataccttaattgg |
| scFv - nt | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagccaggtccaactccaagaaa |
| | | gcggaccgggtcttgtgaagccatcagaaactctttcactgacttgtactgtgagc |
| | | ggagtgtctctccccgattacggggtgtcttggatcagacagccaccggggaaggg |
| | | tctggaatggattggagtgatttggggctctgagactacttactacaattcatccc |
| | | tcaagtcacgcgtcaccatctcaaaggacaactctaagaatcaggtgtcactgaaa |
| | | ctgtcatctgtgaccgcagccgacaccgccgtgtactattgcgctaagcattacta |
| | | ttatggcgggagctacgcaatggattactggggacagggtactctggtcaccgtgt |
| | | ccagccaccaccatcatcaccatcaccat |
| 103101 | 74 | MALPVTALLLPLALLLHAARPeivmtqspatlslspgeratlscrasqdiskylnw |
| CAR11 - | | yqqkpgqaprlliyhtsrlhsgiparfsgsgsgtdytltisslqpedfavyfcqqg |
| Soluble | | ntlpytfgqgtkleikggggsggggsggggsqvqlqesgpglvkpsetlsltctvs |
| scFv - aa | | gvslpdygvswirqppgkglewigviwgsettyynsslksrvtiskdnsknqvslk |
| | | lssvtaadtavyycakhyyyttsyamdywgqgtlvtvsshhhhhhhh |
| 105976 | 100 | atggctctgcccgtgaccgcactcctcctgccactggctctgctgcttcacgccgc |
| CAR 11 | | tcgcccacaagtccagcttcaagaatcagggcctggtctggtgaagccatctgaga |
| Full - nt | | ctctgtccctcacttgcaccgtgagcggagtgtccctcccagactacggagtgagc |
| | | tggattagacagcctcccggaaagggactggagtggatcggagtgatttggggtag |
| | | cgaaaccacttactataactcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccggaggtggcggaagcgaaatcgtgatgacccagagc |
| | | cctgcaaccctgtccctttctcccggggaacgggctaccctttcttgtcgggcatc |
| | | acaagatatctcaaaatacctcaattggtatcaacagaagccgggacaggccccta |
| | | ggcttcttatctaccacacctctcgcctgcatagcgggattcccgcacgctttagc |
| | | gggtctggaagcgggaccgactacactctgaccatctcatctctccagcccgagga |
| | | cttcgccgtctacttctgccagcagggtaacaccctgccgtacaccttcggccagg |
| | | gcaccaagcttgagatcaaaaccactactcccgctccaaggccacccacccctgcc |
| | | ccgaccatcgcctctcagccgctttccctgcgtccggaggcatgtagacccgcagc |
| | | tggtggggccgtgcatacccggggtcttgacttcgcctgcgatatctacatttggg |
| | | cccctctggctggtacttgcggggtcctgctgctttcactcgtgatcactctttac |
| | | tgtaagcgcggtcggaagaagctgctgtacatctttaagcaacccttcatgaggcc |
| | | tgtgcagactactcaagaggaggacggctgttcatgccggttcccagaggaggagg |
| | | aaggcggctgcgaactgcgcgtgaaattcagccgcagcgcagatgctccagcctac |
| | | aagcaggggcagaaccagctctacaacgaactcaatcttggtcggagagaggagta |
| | | cgacgtgctggacaagcggagaggacgggacccagaaatgggcgggaagccgcgca |
| | | gaaagaatccccaagagggcctgtacaacgagctccaaaaggataagatggcagaa |
| | | gcctatagcgagattggtatgaaaggggaacgcagaagaggcaaaggccacgacgg |
| | | actgtaccagggactcagcaccgccaccaaggacacctatgacgctcttcacatgc |
| | | aggccctgccgcctcgg |
| 105976 | 87 | MALPVTALLLPLALLLHAARPQVQLQESGPGLVKPSETLSLTCTVSGVSLPDYGVS |
| CAR 11 | | WIRQPPGKGLEWIGVIWGSETTYYNSSLKSRVTISKDNSKNQVSLKLSSVTAADTA |
| Full - aa | | VYYCAKHYYYGGSYAMDYWGQGTLVTVSSGGGGSGGGGSGGGGSGGGGSEIVMTQS |
| | | PATLSLSPGERATLSCRASQDISKYLNWYQQKPGQAPRLLIYHTSRLHSGIPARFS |
| | | GSGSGTDYTLTISSLQPEDFAVYFCQQGNTLPYTFGQGTKLEIKTTTPAPRPPTPA |
| | | PTIASQPLSLRPEACRPAAGGAVHTRGLDFACDIYIWAPLAGTCGVLLLSLVITLY |
| | | CKRGRKKLLYIFKQPFMRPVQTTQEEDGCSCRFPEEEEGGCELRVKFSRSADAPAY |
| | | KQGQNQLYNELNLGRREEYDVLDKRRGRDPEMGGKPRRKNPQEGLYNELQKDKMAE |
| | | AYSEIGMKGERRRGKGHDGLYQGLSTATKDTYDALHMQALPPR |

CAR12

| | | |
|---|---|---|
| CAR12 | 50 | qvqlqesgpglvkpsetlsltctvsgvslpdygvswirqppgkglewigviwgset |
| scFv | | tyynsslksrvtiskdnsknqvslklssvtaadtavyycakhyyyggsyamdywgq |
| domain | | gtlvtvssggggsggggsggggseivmtqspatlslspgeratlscrasqdiskyl |
| | | nwyqqkpgqaprlliyhtsrlhsgiparfsgsgsgtdytltisslqpedfavyfcq |
| | | qgntlpytfgqgtkleik |
| 103104 | 63 | atggctctgcccgtgaccgcactcctcctgccactggctctgctgcttcacgccgc |
| CAR12 - | | tcgcccacaagtccagcttcaagaatcagggcctggtctggtgaagccatctgaga |
| Soluble | | ctctgtccctcacttgcaccgtgagcggagtgtccctcccagactacggagtgagc |
| scFv - nt | | tggattagacagcctcccggaaagggactggagtggatcggagtgatttggggtag |
| | | cgaaaccacttactataactcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccgaaatcgtgatgacccagagccctgcaaccctgtcc |
| | | ctttctcccggggaacgggctaccctttcttgtcgggcatcacaagatatctcaaa |
| | | atacctcaattggtatcaacagaagccgggacaggcccctaggcttcttatctacc |
| | | acacctctcgcctgcatagcgggattcccgcacgctttagcgggtctggaagcggg |
| | | accgactacactctgaccatctcatctctccagcccgaggacttcgccgtctactt |
| | | ctgccagcagggtaacaccctgccgtacaccttcggccagggcaccaagcttgaga |
| | | tcaaacatcaccaccatcatcaccatcac |
| 103104 | 75 | MALPVTALLLPLALLLHAARPqvqlqesgpglvkpsetlsltctvsgvslpdygvs |
| CAR12 - | | wirqppgkglewigviwgsettyynsslksrvtiskdnsknqvslklssvtaadta |
| Soluble | | vyycakhyyyggsyamdywgqgtlvtvssggggsggggsggggseivmtqspatls |
| scFv -aa | | lspgeratlscrasqdiskylnwyqqkpgqaprlliyhtsrlhsgiparfsgsgsg |
| | | tdytltisslqpedfavyfcqqgntlpytfgqgtkleikhhhhhhhh |
| 105977 | 101 | atggccctccctgtcaccgccctgctgcttccgctggctcttctgctccacgccgc |
| CAR 12 - | | tcggcccgaaattgtgatgacccagtcacccgccactcttagcctttcacccggtg |
| Full - nt | | agcgcgcaaccctgtcttgcagagcctcccaagacatctcaaaataccttaattgg |
| | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagccaggtccaactccaagaaa |
| | | gcggaccgggtcttgtgaagccatcagaaactctttcactgacttgtactgtgagc |
| | | ggagtgtctctccccgattacggggtgtcttggatcagacagccaccggggaaggg |
| | | tctggaatggattggagtgatttggggctctgagactacttactacaactcatccc |
| | | tcaagtcacgcgtcaccatctcaaaggacaactctaagaatcaggtgtcactgaaa |
| | | ctgtcatctgtgaccgcagccgacaccgccgtgtactattgcgctaagcattacta |
| | | ttatggcgggagctacgcaatggattactggggacagggtactctggtcaccgtgt |
| | | ccagcaccactaccccagcaccgaggccacccaccccggctcctaccatcgcctcc |
| | | cagcctctgtccctgcgtccggaggcatgtagacccgcagctggtggggccgtgca |
| | | tacccggggtcttgacttcgcctgcgatatctacatttgggcccctctggctggta |
| | | cttgcggggtcctgctgctttcactcgtgatcactctttactgtaagcgcggtcgg |
| | | aagaagctgctgtacatctttaagcaacccttcatgaggcctgtgcagactactca |
| | | agaggaggacggctgttcatgccggttcccagaggaggaggaaggcggctgcgaac |
| | | tgcgcgtgaaattcagccgcagcgcagatgctccagcctacaagcaggggcagaac |
| | | cagctctacaacgaactcaatcttggtcggagagaggagtacgacgtgctggacaa |
| | | gcggagaggacgggacccagaaatgggcgggaagccgcgcagaaagaatccccaag |
| | | agggcctgtacaacgagctccaaaaggataagatggcagaagcctatagcgagatt |
| | | ggtatgaaaggggaacgcagaagaggcaaaggccacgacggactgtaccagggact |
| | | cagcaccgccaccaaggacacctatgacgctcttcacatgcaggccctgccgcctc |
| | | gg |
| 105977 | 88 | MALPVTALLLPLALLLHAARPEIVMTQSPATLSLSPGERATLSCRASQDISKYLNW |
| CAR 12 - | | YQQKPGQAPRLLIYHTSRLHSGIPARFSGSGSGTDYTLTISSLQPEDFAVYFCQQG |
| Full - aa | | NTLPYTFGQGTKLEIKGGGGSGGGGSGGGGSQVQLQESGPGLVKPSETLSLTCTVS |
| | | GVSLPDYGVSWIRQPPGKGLEWIGVIWGSETTYYNSSLKSRVTISKDNSKNQVSLK |
| | | LSSVTAADTAVYYCAKHYYYGGSYAMDYWGQGTLVTVSSTTTPAPRPPTPAPTIAS |
| | | QPLSLRPEACRPAAGGAVHTRGLDFACDIYIWAPLAGTCGVLLLSLVITLYCKRGR |
| | | KKLLYIFKQPFMRPVQTTQEEDGCSCRFPEEEEGGCELRVKFSRSADAPAYKQGQN |
| | | QLYNELNLGRREEYDVLDKRRGRDPEMGGKPRRKNPQEGLYNELQKDKMAEAYSEI |
| | | GMKGERRRGKGHDGLYQGLSTATKDTYDALHMQALPPR |

CTL019

| | | |
|---|---|---|
| CTL019 - | 22 | atggccctgcccgtcaccgctctgctgctgccccttgctctgcttcttcatgcagc |
| Soluble | | aaggccggacatccagatgacccaaaccacctcatccctctctgcctctcttggag |
| scFv-Histag | | acagggtgaccatttcttgtcgcgccagccaggacatcagcaagtatctgaactgg |
| - nt | | tatcagcagaagccggacggaaccgtgaagctcctgatctaccatacctctcgcct |
| | | gcatagcggcgtgccctcacgcttctctggaagcggatcaggaaccgattattctc |
| | | tcactatttcaaatcttgagcaggaagatattgccacctatttctgccagcagggt |
| | | aataccctgccctacaccttcggaggagggaccaagctcgaaatcaccggtggagg |
| | | aggcagcggcggtggagggtctggtggaggtggttctgaggtgaagctgcaagaat |
| | | caggccctggacttgtggccccttcacagtccctgagcgtgacttgcaccgtgtcc |
| | | ggagtctccctgcccgactacggagtgtcatggatcagacaacctccacggaaagg |
| | | actggaatggctcggtgtcatctggggtagcgaaactacttactacaattcagccc |
| | | tcaaaagcaggctgactattatcaaggacaacagcaagtcccaagtctttcttaag |
| | | atgaactcactccagactgacgacaccgcaatctactattgtgctaagcactacta |
| | | ctacggaggatcctacgctatggattactggggacaaggtacttccgtcactgtct |
| | | cttcacaccatcatcaccatcaccatcac |
| CTL019 - | 76 | MALPVTALLLPLALLLHAARPdiqmtqttsslsaslgdrvtiscrasqdiskylnw |
| Soluble | | yqqkpdgtvklliyhtsrlhsgvpsrfsgsgsgtdysltisnleqediatyfcqqg |
| scFv-Histag | | ntlpytfgggtkleitggggsggggsggggsevklqesgpglvapsqslsvtctvs |
| - aa | | gvslpdygvswirqpprkglewlgviwgsettyynsalksrltiikdnsksqvflk |
| | | mnslqtddtaiyycakhyyyggsyamdywgqgtsvtvsshhhhhhhh |
| CTL019 | 102 | atggccttaccagtgaccgccttgctcctgccgctggccttgctgctccacgccgc |
| Full - nt | | caggccggacatccagatgacacagactacatcctccctgtctgcctctctgggag |
| | | acagagtcaccatcagttgcagggcaagtcaggacattagtaaatatttaaattgg |
| | | tatcagcagaaaccagatggaactgttaaactcctgatctaccatacatcaagatt |
| | | acactcaggagtcccatcaaggttcagtggcagtgggtctggaacagattattctc |
| | | tcaccattagcaacctggagcaagaagatattgccacttacttttgccaacagggt |
| | | aatacgcttccgtacacgttcggaggggggaccaagctggagatcacaggtggcgg |
| | | tggctcgggcggtggtgggtcgggtggcggcggatctgaggtgaaactgcaggagt |
| | | caggacctggcctggtggcgccctcacagagcctgtccgtcacatgcactgtctca |
| | | ggggtctcattacccgactatggtgtaagctggattcgccagcctccacgaaaggg |
| | | tctggagtggctgggagtaatatggggtagtgaaaccacatactataattcagctc |
| | | tcaaatccagactgaccatcatcaaggacaactccaagagccaagttttcttaaaa |
| | | atgaacagtctgcaaactgatgacacagccatttactactgtgccaaacattatta |
| | | ctacggtggtagctatgctatggactactggggccaaggaacctcagtcaccgtct |
| | | cctcaaccacgacgccagcgccgcgaccaccaacaccggcgcccaccatcgcgtcg |
| | | cagcccctgtccctgcgcccagaggcgtgccggccagcggcggggggcgcagtgca |
| | | cacgagggggctggacttcgcctgtgatatctacatctgggcgcccttggccggga |
| | | cttgtggggtccttctcctgtcactggttatcaccctttactgcaaacggggcaga |
| | | aagaaactcctgtatatattcaaacaaccatttatgagaccagtacaaactactca |
| | | agaggaagatggctgtagctgccgatttccagaagaagaagaaggaggatgtgaac |
| | | tgagagtgaagttcagcaggagcgcagacgcccccgcgtacaagcagggccagaac |
| | | cagctctataacgagctcaatctaggacgaagagaggagtacgatgttttggacaa |
| | | gagacgtggccgggaccctgagatggggggaaagccgagaaggaagaaccctcagg |
| | | aaggcctgtacaatgaactgcagaaagataagatggcggaggcctacagtgagatt |
| | | gggatgaaaggcgagcgccggaggggcaaggggcacgatggcctttaccagggtct |
| | | cagtacagccaccaaggacacctacgacgcccttcacatgcaggccctgccccctc |
| | | gc |
| CTL019 | 89 | MALPVTALLLPLALLLHAARPdiqmtqttsslsaslgdrvtiscrasqdiskylnw |
| Full - aa | | yqqkpdgtvklliyhtsrlhsgvpsrfsgsgsgtdysltisnleqediatyfcqqg |
| | | ntlpytfgggtkleitggggsggggsggggsevklqesgpglvapsqslsvtctvs |
| | | gvslpdygvswirqpprkglewlgviwgsettyynsalksrltiikdnsksqvflk |
| | | mnslqtddtaiyycakhyyyggsyamdywgqgtsvtvsstttpaprpptpaptias |
| | | qplslrpeacrpaaggavhtrgldfacdiyiwaplagtcgvlllslvitlyckrgr |
| | | kkllyifkqpfmrpvqttqeedgcscrfpeeeeggcelrvkfsrsadapaykqgqn |
| | | qlynelnlgrreeydvldkrrgrdpemggkprrknpqeglynelqkdkmaeaysei |
| | | gmkgerrrgkghdglyqglstatkdtydalhmqalppr |
| CTL019 | 51 | diqmtqttsslsaslgdrvtiscrasqdiskylnwyqqkpdgtvklliyhtsrlhs |
| scFv | | gvpsrfsgsgsgtdysltisnleqediatyfcqqgntlpytfgggtkleitggggs |
| domain | | ggggsggggsevklqesgpglvapsqslsvtctvsgvslpdygvswirqpprkgle |
| | | wlgviwgsettyynsalksrltiikdnsksqvflkmnslqtddtaiyycakhyyyg |
| | | gsyamdywgqgtsvtvss |

CAR A

| | | |
|---|---|---|
| SEQ ID NO: | CAR A | atgcttctcctggtgacaagccttctgctctgtgagttaccacacccagcattcct |
| 142 | full | cctgatcccagacatccagatgacacagactacatcctccctgtctgcctctctgg |
| | nucleotide | gagacagagtcaccatcagttgcagggcaagtcaggacattagtaaatatttaaat |
| | sequence; | tggtatcagcagaaaccagatggaactgttaaactcctgatctaccatacatcaag |
| | with | attacactcaggagtcccatcaaggttcagtggcagtgggtctggaacagattatt |
| | leader | ctctcaccattagcaacctggagcaagaagatattgccacttacttttgccaacag |
| | | ggtaatacgcttccgtacacgttcggaggggggactaagttggaaataacaggctc |
| | | cacctctggatccggcaagcccggatctggcgagggatccaccaagggcgaggtga |
| | | aactgcaggagtcaggacctggcctggtggcgccctcacagagcctgtccgtcaca |
| | | tgcactgtctcaggggtctcattacccgactatggtgtaagctggattcgccagcc |
| | | tccacgaaagggtctggagtggctgggagtaatatggggtagtgaaaccacatact |
| | | ataattcagctctcaaatccagactgaccatcatcaaggacaactccaagagccaa |
| | | gttttcttaaaaatgaacagtctgcaaactgatgacacagccatttactactgtgc |
| | | caaacattattactacggtggtagctatgctatggactactggggtcaaggaacct |
| | | cagtcaccgtctcctcagcggccgcaattgaagttatgtatcctcctccttaccta |
| | | gacaatgagaagagcaatggaaccattatccatgtgaaagggaaacacctttgtcc |
| | | aagtcccctatttcccggaccttctaagcccttttgggtgctggtggtggttgggg |
| | | gagtcctggcttgctatagcttgctagtaacagtggcctttattattttctgggtg |
| | | aggagtaagaggagcaggctcctgcacagtgactacatgaacatgactccccgccg |
| | | ccccgggcccacccgcaagcattaccagccctatgccccaccacgcgacttcgcag |
| | | cctatcgctccagagtgaagttcagcaggagcgcagacgcccccgcgtaccagcag |
| | | ggccagaaccagctctataacgagctcaatctaggacgaagagaggagtacgatgt |
| | | tttggacaagagacgtggccgggaccctgagatggggggaaagccgagaaggaaga |
| | | accctcaggaaggcctgtacaatgaactgcagaaagataagatggcggaggcctac |
| | | agtgagattgggatgaaaggcgagcgccggaggggcaaggggcacgatggccttta |
| | | ccagggtctcagtacagccaccaaggacacctacgacgcccttcacatgcaggccc |
| | | tgccccctcgc |
| SEQ ID NO: | CAR A- | MLLLVTSLLLCELPHPAFLLIPDIQMTQTTSSLSASLGDRVTISCRASQDISKYLN |
| 143 | full | WYQQKPDGTVKLLIYHTSRLHSGVPSRFSGSGSGTDYSLTISNLEQEDIATYFCQQ |
| | amino | GNTLPYTFGGGTKLEITGSTSGSGKPGSGEGSTKGEVKLQESGPGLVAPSQSLSVT |
| | acid | CTVSGVSLPDYGVSWIRQPPRKGLEWLGVIWGSETTYYNSALKSRLTIIKDNSKSQ |
| | transgene | VFLKMNSLQTDDTAIYYCAKHYYYGGSYAMDYWGQGTSVTVSSAAAIEVMYPPPYL |
| | sequence; | DNEKSNGTIIHVKGKHLCPSPLFPGPSKPFWVLVVVGGVLACYSLLVTVAFIIFWV |
| | with | RSKRSRLLHSDYMNMTPRRPGPTRKHYQPYAPPRDFAAYRSRVKFSRSADAPAYQQ |
| | leader | GQNQLYNELNLGRREEYDVLDKRRGRDPEMGGKPRRKNPQEGLYNELQKDKMAEAY |
| | | SEIGMKGERRRGKGHDGLYQGLSTATKDTYDALHMQALPPR |
| SEQ ID NO: | CAR A- | atgcttctcctggtgacaagccttctgctctgtgagttaccacacccagcattcct |
| 173 | CD19 | cctgatcccagacatccagatgacacagactacatcctccctgtctgcctctctgg |
| | scFv | gagacagagtcaccatcagttgcagggcaagtcaggacattagtaaatatttaaat |
| | nucleotide | tggtatcagcagaaaccagatggaactgttaaactcctgatctaccatacatcaag |
| | sequence | attacactcaggagtcccatcaaggttcagtggcagtgggtctggaacagattatt |
| | with | ctctcaccattagcaacctggagcaagaagatattgccacttacttttgccaacag |
| | leader | ggtaatacgcttccgtacacgttcggaggggggactaagttggaaataacaggctc |
| | | cacctctggatccggcaagcccggatctggcgagggatccaccaagggcgaggtga |
| | | aactgcaggagtcaggacctggcctggtggcgccctcacagagcctgtccgtcaca |
| | | tgcactgtctcaggggtctcattacccgactatggtgtaagctggattcgccagcc |
| | | tccacgaaagggtctggagtggctgggagtaatatggggtagtgaaaccacatact |
| | | ataattcagctctcaaatccagactgaccatcatcaaggacaactccaagagccaa |
| | | gttttcttaaaaatgaacagtctgcaaactgatgacacagccatttactactgtgc |
| | | caaacattattactacggtggtagctatgctatggactactggggtcaaggaacct |
| | | cagtcaccgtctcctca |
| SEQ ID NO: | CAR A- | MLLLVTSLLLCELPHPAFLLIPDIQMTQTTSSLSASLGDRVTISCRASQDISKYLN |
| 144 | CD19 | WYQQKPDGTVKLLIYHTSRLHSGVPSRFSGSGSGTDYSLTISNLEQEDIATYFCQQ |
| | scFv | GNTLPYTFGGGTKLEITGSTSGSGKPGSGEGSTKGEVKLQESGPGLVAPSQSLSVT |
| | amino | CTVSGVSLPDYGVSWIRQPPRKGLEWLGVIWGSETTYYNSALKSRLTIIKDNSKSQ |
| | acid | VFLKMNSLQTDDTAIYYCAKHYYYGGSYAMDYWGQGTSVTVS |
| | sequence; | |
| | with | |
| | leader | |
| SEQ ID NO: | CAR A- | gacatccagatgacacagactacatcctccctgtctgcctctctgggagacagagt |
| 174 | full | caccatcagttgcagggcaagtcaggacattagtaaatatttaaattggtatcagc |
| | nucleotide | agaaaccagatggaactgttaaactcctgatctaccatacatcaagattacactca |
| | sequence; | ggagtcccatcaaggttcagtggcagtgggtctggaacagattattctctcaccat |
| | no | tagcaacctggagcaagaagatattgccacttacttttgccaacagggtaatacgc |
| | leader | ttccgtacacgttcggaggggggactaagttggaaataacaggctccacctctgga |
| | | tccggcaagcccggatctggcgagggatccaccaagggcgaggtgaaactgcagga |
| | | gtcaggacctggcctggtggcgccctcacagagcctgtccgtcacatgcactgtct |
| | | caggggtctcattacccgactatggtgtaagctggattcgccagcctccacgaaag |
| | | ggtctggagtggctgggagtaatatggggtagtgaaaccacatactataattcagc |
| | | tctcaaatccagactgaccatcatcaaggacaactccaagagccaagttttcttaa |
| | | aaatgaacagtctgcaaactgatgacacagccatttactactgtgccaaacattat |
| | | tactacggtggtagctatgctatggactactggggtcaaggaacctcagtcaccgt |
| | | ctcctcagcggccgcaattgaagttatgtatcctcctccttacctagacaatgaga |
| | | agagcaatggaaccattatccatgtgaaagggaaacacctttgtccaagtccccta |
| | | tttcccggaccttctaagcccttttgggtgctggtggtggttgggggagtcctggc |
| | | ttgctatagcttgctagtaacagtggcctttattattttctgggtgaggagtaaga |
| | | ggagcaggctcctgcacagtgactacatgaacatgactccccgccgccccgggccc |
| | | acccgcaagcattaccagccctatgccccaccacgcgacttcgcagcctatcgctc |
| | | cagagtgaagttcagcaggagcgcagacgcccccgcgtaccagcagggccagaacc |
| | | agctctataacgagctcaatctaggacgaagagaggagtacgatgttttggacaag |
| | | agacgtggccgggaccctgagatggggggaaagccgagaaggaagaaccctcagga |
| | | aggcctgtacaatgaactgcagaaagataagatggcggaggcctacagtgagattg |
| | | ggatgaaaggcgagcgccggaggggcaaggggcacgatggcctttaccagggtctc |
| | | agtacagccaccaaggacacctacgacgcccttcacatgcaggccctgccccctcg |
| | | c |
| SEQ ID NO: | CAR A- | DIQMTQTTSSLSASLGDRVTISCRASQDISKYLNWYQQKPDGTVKLLIYHTSRLHS |
| 175 | full | GVPSRFSGSGSGTDYSLTISNLEQEDIATYFCQQGNTLPYTFGGGTKLEITGSTSG |
| | amino | SGKPGSGEGSTKGEVKLQESGPGLVAPSQSLSVTCTVSGVSLPDYGVSWIRQPPRK |
| | acid | GLEWLGVIWGSETTYYNSALKSRLTIIKDNSKSQVFLKMNSLQTDDTAIYYCAKHY |
| | transgene | YYGGSYAMDYWGQGTSVTVSSAAAIEVMYPPPYLDNEKSNGTIIHVKGKHLCPSPL |
| | sequence; | FPGPSKPFWVLVVVGGVLACYSLLVTVAFIIFWVRSKRSRLLHSDYMNMTPRRPGP |
| | no | TRKHYQPYAPPRDFAAYRSRVKFSRSADAPAYQQGQNQLYNELNLGRREEYDVLDK |
| | leader | RRGRDPEMGGKPRRKNPQEGLYNELQKDKMAEAYSEIGMKGERRRGKGHDGLYQGL |
| | | STATKDTYDALHMQALPPR |
| SEQ ID NO: | CAR A- | gacatccagatgacacagactacatcctccctgtctgcctctctgggagacagagt |
| 176 | CD19 | caccatcagttgcagggcaagtcaggacattagtaaatatttaaattggtatcagc |
| | scFv | agaaaccagatggaactgttaaactcctgatctaccatacatcaagattacactca |
| | nucleotide; | ggagtcccatcaaggttcagtggcagtgggtctggaacagattattctctcaccat |
| | no | tagcaacctggagcaagaagatattgccacttacttttgccaacagggtaatacgc |
| | leader | ttccgtacacgttcggaggggggactaagttggaaataacaggctccacctctgga |
| | | tccggcaagcccggatctggcgagggatccaccaagggcgaggtgaaactgcagga |
| | | gtcaggacctggcctggtggcgccctcacagagcctgtccgtcacatgcactgtct |
| | | caggggtctcattacccgactatggtgtaagctggattcgccagcctccacgaaag |
| | | ggtctggagtggctgggagtaatatggggtagtgaaaccacatactataattcagc |
| | | tctcaaatccagactgaccatcatcaaggacaactccaagagccaagttttcttaa |
| | | aaatgaacagtctgcaaactgatgacacagccatttactactgtgccaaacattat |
| | | tactacggtggtagctatgctatggactactggggtcaaggaacctcagtcaccgt |
| | | ctcctca |
| SEQ ID NO: | CAR A- | DIQMTQTTSSLSASLGDRVTISCRASQDISKYLNWYQQKPDGTVKLLIYHTSRLHS |
| 177 | CD19 | GVPSRFSGSGSGTDYSLTISNLEQEDIATYFCQQGNTLPYTFGGGTKLEITGSTSG |
| | scFv | SGKPGSGEGSTKGEVKLQESGPGLVAPSQSLSVTCTVSGVSLPDYGVSWIRQPPRK |
| | amino | GLEWLGVIWGSETTYYNSALKSRLTIIKDNSKSQVFLKMNSLQTDDTAIYYCAKHY |
| | acid | YYGGSYAMDYWGQGTSVTVSS |
| | sequence; | |
| | no | |
| | leader | |

CAR B

| | | |
|---|---|---|
| SEQ ID NO: | CAR B- | ATGCTGCTGCTGGTGACCAGCCTGCTGCTGTGCGAGCTGCCCCACCCCGCCTTTCT |
| 145 | full | GCTGATCCCCGACATCCAGATGACCCAGACCACCTCCAGCCTGAGCGCCAGCCTGG |
| | nucleotide | GCGACCGGGTGACCATCAGCTGCCGGGCCAGCCAGGACATCAGCAAGTACCTGAAC |
| | sequence; | TGGTATCAGCAGAAGCCCGACGGCACCGTCAAGCTGCTGATCTACCACACCAGCCG |
| | with | GCTGCACAGCGGCGTGCCCAGCCGGTTTAGCGGCAGCGGCTCCGGCACCGACTACA |
| | leader | GCCTGACCATCTCCAACCTGGAACAGGAAGATATCGCCACCTACTTTTGCCAGCAG |
| | | GGCAACACACTGCCCTACACCTTTGGCGGCGGAACAAAGCTGGAAATCACCGGCAG |
| | | CACCTCCGGCAGCGGCAAGCCTGGCAGCGGCGAGGGCAGCACCAAGGGCGAGGTGA |
| | | AGCTGCAGGAAAGCGGCCCTGGCCTGGTGGCCCCCAGCCAGAGCCTGAGCGTGACC |
| | | TGCACCGTGAGCGGCGTGAGCCTGCCCGACTACGGCGTGAGCTGGATCCGGCAGCC |
| | | CCCCAGGAAGGGCCTGGAATGGCTGGGCGTGATCTGGGGCAGCGAGACCACCTACT |
| | | ACAACAGCGCCCTGAAGAGCCGGCTGACCATCATCAAGGACAACAGCAAGAGCCAG |
| | | GTGTTCCTGAAGATGAACAGCCTGCAGACCGACGACACCGCCATCTACTACTGCGC |
| | | CAAGCACTACTACTACGGCGGCAGCTACGCCATGGACTACTGGGGCCAGGGCACCA |
| | | GCGTGACCGTGAGCAGCGAATCTAAGTACGGACCGCCCTGCCCCCCTTGCCCTATG |
| | | TTCTGGGTGCTGGTGGTGGTCGGAGGCGTGCTGGCCTGCTACAGCCTGCTGGTCAC |
| | | CGTGGCCTTCATCATCTTTTGGGTGAAACGGGGCAGAAAGAAACTCCTGTATATAT |
| | | TCAAACAACCATTTATGAGACCAGTACAAACTACTCAAGAGGAAGATGGCTGTAGC |
| | | TGCCGATTTCCAGAAGAAGAAGAAGGAGGATGTGAACTGCGGGTGAAGTTCAGCAG |
| | | AAGCGCCGACGCCCCTGCCTACCAGCAGGGCCAGAATCAGCTGTACAACGAGCTGA |
| | | ACCTGGGCAGAAGGGAAGAGTACGACGTCCTGGATAAGCGGAGAGGCCGGGACCCT |
| | | GAGATGGGCGGCAAGCCTCGGCGGAAGAACCCCCAGGAAGGCCTGTATAACGAACT |
| | | GCAGAAAGACAAGATGGCCGAGGCCTACAGCGAGATCGGCATGAAGGGCGAGCGGA |
| | | GGCGGGGCAAGGGCCACGACGGCCTGTATCAGGGCCTGTCCACCGCCACCAAGGAT |
| | | ACCTACGACGCCCTGCACATGCAGGCCCTGCCCCCAAGG |
| SEQ ID NO: | CAR B- | MLLLVTSLLLCELPHPAFLLIPDIQMTQTTSSLSASLGDRVTISCRASQDISKYLN |
| 146 | full | WYQQKPDGTVKLLIYHTSRLHSGVPSRFSGSGSGTDYSLTISNLEQEDIATYFCQQ |
| | transgene | GNTLPYTFGGGTKLEITGSTSGSGKPGSGEGSTKGEVKLQESGPGLVAPSQSLSVT |
| | amino | CTVSGVSLPDYGVSWIRQPPRKGLEWLGVIWGSETTYYNSALKSRLTIIKDNSKSQ |
| | acid | VFLKMNSLQTDDTAIYYCAKHYYYGGSYAMDYWGQGTSVTVSSESKYGPPCPPCPM |
| | sequence; | FWVLVVVGGVLACYSLLVTVAFIIFWVKRGRKKLLYIFKQPFMRPVQTTQEEDGCS |
| | with | CRFPEEEEGGCELRVKFSRSADAPAYQQGQNQLYNELNLGRREEYDVLDKRRGRDP |
| | leader | EMGGKPRRKNPQEGLYNELQKDKMAEAYSEIGMKGERRRGKGHDGLYQGLSTATKD |
| | | TYDALHMQALPPR |
| SEQ ID NO: | CAR B- | ATGCTGCTGCTGGTGACCAGCCTGCTGCTGTGCGAGCTGCCCCACCCCGCCTTTCT |
| 178 | CD19 | GCTGATCCCCGACATCCAGATGACCCAGACCACCTCCAGCCTGAGCGCCAGCCTGG |
| | scFv | GCGACCGGGTGACCATCAGCTGCCGGGCCAGCCAGGACATCAGCAAGTACCTGAAC |
| | nucleotide | TGGTATCAGCAGAAGCCCGACGGCACCGTCAAGCTGCTGATCTACCACACCAGCCG |
| | sequence; | GCTGCACAGCGGCGTGCCCAGCCGGTTTAGCGGCAGCGGCTCCGGCACCGACTACA |
| | with | GCCTGACCATCTCCAACCTGGAACAGGAAGATATCGCCACCTACTTTTGCCAGCAG |
| | leader | GGCAACACACTGCCCTACACCTTTGGCGGCGGAACAAAGCTGGAAATCACCGGCAG |
| | | CACCTCCGGCAGCGGCAAGCCTGGCAGCGGCGAGGGCAGCACCAAGGGCGAGGTGA |
| | | AGCTGCAGGAAAGCGGCCCTGGCCTGGTGGCCCCCAGCCAGAGCCTGAGCGTGACC |
| | | TGCACCGTGAGCGGCGTGAGCCTGCCCGACTACGGCGTGAGCTGGATCCGGCAGCC |
| | | CCCCAGGAAGGGCCTGGAATGGCTGGGCGTGATCTGGGGCAGCGAGACCACCTACT |
| | | ACAACAGCGCCCTGAAGAGCCGGCTGACCATCATCAAGGACAACAGCAAGAGCCAG |
| | | GTGTTCCTGAAGATGAACAGCCTGCAGACCGACGACACCGCCATCTACTACTGCGC |
| | | CAAGCACTACTACTACGGCGGCAGCTACGCCATGGACTACTGGGGCCAGGGCACCA |
| | | GCGTGACCGTGAGCAGC |
| SEQ ID NO: | CAR B- | MLLLVTSLLLCELPHPAFLLIPDIQMTQTTSSLSASLGDRVTISCRASQDISKYLN |
| 147 | CD19 | WYQQKPDGTVKLLIYHTSRLHSGVPSRFSGSGSGTDYSLTISNLEQEDIATYFCQQ |
| | scFv | GNTLPYTFGGGTKLEITGSTSGSGKPGSGEGSTKGEVKLQESGPGLVAPSQSLSVT |
| | amino | CTVSGVSLPDYGVSWIRQPPRKGLEWLGVIWGSETTYYNSALKSRLTIIKDNSKSQ |
| | acid | VFLKMNSLQTDDTAIYYCAKHYYYGGSYAMDYWGQGTSVTVSS |
| | sequence; | |
| | with | |
| | leader | |
| SEQ ID NO: | CAR B- | GACATCCAGATGACCCAGACCACCTCCAGCCTGAGCGCCAGCCTGGGCGACCGGGT |
| 179 | full | GACCATCAGCTGCCGGGCCAGCCAGGACATCAGCAAGTACCTGAACTGGTATCAGC |
| | nucleotide | AGAAGCCCGACGGCACCGTCAAGCTGCTGATCTACCACACCAGCCGGCTGCACAGC |
| | sequence; | GGCGTGCCCAGCCGGTTTAGCGGCAGCGGCTCCGGCACCGACTACAGCCTGACCAT |
| | no | CTCCAACCTGGAACAGGAAGATATCGCCACCTACTTTTGCCAGCAGGGCAACACAC |
| | leader | TGCCCTACACCTTTGGCGGCGGAACAAAGCTGGAAATCACCGGCAGCACCTCCGGC |
| | | AGCGGCAAGCCTGGCAGCGGCGAGGGCAGCACCAAGGGCGAGGTGAAGCTGCAGGA |
| | | AAGCGGCCCTGGCCTGGTGGCCCCCAGCCAGAGCCTGAGCGTGACCTGCACCGTGA |
| | | GCGGCGTGAGCCTGCCCGACTACGGCGTGAGCTGGATCCGGCAGCCCCCCAGGAAG |
| | | GGCCTGGAATGGCTGGGCGTGATCTGGGGCAGCGAGACCACCTACTACAACAGCGC |
| | | CCTGAAGAGCCGGCTGACCATCATCAAGGACAACAGCAAGAGCCAGGTGTTCCTGA |
| | | AGATGAACAGCCTGCAGACCGACGACACCGCCATCTACTACTGCGCCAAGCACTAC |
| | | TACTACGGCGGCAGCTACGCCATGGACTACTGGGGCCAGGGCACCAGCGTGACCGT |
| | | GAGCAGCGAATCTAAGTACGGACCGCCCTGCCCCCCTTGCCCTATGTTCTGGGTGC |
| | | TGGTGGTGGTCGGAGGCGTGCTGGCCTGCTACAGCCTGCTGGTCACCGTGGCCTTC |
| | | ATCATCTTTTGGGTGAAACGGGGCAGAAAGAAACTCCTGTATATATTCAAACAACC |
| | | ATTTATGAGACCAGTACAAACTACTCAAGAGGAAGATGGCTGTAGCTGCCGATTTC |
| | | CAGAAGAAGAAGAAGGAGGATGTGAACTGCGGGTGAAGTTCAGCAGAAGCGCCGAC |
| | | GCCCCTGCCTACCAGCAGGGCCAGAATCAGCTGTACAACGAGCTGAACCTGGGCAG |
| | | AAGGGAAGAGTACGACGTCCTGGATAAGCGGAGAGGCCGGGACCCTGAGATGGGCG |
| | | GCAAGCCTCGGCGGAAGAACCCCCAGGAAGGCCTGTATAACGAACTGCAGAAAGAC |
| | | AAGATGGCCGAGGCCTACAGCGAGATCGGCATGAAGGGCGAGCGGAGGCGGGGCAA |
| | | GGGCCACGACGGCCTGTATCAGGGCCTGTCCACCGCCACCAAGGATACCTACGACG |
| | | CCCTGCACATGCAGGCCCTGCCCCCAAGG |
| SEQ ID NO: | CAR B- | DIQMTQTTSSLSASLGDRVTISCRASQDISKYLNWYQQKPDGTVKLLIYHTSRLHS |
| 180 | full | GVPSRFSGSGSGTDYSLTISNLEQEDIATYFCQQGNTLPYTFGGGTKLEITGSTSG |
| | amino | SGKPGSGEGSTKGEVKLQESGPGLVAPSQSLSVTCTVSGVSLPDYGVSWIRQPPRK |
| | acid | GLEWLGVIWGSETTYYNSALKSRLTIIKDNSKSQVFLKMNSLQTDDTAIYYCAKHY |
| | transgene | YYGGSYAMDYWGQGTSVTVSSESKYGPPCPPCPMFWVLVVVGGVLACYSLLVTVAF |
| | sequence; | IIFWVKRGRKKLLYIFKQPFMRPVQTTQEEDGCSCRFPEEEEGGCELRVKFSRSAD |
| | no | APAYQQGQNQLYNELNLGRREEYDVLDKRRGRDPEMGGKPRRKNPQEGLYNELQKD |
| | leader | KMAEAYSEIGMKGERRRGKGHDGLYQGLSTATKDTYDALHMQALPPR |
| SEQ ID NO: | CAR B- | GACATCCAGATGACCCAGACCACCTCCAGCCTGAGCGCCAGCCTGGGCGACCGGGT |
| 181 | CD19 | GACCATCAGCTGCCGGGCCAGCCAGGACATCAGCAAGTACCTGAACTGGTATCAGC |
| | scFv | AGAAGCCCGACGGCACCGTCAAGCTGCTGATCTACCACACCAGCCGGCTGCACAGC |
| | sequence; | GGCGTGCCCAGCCGGTTTAGCGGCAGCGGCTCCGGCACCGACTACAGCCTGACCAT |
| | no | CTCCAACCTGGAACAGGAAGATATCGCCACCTACTTTTGCCAGCAGGGCAACACAC |
| | leader | TGCCCTACACCTTTGGCGGCGGAACAAAGCTGGAAATCACCGGCAGCACCTCCGGC |
| | | AGCGGCAAGCCTGGCAGCGGCGAGGGCAGCACCAAGGGCGAGGTGAAGCTGCAGGA |
| | | AAGCGGCCCTGGCCTGGTGGCCCCCAGCCAGAGCCTGAGCGTGACCTGCACCGTGA |
| | | GCGGCGTGAGCCTGCCCGACTACGGCGTGAGCTGGATCCGGCAGCCCCCCAGGAAG |
| | | GGCCTGGAATGGCTGGGCGTGATCTGGGGCAGCGAGACCACCTACTACAACAGCGC |
| | | CCTGAAGAGCCGGCTGACCATCATCAAGGACAACAGCAAGAGCCAGGTGTTCCTGA |
| | | AGATGAACAGCCTGCAGACCGACGACACCGCCATCTACTACTGCGCCAAGCACTAC |
| | | TACTACGGCGGCAGCTACGCCATGGACTACTGGGGCCAGGGCACCAGCGTGACCGT |
| | | GAGCAGC |
| SEQ ID NO: | CAR B- | DIQMTQTTSSLSASLGDRVTISCRASQDISKYLNWYQQKPDGTVKLLIYHTSRLHS |
| 182 | CD19 | GVPSRFSGSGSGTDYSLTISNLEQEDIATYFCQQGNTLPYTFGGGTKLEITGSTSG |
| | scFv | SGKPGSGEGSTKGEVKLQESGPGLVAPSQSLSVTCTVSGVSLPDYGVSWIRQPPRK |
| | sequence; | GLEWLGVIWGSETTYYNSALKSRLTIIKDNSKSQVFLKMNSLQTDDTAIYYCAKHY |
| | no | YYGGSYAMDYWGQGTSVTVSS |
| | leader | |
| SEQ ID NO: | CAR | MALPVTALLLPLALLLHAARPEIVMTQSPATLSLSPGERATLSCRASQDISKYLNW |
| 153 | 2A- | YQQKPGQAPRLLIYHTSRLHSGIPARFSGSGSGTDYTLTISSLQPEDFAVYFCQQG |
| | Full | NTLPYTFGQGTKLEIKGGGGSGGGGSGGGGSQVQLQESGPGLVKPSETLSLTCTVS |
| | amino | GVSLPDYGVSWIRQPPGKGLEWIGVIWGSETTYYQSSLKSRVTISKDNSKNQVSLK |
| | acid | LSSVTAADTAVYYCAKHYYYGGSYAMDYWGQGTLVTVSSTTTPAPRPPTPAPTIAS |
| | sequence; | QPLSLRPEACRPAAGGAVHTRGLDFACDIYIWAPLAGTCGVLLLSLVITLYCKRGR |
| | signal | KKLLYIFKQPFMRPVQTTQEEDGCSCRFPEEEEGGCELRVKFSRSADAPAYQQGQN |
| | peptide | QLYNELNLGRREEYDVLDKRRGRDPEMGGKPRRKNPQEGLYNELQKDKMAEAYSEI |
| | underlined | GMKGERRRGKGHDGLYQGLSTATKDTYDALHMQALPPR |
| SEQ ID NO: | CAR | EIVMTQSPATLSLSPGERATLSCRASQDISKYLNWYQQKPGQAPRLLIYHTSRLHS |
| 148 | 2A- | GIPARFSGSGSGTDYTLTISSLQPEDFAVYFCQQGNTLPYTFGQGTKLEIKGGGGS |
| | amino | GGGGSGGGGSQVQLQESGPGLVKPSETLSLTCTVSGVSLPDYGVSWIRQPPGKGLE |
| | acid | WIGVIWGSETTYYQSSLKSRVTISKDNSKNQVSLKLSSVTAADTAVYYCAKHYYYG |
| | sequence; | GSYAMDYWGQGTLVTVSSTTTPAPRPPTPAPTIASQPLSLRPEACRPAAGGAVHTR |
| | no | GLDFACDIYIWAPLAGTCGVLLLSLVITLYCKRGRKKLLYIFKQPFMRPVQTTQEE |
| | signal | DGCSCRFPEEEEGGCELRVKFSRSADAPAYQQGQNQLYNELNLGRREEYDVLDKRR |
| | peptide | GRDPEMGGKPRRKNPQEGLYNELQKDKMAEAYSEIGMKGERRRGKGHDGLYQGLST |
| | | ATKDTYDALHMQALPPR |
| SEQ ID NO: | CAR | atggccctccctgtcaccgccctgctgcttccgctggctcttctgctccacgccgc |
| 154 | 2A full | tcggcccgaaattgtgatgacccagtcacccgccactcttagcctttcacccggtg |
| | nucleic | agcgcgcaaccctgtcttgcagagcctcccaagacatctcaaaataccttaattgg |
| | acid | tatcaacagaagcccggacaggctcctcgccttctgatctaccacaccagccggct |
| | sequence; | ccattctggaatccctgccaggttcagcggtagcggatctgggaccgactacaccc |
| | signal | tcactatcagctcactgcagccagaggacttcgctgtctatttctgtcagcaaggg |
| | peptide | aacaccctgccctacacctttggacagggcaccaagctcgagattaaaggtggagg |
| | and stop | tggcagcggaggaggtgggtccggcggtggaggaagccaggtccaactccaagaaa |
| | codon | gcggaccgggtcttgtgaagccatcagaaactctttcactgacttgtactgtgagc |
| | underlined | ggagtgtctctccccgattacggggtgtcttggatcagacagccaccggggaaggg |
| | | tctggaatggattggagtgatttggggctctgagactacttactaccaatcatccc |
| | | tcaagtcacgcgtcaccatctcaaaggacaactctaagaatcaggtgtcactgaaa |
| | | ctgtcatctgtgaccgcagccgacaccgccgtgtactattgcgctaagcattacta |
| | | ttatggcgggagctacgcaatggattactggggacagggtactctggtcaccgtgt |
| | | ccagcaccactaccccagcaccgaggccacccaccccggctcctaccatcgcctcc |
| | | cagcctctgtccctgcgtccggaggcatgtagacccgcagctggtggggccgtgca |
| | | tacccggggtcttgacttcgcctgcgatatctacatttgggcccctctggctggta |
| | | cttgcggggtcctgctgctttcactcgtgatcactctttactgtaagcgcggtcgg |
| | | aagaagctgctgtacatctttaagcaacccttcatgaggcctgtgcagactactca |
| | | agaggaggacggctgttcatgccggttcccagaggaggaggaaggcggctgcgaac |
| | | tgcgcgtgaaattcagccgcagcgcagatgctccagcctaccagcaggggcagaac |
| | | cagctctacaacgaactcaatcttggtcggagagaggagtacgacgtgctggacaa |
| | | gcggagaggacgggacccagaaatgggcgggaagccgcgcagaaagaatccccaag |
| | | agggcctgtacaacgagctccaaaaggataagatggcagaagcctatagcgagatt |
| | | ggtatgaaaggggaacgcagaagaggcaaaggccacgacggactgtaccagggact |
| | | cagcaccgccaccaaggacacctatgacgctcttcacatgcaggccctgccgcctc |
| | | ggtaa |
| SEQ ID NO: | CAR | atggccctccctgtcaccgccctgctgcttccgctggctcttctgctccacgccgc |
| 155 | 2A | tcggcccgaaattgtgatgacccagtcacccgccactcttagcctttcacccggtg |
| | nucleic | agcgcgcaaccctgtcttgcagagcctcccaagacatctcaaaataccttaattgg |
| | acid | tatcaacagaagcccggacaggctcctcgccttctgatctaccacaccagccggct |
| | sequence; | ccattctggaatccctgccaggttcagcggtagcggatctgggaccgactacaccc |
| | signal | tcactatcagctcactgcagccagaggacttcgctgtctatttctgtcagcaaggg |
| | peptide | aacaccctgccctacacctttggacagggcaccaagctcgagattaaaggtggagg |
| | underlined; | tggcagcggaggaggtgggtccggcggtggaggaagccaggtccaactccaagaaa |
| | no | gcggaccgggtcttgtgaagccatcagaaactctttcactgacttgtactgtgagc |
| | stop | ggagtgtctctccccgattacggggtgtcttggatcagacagccaccggggaaggg |
| | codon | tctggaatggattggagtgatttggggctctgagactacttactaccaatcatccc |
| | | tcaagtcacgcgtcaccatctcaaaggacaactctaagaatcaggtgtcactgaaa |
| | | ctgtcatctgtgaccgcagccgacaccgccgtgtactattgcgctaagcattacta |
| | | ttatggcgggagctacgcaatggattactggggacagggtactctggtcaccgtgt |
| | | ccagcaccactaccccagcaccgaggccacccaccccggctcctaccatcgcctcc |
| | | cagcctctgtccctgcgtccggaggcatgtagacccgcagctggtggggccgtgca |
| | | tacccggggtcttgacttcgcctgcgatatctacatttgggcccctctggctggta |
| | | cttgcggggtcctgctgctttcactcgtgatcactctttactgtaagcgcggtcgg |
| | | aagaagctgctgtacatctttaagcaacccttcatgaggcctgtgcagactactca |
| | | agaggaggacggctgttcatgccggttcccagaggaggaggaaggcggctgcgaac |
| | | tgcgcgtgaaattcagccgcagcgcagatgctccagcctaccagcaggggcagaac |
| | | cagctctacaacgaactcaatcttggtcggagagaggagtacgacgtgctggacaa |
| | | gcggagaggacgggacccagaaatgggcgggaagccgcgcagaaagaatccccaag |
| | | agggcctgtacaacgagctccaaaaggataagatggcagaagcctatagcgagatt |
| | | ggtatgaaaggggaacgcagaagaggcaaaggccacgacggactgtaccagggact |
| | | cagcaccgccaccaaggacacctatgacgctcttcacatgcaggccctgccgcctc |
| | | gg |
| SEQ ID NO: | CAR | gaaattgtgatgacccagtcacccgccactcttagcctttcacccggtgagcgcgc |
| 156 | 2A | aaccctgtcttgcagagcctcccaagacatctcaaaataccttaattggtatcaac |
| | nucleic | agaagcccggacaggctcctcgccttctgatctaccacaccagccggctccattct |
| | acid | ggaatccctgccaggttcagcggtagcggatctgggaccgactacaccctcactat |
| | sequence; | cagctcactgcagccagaggacttcgctgtctatttctgtcagcaagggaacaccc |
| | no | tgccctacacctttggacagggcaccaagctcgagattaaaggtggaggtggcagc |
| | signal | ggaggaggtgggtccggcggtggaggaagccaggtccaactccaagaaagcggacc |
| | peptide; | gggtcttgtgaagccatcagaaactctttcactgacttgtactgtgagcggagtgt |
| | stop | ctctccccgattacggggtgtcttggatcagacagccaccggggaagggtctggaa |
| | codon | tggattggagtgatttggggctctgagactacttactaccaatcatccctcaagtc |
| | underlined | acgcgtcaccatctcaaaggacaactctaagaatcaggtgtcactgaaactgtcat |
| | | ctgtgaccgcagccgacaccgccgtgtactattgcgctaagcattactattatggc |
| | | gggagctacgcaatggattactggggacagggtactctggtcaccgtgtccagcac |
| | | cactaccccagcaccgaggccacccaccccggctcctaccatcgcctcccagcctc |
| | | tgtccctgcgtccggaggcatgtagacccgcagctggtggggccgtgcatacccgg |
| | | ggtcttgacttcgcctgcgatatctacatttgggcccctctggctggtacttgcgg |
| | | ggtcctgctgctttcactcgtgatcactctttactgtaagcgcggtcggaagaagc |
| | | tgctgtacatctttaagcaacccttcatgaggcctgtgcagactactcaagaggag |
| | | gacggctgttcatgccggttcccagaggaggaggaaggcggctgcgaactgcgcgt |
| | | gaaattcagccgcagcgcagatgctccagcctaccagcaggggcagaaccagctct |
| | | acaacgaactcaatcttggtcggagagaggagtacgacgtgctggacaagcggaga |
| | | ggacgggacccagaaatgggcgggaagccgcgcagaaagaatccccaagagggcct |
| | | gtacaacgagctccaaaaggataagatggcagaagcctatagcgagattggtatga |
| | | aaggggaacgcagaagaggcaaaggccacgacggactgtaccagggactcagcacc |
| | | gccaccaaggacacctatgacgctcttcacatgcaggccctgccgcctcggtaa |
| SEQ ID NO: | CAR | gaaattgtgatgacccagtcacccgccactcttagcctttcacccggtgagcgcgc |
| 183 | 2A | aaccctgtcttgcagagcctcccaagacatctcaaaataccttaattggtatcaac |
| | nucleic | agaagcccggacaggctcctcgccttctgatctaccacaccagccggctccattct |
| | acid | ggaatccctgccaggttcagcggtagcggatctgggaccgactacaccctcactat |
| | sequence; | cagctcactgcagccagaggacttcgctgtctatttctgtcagcaagggaacaccc |
| | no | tgccctacacctttggacagggcaccaagctcgagattaaaggtggaggtggcagc |
| | signal | ggaggaggtgggtccggcggtggaggaagccaggtccaactccaagaaagcggacc |
| | peptide; | gggtcttgtgaagccatcagaaactctttcactgacttgtactgtgagcggagtgt |
| | no stop | ctctccccgattacggggtgtcttggatcagacagccaccggggaagggtctggaa |
| | codon | tggattggagtgatttggggctctgagactacttactaccaatcatccctcaagtc |
| | | acgcgtcaccatctcaaaggacaactctaagaatcaggtgtcactgaaactgtcat |
| | | ctgtgaccgcagccgacaccgccgtgtactattgcgctaagcattactattatggc |
| | | gggagctacgcaatggattactggggacagggtactctggtcaccgtgtccagcac |
| | | cactaccccagcaccgaggccacccaccccggctcctaccatcgcctcccagcctc |
| | | tgtccctgcgtccggaggcatgtagacccgcagctggtggggccgtgcatacccgg |
| | | ggtcttgacttcgcctgcgatatctacatttgggcccctctggctggtacttgcgg |
| | | ggtcctgctgctttcactcgtgatcactctttactgtaagcgcggtcggaagaagc |
| | | tgctgtacatctttaagcaacccttcatgaggcctgtgcagactactcaagaggag |
| | | gacggctgttcatgccggttcccagaggaggaggaaggcggctgcgaactgcgcgt |
| | | gaaattcagccgcagcgcagatgctccagcctaccagcaggggcagaaccagctct |
| | | acaacgaactcaatcttggtcggagagaggagtacgacgtgctggacaagcggaga |
| | | ggacgggacccagaaatgggcgggaagccgcgcagaaagaatccccaagagggcct |
| | | gtacaacgagctccaaaaggataagatggcagaagcctatagcgagattggtatga |
| | | aaggggaacgcagaagaggcaaaggccacgacggactgtaccagggactcagcacc |
| | | gccaccaaggacacctatgacgctcttcacatgcaggccctgccgcctcgg |
| SEQ ID NO: | VH | CAGGTCCAGCTGCAGGAATCAGGACCAGGGCTGGTGAAACCTAGCGAAACTCTGAG |
| 157 | | TCTGACTTGTACCGTCTCCGGGGTGTCTCTGCCAGACTACGGCGTGAGCTGGATCA |
| | | GACAGCCCCCTGGCAAGTGCCTGGAGTGGATCGGCGTGATCTGGGGCTCCGAGACC |
| | | ACATACTATCAGAGCTCCCTGAAGTCTCGGGTGACCATCTCCAAGGACAACTCTAA |
| | | GAATCAGGTGAGCCTGAAGCTGTCTAGCGTGACCGCCGCCGATACAGCCGTGTACT |
| | | ATTGTGCCAAGCACTACTATTACGGCGGCTCCTATGCCATGGATTACTGGGGCCAG |
| | | GGCACCCTGGTGACAGTGTCCTCT |
| SEQ ID NO: | VH | CAGGTCCAGCTGCAGGAATCCGGCCCAGGACTGGTGAAGCCTAGCGAGACCCTGTC |
| 158 | | CCTGACCTGCACAGTGAGCGGCGTGTCCCTGCCCGATTACGGCGTGAGCTGGATCA |
| | | GACAGCCCCCTGGCAAGTGTCTGGAGTGGATCGGCGTGATCTGGGGCTCTGAGACC |
| | | ACATACTATCAGTCCTCTCTGAAGAGCAGGGTGACCATCTCTAAGGACAACAGCAA |
| | | GAATCAGGTGTCCCTGAAGCTGAGCTCCGTGACCGCAGCAGATACAGCCGTGTACT |
| | | ATTGCGCCAAGCACTACTATTACGGCGGCTCCTATGCTATGGATTATTGGGGGCAG |
| | | GGCACTCTGGTCACTGTCTCATCA |
| SEQ ID NO: | VH | CAGGTGCAGCTGCAGGAATCTGGACCCGGACTGGTGAAACCTAGTGAAACTCTGTC |
| 159 | | TCTGACTTGTACCGTCTCAGGGGTCTCACTGCCAGACTACGGCGTGTCCTGGATCA |
| | | GACAGCCCCCTGGCAAGTGCCTGGAGTGGATCGGCGTGATCTGGGGCTCTGAGACC |
| | | ACATACTATCAGAGCTCCCTGAAGAGCCGGGTGACCATCTCCAAGGACAACTCTAA |
| | | GAATCAGGTGTCCCTGAAGCTGTCTAGCGTGACCGCCGCCGATACAGCCGTGTACT |
| | | ATTGTGCCAAGCACTACTATTACGGCGGCAGCTATGCCATGGATTACTGGGGCCAG |
| | | GGCACCCTGGTGACAGTGTCCTCT |
| SEQ ID NO: | VH | CAGGTCCAGCTGCAGGAAAGCGGCCCAGGACTGGTGAAGCCTAGCGAGACCCTGTC |
| 160 | | CCTGACCTGCACAGTGAGCGGCGTGTCCCTGCCTGATTACGGCGTGTCCTGGATCA |
| | | GACAGCCCCCTGGCAAGTGTCTGGAGTGGATCGGCGTGATCTGGGGCTCCGAGACC |
| | | ACATACTATCAGTCCTCTCTGAAGTCTAGGGTGACAATCTCTAAGGACAACAGCAA |
| | | GAATCAGGTGAGCCTGAAGCTGAGCTCCGTGACCGCAGCAGATACAGCCGTGTACT |
| | | ATTGTGCCAAGCACTACTATTACGGCGGCTCTTATGCTATGGATTATTGGGGGCAG |
| | | GGCACTCTGGTCACTGTCTCAAGC |
| SEQ ID NO: | VH | CAGGTGCAGCTGCAGGAGAGCGGCCCAGGACTGGTGAAGCCTTCCGAGACACTGTC |
| 161 | | TCTGACCTGTACAGTGAGCGGCGTGTCCCTGCCCGACTACGGCGTGTCCTGGATCA |
| | | GACAGCCACCTGGCAAGGGACTGGAGTGGATCGGCGTGATCTGGGGCAGCGAGACC |
| | | ACATACTATCAGAGCTCCCTGAAGTCCAGGGTGACCATCAGCAAGGACAACTCCAA |
| | | GAATCAGGTGAGCCTGAAGCTGTCTAGCGTGACCGCCGCCGATACAGCCGTGTACT |
| | | ATTGCGCCAAGCACTACTATTACGGCGGCTCCTATGCCATGGATTACTGGGGCCAG |
| | | GGCACCCTGGTCACAGTGTCCTCT |
| SEQ ID NO: | VH | CAGGTGCAGCTGCAGGAGTCTGGCCCAGGACTGGTGAAGCCTTCTGAGACCCTGAG |
| 162 | | CCTGACCTGCACAGTGTCCGGCGTGTCTCTGCCCGATTACGGCGTGTCCTGGATCA |
| | | GACAGCCACCTGGCAAGGGACTGGAGTGGATCGGCGTGATCTGGGGCTCTGAGACC |
| | | ACATACTATCAGTCTAGCCTGAAGAGCCGGGTGACAATCTCCAAGGACAACTCTAA |
| | | GAATCAGGTGTCCCTGAAGCTGTCCTCTGTGACCGCCGCCGATACAGCCGTGTACT |
| | | ATTGTGCCAAGCACTACTATTACGGCGGCAGCTATGCCATGGACTACTGGGGCCAG |
| | | GGCACCCTGGTGACAGTGAGCTCC |
| SEQ ID NO: | VH | CAGGTGCAGCTGCAGGAGTCTGGCCCAGGACTGGTGAAGCCTTCTGAGACCCTGAG |
| 163 | | CCTGACCTGCACAGTGAGCGGCGTGTCCCTGCCCGATTACGGCGTGTCCTGGATCA |
| | | GACAGCCACCTGGCAAGGGACTGGAGTGGATCGGCGTGATCTGGGGCAGCGAGACC |
| | | ACATACTATCAGTCCTCTCTGAAGTCCAGGGTGACAATCTCCAAGGACAACTCTAA |
| | | GAATCAGGTGAGCCTGAAGCTGAGCTCCGTGACCGCAGCAGATACAGCCGTGTACT |
| | | ATTGCGCCAAGCACTACTATTACGGCGGCTCCTATGCCATGGACTACTGGGGCCAG |
| | | GGCACCCTGGTCACAGTGTCTAGC |
| SEQ ID NO: | VH | CAGGTGCAGCTGCAGGAGTCCGGCCCAGGACTGGTGAAGCCTTCCGAGACACTGTC |
| 164 | | TCTGACCTGTACAGTGTCCGGCGTGTCTCTGCCCGACTACGGCGTGAGCTGGATCA |
| | | GACAGCCACCTGGCAAGGGACTGGAGTGGATCGGCGTGATCTGGGGCTCTGAGACC |
| | | ACATACTATCAGTCCTCTCTGAAGAGCCGGGTGACCATCAGCAAGGACAACTCCAA |
| | | GAATCAGGTGTCCCTGAAGCTGAGCTCCGTGACCGCAGCAGATACAGCCGTGTACT |
| | | ATTGCGCCAAGCACTACTATTACGGCGGCAGCTATGCCATGGATTACTGGGGCCAG |
| | | GGCACCCTGGTGACAGTGTCTAGC |
| SEQ ID NO: | VL | GAGATCGTGATGACCCAGAGCCCAGCCACACTGAGCCTGTCCCCAGGAGAGAGGGC |
| 165 | | CACACTGTCTTGTAGAGCCAGCCAGGATATCTCCAAGTATCTGAACTGGTACCAGC |
| | | AGAAGCCTGGACAGGCACCAAGGCTGCTGATCTACCACACCTCTAGACTGCACAGC |
| | | GGCATCCCTGCCAGGTTTTCTGGCAGCGGCTCCGGCACAGACTATACCCTGACAAT |
| | | CTCTAGCCTGCAGCCAGAGGATTTCGCCGTGTACTTTTGTCAGCAGGGCAATACTC |
| | | TGCCATACACCTTTGGATGCGGAACTAAACTGGAAATCAAG |
| SEQ ID NO: | VL | GAAATTGTGATGACCCAGTCCCCCGCTACTCTGTCTCTGTCCCCCGGAGAACGGGC |
| 166 | | TACTCTGTCTTGTCGCGCTTCCCAGGATATTAGCAAGTACCTGAACTGGTATCAGC |
| | | AGAAGCCAGGACAGGCACCAAGGCTGCTGATCTACCACACCTCTCGCCTGCACAGC |
| | | GGCATCCCTGCACGGTTCTCTGGCAGCGGCTCCGGCACAGACTACACCCTGACAAT |
| | | CAGCTCCCTGCAGCCTGAGGATTTCGCCGTGTACTTTTGCCAGCAGGGCAATACCC |
| | | TGCCATATACATTTGGCTGTGGCACCAAGCTGGAGATCAAG |
| SEQ ID NO: | VL | GAGATCGTGATGACCCAGTCCCCAGCCACACTGAGCCTGTCCCCAGGAGAGAGGGC |
| 167 | | CACCCTGTCTTGTAGAGCCAGCCAGGATATCTCCAAGTATCTGAACTGGTACCAGC |
| | | AGAAGCCTGGACAGGCACCAAGGCTGCTGATCTACCACACCTCTAGACTGCACAGC |
| | | GGCATCCCTGCCAGGTTTTCTGGCAGCGGCTCCGGCACAGACTATACCCTGACAAT |
| | | CTCTAGCCTGCAGCCAGAGGATTTCGCCGTGTACTTTTGTCAGCAGGGAAATACTC |
| | | TGCCATACACCTTTGGATGCGGAACTAAACTGGAAATCAAG |
| SEQ ID NO: | VL | GAGATTGTGATGACCCAGTCCCCCGCCACCCTGAGTCTGAGCCCCGGAGAACGAGC |
| 168 | | TACCCTGAGTTGCCGAGCTTCCCAGGACATTTCCAAGTACCTGAACTGGTATCAGC |
| | | AGAAGCCAGGACAGGCACCAAGGCTGCTGATCTACCACACCTCTCGCCTGCACAGC |
| | | GGCATCCCAGCACGGTTCTCTGGCAGCGGCTCCGGCACAGACTACACCCTGACAAT |
| | | CAGCTCCCTGCAGCCTGAGGATTTCGCCGTGTACTTTTGCCAGCAGGGCAATACCC |
| | | TGCCATATACATTTGGCTGTGGCACCAAGCTGGAGATCAAG |
| SEQ ID NO: | VL | GAGATCGTGATGACCCAGTCTCCAGCCACACTGTCTCTGAGCCCAGGAGAGAGGGC |
| 169 | | CACCCTGTCTTGCCGCGCCAGCCAGGATATCTCCAAGTATCTGAACTGGTACCAGC |
| | | AGAAGCCAGGACAGGCACCAAGGCTGCTGATCTACCACACCTCTCGCCTGCACAGC |
| | | GGCATCCCAGCACGGTTCTCCGGCTCTGGCAGCGGCACAGACTACACCCTGACAAT |
| | | CTCCTCTCTGCAGCCCGAGGATTTCGCCGTGTATTTTTGCCAGCAGGGCAATACCC |
| | | TGCCTTACACATTTGGCCAGGGCACCAAGCTGGAGATCAAG |
| SEQ ID NO: | VL | GAGATCGTGATGACCCAGAGCCCAGCCACACTGAGCCTGTCCCCAGGAGAGAGGGC |
| 170 | | CACCCTGAGCTGCAGAGCCTCCCAGGATATCTCTAAGTATCTGAACTGGTACCAGC |
| | | AGAAGCCTGGACAGGCACCAAGGCTGCTGATCTACCACACCAGCAGACTGCACTCC |
| | | GGCATCCCTGCAAGGTTCTCTGGCAGCGGCTCCGGCACAGACTACACCCTGACAAT |
| | | CTCTAGCCTGCAGCCTGAGGATTTCGCCGTGTATTTTTGTCAGCAGGGCAATACCC |
| | | TGCCATACACATTTGGCCAGGGCACCAAGCTGGAGATCAAG |
| SEQ ID NO: | VL | GAGATCGTGATGACCCAGAGCCCAGCCACACTGTCTCTGAGCCCAGGAGAGAGGGC |
| 171 | | CACCCTGAGCTGTCGCGCCTCCCAGGATATCTCTAAGTATCTGAACTGGTACCAGC |
| | | AGAAGCCAGGACAGGCACCAAGGCTGCTGATCTACCACACCAGCCGCCTGCACTCC |
| | | GGCATCCCAGCACGGTTCTCCGGCTCTGGCAGCGGCACAGACTACACCCTGACAAT |
| | | CTCCTCTCTGCAGCCCGAGGATTTCGCCGTGTATTTTTGCCAGCAGGGCAATACCC |
| | | TGCCTTACACATTTGGCCAGGGCACCAAGCTGGAGATCAAG |
| SEQ ID NO: | VL | GAGATCGTGATGACCCAGTCTCCAGCCACACTGAGCCTGTCCCCAGGAGAGAGGGC |
| 172 | | CACCCTGTCTTGCAGAGCCAGCCAGGATATCTCCAAGTATCTGAACTGGTACCAGC |
| | | AGAAGCCTGGACAGGCACCAAGGCTGCTGATCTACCACACCTCTAGACTGCACAGC |
| | | GGCATCCCAGCAAGGTTCTCTGGCAGCGGCTCCGGCACAGACTACACCCTGACAAT |
| | | CTCTAGCCTGCAGCCTGAGGATTTCGCCGTGTATTTTTGCCAGCAGGGCAATACCC |
| | | TGCCATACACATTTGGCCAGGGCACCAAGCTGGAGATCAAG |
| SEQ ID NO: | Anti- | QVQLQESGPGLVKPSETLSLTCTVSGVSLPDYGVSWIRQPPGKGLEWIGVIWGSET |
| 149 | CD19 | TYYQSSLKSRVTISKDNSKNQVSLKLSSVTAADTAVYYCAKHYYYGGSYAMDYWGQ |
| | VH | GTLVTVSS |
| SEQ ID NO: | Anti- | EIVMTQSPATLSLSPGERATLSCRASQDISKYLNWYQQKPGQAPRLLIYHTSRLHS |
| 150 | CD19 | GIPARFSGSGSGTDYTLTISSLQPEDFAVYFCQQGNTLPYTFGQGTKLEIK |
| | VL | |
| SEQ ID NO: | VH | QVQLQESGPGLVKPSETLSLTCTVSGVSLPDYGVSWIRQPPGKCLEWIGVIWGSET |
| 151 | | TYYQSSLKSRVTISKDNSKNQVSLKLSSVTAADTAVYYCAKHYYYGGSYAMDYWGQ |
| | | GTLVTVSS |
| SEQ ID NO: | VL | EIVMTQSPATLSLSPGERATLSCRASQDISKYLNWYQQKPGQAPRLLIYHTSRLHS |
| 152 | | GIPARFSGSGSGTDYTLTISSLQPEDFAVYFCQQGNTLPYTFGCGTKLEIK |

In some embodiments, the antigen binding domain comprises a HC CDR1, a HC CDR2, and a HC CDR3 of any heavy chain binding domain amino acid sequences listed in Table 3A. In embodiments, the antigen binding domain further comprises a LC CDR1, a LC CDR2, and a LC CDR3. In embodiments, the antigen binding domain comprises a LC CDR1, a LC CDR2, and a LC CDR3 of any light chain binding domain amino acid sequences listed in Table 3B.

In some embodiments, the antigen binding domain comprises one, two or all of LC CDR1, LC CDR2, and LC CDR3 of any light chain binding domain amino acid sequences listed in Table 3B, and one, two or all of HC CDR1, HC CDR2, and HC CDR3 of any heavy chain binding domain amino acid sequences listed in Table 3A.

In an embodiment, the antigen binding domain (e.g., an scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided in Table 3, or a sequence with at least 95%, e.g., 95-99%, identity with an amino acid sequence of Table 3; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided in Table 3, or a sequence with 95-99% identity to an amino acid sequence of Table 3.

In some embodiments, the CAR is a CD19 CAR comprising an antigen binding domain which comprises an scFv amino acid sequence provided in Table 3. In some embodiments, the CD19 CAR comprises an scFv amino acid sequence of any one of SEQ ID NO: 39-51, SEQ ID NO: 144 or SEQ ID NO: 147, or a sequence with at least 80%, 85%, 90%, 95%, or 99% identity thereto.

In some embodiments, the CAR is a CD19 CAR comprising a CAR amino acid sequence provided in Table 3. In some embodiments, the CD19 CAR comprises the amino acid sequence of any one of SEQ ID NO: 77-89, SEQ ID NO: 145 or SEQ ID NO: 146, or a sequence with at least 80%, 85%, 90%, 95%, or 99% identity thereto.

In some embodiments, the CDRs are defined according to the Kabat numbering scheme, the Chothia numbering scheme, or a combination thereof.

The sequences of humanized CDR sequences of the scFv domains are shown in Table 3A for the heavy chain variable domains and in Table 3B for the light chain variable domains. “ID” stands for the respective SEQ ID NO for each CDR.

TABLE 3A
Heavy Chain Variable Domain CDRs (Kabat)

| Candidate | FW | HCDR1 | ID | HCDR2 | ID | HCDR3 | ID |
|---|---|---|---|---|---|---|---|
| murine_ | | DYGVS | 133 | VIWGSET | 134 | HYYYGGS | 138 |
| CART 19 | | | | TYYNSAL | | YAMDY | |
| | | | | KS | | | |
| humanized_ | VH4 | DYGVS | 133 | VIWGSET | 135 | HYYYGGS | 138 |
| CART19 a | | | | TYYSSSL | | YAMDY | |
| | | | | KS | | | |
| humanized_ | VH4 | DYGVS | 133 | VIWGSET | 136 | HYYYGGS | 138 |
| CART19 b | | | | TYYQSSL | | YAMDY | |
| | | | | KS | | | |
| humanized_ | VH4 | DYGVS | 133 | VIWGSET | 137 | HYYYGGS | 138 |
| CART19 c | | | | TYYNSSL | | YAMDY | |
| | | | | KS | | | |

TABLE 3B
Light Chain Variable Domain CDRs

| Candidate | FW | LCDR1 | ID | LCDR2 | ID | LCDR3 | ID |
|---|---|---|---|---|---|---|---|
| murine_ | | RASQDIS | 139 | HTSRLHS | 140 | QQGNTLP | 141 |
| CART 19 | | KYLN | | | | YT | |
| humanized_ | VK3 | RASQDIS | 139 | HTSRLHS | 140 | QQGNTLP | 141 |
| CART19 a | | KYLN | | | | YT | |
| humanized_ | VK3 | RASQDIS | 139 | HTSRLHS | 140 | QQGNTLP | 141 |
| CART19 b | | KYLN | | | | YT | |
| humanized_ | VK3 | RASQDIS | 139 | HTSRLHS | 140 | QQGNTLP | 141 |
| CART19 c | | KYLN | | | | YT | |

Co-Expression of CAR with Other Molecules or Agents

Co-Expression of a Second CAR

In one aspect, the CAR-expressing cell described herein can further comprise a second CAR, e.g., a second CAR that includes a different antigen binding domain, e.g., to the same target (e.g., CD19) or a different target (e.g., a target other than CD19, e.g., a target described herein). In one embodiment, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. Placement of a costimulatory signaling domain, e.g., 4-1BB, CD28, CD27, OX-40 or ICOS, onto the first CAR, and the primary signaling domain, e.g., CD3 zeta, on the second CAR can limit the CAR activity to cells where both targets are expressed. In one embodiment, the CAR expressing cell comprises a first CAR that includes an antigen binding domain, a transmembrane domain and a costimulatory domain and a second CAR that targets another antigen and includes an antigen binding domain, a transmembrane domain and a primary signaling domain. In another embodiment, the CAR expressing cell comprises a first CAR that includes an antigen binding domain, a transmembrane domain and a primary signaling domain and a second CAR that targets another antigen and includes an antigen binding domain to the antigen, a transmembrane domain and a costimulatory signaling domain.

In one embodiment, the CAR-expressing cell comprises an XCAR described herein and an inhibitory CAR. In one embodiment, the inhibitory CAR comprises an antigen binding domain that binds an antigen found on normal cells but not cancer cells, e.g., normal cells that also express X. In one embodiment, the inhibitory CAR comprises the antigen binding domain, a transmembrane domain and an intracellular domain of an inhibitory molecule. For example, the intracellular domain of the inhibitory CAR can be an intracellular domain of PD1, PD-L1, PD-L2, CTLA4, TIM3, CEACAM (CEACAM-1, CEACAM-3, and/or CEACAM-5), LAGS, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF (e.g., TGF beta).

In one embodiment, when the CAR-expressing cell comprises two or more different CARs, the antigen binding domains of the different CARs can be such that the antigen binding domains do not interact with one another. For example, a cell expressing a first and second CAR can have an antigen binding domain of the first CAR, e.g., as a fragment, e.g., an scFv, that does not form an association with the antigen binding domain of the second CAR, e.g., the antigen binding domain of the second CAR is a VHH.

In some embodiments, the antigen binding domain comprises a single domain antigen binding (SDAB) molecules include molecules whose complementary determining regions are part of a single domain polypeptide. Examples include, but are not limited to, heavy chain variable domains, binding molecules naturally devoid of light chains, single domains derived from conventional 4-chain antibodies, engineered domains and single domain scaffolds other than those derived from antibodies. SDAB molecules may be any of the art, or any future single domain molecules. SDAB molecules may be derived from any species including, but not limited to mouse, human, camel, llama, lamprey, fish, shark, goat, rabbit, and bovine. This term also includes naturally occurring single domain antibody molecules from species other than Camelidae and sharks.

In one aspect, an SDAB molecule can be derived from a variable region of the immunoglobulin found in fish, such as, for example, that which is derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov (2005) Protein Sci. 14:2901-2909.

According to another aspect, an SDAB molecule is a naturally occurring single domain antigen binding molecule known as heavy chain devoid of light chains. Such single domain molecules are disclosed in WO 9404678 and Hamers-Casterman, C. et al. (1993) Nature 363:446-448, for example. For clarity reasons, this variable domain derived from a heavy chain molecule naturally devoid of light chain is known herein as a VHH or nanobody to distinguish it from the conventional VH of four chain immunoglobulins. Such a VHH molecule can be derived from Camelidae species, for example in camel, llama, dromedary, alpaca and guanaco. Other species besides Camelidae may produce heavy chain molecules naturally devoid of light chain; such VHHs are within the scope of the invention.

The SDAB molecules can be recombinant, CDR-grafted, humanized, camelized, de-immunized and/or in vitro generated (e.g., selected by phage display).

It has also been discovered, that cells having a plurality of chimeric membrane embedded receptors comprising an antigen binding domain that interactions between the antigen binding domain of the receptors can be undesirable, e.g., because it inhibits the ability of one or more of the antigen binding domains to bind its cognate antigen. Accordingly, disclosed herein are cells having a first and a second non-naturally occurring chimeric membrane embedded receptor comprising antigen binding domains that minimize such interactions. Also disclosed herein are nucleic acids encoding a first and a second non-naturally occurring chimeric membrane embedded receptor comprising an antigen binding domains that minimize such interactions, as well as methods of making and using such cells and nucleic acids. In an embodiment the antigen binding domain of one of the first and the second non-naturally occurring chimeric membrane embedded receptor, comprises an scFv, and the other comprises a single VH domain, e.g., a camelid, shark, or lamprey single VH domain, or a single VH domain derived from a human or mouse sequence.

In some embodiments, a composition herein comprises a first and second CAR, wherein the antigen binding domain of one of the first and the second CAR does not comprise a variable light domain and a variable heavy domain. In some embodiments, the antigen binding domain of one of the first and the second CAR is an scFv, and the other is not an scFv. In some embodiments, the antigen binding domain of one of the first and the second CAR comprises a single VH domain, e.g., a camelid, shark, or lamprey single VH domain, or a single VH domain derived from a human or mouse sequence. In some embodiments, the antigen binding domain of one of the first and the second CAR comprises a nanobody. In some embodiments, the antigen binding domain of one of the first and the second CAR comprises a camelid VHH domain.

In some embodiments, the antigen binding domain of one of the first and the second CAR comprises an scFv, and the other comprises a single VH domain, e.g., a camelid, shark, or lamprey single VH domain, or a single VH domain derived from a human or mouse sequence. In some embodiments, the antigen binding domain of one of the first and the second CAR comprises an scFv, and the other comprises a nanobody. In some embodiments, the antigen binding domain of one of the first and the second CAR comprises an scFv, and the other comprises a camelid VHH domain.

In some embodiments, when present on the surface of a cell, binding of the antigen binding domain of the first CAR to its cognate antigen is not substantially reduced by the presence of the second CAR. In some embodiments, binding of the antigen binding domain of the first CAR to its cognate antigen in the presence of the second CAR is at least 85%, 90%, 95%, 96%, 97%, 98% or 99%, e.g., 85%, 90%, 95%, 96%, 97%, 98% or 99% of binding of the antigen binding domain of the first CAR to its cognate antigen in the absence of the second CAR.

In some embodiments, when present on the surface of a cell, the antigen binding domains of the first and the second CAR, associate with one another less than if both were scFv antigen binding domains. In some embodiments, the antigen binding domains of the first and the second CAR, associate with one another at least 85%, 90%, 95%, 96%, 97%, 98% or 99% less than, e.g., 85%, 90%, 95%, 96%, 97%, 98% or 99% less than if both were scFv antigen binding domains.

Co-Expression of an Agent that Enhances CAR Activity

In another aspect, the CAR-expressing cell described herein can further express another agent, e.g., an agent that enhances the activity or fitness of a CAR-expressing cell.

For example, in one embodiment, the agent can be an agent which inhibits a molecule that modulates or regulates, e.g., inhibits, T cell function. In some embodiments, the molecule that modulates or regulates T cell function is an inhibitory molecule. Inhibitory molecules, e.g., PD1, can, in some embodiments, decrease the ability of a CAR-expressing cell to mount an immune effector response. Examples of inhibitory molecules include PD1, PD-L1, CTLA4, TIM3, LAGS, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, or TGF beta.

In embodiments, an agent, e.g., an inhibitory nucleic acid, e.g., a dsRNA, e.g., an siRNA or shRNA; or e.g., an inhibitory protein or system, e.g., a clustered regularly interspaced short palindromic repeats (CRISPR), a transcription-activator like effector nuclease (TALEN), or a zinc finger endonuclease (ZFN), e.g., as described herein, can be used to inhibit expression of a molecule that modulates or regulates, e.g., inhibits, T-cell function in the CAR-expressing cell. In an embodiment the agent is an shRNA, e.g., an shRNA described herein. In an embodiment, the agent that modulates or regulates, e.g., inhibits, T-cell function is inhibited within a CAR-expressing cell. For example, a dsRNA molecule that inhibits expression of a molecule that modulates or regulates, e.g., inhibits, T-cell function is linked to the nucleic acid that encodes a component, e.g., all of the components, of the CAR.

In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, PD-L1, CTLA4, TIM3, LAGS, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, or TGF beta, or a fragment of any of these (e.g., at least a portion of an extracellular domain of any of these), and a second polypeptide which is an intracellular signaling domain described herein (e.g., comprising a costimulatory domain (e.g., 41BB, CD27 or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of an extracellular domain of PD1), and a second polypeptide of an intracellular signaling domain described herein (e.g., a CD28 signaling domain described herein and/or a CD3 zeta signaling domain described herein). PD1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765-75). Two ligands for PD1, PD-L1 and PD-L2 have been shown to downregulate T cell activation upon binding to PD1 (Freeman et a. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD1 with PD-L1.

In one embodiment, the agent comprises the extracellular domain (ECD) of an inhibitory molecule, e.g., Programmed Death 1 (PD1), can be fused to a transmembrane domain and intracellular signaling domains such as 41BB and CD3 zeta (also referred to herein as a PD1 CAR). In one embodiment, the PD1 CAR, when used in combinations with an XCAR described herein, improves the persistence of the T cell. In one embodiment, the CAR is a PD1 CAR comprising the extracellular domain of PD1 indicated as underlined in SEQ ID NO: 105. In one embodiment, the PD1 CAR comprises the amino acid sequence of SEQ ID NO:105.

(SEQ ID NO: 105)
Malpvtalllplalllhaarppgwfldspdrpwnp
ptfspallvvtegdnatftcsfsntsesfvlnwyr
mspsnqtdklaafpedrsapggderfrvtqlpngr
dfhmsvvrarrndsgtylcgaislapkaqikeslr
aelrvterraevptahpspsprpagqfqtlvtttp
aprpptpaptiasqplslrpeacrpaaggavhtrg
ldfacdiyiwaplagtcgvlllslvitlyckrgrk
kllyifkqpfmrpvqttqeedgcscrfpeeeeggc
elrvkfsrsadapaykqgqnqlynelnlgrreeyd
vldkrrgrdpemggkprrknpqeglynelqkdkma
eayseigmkgerrrgkghdglyqglstatkdtyda
lhmqalppr.

In one embodiment, the PD1 CAR comprises the amino acid sequence provided below (SEQ ID NO:106).

(SEQ ID NO: 106)
pgwfldspdrpwnpptfspallvvtegdnatftcs
fsntsesfvlnwyrmspsngtdklaafpedrsqpg
gderfrvtalpngrdfhmsvvrarrndsgtylcga
islapkaqikeslraelrvterraevptahpspsp
rpagqfqtlvtttpaprpptpaptiasqplsirpe
acrpaaggavhtrgldfacdiyiwaplagtcgvll
lslvitlyckrgrkkllyifkqpfmrpvqttqeed
gcscrfpeeeeggcelrvkfsrsadapaykqgqnq
lynelnlgrreeydvldkrrgrdpemggkprrknp
qeglynelqkdkmaeayseigmkgerrrgkghdgl
yqglstatkdtydalhmqalppr.

In one embodiment, the agent comprises a nucleic acid sequence encoding the PD1 CAR, e.g., the PD1 CAR described herein. In one embodiment, the nucleic acid sequence for the PD1 CAR is shown below, with the PD1 ECD underlined below in SEQ ID NO: 107

(SEQ ID NO: 107)
atggccctccctgtcactgccctgcttctccccct
cgcactcctgctccacgccgctagaccacccggat
ggtttctggactctccggatcgcccgtggaatccc
ccaaccttctcaccggcactcttggttgtgactga
gggcgataatgcgaccttcacgtgctcgttctcca
acacctccgaatcattcgtgctgaactggtaccgc
atgagcccgtcaaaccagaccgacaagctcgccgc
gtttccggaagatcggtcgcaaccgggacaggatt
gtcggttccgcgtgactcaactgccgaatggcaga
gacttccacatgagcgtggtccgcgctaggcgaaa
cgactccgggacctacctgtgcggagccatctcgc
tggcgcctaaggcccaaatcaaagagagcttgagg
gccgaactgagagtgaccgagcgcagagctgaggt
gccaactgcacatccatccccatcgcctcggcctg
cggggcagtttcagaccctggtcacgaccactccg
gcgccgcgcccaccgactccggccccaactatcgc
gagccagcccctgtcgctgaggccggaagcatgcc
gccctgccgccggaggtgctgtgcatacccgggga
ttggacttcgcatgcgacatctacatttgggctcc
tctcgccggaacttgtggcgtgctccttctgtccc
tggtcatcaccctgtactgcaagcggggtcggaaa
aagcttctgtacattttcaagcagcccttcatgag
gcccgtgcaaaccacccaggaggaggacggttgct
cctgccggttccccgaagaggaagaaggaggttgc
gagctgcgcgtgaagttctcccggagcgccgacgc
ccccgcctataagcagggccagaaccagctgtaca
acgaactgaacctgggacggcgggaagagtacgat
gtgctggacaagcggcgcggccgggaccccgaaat
gggcgggaagcctagaagaaagaaccctcaggaag
gcctgtataacgagctgcagaaggacaagatggcc
gaggcctactccgaaattgggatgaagggagagcg
gcggaggggaaaggggcacgacggcctgtaccaag
gactgtccaccgccaccaaggacacatacgatgcc
ctgcacatgcaggcccttccccctcgc.

In another example, in one embodiment, the agent which enhances the activity of a CAR-expressing cell can be a costimulatory molecule or costimulatory molecule ligand. Examples of costimulatory molecules include MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, e.g., as described herein. Examples of costimulatory molecule ligands include CD80, CD86, CD40L, ICOSL, CD70, OX40L, 4-1BBL, GITRL, and LIGHT. In embodiments, the costimulatory molecule ligand is a ligand for a costimulatory molecule different from the costimulatory molecule domain of the CAR. In embodiments, the costimulatory molecule ligand is a ligand for a costimulatory molecule that is the same as the costimulatory molecule domain of the CAR. In an embodiment, the costimulatory molecule ligand is 4-1BBL. In an embodiment, the costimulatory ligand is CD80 or CD86. In an embodiment, the costimulatory molecule ligand is CD70. In embodiments, a CAR-expressing immune effector cell described herein can be further engineered to express one or more additional costimulatory molecules or costimulatory molecule ligands.

Co-Expression of CAR with a Chemokine Receptor

In embodiments, the CAR-expressing cell described herein, e.g., CD19 CAR-expressing cell, further comprises a chemokine receptor molecule. Transgenic expression of chemokine receptors CCR2b or CXCR2 in T cells enhances trafficking to CCL2- or CXCL1-secreting solid tumors including melanoma and neuroblastoma (Craddock et al., J Immunother. 2010 October; 33(8):780-8 and Kershaw et al., Hum Gene Ther. 2002 Nov. 1; 13(16):1971-80). Thus, without wishing to be bound by theory, it is believed that chemokine receptors expressed in CAR-expressing cells that recognize chemokines secreted by tumors, e.g., solid tumors, can improve homing of the CAR-expressing cell to the tumor, facilitate the infiltration of the CAR-expressing cell to the tumor, and enhances antitumor efficacy of the CAR-expressing cell. The chemokine receptor molecule can comprise a naturally occurring or recombinant chemokine receptor or a chemokine-binding fragment thereof. A chemokine receptor molecule suitable for expression in a CAR-expressing cell (e.g., CAR-Tx) described herein include a CXC chemokine receptor (e.g., CXCR1, CXCR2, CXCR3, CXCR4, CXCR5, CXCR6, or CXCR7), a CC chemokine receptor (e.g., CCR1, CCR2, CCR3, CCR4, CCR5, CCR6, CCR7, CCR8, CCR9, CCR10, or CCR11), a CX3C chemokine receptor (e.g., CX3CR1), a XC chemokine receptor (e.g., XCR1), or a chemokine-binding fragment thereof. In one embodiment, the chemokine receptor molecule to be expressed with a CAR described herein is selected based on the chemokine(s) secreted by the tumor. In one embodiment, the CAR-expressing cell described herein further comprises, e.g., expresses, a CCR2b receptor or a CXCR2 receptor. In an embodiment, the CAR described herein and the chemokine receptor molecule are on the same vector or are on two different vectors. In embodiments where the CAR described herein and the chemokine receptor molecule are on the same vector, the CAR and the chemokine receptor molecule are each under control of two different promoters or are under the control of the same promoter.

### Nucleic Acid Constructs Encoding a CAR

The present invention also provides an immune effector cell, e.g., made by a method described herein, that includes a nucleic acid molecules encoding one or more CAR constructs described herein. In one aspect, the nucleic acid molecule is provided as a messenger RNA transcript. In one aspect, the nucleic acid molecule is provided as a DNA construct.

The nucleic acid molecules described herein can be a DNA molecule, an RNA molecule, or a combination thereof. In one embodiment, the nucleic acid molecule is an mRNA encoding a CAR polypeptide as described herein. In other embodiments, the nucleic acid molecule is a vector that includes any of the aforesaid nucleic acid molecules.

In one aspect, the antigen binding domain of a CAR of the invention (e.g., a scFv) is encoded by a nucleic acid molecule whose sequence has been codon optimized for expression in a mammalian cell. In one aspect, entire CAR construct of the invention is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

Accordingly, in one aspect, an immune effector cell, e.g., made by a method described herein, includes a nucleic acid molecule encoding a chimeric antigen receptor (CAR), wherein the CAR comprises an antigen binding domain that binds to a tumor antigen described herein, a transmembrane domain (e.g., a transmembrane domain described herein), and an intracellular signaling domain (e.g., an intracellular signaling domain described herein) comprising a stimulatory domain, e.g., a costimulatory signaling domain (e.g., a costimulatory signaling domain described herein) and/or a primary signaling domain (e.g., a primary signaling domain described herein, e.g., a zeta chain described herein).

The present invention also provides vectors in which a nucleic acid molecule encoding a CAR, e.g., a nucleic acid molecule described herein, is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (ψ), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In another embodiment, the vector comprising the nucleic acid encoding the desired CAR is an adenoviral vector (A5/35). In another embodiment, the expression of nucleic acids encoding CARs can be accomplished using of transposons such as sleeping beauty, crisper, CAS9, and zinc finger nucleases. See below June et al. 2009 Nature Reviews Immunology 9.10: 704-716, is incorporated herein by reference.

In brief summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. Exemplary promoters include the CMV IE gene, EF-1α, ubiquitin C, or phosphoglycerokinase (PGK) promoters.

An example of a promoter that is capable of expressing a CAR encoding nucleic acid molecule in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving CAR expression from nucleic acid molecules cloned into a lentiviral vector. See, e.g., Milone et al., Mol. Ther. 17(8): 1453-1464 (2009). In one aspect, the EF1a promoter comprises the sequence provided in the Examples.

Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1α promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Another example of a promoter is the phosphoglycerate kinase (PGK) promoter. In embodiments, a truncated PGK promoter (e.g., a PGK promoter with one or more, e.g., 1, 2, 5, 10, 100, 200, 300, or 400, nucleotide deletions when compared to the wild-type PGK promoter sequence) may be desired.

The nucleotide sequences of exemplary PGK promoters are provided below.

WT PGK Promoter:
(SEQ ID NO: 109)
ACCCCTCTCTCCAGCCACTAAGCCAGTTGCTCCCT
CGGCTGACGGCTGCACGCGAGGCCTCCGAACGTCT
TACGCCTTGTGGCGCGCCCGTCCTTGTCCCGGGTG
TGATGGCGGGGTGTGGGGCGGAGGGCGTGGCGGGG
AAGGGCCGGCGACGAGAGCCGCGCGGGACGACTCG
TCGGCGATAACCGGTGTCGGGTAGCGCCAGCCGCG
CGACGGTAACGAGGGACCGCGACAGGCAGACGCTC
CCATGATCACTCTGCACGCCGAAGGCAAATAGTGC
AGGCCGTGCGGCGCTTGGCGTTCCTTGGAAGGGCT
GAATCCCCGCCTCGTCCTTCGCAGCGGCCCCCCGG
GTGTTCCCATCGCCGCTTCTAGGCCCACTGCGACG
CTTGCCTGCACTTCTTACACGCTCTGGGTCCCAGC
CGCGGCGACGCAAAGGGCCTTGGTGCGGGTCTCGT
CGGCGCAGGGACGCGTTTGGGTCCCGACGGAACCT
TTTCCGCGTTGGGGTTGGGGCACCATAAGCT
Exemplary truncated PGK Promoters:
PGK100:
(SEQ ID NO: 110)
ACCCCTCTCTCCAGCCACTAAGCCAGTTGCTCCCT
CGGCTGACGGCTGCACGCGAGGCCTCCGAACGTCT
TACGCCTTGTGGCGCGCCCGTCCTTGTCCCGGGTG
TGATGGCGGGGTG
PGK200:
(SEQ ID NO: 111)
ACCCCTCTCTCCAGCCACTAAGCCAGTTGCTCCCT
CGGCTGACGGCTGCACGCGAGGCCTCCGAACGTCT
TACGCCTTGTGGCGCGCCCGTCCTTGTCCCGGGTG
TGATGGCGGGGTGTGGGGCGGAGGGCGTGGCGGGG
AAGGGCCGGCGACGAGAGCCGCGCGGGACGACTCG
TCGGCGATAACCGGTGTCGGGTAGCGCCAGCCGCG
CGACGGTAACG
PGK300:
(SEQ ID NO: 112)
ACCCCTCTCTCCAGCCACTAAGCCAGTTGCTCCCT
CGGCTGACGGCTGCACGCGAGGCCTCCGAACGTCT
TACGCCTTGTGGCGCGCCCGTCCTTGTCCCGGGTG
TGATGGCGGGGTGTGGGGCGGAGGGCGTGGCGGGG
AAGGGCCGGCGACGAGAGCCGCGCGGGACGACTCG
TCGGCGATAACCGGTGTCGGGTAGCGCCAGCCGCG
CGACGGTAACGAGGGACCGCGACAGGCAGACGCTC
CCATGATCACTCTGCACGCCGAAGGCAAATAGTGC
AGGCCGTGCGGCGCTTGGCGTTCCTTGGAAGGGCT
GAATCCCCG
PGK400:
(SEQ ID NO: 113)
ACCCCTCTCTCCAGCCACTAAGCCAGTTGCTCCCT
CGGCTGACGGCTGCACGCGAGGCCTCCGAACGTCT
TACGCCTTGTGGCGCGCCCGTCCTTGTCCCGGGTG
TGATGGCGGGGTGTGGGGCGGAGGGCGTGGCGGGG
AAGGGCCGGCGACGAGAGCCGCGCGGGACGACTCG
TCGGCGATAACCGGTGTCGGGTAGCGCCAGCCGCG
CGACGGTAACGAGGGACCGCGACAGGCAGACGCTC
CCATGATCACTCTGCACGCCGAAGGCAAATAGTGC
AGGCCGTGCGGCGCTTGGCGTTCCTTGGAAGGGCT
GAATCCCCGCCTCGTCCTTCGCAGCGGCCCCCCGG
GTGTTCCCATCGCCGCTTCTAGGCCCACTGCGACG
CTTGCCTGCACTTCTTACACGCTCTGGGTCCCAGC
CG

A vector may also include, e.g., a signal sequence to facilitate secretion, a polyadenylation signal and transcription terminator (e.g., from Bovine Growth Hormone (BGH) gene), an element allowing episomal replication and replication in prokaryotes (e.g. SV40 origin and ColE1 or others known in the art) and/or elements to allow selection (e.g., ampicillin resistance gene and/or zeocin marker).

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In embodiments, the vector may comprise two or more nucleic acid sequences encoding a CAR, e.g., a CAR described herein, e.g., a CD19 CAR, and a second CAR, e.g., an inhibitory CAR or a CAR that specifically binds to an antigen other than CD19. In such embodiments, the two or more nucleic acid sequences encoding the CAR are encoded by a single nucleic molecule in the same frame and as a single polypeptide chain. In this aspect, the two or more CARs, can, e.g., be separated by one or more peptide cleavage sites. (e.g., an auto-cleavage site or a substrate for an intracellular protease). Examples of peptide cleavage sites include T2A, P2A, E2A, or F2A sites.

Methods of introducing and expressing genes into a cell are known in the art. In the context of an expression vector, the vector can be readily introduced into a host cell, e.g., mammalian, bacterial, yeast, or insect cell by any method, e.g., one known in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). A suitable method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant nucleic acid sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Natural Killer Cell Receptor (NKR) CARs

In an embodiment, the CAR molecule described herein comprises one or more components of a natural killer cell receptor (NKR), thereby forming an NKR-CAR. The NKR component can be a transmembrane domain, a hinge domain, or a cytoplasmic domain from any of the following natural killer cell receptors: killer cell immunoglobulin-like receptor (KIR), e.g., KIR2DL1, KIR2DL2/L3, KIR2DL4, KIR2DL5A, KIR2DL5B, KIR2DS1, KIR2DS2, KIR2DS3, KIR2DS4, DIR2DS5, KIR3DL1/S1, KIR3DL2, KIR3DL3, KIR2DP1, and KIR3DP1; natural cytotoxicity receptor (NCR), e.g., NKp30, NKp44, NKp46; signaling lymphocyte activation molecule (SLAM) family of immune cell receptors, e.g., CD48, CD229, 2B4, CD84, NTB-A, CRACC, BLAME, and CD2F-10; Fc receptor (FcR), e.g., CD16, and CD64; and Ly49 receptors, e.g., LY49A, LY49C. The NKR-CAR molecules described herein may interact with an adaptor molecule or intracellular signaling domain, e.g., DAP12. Exemplary configurations and sequences of CAR molecules comprising NKR components are described in International Publication No. WO2014/145252, the contents of which are hereby incorporated by reference.

### Split CAR

In some embodiments, the CAR-expressing cell uses a split CAR. The split CAR approach is described in more detail in publications WO2014/055442 and WO2014/055657. Briefly, a split CAR system comprises a cell expressing a first CAR having a first antigen binding domain and a costimulatory domain (e.g., 41BB), and the cell also expresses a second CAR having a second antigen binding domain and an intracellular signaling domain (e.g., CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens.

### Strategies for Regulating Chimeric Antigen Receptors

In some embodiments, a regulatable CAR (RCAR) where the CAR activity can be controlled is desirable to optimize the safety and efficacy of a CAR therapy. There are many ways CAR activities can be regulated. For example, inducible apoptosis using, e.g., a caspase fused to a dimerization domain (see, e.g., Di Stasa et al., N Engl. J. Med. 2011 Nov. 3; 365(18):1673-1683), can be used as a safety switch in the CAR therapy of the instant invention. In one embodiment, the cells (e.g., T cells or NK cells) expressing a CAR of the present invention further comprise an inducible apoptosis switch, wherein a human caspase (e.g., caspase 9) or a modified version is fused to a modification of the human FKB protein that allows conditional dimerization. In the presence of a small molecule, such as a rapalog (e.g., AP 1903, AP20187), the inducible caspase (e.g., caspase 9) is activated and leads to the rapid apoptosis and death of the cells (e.g., T cells or NK cells) expressing a CAR of the present invention. Examples of a caspase-based inducible apoptosis switch (or one or more aspects of such a switch) have been described in, e.g., US2004040047; US20110286980; US20140255360; WO1997031899; WO2014151960; WO2014164348; WO2014197638; WO2014197638; all of which are incorporated by reference herein.

In another example, CAR-expressing cells can also express an inducible Caspase-9 (iCaspase-9) molecule that, upon administration of a dimerizer drug (e.g., rimiducid (also called AP1903 (Bellicum Pharmaceuticals) or AP20187 (Ariad)) leads to activation of the Caspase-9 and apoptosis of the cells. The iCaspase-9 molecule contains a chemical inducer of dimerization (CID) binding domain that mediates dimerization in the presence of a CID. This results in inducible and selective depletion of CAR-expressing cells. In some cases, the iCaspase-9 molecule is encoded by a nucleic acid molecule separate from the CAR-encoding vector(s). In some cases, the iCaspase-9 molecule is encoded by the same nucleic acid molecule as the CAR-encoding vector. The iCaspase-9 can provide a safety switch to avoid any toxicity of CAR-expressing cells. See, e.g., Song et al. Cancer Gene Ther. 2008; 15(10):667-75; Clinical Trial Id. No. NCT02107963; and Di Stasi et al. N. Engl. J. Med. 2011;

Alternative strategies for regulating the CAR therapy of the instant invention include utilizing small molecules or antibodies that deactivate or turn off CAR activity, e.g., by deleting CAR-expressing cells, e.g., by inducing antibody dependent cell-mediated cytotoxicity (ADCC). For example, CAR-expressing cells described herein may also express an antigen that is recognized by molecules capable of inducing cell death, e.g., ADCC or complement-induced cell death. For example, CAR expressing cells described herein may also express a receptor capable of being targeted by an antibody or antibody fragment. Examples of such receptors include EpCAM, VEGFR, integrins (e.g., integrins αvβ3, α4, αI¾β3, α4β7, α5β1, αvβ3, αv), members of the TNF receptor superfamily (e.g., TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD1 1, CD1 1 a/LFA-1, CD15, CD18/ITGB2, CD19, CD20, CD22, CD23/lgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (e.g., versions preserving one or more extracellular epitopes but lacking one or more regions within the cytoplasmic domain).

For example, a CAR-expressing cell described herein may also express a truncated epidermal growth factor receptor (EGFR) which lacks signaling capacity but retains the epitope that is recognized by molecules capable of inducing ADCC, e.g., cetuximab (ERBITUX®), such that administration of cetuximab induces ADCC and subsequent depletion of the CAR-expressing cells (see, e.g., WO2011/056894, and Jonnalagadda et al., Gene Ther. 2013; 20(8)853-860). Another strategy includes expressing a highly compact marker/suicide gene that combines target epitopes from both CD32 and CD20 antigens in the CAR-expressing cells described herein, which binds rituximab, resulting in selective depletion of the CAR-expressing cells, e.g., by ADCC (see, e.g., Philip et al., Blood. 2014; 124(8)1277-1287). Other methods for depleting CAR-expressing cells described herein include administration of CAMPATH, a monoclonal anti-CD52 antibody that selectively binds and targets mature lymphocytes, e.g., CAR-expressing cells, for destruction, e.g., by inducing ADCC. In other embodiments, the CAR-expressing cell can be selectively targeted using a CAR ligand, e.g., an anti-idiotypic antibody. In some embodiments, the anti-idiotypic antibody can cause effector cell activity, e.g., ADCC or ADC activities, thereby reducing the number of CAR-expressing cells. In other embodiments, the CAR ligand, e.g., the anti-idiotypic antibody, can be coupled to an agent that induces cell killing, e.g., a toxin, thereby reducing the number of CAR-expressing cells. Alternatively, the CAR molecules themselves can be configured such that the activity can be regulated, e.g., turned on and off, as described below.

In other embodiments, a CAR-expressing cell described herein may also express a target protein recognized by the T cell depleting agent. In one embodiment, the target protein is CD20 and the T cell depleting agent is an anti-CD20 antibody, e.g., rituximab. In such embodiment, the T cell depleting agent is administered once it is desirable to reduce or eliminate the CAR-expressing cell, e.g., to mitigate the CAR induced toxicity. In other embodiments, the T cell depleting agent is an anti-CD52 antibody, e.g., alemtuzumab, as described in the Examples herein.

In other embodiments, an RCAR comprises a set of polypeptides, typically two in the simplest embodiments, in which the components of a standard CAR described herein, e.g., an antigen binding domain and an intracellular signalling domain, are partitioned on separate polypeptides or members. In some embodiments, the set of polypeptides include a dimerization switch that, upon the presence of a dimerization molecule, can couple the polypeptides to one another, e.g., can couple an antigen binding domain to an intracellular signalling domain. In one embodiment, a CAR of the present invention utilizes a dimerization switch as those described in, e.g., WO2014127261, which is incorporated by reference herein. Additional description and exemplary configurations of such regulatable CARs are provided herein and in, e.g., paragraphs 527-551 of International Publication No. WO 2015/090229 filed Mar. 13, 2015, which is incorporated by reference in its entirety. In some embodiments, an RCAR involves a switch domain, e.g., a FKBP switch domain, as set out SEQ ID NO: 114, or comprise a fragment of FKBP having the ability to bind with FRB, e.g., as set out in SEQ ID NO: 115. In some embodiments, the RCAR involves a switch domain comprising a FRB sequence, e.g., as set out in SEQ ID NO: 116, or a mutant FRB sequence, e.g., as set out in any of SEQ ID Nos. 117-122 in Table 13A.

(SEQ ID NO: 114)
DVPDYASLGGPSSPKKKRKVSRGVQVETISPGDGR
TFPKRGQTCVVHYTGMLEDGKKFDSSRDRNKPFKF
MLGKQEVIRGWEEGVAQMSVGQRAKLTISPDYAYG
ATGHPGIIPPHATLVFDVELLKLETSY
(SEQ ID NO: 115)
VQVETISPGDGRTFPKRGQTCVVHYTGMLEDGKKF
DSSRDRNKPFKFMLGKQEVIRGWEEGVAQMSVGQR
AKLTISPDYAYGATGHPGIIPPHATLVFDVELLKL
ETS
(SEQ ID NO: 116)
ILWHEMWHEGLEEASRLYFGERNVKGMFEVLEPLH
AMMERGPQTLKETSFNQAYGRDLMEAQEWCRKYMK
SGNVKDLTQAWDLYYHVFRRISK

TABLE 13A
Exemplary mutant FRB having increased
affinity for a dimerization molecule.

| | | SEQ |
|---|---|---|
| | | ID |
| FRB mutant | Amino Acid Sequence | NO: |
| E2032I mutant | ILWHEMWHEGLIEASRLYFG | 117 |
| | ERNVKGMFEVLEPLHAMMER | |
| | GPQTLKETSFNQAYGRDLME | |
| | AQEWCRKYMKSGNVKDLTQA | |
| | WDLYYHVFRRISKTS | |
| E2032L mutant | ILWHEMWHEGLLEASRLYFG | 118 |
| | ERNVKGMFEVLEPLHAMMER | |
| | GPQTLKETSFNQAYGRDLME | |
| | AQEWCRKYMKSGNVKDLTQA | |
| | WDLYYHVFRRISKTS | |
| T2098L mutant | ILWHEMWHEGLEEASRLYFG | 119 |
| | ERNVKGMFEVLEPLHAMMER | |
| | GPQTLKETSFNQAYGRDLME | |
| | AQEWCRKYMKSGNVKDLLQA | |
| | WDLYYHVFRRISKTS | |
| E2032, T2098 | ILWHEMWHEGL [symbol] EASRLYFG | 120 |
| mutant | ERNVKGMFEVLEPLHAMMER | |
| | GPQTLKETSFNQAYGRDLME | |
| | AQEWCRKYMKSGNVKDL [symbol] QA | |
| | WDLYYHVFRRISKTS | 121 |
| E2032I, T2098L | ILWHEMWHEGLIEASRLYFG | |
| | ERNVKGMFEVLEPLHAMMER | |
| | GPQTLKETSFNQAYGRDLME | |
| | AQEWCRKYMKSGNVKDLLQA | |
| mutant | WDLYYHVFRRISKTS | 122 |
| E2032L, T2098L | ILWHEMWHEGLLEASRLYFG | |
| mutant | ERNVKGMFEVLEPLHAMMER | |
| | GPQTLKETSFNQAYGRDLME | |
| | AQEWCRKYMKSGNVKDLLQA | |
| | WDLYYHVFRRISKTS | |

### RNA Transfection

Disclosed herein are methods for producing an in vitro transcribed RNA CAR. RNA CAR and methods of using the same are described, e.g., in paragraphs 553-570 of in International Application WO2015/142675, filed Mar. 13, 2015, which is herein incorporated by reference in its entirety.

An immune effector cell can include a CAR encoded by a messenger RNA (mRNA). In one aspect, the mRNA encoding a CAR described herein is introduced into an immune effector cell, e.g., made by a method described herein, for production of a CAR-expressing cell.

In one embodiment, the in vitro transcribed RNA CAR can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired temple for in vitro transcription is a CAR described herein. For example, the template for the RNA CAR comprises an extracellular region comprising a single chain variable domain of an antibody to a tumor associated antigen described herein; a hinge region (e.g., a hinge region described herein), a transmembrane domain (e.g., a transmembrane domain described herein such as a transmembrane domain of CD8a); and a cytoplasmic region that includes an intracellular signaling domain, e.g., an intracellular signaling domain described herein, e.g., comprising the signaling domain of CD3-zeta and the signaling domain of 4-1BB.

In one embodiment, the DNA to be used for PCR contains an open reading frame. The DNA can be from a naturally occurring DNA sequence from the genome of an organism. In one embodiment, the nucleic acid can include some or all of the 5′ and/or 3′ untranslated regions (UTRs). The nucleic acid can include exons and introns. In one embodiment, the DNA to be used for PCR is a human nucleic acid sequence. In another embodiment, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In one embodiment, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA in embodiments has 5′ and 3′ UTRs. In one embodiment, the 5′ UTR is between one and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be 5′UTR of an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In an embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However, polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (SEQ ID NO: 123) (size can be 50-5000 T (SEQ ID NO: 32)), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines (e.g., SEQ ID NO: 33).

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides (SEQ ID NO: 34) results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to RNA molecules. In an embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70

### Non-Viral Delivery Methods

In some aspects, non-viral methods can be used to deliver a nucleic acid encoding a CAR described herein into a cell or tissue or a subject.

In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. For example, a transposon comprises a DNA sequence made up of inverted repeats flanking genes for transposition.

Exemplary methods of nucleic acid delivery using a transposon include a Sleeping Beauty transposon system (SBTS) and a piggyBac (PB) transposon system. See, e.g., Aronovich et al. Hum. Mol. Genet. 20.R1 (2011):R14-20; Singh et al. Cancer Res. 15 (2008):2961-2971; Huang et al. Mol. Ther. 16 (2008):580-589; Grabundzija et al. Mol. Ther. 18 (2010):1200-1209; Kebriaei et al. Blood. 122.21 (2013):166; Williams. Molecular Therapy 16.9 (2008):1515-16; Bell et al. Nat. Protoc. 2.12 (2007):3153-65; and Ding et al. Cell. 122.3 (2005):473-83, all of which are incorporated herein by reference.

The SBTS includes two components: 1) a transposon containing a transgene and 2) a source of transposase enzyme. The transposase can transpose the transposon from a carrier plasmid (or other donor DNA) to a target DNA, such as a host cell chromosome/genome. For example, the transposase binds to the carrier plasmid/donor DNA, cuts the transposon (including transgene(s)) out of the plasmid, and inserts it into the genome of the host cell. See, e.g., Aronovich et al. supra.

Exemplary transposons include a pT2-based transposon. See, e.g., Grabundzija et al. Nucleic Acids Res. 41.3(2013):1829-47; and Singh et al. Cancer Res. 68.8(2008): 2961-2971, all of which are incorporated herein by reference. Exemplary transposases include a Tc1/mariner-type transposase, e.g., the SB10 transposase or the SB11 transposase (a hyperactive transposase which can be expressed, e.g., from a cytomegalovirus promoter). See, e.g., Aronovich et al.; Kebriaei et al.; and Grabundzija et al., all of which are incorporated herein by reference.

Use of the SBTS permits efficient integration and expression of a transgene, e.g., a nucleic acid encoding a CAR described herein. Provided herein are methods of generating a cell, e.g., T cell or NK cell, that stably expresses a CAR described herein, e.g., using a transposon system such as SBTS.

In accordance with methods described herein, in some embodiments, one or more nucleic acids, e.g., plasmids, containing the SBTS components are delivered to a cell (e.g., T or NK cell). For example, the nucleic acid(s) are delivered by standard methods of nucleic acid (e.g., plasmid DNA) delivery, e.g., methods described herein, e.g., electroporation, transfection, or lipofection. In some embodiments, the nucleic acid contains a transposon comprising a transgene, e.g., a nucleic acid encoding a CAR described herein. In some embodiments, the nucleic acid contains a transposon comprising a transgene (e.g., a nucleic acid encoding a CAR described herein) as well as a nucleic acid sequence encoding a transposase enzyme. In other embodiments, a system with two nucleic acids is provided, e.g., a dual-plasmid system, e.g., where a first plasmid contains a transposon comprising a transgene, and a second plasmid contains a nucleic acid sequence encoding a transposase enzyme. For example, the first and the second nucleic acids are co-delivered into a host cell.

In some embodiments, cells, e.g., T or NK cells, are generated that express a CAR described herein by using a combination of gene insertion using the SBTS and genetic editing using a nuclease (e.g., Zinc finger nucleases (ZFNs), Transcription Activator-Like Effector Nucleases (TALENs), the CRISPR/Cas system, or engineered meganuclease re-engineered homing endonucleases).

In some embodiments, use of a non-viral method of delivery permits reprogramming of cells, e.g., T or NK cells, and direct infusion of the cells into a subject. Advantages of non-viral vectors include but are not limited to the ease and relatively low cost of producing sufficient amounts required to meet a patient population, stability during storage, and lack of immunogenicity.

### Methods of Manufacture/Production

In some embodiments, the methods disclosed herein further include administering a T cell depleting agent after treatment with the cell (e.g., an immune effector cell as described herein), thereby reducing (e.g., depleting) the CAR-expressing cells (e.g., the CD19CAR-expressing cells). Such T cell depleting agents can be used to effectively deplete CAR-expressing cells (e.g., CD19CAR-expressing cells) to mitigate toxicity. In some embodiments, the CAR-expressing cells were manufactured according to a method herein, e.g., assayed (e.g., before or after transfection or transduction) according to a method herein.

In some embodiments, the T cell depleting agent is administered one, two, three, four, or five weeks after administration of the cell, e.g., the population of immune effector cells, described herein.

In one embodiment, the T cell depleting agent is an agent that depletes CAR-expressing cells, e.g., by inducing antibody dependent cell-mediated cytotoxicity (ADCC) and/or complement-induced cell death. For example, CAR-expressing cells described herein may also express an antigen (e.g., a target antigen) that is recognized by molecules capable of inducing cell death, e.g., ADCC or complement-induced cell death. For example, CAR expressing cells described herein may also express a target protein (e.g., a receptor) capable of being targeted by an antibody or antibody fragment. Examples of such target proteins include, but are not limited to, EpCAM, VEGFR, integrins (e.g., integrins αvβ3, α4, αI¾β3, α4β7, α5β1, αvβ3, αv), members of the TNF receptor superfamily (e.g., TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/lgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (e.g., versions preserving one or more extracellular epitopes but lacking one or more regions within the cytoplasmic domain).

In some embodiments, the CAR expressing cell co-expresses the CAR and the target protein, e.g., naturally expresses the target protein or is engineered to express the target protein. For example, the cell, e.g., the population of immune effector cells, can include a nucleic acid (e.g., vector) comprising the CAR nucleic acid (e.g., a CAR nucleic acid as described herein) and a nucleic acid encoding the target protein.

In one embodiment, the T cell depleting agent is a CD52 inhibitor, e.g., an anti-CD52 antibody molecule, e.g., alemtuzumab.

In other embodiments, the cell, e.g., the population of immune effector cells, expresses a CAR molecule as described herein (e.g., CD19CAR) and the target protein recognized by the T cell depleting agent. In one embodiment, the target protein is CD20. In embodiments where the target protein is CD20, the T cell depleting agent is an anti-CD20 antibody, e.g., rituximab.

In further embodiments of any of the aforesaid methods, the methods further include transplanting a cell, e.g., a hematopoietic stem cell, or a bone marrow, into the mammal.

In another aspect, the invention features a method of conditioning a mammal prior to cell transplantation. The method includes administering to the mammal an effective amount of the cell comprising a CAR nucleic acid or polypeptide, e.g., a CD19 CAR nucleic acid or polypeptide. In some embodiments, the cell transplantation is a stem cell transplantation, e.g., a hematopoietic stem cell transplantation, or a bone marrow transplantation. In other embodiments, conditioning a subject prior to cell transplantation includes reducing the number of target-expressing cells in a subject, e.g., CD19-expressing normal cells or CD19-expressing cancer cells.

### Activation and Expansion of Immune Effector Cells (e.g., T Cells)

Immune effector cells such as T cells generated or enriched by the methods described herein may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, a population of immune effector cells, e.g., T regulatory cell depleted cells, may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain aspects, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one aspect, the agent providing the costimulatory signal is bound to a cell surface and the agent providing the primary activation signal is in solution or coupled to a surface. In certain aspects, both agents can be in solution. In one aspect, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one aspect, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts. In one aspect, a 1:1 ratio of each antibody bound to the beads for CD4+ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular aspect an increase of from about 1 to about 3 fold is observed as compared to the expansion observed using a ratio of 1:1. In one aspect, the ratio of CD3:CD28 antibody bound to the beads ranges from 100:1 to 1:100 and all integer values there between. In one aspect, more anti-CD28 antibody is bound to the particles than anti-CD3 antibody, i.e., the ratio of CD3:CD28 is less than one. In certain aspects, the ratio of anti CD28 antibody to anti CD3 antibody bound to the beads is greater than 2:1. In one particular aspect, a 1:100 CD3:CD28 ratio of antibody bound to beads is used. In one aspect, a 1:75 CD3:CD28 ratio of antibody bound to beads is used. In a further aspect, a 1:50 CD3:CD28 ratio of antibody bound to beads is used. In one aspect, a 1:30 CD3:CD28 ratio of antibody bound to beads is used. In one aspect, a 1:10 CD3:CD28 ratio of antibody bound to beads is used. In one aspect, a 1:3 CD3:CD28 ratio of antibody bound to the beads is used. In yet one aspect, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain aspects the ratio of cells to particles ranges from 1:100 to 100:1 and any integer values in-between and in further aspects the ratio comprises 1:9 to 9:1 and any integer values in between, can also be used to stimulate T cells. The ratio of anti-CD3- and anti-CD28-coupled particles to T cells that result in T cell stimulation can vary as noted above, however certain suitable values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one suitable ratio being at least 1:1 particles per T cell. In one aspect, a ratio of particles to cells of 1:1 or less is used. In one particular aspect, a suitable particle: cell ratio is 1:5. In further aspects, the ratio of particles to cells can be varied depending on the day of stimulation. For example, in one aspect, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular aspect, the ratio of particles to cells is 1:1 on the first day of stimulation and adjusted to 1:5 on the third and fifth days of stimulation. In one aspect, particles are added on a daily or every other day basis to a final ratio of 1:1 on the first day, and 1:5 on the third and fifth days of stimulation. In one aspect, the ratio of particles to cells is 2:1 on the first day of stimulation and adjusted to 1:10 on the third and fifth days of stimulation. In one aspect, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type. In one aspect, the most typical ratios for use are in the neighborhood of 1:1, 2:1 and 3:1 on the first day.

In further aspects, the cells, such as T cells, are combined with agent-coated beads, the beads and the cells are subsequently separated, and then the cells are cultured. In an alternative aspect, prior to culture, the agent-coated beads and cells are not separated but are cultured together. In a further aspect, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one aspect the cells (for example, 10⁴ to 10⁹ T cells) and beads (for example, DYNABEADS® M-450 CD3/CD28 T paramagnetic beads at a ratio of 1:1) are combined in a buffer, for example PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain aspects, it may be desirable to significantly decrease the volume in which particles and cells are mixed together (i.e., increase the concentration of cells), to ensure maximum contact of cells and particles. For example, in one aspect, a concentration of about 10 billion cells/ml, 9 billion/ml, 8 billion/ml, 7 billion/ml, 6 billion/ml, 5 billion/ml, or 2 billion cells/ml is used. In one aspect, greater than 100 million cells/ml is used. In a further aspect, a concentration of cells of 10, 15, 20, 25, 30, 35, 40, 45, or 50 million cells/ml is used. In yet one aspect, a concentration of cells from 75, 80, 85, 90, 95, or 100 million cells/ml is used. In further aspects, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain aspects. For example, using high concentration of cells allows more efficient selection of CD8+ T cells that normally have weaker CD28 expression.

In one embodiment, cells transduced with a nucleic acid encoding a CAR, e.g., a CAR described herein, e.g., a CD19 CAR described herein, are expanded, e.g., by a method described herein. In one embodiment, the cells are expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells are expanded for a period of 4 to 9 days. In one embodiment, the cells are expanded for a period of 8 days or less, e.g., 7, 6 or 5 days. In one embodiment, the cells are expanded in culture for 5 days, and the resulting cells are more potent than the same cells expanded in culture for 9 days under the same culture conditions. Potency can be defined, e.g., by various T cell functions, e.g. proliferation, target cell killing, cytokine production, activation, migration, or combinations thereof. In one embodiment, the cells, e.g., a CD19 CAR cell described herein, expanded for 5 days show at least a one, two, three or four fold increase in cells doublings upon antigen stimulation as compared to the same cells expanded in culture for 9 days under the same culture conditions. In one embodiment, the cells, e.g., the cells expressing a CD19 CAR described herein, are expanded in culture for 5 days, and the resulting cells exhibit higher proinflammatory cytokine production, e.g., IFN-γ and/or GM-CSF levels, as compared to the same cells expanded in culture for 9 days under the same culture conditions. In one embodiment, the cells, e.g., a CD19 CAR cell described herein, expanded for 5 days show at least a one, two, three, four, five, ten fold or more increase in pg/ml of proinflammatory cytokine production, e.g., IFN-γ and/or GM-CSF levels, as compared to the same cells expanded in culture for 9 days under the same culture conditions.

Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In one embodiment, the cells are expanded in an appropriate media (e.g., media described herein) that includes one or more interleukin that result in at least a 200-fold (e.g., 200-fold, 250-fold, 300-fold, 350-fold) increase in cells over a 14 day expansion period, e.g., as measured by a method described herein such as flow cytometry. In one embodiment, the cells are expanded in the presence IL-15 and/or IL-7 (e.g., IL-15 and IL-7).

In embodiments, methods described herein, e.g., CAR-expressing cell manufacturing methods, comprise removing T regulatory cells, e.g., CD25+ T cells, from a cell population, e.g., using an anti-CD25 antibody, or fragment thereof, or a CD25-binding ligand, IL-2. Methods of removing T regulatory cells, e.g., CD25+ T cells, from a cell population are described herein. In embodiments, the methods, e.g., manufacturing methods, further comprise contacting a cell population (e.g., a cell population in which T regulatory cells, such as CD25+ T cells, have been depleted; or a cell population that has previously contacted an anti-CD25 antibody, fragment thereof, or CD25-binding ligand) with IL-15 and/or IL-7. For example, the cell population (e.g., that has previously contacted an anti-CD25 antibody, fragment thereof, or CD25-binding ligand) is expanded in the presence of IL-15 and/or IL-7.

In some embodiments a CAR-expressing cell described herein is contacted with a composition comprising a interleukin-15 (IL-15) polypeptide, a interleukin-15 receptor alpha (IL-15Ra) polypeptide, or a combination of both a IL-15 polypeptide and a IL-15Ra polypeptide e.g., hetIL-15, during the manufacturing of the CAR-expressing cell, e.g., ex vivo. In embodiments, a CAR-expressing cell described herein is contacted with a composition comprising a IL-15 polypeptide during the manufacturing of the CAR-expressing cell, e.g., ex vivo. In embodiments, a CAR-expressing cell described herein is contacted with a composition comprising a combination of both a IL-15 polypeptide and a IL-15 Ra polypeptide during the manufacturing of the CAR-expressing cell, e.g., ex vivo. In embodiments, a CAR-expressing cell described herein is contacted with a composition comprising hetIL-15 during the manufacturing of the CAR-expressing cell, e.g., ex vivo.

In one embodiment the CAR-expressing cell described herein is contacted with a composition comprising hetIL-15 during ex vivo expansion. In an embodiment, the CAR-expressing cell described herein is contacted with a composition comprising an IL-15 polypeptide during ex vivo expansion. In an embodiment, the CAR-expressing cell described herein is contacted with a composition comprising both an IL-15 polypeptide and an IL-15Ra polypeptide during ex vivo expansion. In one embodiment the contacting results in the survival and proliferation of a lymphocyte subpopulation, e.g., CD8+ T cells.

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once a CAR described herein is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of a CAR of the present invention are described in further detail below

Western blot analysis of CAR expression in primary T cells can be used to detect the presence of monomers and dimers, e.g., as described in paragraph 695 of International Application WO2015/142675, filed Mar. 13, 2015, which is herein incorporated by reference in its entirety.

In vitro expansion of CAR⁺ T cells following antigen stimulation can be measured by flow cytometry. For example, a mixture of CD4⁺ and CD8⁺ T cells are stimulated with αCD3/αCD28 aAPCs followed by transduction with lentiviral vectors expressing GFP under the control of the promoters to be analyzed. Exemplary promoters include the CMV IE gene, EF-1α, ubiquitin C, or phosphoglycerokinase (PGK) promoters. GFP fluorescence is evaluated on day 6 of culture in the CD4⁺ and/or CD8⁺ T cell subsets by flow cytometry. See, e.g., Milone et al., Molecular Therapy 17(8): 1453-1464 (2009). Alternatively, a mixture of CD4⁺ and CD8⁺ T cells are stimulated with αCD3/αCD28 coated magnetic beads on day 0, and transduced with CAR on day 1 using a bicistronic lentiviral vector expressing CAR along with eGFP using a 2A ribosomal skipping sequence. Cultures are re-stimulated with either a cancer associated antigen as described herein⁺ K562 cells (K562-expressing a cancer associated antigen as described herein), wild-type K562 cells (K562 wild type) or K562 cells expressing hCD32 and 4-1BBL in the presence of antiCD3 and anti-CD28 antibody (K562-BBL-3/28) following washing. Exogenous IL-2 is added to the cultures every other day at 100 IU/ml. GFP⁺ T cells are enumerated by flow cytometry using bead-based counting. See, e.g., Milone et al., Molecular Therapy 17(8): 1453-1464 (2009).

Sustained CAR⁺ T cell expansion in the absence of re-stimulation can also be measured. See, e.g., Milone et al., Molecular Therapy 17(8): 1453-1464 (2009). Briefly, mean T cell volume (fl) is measured on day 8 of culture using a Coulter Multisizer III particle counter, a Nexcelom Cellometer Vision or Millipore Scepter, following stimulation with αCD3/αCD28 coated magnetic beads on day 0, and transduction with the indicated CAR on day 1.

Animal models can also be used to measure a CAR-expressing cell activity, e.g., as described in paragraph 698 of International Application WO2015/142675, filed Mar. 13, 2015, which is herein incorporated by reference in its entirety.

Dose dependent CAR treatment response can be evaluated, e.g., as described in paragraph 699 of International Application WO2015/142675, filed Mar. 13, 2015, which is herein incorporated by reference in its entirety.

Assessment of cell proliferation and cytokine production has been previously described, as described in paragraph 700 of International Application WO2015/142675, filed Mar. 13, 2015, which is herein incorporated by reference in its entirety.

Cytotoxicity can be assessed by a standard 51Cr-release assay, e.g., as described in paragraph 701 of International Application WO2015/142675, filed Mar. 13, 2015, which is herein incorporated by reference in its entirety.

Cytotoxicity can also be assessed by measuring changes in adherent cell's electrical impedance, e.g., using an xCELLigence real time cell analyzer (RTCA). In some embodiments, cytotoxicity is measured at multiple time points.

Imaging technologies can be used to evaluate specific trafficking and proliferation of CARs in tumor-bearing animal models, e.g., as described in paragraph 702 of International Application WO2015/142675, filed Mar. 13, 2015, which is herein incorporated by reference in its entirety.

Other assays, including those described in the Example section herein as well as those that are known in the art can also be used to evaluate the CARs described herein.

Alternatively, or in combination to the methods disclosed herein, methods and compositions for one or more of: detection and/or quantification of CAR-expressing cells (e.g., in vitro or in vivo (e.g., clinical monitoring)); immune cell expansion and/or activation; and/or CAR-specific selection, that involve the use of a CAR ligand, are disclosed. In one exemplary embodiment, the CAR ligand is an antibody that binds to the CAR molecule, e.g., binds to the extracellular antigen binding domain of CAR (e.g., an antibody that binds to the antigen binding domain, e.g., an anti-idiotypic antibody; or an antibody that binds to a constant region of the extracellular binding domain). In other embodiments, the CAR ligand is a CAR antigen molecule (e.g., a CAR antigen molecule as described herein).

In one aspect, a method for detecting and/or quantifying CAR-expressing cells is disclosed. For example, the CAR ligand can be used to detect and/or quantify CAR-expressing cells in vitro or in vivo (e.g., clinical monitoring of CAR-expressing cells in a patient, or dosing a patient). The method includes:

providing the CAR ligand (optionally, a labelled CAR ligand, e.g., a CAR ligand that includes a tag, a bead, a radioactive or fluorescent label);

acquiring the CAR-expressing cell (e.g., acquiring a sample containing CAR-expressing cells, such as a manufacturing sample or a clinical sample);

contacting the CAR-expressing cell with the CAR ligand under conditions where binding occurs, thereby detecting the level (e.g., amount) of the CAR-expressing cells present. Binding of the CAR-expressing cell with the CAR ligand can be detected using standard techniques such as FACS, ELISA and the like.

In another aspect, a method of expanding and/or activating cells (e.g., immune effector cells) is disclosed. The method includes:

providing a CAR-expressing cell (e.g., a first CAR-expressing cell or a transiently expressing CAR cell);

contacting said CAR-expressing cell with a CAR ligand, e.g., a CAR ligand as described herein), under conditions where immune cell expansion and/or proliferation occurs, thereby producing the activated and/or expanded cell population.

In certain embodiments, the CAR ligand is present on a substrate (e.g., is immobilized or attached to a substrate, e.g., a non-naturally occurring substrate). In some embodiments, the substrate is a non-cellular substrate. The non-cellular substrate can be a solid support chosen from, e.g., a plate (e.g., a microtiter plate), a membrane (e.g., a nitrocellulose membrane), a matrix, a chip or a bead. In embodiments, the CAR ligand is present in the substrate (e.g., on the substrate surface). The CAR ligand can be immobilized, attached, or associated covalently or non-covalently (e.g., cross-linked) to the substrate. In one embodiment, the CAR ligand is attached (e.g., covalently attached) to a bead. In the aforesaid embodiments, the immune cell population can be expanded in vitro or ex vivo. The method can further include culturing the population of immune cells in the presence of the ligand of the CAR molecule, e.g., using any of the methods described herein.

In other embodiments, the method of expanding and/or activating the cells further comprises addition of a second stimulatory molecule, e.g., CD28. For example, the CAR ligand and the second stimulatory molecule can be immobilized to a substrate, e.g., one or more beads, thereby providing increased cell expansion and/or activation.

In yet another aspect, a method for selecting or enriching for a CAR expressing cell is provided. The method includes contacting the CAR expressing cell with a CAR ligand as described herein; and selecting the cell on the basis of binding of the CAR ligand.

In yet other embodiments, a method for depleting, reducing and/or killing a CAR expressing cell is provided. The method includes contacting the CAR expressing cell with a CAR ligand as described herein; and targeting the cell on the basis of binding of the CAR ligand, thereby reducing the number, and/or killing, the CAR-expressing cell. In one embodiment, the CAR ligand is coupled to a toxic agent (e.g., a toxin or a cell ablative drug). In another embodiment, the anti-idiotypic antibody can cause effector cell activity, e.g., ADCC or ADC activities.

Exemplary anti-CAR antibodies that can be used in the methods disclosed herein are described, e.g., in WO 2014/190273 and by Jena et al., “Chimeric Antigen Receptor (CAR)-Specific Monoclonal Antibody to Detect CD19-Specific T cells in Clinical Trials”, PLOS March 2013 8:3 e57838, the contents of which are incorporated by reference.

In some aspects and embodiments, the compositions and methods herein are optimized for a specific subset of T cells, e.g., as described in US Serial No. PCT/US2015/043219 filed Jul. 31, 2015, the contents of which are incorporated herein by reference in their entirety. In some embodiments, the optimized subsets of T cells display an enhanced persistence compared to a control T cell, e.g., a T cell of a different type (e.g., CD8+ or CD4+) expressing the same construct.

In some embodiments, a CD4+ T cell comprises a CAR described herein, which CAR comprises an intracellular signaling domain suitable for (e.g., optimized for, e.g., leading to enhanced persistence in) a CD4+ T cell, e.g., an ICOS domain. In some embodiments, a CD8+ T cell comprises a CAR described herein, which CAR comprises an intracellular signaling domain suitable for (e.g., optimized for, e.g., leading to enhanced persistence of) a CD8+ T cell, e.g., a 4-1BB domain, a CD28 domain, or another costimulatory domain other than an ICOS domain. In some embodiments, the CAR described herein comprises an antigen binding domain described herein, e.g., a CAR comprising an antigen binding domain.

In an aspect, described herein is a method of treating a subject, e.g., a subject having cancer. The method includes administering to said subject, an effective amount of:

1) a CD4+ T cell comprising a CAR (the CARCD4+) comprising:

an antigen binding domain, e.g., an antigen binding domain described herein;

a transmembrane domain; and

an intracellular signaling domain, e.g., a first costimulatory domain, e.g., an ICOS domain; and

2) a CD8+ T cell comprising a CAR (the CARCD8+) comprising:

an antigen binding domain, e.g., an antigen binding domain described herein;

a transmembrane domain; and

an intracellular signaling domain, e.g., a second costimulatory domain, e.g., a 4-1BB domain, a CD28 domain, or another costimulatory domain other than an ICOS domain;

wherein the CARCD4+ and the CARCD8+ differ from one another.

Optionally, the method further includes administering:

3) a second CD8+ T cell comprising a CAR (the second CARCD8+) comprising:

an antigen binding domain, e.g., an antigen binding domain described herein;

a transmembrane domain; and

an intracellular signaling domain, wherein the second CARCD8+ comprises an intracellular signaling domain, e.g., a costimulatory signaling domain, not present on the CARCD8+, and, optionally, does not comprise an ICOS signaling domain.

### Biopolymer Delivery Methods

In some embodiments, one or more CAR-expressing cells as disclosed herein can be administered or delivered to the subject via a biopolymer scaffold, e.g., a biopolymer implant. Biopolymer scaffolds can support or enhance the delivery, expansion, and/or dispersion of the CAR-expressing cells described herein. A biopolymer scaffold comprises a biocompatible (e.g., does not substantially induce an inflammatory or immune response) and/or a biodegradable polymer that can be naturally occurring or synthetic. Exemplary biopolymers are described, e.g., in paragraphs 1004-1006 of International Application WO2015/142675, filed Mar. 13, 2015, which is herein incorporated by reference in its entirety.

### Pharmaceutical Compositions and Treatments

In some aspects, the disclosure provides a method of treating a subject, comprising administering CAR-expressing cells produced as described herein, optionally in combination with one or more other therapies. In some aspects, the disclosure provides a method of treating a subject, comprising administering a reaction mixture comprising CAR-expressing cells as described herein, optionally in combination with one or more other therapies. In some aspects, the disclosure provides a method of shipping or receiving a reaction mixture comprising CAR-expressing cells as described herein. In some aspects, the disclosure provides a method of treating a subject, comprising receiving a CAR-expressing cell that was produced as described herein, and further comprising administering the CAR-expressing cell to the subject, optionally in combination with one or more other therapies. In some aspects, the disclosure provides a method of treating a subject, comprising producing a CAR-expressing cell as described herein, and further comprising administering the CAR-expressing cell to the subject, optionally in combination with one or more other therapies. The other therapy may be, e.g., a cancer therapy such as chemotherapy.

In an embodiment, cells expressing a CAR described herein are administered to a subject in combination with a molecule that decreases the Treg cell population. Methods that decrease the number of (e.g., deplete) Treg cells are known in the art and include, e.g., CD25 depletion, cyclophosphamide administration, modulating GITR function. Without wishing to be bound by theory, it is believed that reducing the number of Treg cells in a subject prior to apheresis or prior to administration of a CAR-expressing cell described herein reduces the number of unwanted immune cells (e.g., Tregs) in the tumor microenvironment and reduces the subject's risk of relapse.

In one embodiment, a therapy described herein, e.g., a CAR-expressing cell, is administered to a subject in combination with a molecule targeting GITR and/or modulating GITR functions, such as a GITR agonist and/or a GITR antibody that depletes regulatory T cells (Tregs). In embodiments, cells expressing a CAR described herein are administered to a subject in combination with cyclophosphamide. In one embodiment, the GITR binding molecules and/or molecules modulating GITR functions (e.g., GITR agonist and/or Treg depleting GITR antibodies) are administered prior to the CAR-expressing cell. For example, in one embodiment, a GITR agonist can be administered prior to apheresis of the cells. In embodiments, cyclophosphamide is administered to the subject prior to administration (e.g., infusion or re-infusion) of the CAR-expressing cell or prior to apheresis of the cells. In embodiments, cyclophosphamide and an anti-GITR antibody are administered to the subject prior to administration (e.g., infusion or re-infusion) of the CAR-expressing cell or prior to apheresis of the cells. In one embodiment, the subject has cancer (e.g., a solid cancer or a hematological cancer such as ALL or CLL). In one embodiment, the subject has CLL. In embodiments, the subject has ALL. In embodiments, the subject has a solid cancer, e.g., a solid cancer described herein. Exemplary GITR agonists include, e.g., GITR fusion proteins and anti-GITR antibodies (e.g., bivalent anti-GITR antibodies) such as, e.g., a GITR fusion protein described in U.S. Pat. No. 6,111,090, European Patent No.: 090505B1, U.S. Pat. No. 8,586,023, PCT Publication Nos.: WO 2010/003118 and 2011/090754, or an anti-GITR antibody described, e.g., in U.S. Pat. No. 7,025,962, European Patent No.: 1947183B1, U.S. Pat. Nos. 7,812,135, 8,388,967, 8,591,886, European Patent No.: EP 1866339, PCT Publication No.: WO 2011/028683, PCT Publication No.: WO 2013/039954, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, PCT Publication No.: WO2006/083289, PCT Publication No.: WO 2005/115451, U.S. Pat. No. 7,618,632, and PCT Publication No.: WO 2011/051726.

In one embodiment, a CAR expressing cell described herein is administered to a subject in combination with a GITR agonist, e.g., a GITR agonist described herein. In one embodiment, the GITR agonist is administered prior to the CAR-expressing cell. For example, in one embodiment, the GITR agonist can be administered prior to apheresis of the cells. In one embodiment, the subject has CLL.

The methods described herein can further include formulating a CAR-expressing cell in a pharmaceutical composition. Pharmaceutical compositions may comprise a CAR-expressing cell, e.g., a plurality of CAR-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions can be formulated, e.g., for intravenous administration.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-cancer effective amount,” “a cancer-inhibiting effective amount,” or “therapeutic amount” is indicated, the precise amount of the compositions to be administered can be determined by a physician with consideration of individual differences in age, weight, tumor size, extent of infection or metastasis, and condition of the patient (subject). It can generally be stated that a pharmaceutical composition comprising the immune effector cells (e.g., T cells, NK cells) described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988).

In some embodiments, a dose of CAR cells (e.g., CD19 CAR cells) comprises about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD19 CAR cells) comprises at least about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD19 CAR cells) comprises up to about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD19 CAR cells) comprises about 1.1×10⁶-1.8×10⁷ cells/kg. In some embodiments, a dose of CAR cells (e.g., CD19 CAR cells) comprises about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (e.g., CD19 CAR cells) comprises at least about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (e.g., CD19 CAR cells) comprises up to about 1×10⁷, 2×10⁷, 5×10⁷, 1×10⁸, 2×10⁸, 5×10⁸, 1×10⁹, 2×10⁹, or 5×10⁹ cells.

In certain aspects, it may be desired to administer activated immune effector cells (e.g., T cells, NK cells) to a subject and then subsequently redraw blood (or have an apheresis performed), activate immune effector cells (e.g., T cells, NK cells) therefrom, and reinfuse the subject with these activated and expanded immune effector cells (e.g., T cells, NK cells). This process can be carried out multiple times every few weeks. In certain aspects, immune effector cells (e.g., T cells, NK cells) can be activated from blood draws of from 10 cc to 400 cc. In certain aspects, immune effector cells (e.g., T cells, NK cells) are activated from blood draws of 20 cc, 30 cc, 40 cc, 50 cc, 60 cc, 70 cc, 80 cc, 90 cc, or 100 cc.

The administration of the subject compositions may be carried out in any convenient manner. The compositions described herein may be administered to a subject trans arterially, subcutaneously, intradermally, intratumorally, intranodally, intramedullary, intramuscularly, by intravenous (i.v.) injection, or intraperitoneally, e.g., by intradermal or subcutaneous injection. The compositions of immune effector cells (e.g., T cells, NK cells) may be injected directly into a tumor, lymph node, or site of infection.

Exemplification of embodiments disclosed herein, e.g., are described in Examples 1-10, on pages 204-219 of International Application WO 2007/117112 filed on Dec. 27, 2016, which is hereby expressly incorporated by reference, including the figures and figure legends associated with said Examples.

### Indications

In some aspects, the disclosure provides a method of treating a subject having, or at risk of having a lymphoma (e.g., a lymphoma disclosed herein, e.g. DLBCL or FL), comprising responsive to a determination, e.g., prediction, of a lesion-level treatment response to a therapy comprising a Chimeric Antigen Receptor 19 (CAR19) immune effector cell (“CAR19 therapy”), administering the CAR19 therapy to the subject, thereby treating the subject.

In other aspects, the disclosure provides a method of evaluating a subject having, or at risk of having a lymphoma (e.g., a lymphoma disclosed herein,), comprising determining, e.g., predicting, of a lesion-level treatment response to a therapy comprising a Chimeric Antigen Receptor 19 (CAR19) immune effector cell (“CAR19 therapy”), with a neural network. In an embodiment, determining comprises: acquiring, e.g., receiving, an image of a lesion of the subject (“acquired image”); and/or processing the image with the neural network (“processed image”). In an embodiment, the neural network outputs a classification result indicating the lesion-level treatment response to the CAR19 therapy.

In some aspects, the subject has or has been identified as having a lymphoma, e.g., a relapsed and/or refractory lymphoma. In some embodiment, the lymphoma is chosen from: DLBCL, follicular lymphoma (FL), mantle cell lymphoma (MCL), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, Marginal zone lymphoma, multiple myeloma, myelodysplasia and myelodysplastic syndrome, non-Hodgkin lymphoma, Hodgkin lymphoma, or plasmablastic lymphoma. In some embodiments, the lymphoma is DLBCL (e.g., relapsed and/or refractory DLBCL). In some embodiments, the lymphoma is follicular lymphoma (FL).

The term “relapse” as used herein refers to reappearance of a disease (e.g., cancer) after an initial period of responsiveness, e.g., after prior treatment with a therapy, e.g., cancer therapy (e.g., complete response or partial response). The initial period of responsiveness may involve the level of cancer cells falling below a certain threshold, e.g., below 20%, 15%, 10%, 5%, 4%, 3%, 2%, or 1%. The reappearance may involve the level of cancer cells rising above a certain threshold, e.g., above 20%, 15%, 10%, 5%, 4%, 3%, 2%, or 1%.

“Refractory” as used herein refers to a disease, e.g., cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

### DLBCL and Relapsed/Refractory DLBCL

In some embodiments, the subject has DLBCL. In some embodiments the subject has relapsed or refractory DLBCL. In some embodiments, the subject is at least 18 years of age.

In some embodiments, the subject having DLBCL, e.g., relapsed or refractory DLBCL has previously been administered one or more of: an anti-CD20 therapy, an anthracycline based chemotherapy or stem cell therapy, e.g., allogeneic or autologous SCT, e.g., as described herein, as a, e.g., first, second or third line therapy. In some embodiments, the subject has no response to, e.g., relapsed, refractory, has progressive disease, or has failed, the first, second or third line therapy.

In some embodiments, a subject having relapsed or refractory DLBCL is administered a combination therapy comprising a BTK inhibitor, e.g., ibrutinib, and a CAR-expressing cell, e.g., according to a dosage regimen described herein. In some embodiments, the subject has previously been treated with a BTK inhibitor, e.g., for at least 4-6 weeks or 8-10 weeks.

In some embodiments, the subject is administered the BTK inhibitor, e.g., daily, prior to apheresis, e.g., at least about 21 days, e.g., 21-30 days, e.g., 28 days prior to apheresis. In some embodiments, the subject is administered the BTK inhibitor for at least about 21 days, e.g., 10-100 days, after apheresis and prior to CAR therapy administration, e.g., infusion.

In some embodiments, the subject is administered the BTK inhibitor concurrently with or after apheresis. In some embodiments, the subject is administered the BTK inhibitor for at least about 21 days, e.g., 10-100 days, after apheresis and prior to CAR therapy administration, e.g., infusion. In some embodiments, the subject is continuously administered with a BTK inhibitor, e.g., at a dose of 560 mg, daily. In some embodiments, the subject is administered 0.6-6.0×10⁸ CAR expressing cells.

In some embodiments, the subject is administered lymphodepletion after initiation of the BTK inhibitor, but prior to administration of the CAR therapy. In some embodiments, the lymphodepletion comprises administering cyclophosphamide and fludarabine. In some embodiments, the lymphodepletion comprises administering 500 mg/m2 cyclophosphamide daily for 2 days and 30 mg/m2 fludarabine daily for 3 days. In some embodiments, the lymphodepletion comprises administering 250 mg/m2 cyclophosphamide daily for 3 days, and 25 mg/m2 fludarabine daily for 3 days. In some embodiments, the lymphodepletion begins with the administration of the first dose of fludarabine. In some embodiments, cyclophosphamide and fludarabine are administered on the same day. In some embodiments, cyclophosphamide and fludarabine are not administered on the same day. In some embodiments, the daily dosages are administered on consecutive days. In embodiments, the lymphodepletion comprises administering bendamustine. In some embodiments, bendamustine is administered daily, e.g., twice daily, at a dosage of about 75-125 mg/m2 (e.g., 75-100 or 100-125 mg/m², e.g., about 90 mg/m²), e.g., intravenously. In some embodiments, bendamustine is administered at dosage of 90 mg/m² daily, e.g., for 2 days. In some embodiments, the subject has a cancer, e.g., a hematological cancer as described herein.

In embodiments, the subject is administered a first lymphodepletion regimen and/or a second lymphodepletion regimen. In embodiments, the first lymphodepletion regimen is administered before the second lymphodepletion regimen. In embodiments, the second lymphodepletion regimen is administered before the first lymphodepletion regimen. In embodiments, the first lymphodepletion regimen comprises cyclophosphamide and fludarabine, e.g., 250 mg/m2 cyclophosphamide daily for 3 days, and 25 mg/m2 fludarabine daily for 3 days. In embodiments, the second lymphodepletion regimen comprises bendamustine, e.g., 90 mg/m² daily, e.g., for 2 days. In embodiments, the second lymphodepletion regimen is administered as an alternate lymphodepletion regimen, e.g., if a subject has experienced adverse effects, e.g., Grade 4 hemorrhagic cystitis, to a lymphodepletion regimen comprising cyclophosphamide. In some embodiments, the lymphoma is a DLBCL, e.g., a relapsed or refractory DLBCL (e.g., r/r DLBCL), e.g., a CD19+r/r DLBCL. In some embodiments, the subject is an adult and the lymphoma is an r/r DLBCL.

In some embodiments, a subject administered a therapy described herein, e.g., a therapy comprising a CAR-expressing therapy, e.g., a therapy comprising a CAR19-expressing therapy (e.g., a CAR19-expressing therapy in combination with a BTK inhibitor or a PD-1 inhibitor), has previously received, e.g., been administered, one or more lines of therapy, e.g., 2, 3, 4, or 5 or more lines of therapy (e.g., one or more therapies as described herein) and/or the subject was not eligible for or had failed stem cell therapy (SCT), e.g., autologous or allogeneic SCT. In some embodiments the subject has previously received 2 or more lines of therapy comprising rituximab and anthracycline. In some embodiments, the subject was not eligible for or had failed autologous SCT. In some embodiments, administration of a CAR19-expressing therapy (e.g., in combination with a BTK inhibitor or a PD-1 inhibitor) to the subject who has previously undergone 2 or more lines of therapy and/or was not eligible for or had failed autologous SCT results in a response, e.g., a high response rate and/or a durable response to the therapy, e.g., therapy comprising a CAR19-expressing therapy (e.g., in combination with a BTK inhibitor or a PD-1 inhibitor). In some embodiments, the subject has a hematological cancer, e.g., DLBCL, e.g., relapsed and/or refractory DLBCL.

### Follicular Lymphoma

In some embodiments, the subject has follicular lymphoma (FL). In some embodiments, FL is also referred to as a Non-Hodgkin lymphoma. In some embodiments, the subject has relapsed or refractory FL. In some embodiments, the FL can be classified as a Stage I lymphoma, a Stage II lymphoma, a Stage III lymphoma or a Stage IV lymphoma. FL and standard of care for FL is described in Luminaari S et al. (2012); Rev Bras Hematol Hemoter. 34(1):54-59, the entire contents of which is hereby incorporated by reference.

In some embodiments, the subject having FL, e.g., relapsed or refractory FL, has previously been administered one or more of: a chemotherapy, immunotherapy, radiation therapy or radioimmunotherapy, e.g., as a first, second, or third line therapy. In some embodiments, the subject has been administered: an anti-CD20 therapy (e.g., rituximab); an anthracycline based chemotherapy; a stem cell therapy, e.g., allogeneic or autologous SCT; or a radioimmunotherapy. In some embodiments, the subject has no response to, e.g., relapsed, refractory, has progressive disease, or has failed, the first, second or third line therapy.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1: Deep-Learning-Based Image Analysis of Pre-Treatment Diagnostic CT and PET lCT Images for Predicting Treatment Response to Chimeric Antigen Receptor (CAR) T Cell Therapy in Lymphoma

This Example describes the feasibility of applying a deep-learning (DL)-based image analysis methodology on pre-treatment medical images to predict lesion-level treatment responses to chimeric antigen receptor (CAR) T-cell therapy in patients (or subjects) with lymphoma. The lesion-level treatment response prediction is followed by a rule-based reasoning methodology for patient-level response prediction to CAR T-cell therapy.

### Materials and Methods

### Study Cohort and Data Sets

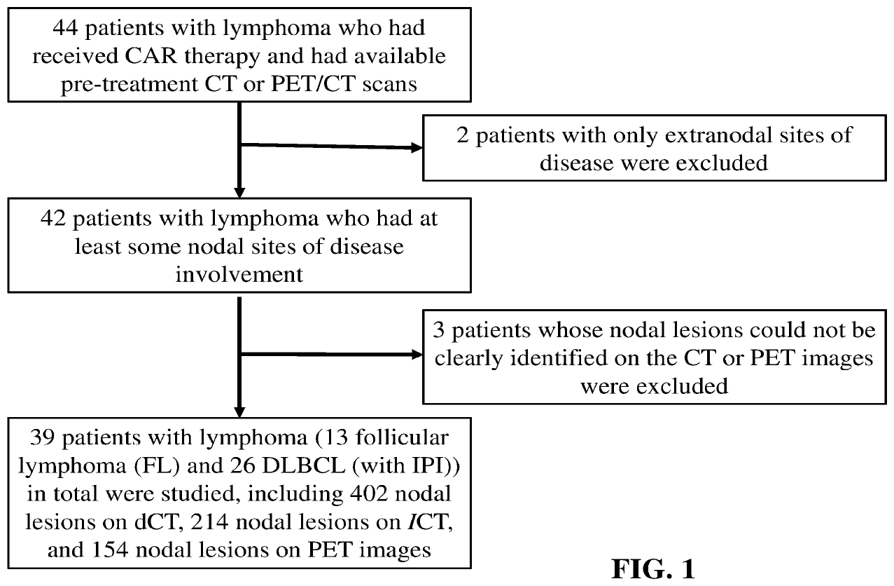

Pre-treatment diagnostic CT images and PET lCT images of the neck, chest, abdomen, and pelvis previously obtained for clinical purposes in 39 (27M, 12F, 56.2±12.2 years) adult patients with lymphoma (with relapsed or refractory DLBCL or FL) who subsequently underwent CAR T-cell treatment with tisagenlecleucel (targeting CD19+ cells) were utilized in this study. This study included response prediction at the lesion-level and patient level. 26 patients (20M, 6F; median age 57 years (range 28-74))) with DLBCL and 13 patients (7M, 6F; median age 62 years (range 43-72)) with FL were assessed for patient-level response prediction. The patient inclusion and exclusion schema is shown in FIG. 1.

The identification of all individual lymph node disease sites was performed using pre-treatment images followed by determination of ground truth lesion-level responses for all individual nodal lesions via comparison of pre-treatment and post-treatment images by an expert radiologist (DAT) with over 20 years of experience in the interpretation of CT, magnetic resonance imaging (MRI), and PET images. Lesion-level response to treatment was defined by interval decrease in size or metabolic activity or interval resolution of a lesion between pretreatment and post-treatment images, whereas lesion-level non-response was defined as lack of change or interval increase in size or metabolic activity of a lesion. The post-treatment dCT and PET lCT images utilized to determine ground truth lesion-level responses had been previously acquired 94.0±33.2 days after pre-treatment images. Extranodal lesion sites as well as splenic and Waldeyer's ring nodal tissue lesion sites were not considered given the small numbers of lesions encountered in these anatomic locations. Patient-level International Prognostic Index (IPI) score was also calculated at the pre-treatment scan time point for those patients with DLBCL, with scores ranging from 0-5 (where 1 point is assigned for the presence of each of the following factors: age >60, ECOG performance status >1, elevated serum lactate dehydrogenase (LDH) level, disease stage III or IV, and presence of >1 extranodal site of disease [20, 21]). For patients with FL, a risk score (0 through 5) which is named as follicular lymphoma international prognostic index (FLIPI), was computed by summing the number of risk factors (six in total, including sex, age, B symptoms, number of extranodal sites, serum LDH, and ESR) present in a single patient at time of diagnosis [21].

Patients were categorized into three groups according to lesion responses detected on post-treatment CT or PET lCT images: (1) full responders (F-R), where all detected lesions demonstrated a response to treatment (i.e., either interval decrease in size, interval decrease in metabolic activity, or interval resolution); (2) full non-responders (F-NR), where all detected lesions demonstrated no response to treatment (i.e., either lack of change or interval increase in size or metabolic activity); and (3) partial responders (P-R), where some detected lesions demonstrated a response and others did not. The response categories of patients at the lesion-level and patient-level based on the different imaging modalities of dCT, lCT, and PET is shown in Table 10. In addition, patient-level response status based on post-treatment scans that had been acquired up to 12 months after pre-treatment scans was determined for all patients.

The main study aim was to assess the feasibility of prediction of treatment response at the lesion level using a DL image analysis methodology, and following this, the feasibility of prediction of treatment response at the patient level using a rule-based reasoning approach.

TABLE 10
Summary of response categories of lymphoma patients who received CAR
T-cell therapy and of lesions assessed. dCT = diagnostic computed
tomography images, lCT = low-dose computed tomography images from
PET/CT scans, PET = positron emission tomography images from PET/CT scans.

| | dCT | | lCT | | PET | |
|---|---|---|---|---|---|---|
| Patient | Number | | Number | | Number | |
| response | of lesions | | of lesions | | of lesions | |
| category | (lymph | Number | (lymph | Number | (lymph | Number |
| groups | nodes) | of patients | nodes) | of patients | nodes) | of patients |
| Full | 209 | 13 | 142 | 12 | 93 | 9 |
| responders | | | | | | |
| (F-R) | | | | | | |
| Full non- | 62 | 8 | 27 | 4 | 16 | 2 |
| responders | | | | | | |
| (F-NR) | | | | | | |
| Partial | 76 | 7 | 22 | 6 | 21 | 6 |
| responders | | | | | | |
| (P-R), | | | | | | |
| responding | | | | | | |
| lesions | | | | | | |
| Partial | 55 | | 23 | | 24 | |
| responders | | | | | | |
| (P-NR), non- | | | | | | |
| responding | | | | | | |
| lesions | | | | | | |
| Total | 402 | 28 | 214 | 22 | 154 | 17 |

### VOI Settings

Two types of prediction tasks were tested: one based on a 3D volume of interest (VOI) as a rectangular box defined around the lesion and another by utilizing the whole axial slice passing through the mid-portions of the individual abnormal lymph node. In total, 770 (402 by dCT+214 by lCT+154 by PET) lymph node lesions, as shown in Table 10, were assessed in this study. Among these lesions, 649 VOIs were placed around individual nodal lesions including 383 VOIs in pre-treatment dCT images, 158 VOIs in pre-treatment lCT images, and 108 VOIs in pre-treatment PET images. For the remaining 121 lymph nodes, it was difficult to set up a reliable bounding box (VOI) surrounding the lymph node since the superior and inferior slices covering the lymph node were not clear. For those lesions, response prediction was performed by only using whole image slices as input.

The VOI operation was performed using CAVASS software [22] by setting up one rectangular box around the object/lymph node in 3D space. Every lesion was labeled with a 0 or 1 based on post-treatment response, where 0 indicated a lesion without response to treatment and 1 indicated a lesion with response to treatment. In particular, for the 1 VOI-slice input scenario, a rectangular box was placed on the axial image slice with maximum cross-sectional area through a given abnormal lymph node, where the left-right and anterior-posterior margins were placed outside of the lymph node halfway from the lymph node center in the left-right and anterior-posterior directions, respectively. For the 3 VOI-slices input scenario, the rectangular box was then propagated superiorly to one contiguous slice and inferiorly to one other contiguous slice.

In addition to using the VOIs as training and testing samples for the neural network, whole axial slices passing through the mid-portions of the individual abnormal lymph nodes as samples were also tested in order to investigate the differences between lesion-level response prediction using a VOI-based approach and a whole slice-based (non-VOI) approach. The axial slices in this experiment were carefully selected to avoid having any two different lesions within the same whole axial slice.

In total, 770 lymph node lesions (402 by diagnostic computed tomography (dCT); 214 by low-dose CT (lCT); 154 by positron emission tomography (PET) were assessed (Table S1). Among these lesions, 649 VOIs were placed around individual nodal lesions including 383 VOIs on dCT images, 158 VOIs on lCT images, and 108 VOIs on PET images. For the remaining 121 nodal lesions, it was difficult to reliably place surrounding VOIs as the superior and inferior slices through the lymph nodes were unclear. For those lesions, response predictions were performed using only whole-image slices as input.

These steps were performed on dCT images, lCT images, and PET images separately. The purpose of the above VOI operation was to investigate the difference between lesion-level treatment response prediction using a VOI-based approach compared to prediction using a whole slice-based (non-VOI) approach.

### Deep Learning Structure

In this study, transfer learning was employed for the prediction of treatment response. Transfer learning has been widely utilized in many DL applications, especially when there are limited training samples. Generally, transfer learning is performed by loading a pre-trained neural network, followed by modifying its output layers/decision layers into layers for a specific classification purpose, and then retraining the whole network with specific training samples. The flow chart of using transfer learning for this outcome prediction study is shown in FIG. 2. Although only the pre-trained neural network “AlexNet” [19] was used here, the same framework can be easily configured using other more recent pre-trained neural networks such as VGG16 [23] or ResNet [24]. AlexNet was selected for use in this study as it has a simpler structure (with only 5 convolutional layers) and is more easily retrained to test the proposed approach.

### Adaptation of AlexNet for Lesion-Level Response Prediction

AlexNet was pre-trained on ImageNet data set in this study, which has 1 million images for image classification and 1000 image classes in total (involving natural and man-made objects). In general, AlexNet has 5 convolutional layers followed by rectified linear unit (ReLu) and Max Pooling layers, followed by 3 fully connected layers, another two ReLu and Dropout layers, and then a Soft-max layer before the final output layer for 1000 classes. In this study, lesion-level treatment response was predicted as a binary classification as follows. The last three layers of the pre-trained network were previously configured for 1000 classes. All layers were extracted, except for the last three layers, from the pre-trained network. In other words, the feature layers were transferred from the pre-trained network to the new classification task by replacing the last three layers with a fully connected layer, a Soft-max layer, and a binary classification output layer. The options for the new fully connected layer such as number of epochs, batch size, etc., were specified according to the new training data.

For AlexNet, different batch sizes of 5, 10, 20, and 30 were tested, with number of epochs of 40, 80, 100, and 200, using an initial learning rate for training as 10⁻⁴, and adopting a stochastic-gradient-descent-with-momentum algorithm for parameter optimization [25]. Image pre-processing operations included subtracting the mean and down-sampling using bilinear interpolation to 224×224 pixels from the original resolution.

### Data Augmentation and Neural Network Retraining for Lesion-Level Response Prediction

The set of 383 VOI samples were divided from pre-treatment dCT data sets into training, validation, and testing data sets in the ratio of 6:2:2. Due to the limited training samples, a data augmentation technique [26] that is widely used and has been shown to be useful to improve training performance was adopted [27]. Data augmentation helped to prevent the network from overfitting and memorizing the exact details of the training images. All input images/slices were automatically resized into 224×224 size according to AlexNet input requirements. The data sets were then divided into training, validation, and testing data sets in the ratio of 6:2:2 (Table 11). Augmentation operations on the training images included random flips of the training images along the vertical axis in the image slice (antero-posterior direction), random translations of up to 10 pixels horizontally and vertically (antero-posterior and right-to-left direction) within the slice plane, and affine operations scale change (magnification and minification) and rotation within the slice plane. For validation and testing data, only the validation and test images were resized without performing further data augmentation, since both were used to estimate the performance of the trained neural network. Due to the fixed requirement of the size of the inputs of AlexNet, the following 5 scenarios were considered: Single VOI-restricted image slice passing through the mid-portions of lesions (1 VOI-slice), three contiguous VOI-restricted image slices passing through the mid-portions of lesions (3 VOI-slices), single whole image slice passing through the mid-portions of lesions (1 whole-slice), three contiguous whole image slices passing through the mid-portions of lesions (3 whole-slices), and combined single VOI-restricted and single whole-image slices passing through the mid-portions of lesions in two channels of one input sample (combined-slices). Furthermore, each scenario included the 3 modalities of dCT, lCT, and PET. Thus, the total number of tested scenarios was 5 scenarios×3 modalities=15.

For treatment response prediction from lCT/PET (i.e., PET lCT scan images), an incremental transfer learning approach was further investigated by loading the network pre-trained via dCT and then retraining it with lCT and PET separately. For this retraining operation, the ratio of 6:2:2 was used for training, validation, and testing.

To improve test statistics, multi-fold cross validation was carried out by repeating each experiment 10 times for a different combination of 6:2:2 (training:validation:testing) data set division. Considering all scenarios, modalities, and the hyper parameters of 4 batch sizes and 4 epochs utilized, a total of 3,040 experiments were conducted (including 2400 from transfer learning and 640 from incremental learning). The strategy for transfer learning and incremental learning utilized in this study is summarized in FIG. 3.

### Evaluation of Lesion-Level Prediction and Statistical Analysis

Accuracy, specificity, and sensitivity of the lesion-level prediction task and also the area under the curve (AUC) for the receiver operating characteristic (ROC) curve were estimated. A two-sided t-test was utilized to compare different experimental results derived from different scenarios, different parameters, and different imaging modalities. A p value of <0.05 was considered to be statistically significant. Table 11 shows the numbers of samples of lesions used for training and testing in each experiment.

TABLE 11
Experiments for transfer learning for lesion-level treatment response prediction.

| Imaging | | Experiments and | | |
|---|---|---|---|---|
| modalities | | scenarios | Data sets | Methods |
| dCT | | Batch size [5, 10, 20, | 0.6:0.2:0.2 | Transfer learning with |
| | | 30], | Training:validation: | pre-trained AlexNet |
| | | | testing among 383 | |
| | | | samples from dCT | |
| PET/CT | lCT | epochs [40 80 100 200] | 0.6:0.2:0.2 | Transfer learning with pre- |
| | PET | 1 VOI slice, 3 VOI | Training:validation: | trained AlexNet plus |
| | | slices, 1 whole slice, | testing among 158 | Incremental learning from |
| | | 3 whole slices, and | samples from lCT | pre-trained network for |
| | | combined-slices | | dCT |
| | | | 0.6:0.2:0.2 | Transfer learning with pre- |
| | | | Training:validation: | trained AlexNet plus |
| | | | testing among 108 | Incremental learning from |
| | | | samples from | pre- |
| | | | PET | trained network for dCT |

dCT = diagnostic computed tomography, lCT = low-dose computed tomography, PET = positron emission tomography, VOI = volume of interest.

### Evaluation of Patient-Level Prediction and Statistical Analysis

Due to the relatively small number of patients, instead of using machine learning or a regression approach, patient-level response analysis was performed by applying a rule-based reasoning approach on lesion-level prediction results from the DL network. Patients with DLBCL (26 in total, 8 with IPI score 1, 11 with IPI score 2, 4 with IPI score 3, and 3 with IPI score 4) and FL (13 in total, 2 with FLIPI score 1, 2 with FLIPI score 2, 4 with FLIPI score 3, 3 with FLIPI score 4, and 2 with FLIPI score 5) were utilized in patient-level response prediction in this study. Due to the small number of patients, only accuracy, specificity, and sensitivity were estimated and not AUC.

The predicted response for every lesion was first achieved by performing a leave-one-out (LOO) experiment at the lesion level. Then, after lesion-level response was predicted using transfer learning, two rules, the “All” rule and the “Majority” rule, were utilized to determine patient-level response as follows. For the “All” rule, a patient responder is one in whom all lesions have responded, and a patient non-responder is one in whom at least one lesion has not responded. For the “Majority” rule, a patient responder is one in whom the majority of lesions (using thresholds of either 60% or 70% of all lesions) have responded, and a patient non-responder is one in whom the majority of lesions (using thresholds of either 60% or 70% of all lesions) have not responded.

The reference standard for patient-level response (responder/non-responder) was based on the findings on cross-sectional imaging scans acquired up to 12-months after the date of pre-treatment scans, which included patients with DLBCL (10 responder patients and 16 non-responder patients) and patients with FL (8 responder patients and 5 non-responder patients). Since patient-level response assessment based on IPI (for patients with DLBCL) and FLIPI (for patients with FL) scores is currently used in clinical practice, we compared the performance of these clinical methods to our rule-based method in the 26 patients with DLBCL (10 responders and 16 non-responders). For the IPI method, DLBCL patients were categorized into responder and non-responder groups by using different score thresholds based on the number of IPI risk factors (of IPI ≤1, IPI ≤2, and IPI ≤3), where the lower score groups were considered as the responder groups. The accuracy, sensitivity, and specificity of the patient-level prediction task were then evaluated for rule-based and IPI-based approaches, utilizing Pearson's chi-square test for statistical comparisons. For the FLIPI method, patients were categorized into responder and non-responder groups by using different score thresholds (of FLIPI ≤1, FLIPI ≤2, FLIPI ≤3, and FLIPI ≤4), where the lower score groups were considered as the responder groups.

Computing System

The transfer learning program was implemented in MATLAB 2018b and run under Ubuntu16.04 OS on two computer systems including one PC with 64 GB RAM, 4×Icore-7 CPUs, and two Nvidia 1080Ti GPU cards with 22 GB GPU RAM in total, and another PC with 24 GB RAM, 4×Icore-7 CPUs, and one Nvidia TITAN V GPU card with 12 GB GPU RAM.

### Results

Transfer Learning for Lesion-Level Response Prediction from Pre-Treatment dCT, lCT, and PET Images

The diagnostic performance results of lesion-level response prediction using transfer learning for the five scenarios from the dCt, lCT, and PET imaging modalities are shown in Table 12 (with p values provided in Table 13). In general, the predictive, diagnostic performances of the 1 VOI slice and 3 VOI-slices input scenarios on dCt, lCT, and PET were substantially lower than those of the corresponding whole slice-based scenarios.

For example, the accuracy of 1 VOI-slice vs. 1 whole-slice input from dCT was 0.68±0.05 vs. 0.82±0.05, respectively (p<0.0001) with AUC 0.59±0.04 vs. 0.91±0.03, respectively (p<0.0001), and the accuracy of 3 VOI-slices vs. 3 whole-slices input from dCT was 0.65±0.05 vs. 0.84±0.05, respectively (p<0.0001) with AUC 0.52±0.07 vs. 0.90±0.05, respectively (p<0.0001). The predictive performances of 1 whole-slice and 3 whole-slices inputs from dCT (AUC 0.91±0.03 vs. 0.90±0.05, respectively, p=0.435) were similar, as were those from lCT (AUC 0.92±0.08 vs. 0.94±0.07, respectively, p=0.66) and from PET (AUC 0.93±0.07 vs. 0.95±0.06, respectively, p=0.46).

The predictive performances of combined-slices input from dCT, lCT, or PET were not statistically different from those based on 1 whole-slice input. For dCT, lesion-level response prediction using 1 whole-slice input had accuracy 0.82±0.05, sensitivity 0.87±0.07, specificity 0.77±0.12, and AUC 0.91±0.03. For lCT, lesion-level response prediction using 1 whole-slice input had accuracy 0.91±0.06, sensitivity 0.94±0.06, specificity 0.75±0.32, and AUC 0.92±0.08. For PET, 1 whole-slice input had accuracy 0.87±0.06, sensitivity 0.90±0.06, specificity 0.77±0.19, and AUC 0.93±0.07. Although the accuracy of 1 whole-slice input from dCT was lower than the accuracy from lCT (p=0.002) and PET (p=0.08), the AUC and specificity of 1 whole-slice input did not statistically differ between dCT, lCT, and PET. There were no significant differences in lesion-level response prediction accuracy or AUC between transfer learning and incremental learning approaches using 1 whole-slice input from lCT or PET.

TABLE 12
Diagnostic performance of transfer learning on test data sets for lesion-level treatment response prediction
(using 40 epochs and batch size 5). dCT = diagnostic computed tomography, lCT = low-dose
computed tomography, PET = positron emission tomography, Acc = accuracy, Sens = sensitivity,
Spec = specificity, AUC = area under the curve. Mean and standard deviation values were listed (mean ± SD).

| | dCT | | | | lCT | | | |
|---|---|---|---|---|---|---|---|---|
| Scenario | Acc | Sens | Spec | AUC | Acc | Sens | Spec | AUC |
| 1 | 0.68 ± 0.05 | 0.70 ± 0.02 | 0.58 ± 0.16 | 0.59 ± 0.04 | 0.68 ± 0.05 | 0.70 ± 0.02 | 0.58 ± 0.16 | 0.60 ± 0.11 |
| VOI- | | | | | | | | |
| slice | | | | | | | | |
| 1 | 0.82 ± 0.05 | 0.87 ± 0.07 | 0.77 ± 0.12 | 0.91 ± 0.03 | 0.91 ± 0.06 | 0.94 ± 0.06 | 0.75 ± 0.32 | 0.92 ± 0.08 |
| whole- | | | | | | | | |
| slice | | | | | | | | |
| 3 | 0.65 ± 0.05 | 0.68 ± 0.02 | 0.52 ± 0.21 | 0.52 ± 0.07 | 0.79 ± 0.06 | 0.84 ± 0.02 | 0.21 ± 0.33 | 0.53 ± 0.17 |
| VOI- | | | | | | | | |
| slices | | | | | | | | |
| 3 | 0.84 ± 0.05 | 0.90 ± 0.04 | 0.76 ± 0.12 | 0.90 ± 0.05 | 0.90 ± 0.05 | 0.95 ± 0.04 | 0.74 ± 0.20 | 0.94 ± 0.07 |
| whole- | | | | | | | | |
| slices | | | | | | | | |
| Combined | 0.84 ± 0.04 | 0.89 ± 0.03 | 0.76 ± 0.10 | 0.91 ± 0.03 | 0.93 ± 0.03 | 0.96 ± 0.03 | 0.83 ± 0.14 | 0.98 ± 0.02 |
| slices | | | | | | | | |

| | PET | | | |
|---|---|---|---|---|
| Scenario | Acc | Sens | Spec | AUC |
| 1 | 0.68 ± 0.05 | 0.70 ± 0.02 | 0.58 ± 0.16 | 0.51 ± 0.14 |
| VOI- | | | | |
| slice | | | | |
| 1 | 0.87 ± 0.06 | 0.90 ± 0.06 | 0.77 ± 0.19 | 0.93 ± 0.07 |
| whole- | | | | |
| slice | | | | |
| 3 | 0.65 ± 0.02 | 0.68 ± 0.01 | 0.48 ± 0.09 | 0.53 ± 0.07 |
| VOI- | | | | |
| slices | | | | |
| 3 | 0.90 ± 0.07 | 0.95 ± 0.05 | 0.81 v0.19 | 0.95 ± 0.06 |
| whole- | | | | |
| slices | | | | |
| Combined | 0.87 ± 0.07 | 0.93 ± 0.07 | 0.73 ± 0.16 | 0.92 ± 0.08 |
| slices | | | | |

TABLE 13
P values of t-test comparisons of diagnostic performance between 5 input scenarios for lesion-level treatment response prediction
in lymphoma. dCT = diagnostic computed tomography, lCT = low-dose computed tomography, PET = positron emission
tomography, VOI = volume of interest, Acc = accuracy, Sens = sensitivity, Spec = specificity, AUC = area under the curve.

| | dCT | | | | lCT | | | | PET | | | |
|---|---|---|---|---|---|---|---|---|---|---|---|---|
| Testing | Acc | Sens | Spec | AUC | Acc | Sens | Spec | AUC | Acc | Sens | Spec | AUC |
| 1 VOI-slice | <0.0001 | <0.0001 | 0.013 | <0.0001 | 0.003 | 0.001 | 0.1687 | <0.0001 | <0.0001 | <0.0001 | 0.002 | <0.0001 |
| vs. | | | | | | | | | | | | |
| 1 whole-slice | | | | | | | | | | | | |
| 1 VOI-slice | 0.308 | 0.049 | 0.442 | 0.016 | 0.247 | 0.108 | 0.12 | 0.2354 | <0.0001 | <0.0001 | 0.036 | 0.653 |
| vs. | | | | | | | | | | | | |
| 3 VOI-slices | | | | | | | | | | | | |
| 1 VOI-slice | <0.0001 | <0.0001 | 0.017 | <0.0001 | 0.005 | <0.0001 | 0.149 | <0.0001 | <0.0001 | <0.001 | 0.0015 | <0.0001 |
| vs. | | | | | | | | | | | | |
| 3 whole-slices | | | | | | | | | | | | |
| 1 whole-slice | <0.0001 | <0.0001 | 0.006 | <0.0001 | 0.0002 | 0.0003 | 0.002 | <0.0001 | <0.001 | <0.001 | 0.0006 | <0.0001 |
| vs. | | | | | | | | | | | | |
| 3 VOI-slices | | | | | | | | | | | | |
| 1 whole-slice | 0.446 | 0.192 | 0.880 | 0.528 | 0.602 | 0.493 | 0.946 | 0.657 | 0.287 | 0.055 | 0.638 | 0.464 |
| vs. | | | | | | | | | | | | |
| 3 whole-slices | | | | | | | | | | | | |
| 3 VOI-slices | <0.0001 | <0.0001 | 0.007 | <0.0001 | 0.00021 | <0.0001 | 0.001 | <0.0001 | <0.0001 | <0.0001 | 0.0002 | <0.0001 |
| vs. | | | | | | | | | | | | |
| 3 whole-slices | | | | | | | | | | | | |
| 1 whole-slice | 0.41 | 0.30 | 0.76 | 0.98 | 0.36 | 0.35 | 0.46 | 0.07 | 0.91 | 0.31 | 0.51 | 0.70 |
| vs. | | | | | | | | | | | | |
| combined- | | | | | | | | | | | | |
| slices | | | | | | | | | | | | |

The prediction performances using dCT, lCT, and PET were not statistically significantly different from each other. In particular, the AUC of 1-slice from dCT, lCT, or PET was 0.91, 0.92 and 0.93, respectively, with p values of 0.71 (dCT vs. lCT), 0.42 (dCT vs. PET), and 0.79 (lCT vs. PET), and the AUC using 3-slices from dCT, lCT, or PET was 0.90, 0.94, and 0.95, respectively, with p values of 0.18 (dCT vs. lCT), 0.05 (dCT vs. PET) and 0.61 (lCT vs. PET). All p values are shown in Table 14.

TABLE 14
p values of t-test comparisons of diagnostic performance of 3 imaging modalities
(dCT, lCT, PET) using 1 whole-slice & 3 whole-slices scenarios for lesion-level
treatment response prediction. dCT = diagnostic computed tomography,
lCT = low-dose computed tomography, PET = positron emission tomography,
Acc = accuracy, Sens = sensitivity, Spec = specificity, AUC = area under the curve.

| 1 whole- | | | | | 3 whole- | | | | |
|---|---|---|---|---|---|---|---|---|---|
| slice | Acc | Sens | Spec | AUC | slices | Acc | Sens | Spec | AUC |
| dCT vs. | 0.002 | 0.027 | 0.89 | 0.71 | dCT vs. | 0.01 | 0.01 | 0.85 | 0.18 |
| lCT | | | | | lCT | | | | |
| dCT vs. | 0.08 | 0.27 | 0.92 | 0.42 | dCT vs. | 0.04 | 0.02 | 0.44 | 0.05 |
| PET | | | | | PET | | | | |
| lCT vs. | 0.11 | 0.19 | 0.85 | 0.79 | lCT vs. | 1.00 | 0.97 | 0.43 | 0.61 |
| PET | | | | | PET | | | | |

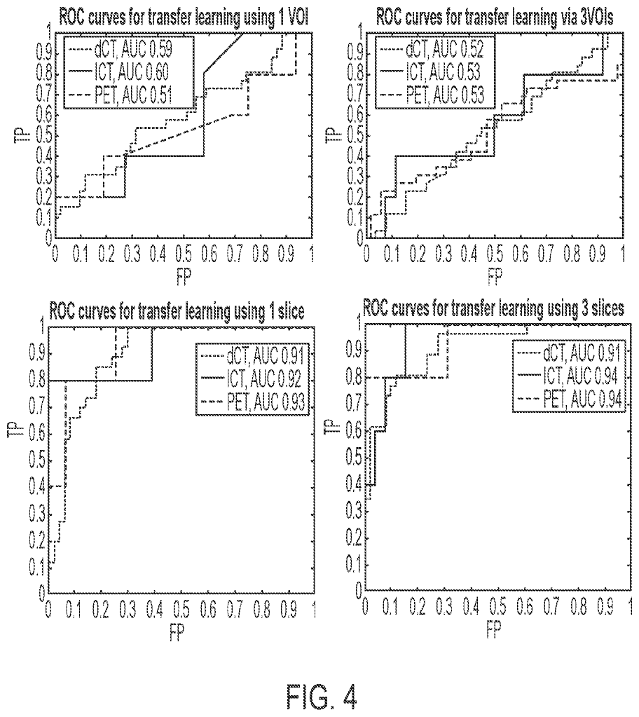

ROC curves for the diagnostic performances of lesion-level treatment response prediction using transfer learning for four scenarios (1 VOI slice, 1 whole-slice, 3 VOI slices, and 3 whole-slices) using three different imaging modalities (dCT, lCT, and PET) are shown in FIG. 4.

The Scenario of 1 VOI Slice Plus 1 Whole-Slice as One Input (Combined Slices) Vs. 1 Whole-Slice

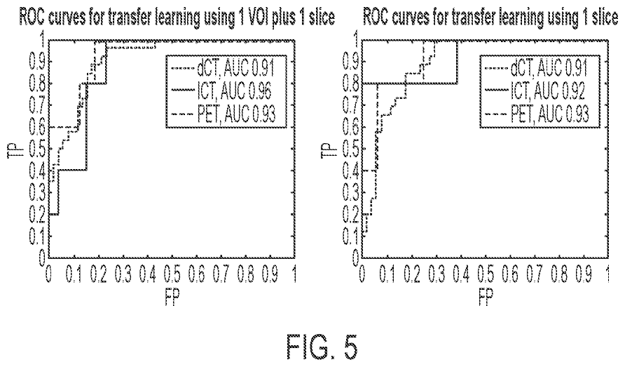

ROC curves for the diagnostic performance of lesion-level treatment response prediction using transfer learning for the scenarios of 1 VOI slice plus 1 whole-slice and 1 whole-slice are shown in FIG. 5. Considering AUC, the diagnostic performances of 1 VOI slice plus 1 whole-slice using dCT, lCT, or PET were not statistically significantly different from those of 1 whole-slice (Table 15). Details of the results of the five scenarios (1 VOI slice, 1 whole-slice, 3 VOI slices, 3 whole-slices, and 1 VOI slice plus 1 whole-slice (combined slices)) using three different imaging modalities are summarized in Table 16 (for dCT), Table 17 (for lCT), and Table 18 (for PET).

TABLE 15
T-test comparisons of diagnostic performance of transfer learning on testing data sets (using 40 epochs and
batch size 5) for lesion-level treatment response prediction between scenarios 1 VOI slice plus 1 whole-slice
(combined slices) vs. 1 whole-slice for 3 different imaging modalities (dCT, lCT, and PET). dCT = diagnostic
computed tomography, lCT = low-dose computed tomography, PET = positron emission tomography,
Acc = accuracy, Sens = sensitivity, Spec = specificity, AUC = area under the curve, VOI =
volume of interest. Mean and standard deviation values were listed (mean ± SD).

| | dCT | | | | lCT | | | |
|---|---|---|---|---|---|---|---|---|
| Scenarios | Acc | Sens | Spec | AUC | Acc | Sens | Spec | AUC |
| 1 VOI | 0.84 ± 0.04 | 0.89 ± 0.03 | 0.76 ± 0.10 | 0.91 ± 0.03 | 0.93 ± 0.03 | 0.96 ± 0.03 | 0.83 ± 0.14 | 0.98 ± 0.02 |
| plus 1 | | | | | | | | |
| whole-slice | | | | | | | | |
| (combined | | | | | | | | |
| slices) | | | | | | | | |
| 1 whole- | 0.82 ± 0.05 | 0.87 ± 0.07 | ±0.77 ± 0.12 | ±0.91 ± 0.03 | 0.91 ± 0.06 | 0.94 ± 0.06 | 0.75 ± 0.32 | 0.92 ± 0.08 |
| slice | | | | | | | | |
| p values | 0.41 | 0.30 | 0.76 | 0.98 | 0.36 | 0.35 | 0.46 | 0.07 |

| | PET | | | |
|---|---|---|---|---|
| Scenarios | Acc | Sens | Spec | AUC |
| 1 VOI | 0.87 ± 0.07 | 0.93 ± 0.07 | 0.73 ± 0.16 | 0.92 v0.08 |
| plus 1 | | | | |
| whole-slice | | | | |
| (combined | | | | |
| slices) | | | | |
| 1 whole- | 0.87 ± 0.06 | 0.90 ± 0.06 | 0.77 ± 0.19 | 0.93 ± 0.07 |
| slice | | | | |
| p values | 0.91 | 0.31 | 0.51 | 0.70 |

TABLE 16
Diagnostic performance of lesion-level treatment response prediction
(using 40 epochs and batch size 5) based on input from 5 scenarios
(1 VOI slice, 1 whole-slice, 3 VOI slices, 3 wholeslices, 1 VOI slice
plus 1 whole-slice (combined slices)) from diagnostic CT images.

| Scenario | | Accuracy | Sensitivity | Specificity | AUC |
|---|---|---|---|---|---|
| 1 VOI- | Training | 0.81 ± | 0.79 ± | 0.93 ± 0.05 | 0.83 ± 0.08 |
| slice | | 0.04 | 0.05 | | |
| | Validation | 0.66 ± | 0.69 ± | 0.52 ± 0.16 | 0.58 ± 0.06 |
| | | 0.04 | 0.02 | | |
| | Testing | 0.68 ± | 0.70 ± | 0.58 ± 0.16 | 0.59 ± 0.04 |
| | | 0.05 | 0.02 | | |
| 1 | Training | 0.90 ± | 0.92 ± | 0.87 ± 0.09 | 0.97 ± 0.01 |
| whole- | | 0.03 | 0.05 | | |
| slice | Validation | 0.81 ± | 0.85 ± | 0.76 ± 0.11 | 0.89 ± 0.03 |
| | | 0.04 | 0.04 | | |
| | Testing | 0.82 ± | 0.87 ± | 0.77 ± 0.12 | 0.91 ± 0.03 |
| | | 0.05 | 0.07 | | |
| 3 VOI- | Training | 0.77 ± | 0.75 ± | 0.89 ± 0.05 | 0.80 ± 0.04 |
| slices | | 0.03 | 0.03 | | |
| | Validation | 0.65 ± | 0.68 ± | 0.46 ± 0.12 | 0.55 ± 0.06 |
| | | 0.03 | 0.02 | | |
| | Testing | 0.65 ± | 0.68 ± | 0.52 ± 0.21 | 0.52 ± 0.07 |
| | | 0.05 | 0.02 | | |
| 3 whole- | Training | 0.94 ± | 0.96 ± | 0.91 ± 0.06 | 0.98 ± 0.01 |
| slices | | 0.02 | 0.03 | | |
| | Validation | 0.83 ± | 0.90 ± | 0.74 ± 0.09 | 0.90 ± 0.03 |
| | | 0.04 | 0.05 | | |
| | Testing | 0.84 ± | 0.90 ± | 0.76 ± 0.12 | 0.90 ± 0.05 |
| | | 0.05 | 0.04 | | |
| 1 VOI | Training | 0.95 ± | 0.97 ± | 0.93 ± 0.05 | 0.99 ± 0.01 |
| slice | | 0.03 | 0.03 | | |
| plus | Validation | 0.83 ± | 0.87 ± | 0.78 ± 0.07 | 0.90 ± 0.04 |
| 1 whole- | | 0.03 | 0.04 | | |
| slice | Testing | 0.84 ± | 0.89 ± | 0.76 ± 0.10 | 0.91 ± 0.03 |
| (combined | | 0.04 | 0.03 | | |
| slices | | | | | |

VOI = volume of interest, AUC = area under the curve, SD = standard deviation.
Mean and standard deviation values were listed (mean ± SD).

TABLE 17
Diagnostic performance of lesion-level treatment response prediction
(using 40 epochs and batch size 5) based on input from 5 scenarios (1
VOI slice, 1 whole-slice, 3 VOI slices, 3 whole-slices, 1 VOI slice plus
1 whole-slice (combined slices)) from low-dose CT images of PET/CT.

| Scenario | | Accuracy | Sensitivity | Specificity | AUC |
|---|---|---|---|---|---|
| 1 VOI- | Training | 0.81 ± 0.04 | 0.79 ± | 0.93 ± 0.05 | 0.96 ± |
| slice | | | 0.05 | | 0.04 |
| | Validation | 0.66 ± 0.04 | 0.69 ± | 0.52 ± 0.16 | 0.51 ± |
| | | | 0.02 | | 0.13 |
| | Testing | 0.68 ± 0.05 | 0.70 ± | 0.58 ± 0.16 | 0.60 ± |
| | | | 0.02 | | 0.11 |
| 1 | Training | 0.98 ± 0.01 | 0.99 ± | 0.97 ± 0.05 | 1.00 ± |
| whole- | | | 0.01 | | 0.00 |
| slice | Validation | 0.90 ± 0.04 | 0.93 ± | 0.78 ± 0.21 | 0.92 ± |
| | | | 0.04 | | 0.06 |
| | Testing | 0.91 ± 0.06 | 0.94 ± | 0.75 ± 0.32 | 0.92 ± |
| | | | 0.06 | | 0.08 |
| 3 VOI- | Training | 0.98 ± 0.01 | 0.98 ± | 0.97 ± 0.06 | 0.99 ± |
| slices | | | 0.02 | | 0.01 |
| | Validation | 0.81 ± 0.08 | 0.85 ± | 0.34 ± 0.14 | 0.51 ± |
| | | | 0.03 | | 0.19 |
| | Testing | 0.79 ± 0.06 | 0.84 ± | 0.21 ± 0.03 | 0.53 ± |
| | | | 0.02 | | 0.17 |
| 3 | Training | 0.99 ± 0.01 | 0.99 ± | 0.98 ± 0.04 | 1.00 ± |
| whole- | | | 0.01 | | 0.00 |
| slices | Validation | 0.93 ± 0.05 | 0.96 ± | 0.84 ± 0.20 | 0.95 ± |
| | | | 0.03 | | 0.06 |
| | Testing | 0.90 ± 0.05 | 0.95 ± | 0.74 ± 0.20 | 0.94 ± |
| | | | 0.04 | | 0.07 |
| 1 VOI | Training | 0.98 ± 0.02 | 0.99 ± | 0.96 ± 0.06 | 1.00 ± |
| slice | | | 0.01 | | 0.00 |
| plus | Validation | 0.94 ± 0.03 | 0.96 ± | 0.86 ± 0.15 | 0.97 ± |
| 1 whole- | | | 0.03 | | 0.03 |
| slice | Testing | 0.93 ± 0.03 | 0.96 ± | 0.83 ± 0.14 | 0.98 ± |
| (combined | | | 0.03 | | 0.02 |
| slices) | | | | | |

VOI = volume of interest, AUC = area under the curve.
Mean and standard deviation values were listed (mean ± SD).

TABLE 18
Diagnostic performance of lesion-level treatment response prediction
(using 40 epochs and batch size 5) based on input from 5 scenarios
(1 VOI slice, 1 whole-slice, 3 VOI slices, 3 whole-slices, 1 VOI slice
plus 1 whole-slice (combined slices)) from PET images of PET/CT.

| Scenario | | Accuracy | Sensitivity | Specificity | AUC |
|---|---|---|---|---|---|
| 1 VOI- | Training | 0.81 ± | 0.79 ± | 0.93 ± | 0.91 ± |
| slice | | 0.04 | 0.05 | 0.05 | 0.01 |
| | Validation | 0.66 ± | 0.69 ± | 0.52 ± | 0.59 ± |
| | | 0.04 | 0.02 | 0.16 | 0.13 |
| | Testing | 0.68 ± | 0.70 ± | 0.58 ± | 0.51 ± |
| | | 0.05 | 0.02 | 0.16 | 0.14 |
| 1 whole- | Training | 0.97 ± | 0.97 ± | 0.95 ± | 1.00 ± |
| slice | | 0.03 | 0.03 | 0.07 | 0.00 |
| | Validation | 0.89 ± | 0.94 ± | 0.75 ± | 0.93 ± |
| | | 0.03 | 0.04 | 0.11 | 0.06 |
| | Testing | 0.87 ± | 0.90 ± | 0.77 ± | 0.93 ± |
| | | 0.06 | 0.06 | 0.19 | 0.07 |
| 3 VOI- | Training | 0.77 ± | 0.75 ± | 0.92 ± | 0.80 ± |
| slices | | 0.01 | 0.01 | 0.08 | 0.04 |
| | Validation | 0.66 ± | 0.68 ± | 0.56 ± | 0.53 ± |
| | | 0.03 | 0.02 | 0.21 | 0.07 |
| | Testing | 0.65 ± | 0.68 ± | 0.48 ± | 0.53 ± |
| | | 0.02 | 0.01 | 0.09 | 0.07 |
| 3 whole- | Training | 0.95 ± | 0.98 ± | 0.90 ± | 0.99 ± |
| slices | | 0.02 | 0.02 | 0.07 | 0.01 |
| | Validation | 0.89 ± | 0.93 ± | 0.78 ± | 0.92 ± |
| | | 0.06 | 0.04 | 0.17 | 0.05 |
| | Testing | 0.90 ± | 0.95 ± | 0.81 ± | 0.95 ± |
| | | 0.07 | 0.05 | 0.19 | 0.06 |
| 1 VOI | Training | 0.98 ± | 0.99 ± | 0.97 ± | 1.00 ± |
| slice | | 0.02 | 0.02 | 0.05 | 0.01 |
| plus 1 | Validation | 0.81 ± | 0.90 ± | 0.59 ± | 0.86 ± |
| whole- | | 0.09 | 0.06 | 0.18 | 0.10 |
| slice | Testing | 0.87 ± | 0.93 ± | 0.73 ± | 0.92 ± |
| (combined | | 0.07 | 0.07 | 0.16 | 0.08 |
| slices) | | | | | |

VOI = volume of interest, AUC = area under the curve.
Mean and standard deviation values were listed (mean ± SD).

Incremental Learning for Lesion-Level Response Prediction from Pre-Treatment Low-Dose CT and PET Images

TABLE 19
Diagnostic performance of incremental learning on test data sets for lesion-level treatment response
prediction on low-dose CT (lCT) and positron emission tomography (PET) imaging modalities. lCT =
low-dose computed tomography, PET = positron emission tomography, Acc = accuracy, Sens =
sensitivity, Spec = specificity, AUC = area under the curve. Mean and standard deviation values were listed (mean ± SD).

| | 1 whole-slice | | | | 3 whole-slices | | | |
|---|---|---|---|---|---|---|---|---|
| | Acc | Sens | Spec | AUC | Acc | Sens | Spec | AUC |
| Incremental | 0.93 ± 0.03 | 0.96 ± 0.03 | 0.82 ± 0.16 | 0.96 ± 0.04 | 0.98 ± 0.02 | 0.99 ± 0.02 | 0.93 ± 0.09 | 0.99 ± 0.02 |
| learning on | | | | | | | | |
| lCT | | | | | | | | |
| Transfer | 0.91 ± 0.06 | 0.94 ± 0.06 | 0.75 ± 0.32 | 0.92 ± 0.08 | 0.90 ± 0.05 | 0.95 ± 0.04 | 0.74 ± 0.20 | 0.94 ± 0.07 |
| learning on | | | | | | | | |
| lCT | | | | | | | | |
| p values | 0.46 | 0.37 | 0.52 | 0.19 | <0.001 | 0.02 | 0.02 | 0.05 |
| Incremental | 0.86 ± 0.07 | 0.95 ± 0.04 | 0.70 ± 0.18 | 0.96 ± 0.04 | 0.86 ± 0.07 | 0.91 ± 0.04 | 0.72 ± 0.16 | 0.92 ± 0.06 |
| learning on | | | | | | | | |
| PET | | | | | | | | |
| Transfer | 0.87 ± 0.06 | 0.90 ± 0.06 | 0.77 ± 0.19 | 0.93 ± 0.07 | 0.90 ± 0.07 | 0.95 ± 0.05 | 0.81 ± 0.19 | 0.95 ± 0.06 |
| learning on | | | | | | | | |
| PET | | | | | | | | |
| p values | 0.88 | 0.04 | 0.36 | 0.30 | 0.20 | 0.05 | 0.26 | 0.18 |

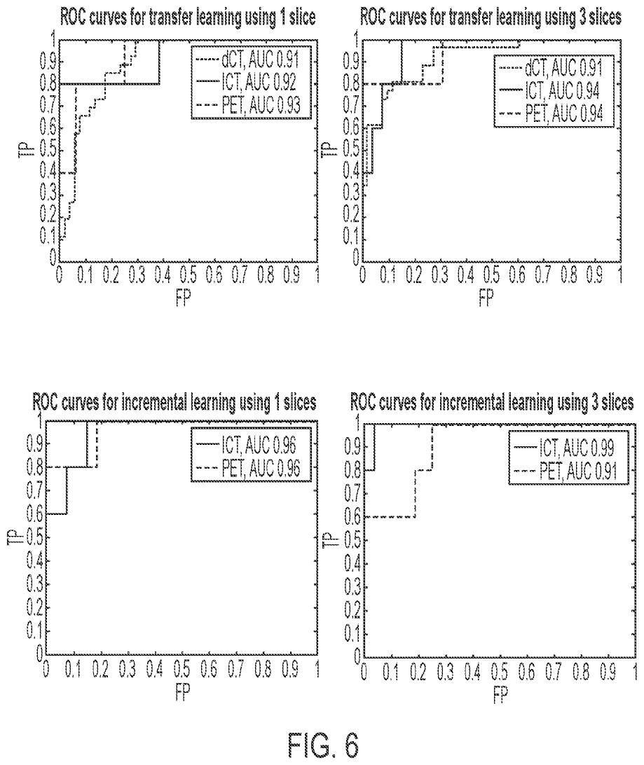

Table 19 summarizes the diagnostic performance of incremental learning on lCT and PET imaging modalities as well as comparisons with the results based on transfer learning (from Alexnet) to lCT and PET using the same batch size (5) and number of epochs (40). There was no statistically significant difference in lesion-level treatment response prediction between transfer learning from dCT to lCT or PET compared to incremental learning on lCT or PET. For example, P values of AUC for incremental learning vs. transfer learning using 1 whole-slice from lCT and PET were 0.19 and 0.30, respectively. ROC curves for diagnostic performance of lesion-level treatment response prediction using incremental learning vs. transfer learning for 1 whole-slice and 3 whole-slice scenarios on dCT, lCT, and PET images are shown in FIG. 6.

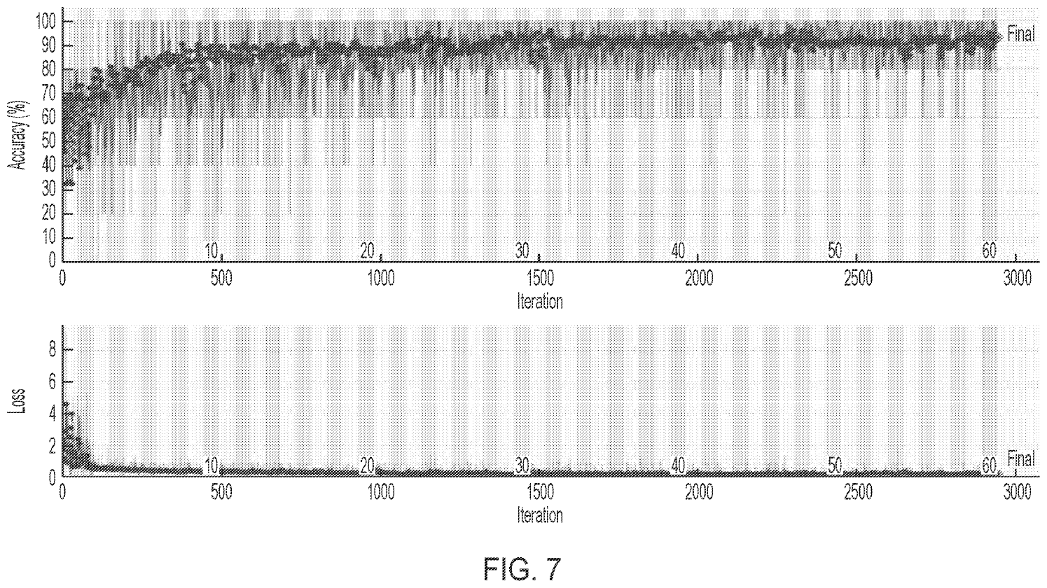

The diagnostic performance of lesion-level treatment response prediction based on input from 1 whole-slice dCT based on different parameters of batch size (B) and number of epochs (E) is shown in supplemental Table 20. The program was run 10 times, and each time the whole data set was randomly separated into 60% training, 20% validation, and 20% testing, where the testing data sets were independent of the training and validation data sets. The prediction results were robust and without observed statistically significant differences in AUC based on different parameters (in particular, with the maximum change in E [40, 80, 100, 200] and B [5, 10, 20, 30]). For example, the accuracy was 0.82 vs. 0.83 for B5 E40 vs. B30 E200, respectively (p=0.70) and the AUC was 0.91 vs. 0.89 for B5 E40 vs. B30 E200, respectively (p=0.14) (see Table 21). FIG. 7 shows that the neural network was well-trained with both training/validation curves from one of the 10 repeat experiments with E=80 and B=5.

TABLE 20
Diagnostic performance of lesion-level treatment response prediction using transfer learning on 1 whole-slice dCT based on
different parameters of batch size (B) and number of epochs (E), dCT = diagnostic computed tomography, Acc = accuracy,
Sens = sensitivity, Spec = specificity, AUC = area under the curve. Mean and standard deviation values were listed (mean ± SD).

| Test | Acc | Sens | Spec | AUC | Test | Acc | Sens | Spec | AUC | Test | Acc | Sens | Spec | AUC | Test | Acc | Sens | Spec | AUC |
|---|---|---|---|---|---|---|---|---|---|---|---|---|---|---|---|---|---|---|---|
| B5 | 0.82 | 0.87 | 0.77 | 0.91 | B10 | 0.82 | 0.89 | 0.71 | 0.91 | B20 | 0.81 | 0.86 | 0.71 | 0.89 | B30 | 0.79 | 0.86 | 0.68 | 0.85 |
| E40 | ±0.05 | ±0.07 | ±0.12 | ±0.03 | E40 | ±0.04 | ±0.05 | ±0.07 | ±0.02 | E40 | ±0.03 | ±0.05 | ±0.05 | ±0.03 | E40 | ±0.06 | ±0.04 | ±0.11 | +0.05 |
| B5 | 0.82 | 0.84 | 0.77 | 0.89 | B10 | 0.85 | 0.89 | 0.79 | 0.90 | B20 | 0.85 | 0.89 | 0.77 | 0.89 | B30 | 0.82 | 0.87 | 0.73 | 0.87 |
| E80 | ±0.03 | ±0.03 | ±0.09 | ±0.05 | E80 | ±0.03 | ±0.04 | ±0.05 | ±0.03 | E80 | ±0.04 | ±0.05 | ±0.07 | ±0.04 | E80 | ±0.06 | ±0.04 | ±0.09 | +0.05 |
| B5 | 0.85 | 0.88 | 0.78 | 0.93 | B10 | 0.85 | 0.88 | 0.82 | 0.91 | B20 | 0.84 | 0.88 | 0.76 | 0.89 | B30 | 0.82 | 0.87 | 0.72 | 0.87 |
| E100 | ±0.03 | ±0.04 | ±0.06 | ±0.02 | E100 | ±0.03 | ±0.04 | ±0.09 | ±0.04 | E100 | ±0.02 | ±0.04 | ±0.03 | ±0.03 | E100 | ±0.03 | ±0.04 | ±0.05 | +0.04 |
| B5 | 0.87 | 0.90 | 0.83 | 0.93 | B10 | 0.87 | 0.90 | 0.83 | 0.89 | B20 | 0.82 | 0.87 | 0.74 | 0.90 | B30 | 0.83 | 0.86 | 0.78 | 0.89 |
| E200 | ±0.04 | ±0.05 | ±0.08 | ±0.02 | E200 | ±0.05 | ±0.04 | ±0.08 | ±0.05 | E200 | ±0.02 | ±0.03 | ±0.05 | ±0.04 | E200 | ±0.03 | ±0.03 | ±0.08 | +0.03 |

TABLE 21
p values of t-test comparisons of diagnostic performance of transfer
learning for lesion-level treatment response prediction results in Table S6.

| p values | Acc | Sens | Spec | AUC |
|---|---|---|---|---|
| B5 E40 vs. B5 E200 | 0.03 | 0.28 | 0.19 | 0.21 |
| B10 E40 vs. B10 | 0.03 | 0.40 | 0.02 | 0.19 |
| E200 | | | | |
| B20 E40 vs. B20 | 0.16 | 0.20 | 0.29 | 0.65 |
| E200 | | | | |
| B30 E40 vs. B30 | 0.12 | 0.88 | 0.08 | 0.12 |
| E200 | | | | |
| B5 E200 vs. B30 | 0.06 | 0.21 | 0.29 | 0.09 |
| E200 | | | | |
| B5 E40 vs. B30 | 0.70 | 0.57 | 0.95 | 0.14 |
| E200 | | | | |
| B5 E40 vs. B30 E40 | 0.24 | 0.87 | 0.09 | 0.01 |
| B5 E80 vs. B30 E80 | 0.003 | 0.009 | 0.0003 | 0.26 |
| B5 E100 vs. B30 | 0.03 | 0.51 | 0.02 | 0.001 |
| E100 | | | | |
| B5 E200 vs. B30 | 0.02 | 0.05 | 0.12 | 0.002 |

E200

B = batch size, E = number of epochs, Acc = accuracy, Sens = sensitivity, Spec = specificity, AUC = area under the curve.

### Patient-Level Treatment Response Prediction Results1 Whole-Slice Input Scenario

The results of patient-level response prediction using the rule-based reasoning approach from dCT, lCT, and PET relative to the reference standard of 12-month post-treatment patient-level response status are shown in Table 22. These were derived from lesion-level response predictions based on the 1 whole-slice input scenario and transfer learning. (Comparable results based on the 3 whole-slices input scenario are reported separately in supplemental Table 23).

Patient-level response prediction for all patients from dCT based on the “Majority 60%” rule had accuracy 0.79, sensitivity 0.83, and specificity 0.75, which was not significantly different than that from lCT (with accuracy 0.65, sensitivity 0.60, and specificity 0.75) (p=0.80) and PET (with accuracy 0.56, sensitivity 0.55, and specificity 0.57) (p=0.87). In addition, patient level response prediction for DLBCL patients from dCT based on the “Majority 60%” rule had accuracy 0.81, sensitivity 0.75, and specificity 0.88, which was significantly better than the best IPI-based patient level response prediction using an IPI risk factor threshold of <1 (with accuracy 0.54, sensitivity 0.38, and specificity 0.61) (p=0.046).

For dCT, the accuracy of the “Majority 60%” rule (0.79) was not statistically significantly different than that of the “Majority 70%” rule (0.71) (p=0.38) but was statistically significantly greater than that of the “All” rule (0.61) (p=0.027). For lCT, the accuracies of the “Majority 60%” and “Majority 70%” rules (0.65) were identical and not statistically significantly different than that of the “All” rule (0.52) (p=0.20).

For PET, the accuracies of the “Majority 70%” and “All” rules (0.61) were identical and not statistically significantly different than that of the “Majority 60%” rule (0.56) (p=0.73).

TABLE 22
Diagnostic performance of patient-level treatment response prediction in lymphoma
using rule-based reasoning approach (from lesion-level response predictions using
1 whole-slice input scenario, 3 image modalities, and transfer learning) compared
to International Prognostic Index risk factors for diffuse large B-cell lymphoma
(DLBCL) patients. Note that results are shown for entire subject cohort (All) and
for DLBCL subject cohort. dCT = diagnostic computed tomography, lCT =
low-dose computed tomography, PET = positron emission tomography, IPI =
International Prognostic Index, Acc = accuracy, Sens = sensitivity, Spec = specificity.

| | | Patient response with | | | Patient response with | | | | | |
|---|---|---|---|---|---|---|---|---|---|---|
| | | “All” Rule | | | “Majority” Rule | | | | | |
| | | All lesions | | | At least “60%” | | | At least “70%” | | |
| Subject | | responded | | | lesions responded | | | lesions responded | | |
| cohort | Modality | Acc | Sens | Spec | Acc | Sens | Spec | Acc | Sens | Spec |
| All | dCT | 0.61 | 1.00 | 0.56 | 0.79 | 0.83 | 0.75 | 0.71 | 0.88 | 0.65 |
| | lCT | 0.52 | 0.50 | 0.54 | 0.65 | 0.60 | 0.75 | 0.65 | 0.60 | 0.75 |
| | PET | 0.61 | 0.60 | 0.63 | 0.56 | 0.55 | 0.57 | 0.61 | 0.58 | 0.67 |
| DLBCL | dCT | 0.69 | 1.00 | 0.64 | 0.81 | 0.75 | 0.88 | 0.75 | 0.80 | 0.73 |
| | lCT | 0.56 | 0.44 | 0.67 | 0.56 | 0.45 | 0.71 | 0.56 | 0.45 | 0.71 |
| | PET | 0.57 | 0.50 | 0.67 | 0.43 | 0.38 | 0.50 | 0.50 | 0.44 | 0.60 |

| | |
|---|---|
| IPI ≤ 1 | Acc = 0.54; Sens = 0.38; Spec = 0.61 |
| IPI ≤ 2 | Acc = 0.42; Sens = 0.37; Spec = 0.57 |
| IPI ≤ 3 | Acc = 0.27; Sens = 0.30; Spec = 0.00 |

Patient-Level Treatment Response Prediction Result: 3 Whole Slices Input Scenario

The results of patient-level response prediction in all 39 patients using the rule-based reasoning approach from dCT, lCT, and PET relative to the reference standard of 12-month post-treatment patient-level response status are shown in Table 23. These were derived from lesion-level response predictions based on the 3 whole-slices input scenario and transfer learning. In addition, for the 26 patients with diffuse large B cell lymphoma (DLBCL), these results from the rule-based reasoning approach were compared to those from the International Prognostic Index (IPI)-based approach, utilizing Pearson's chi-square test for statistical comparison. For the 3 whole-slices input scenario, patient-level response prediction for all patients from dCT based on the “Majority 60%” rule had accuracy 0.71, sensitivity 0.67, and specificity 0.80, which was not statistically significantly different than that from lCT (with accuracy 0.61, sensitivity 0.57, and specificity 0.67) (p=0.40) and PET (with accuracy 0.47, sensitivity 0.45, and specificity 0.50) (p=0.26). In addition, for the 3 whole-slice scenario, patient-level response prediction for DLBCL patients from dCT based on the “Majority 60%” rule had accuracy 0.69, sensitivity 0.60, and specificity 0.83, which was statistically significantly greater than the best IPI-based patient-level response prediction using an IPI score threshold of <1 (with accuracy 0.54, sensitivity 0.38, and specificity 0.61) (p=0.047).

TABLE 23
Diagnostic performance of patient-level treatment response prediction in lymphoma
using rule-based reasoning approach (from lesion-level response predictions using
3 whole-slices input scenario, 3 image modalities, and transfer learning) compared
to International Prognostic Index risk factors for diffuse large B-cell lymphoma
(DLBCL) patients. Note that results are shown for entire subject cohort (All)
and for DLBCL subject cohort. dCT = diagnostic computed tomography, lCT =
low-dose computed tomography, PET = positron emission tomography, IPI =
International Prognostic Index, Acc = accuracy, Sens = sensitivity, Spec = specificity.

| | | Patient response with | | | Patient response with | | | | | |
|---|---|---|---|---|---|---|---|---|---|---|
| | | “All” Rule | | | “Majority” Rule | | | | | |
| | | All lesions | | | At least “60%” | | | At least “70%” | | |
| Subject | | responded | | | lesions responded | | | lesions responded | | |
| cohort | Modality | Acc | Sens | Spec | Acc | Sens | Spec | Acc | Sens | Spec |
| All | dCT | 0.64 | 0.75 | 0.60 | 0.71 | 0.67 | 0.80 | 0.75 | 0.82 | 0.71 |
| | lCT | 0.43 | 0.40 | 0.46 | 0.61 | 0.57 | 0.67 | 0.57 | 0.54 | 0.60 |
| | PET | 0.35 | 0.29 | 0.40 | 0.47 | 0.45 | 0.50 | 0.41 | 0.40 | 0.43 |
| DLBCL | dCT | 0.69 | 0.75 | 0.67 | 0.69 | 0.60 | 0.83 | 0.81 | 1.00 | 0.75 |
| | lCT | 0.50 | 0.40 | 0.63 | 0.56 | 0.45 | 0.71 | 0.56 | 0.45 | 0.71 |
| | PET | 0.38 | 0.29 | 0.50 | 0.38 | 0.33 | 0.50 | 0.38 | 0.33 | 0.50 |

| | |
|---|---|
| IPI ≤ 1 | Acc = 0.54; Sens = 0.38; Spec = 0.61 |
| IPI ≤ 2 | Acc = 0.42; Sens = 0.37; Spec = 0.57 |
| IPI ≤ 3 | Acc = 0.27; Sens = 0.30; Spec = 0.00 |

### Discussion

The medical image-based approach to determine personalized response prediction to CAR T-cell therapies has certain unique advantages including: the use of pre-existing diagnostic imaging data sets previously acquired for clinical purposes, the lack of invasiveness, the extraction of regional phenotypic information from disease sites throughout the body that may be heterogeneous, and production-mode efficiency. As such, the feasibility of a novel deep learning image analysis methodology applied to pre-treatment diagnostic CT, low-dose CT, and FDG-PET images to predict lesion-level treatment response to CAR T-cell therapy in patients with lymphoma was investigated. Based on this analysis, a rule-based reasoning approach was used to assess the feasibility of predicting patient-level treatment response. The approach disclosed herein has not yet been studied in the clinical context. Transfer learning was first adopted for the prediction of lesion-level treatment response on diagnostic CT, and then incremental learning was utilized for the prediction of lesion-level treatment response on low-dose CT and PET. The results from different experiments (over 3,000) were subsequently reported and compared.

Lesion-level treatment response prediction based on the input from single whole diagnostic CT slices passing through the mid-portions of abnormal lymph nodes had an accuracy of 82%±5%, sensitivity 87%±7%, specificity 77%±12%, and AUC 0.91±0.03, and seemed to be stable to alterations of various hyper-parameters utilized in the neural network. Transfer learning on low-dose CT and on PET images had similar diagnostic performances as to that on diagnostic CT images.

Incremental learning for lesion-level treatment response prediction on single whole slice low-dose CT achieved an accuracy of 93%±3%, sensitivity of 96%±3%, specificity of 82%±16%, and AUC 0.96±0.04, which were not statistically significantly different from the results of transfer learning. Similarly, incremental learning for lesion-level treatment response prediction on single whole slice PET had an accuracy of 86%±7%, sensitivity of 95%±4%, specificity of 70%±18%, and AUC 0.96±0.04, which again were not statistically significantly different from those of transfer learning.

Recent research on outcome prediction in patients with DLBCL was based on clinical and pathologic information, such as disclosed in [28, 29], where regression and machine learning methods were utilized. In the disclosure of [28], a model for predicting health outcome in patients with DLBCL treated with standard of care by using lasso logistic regression was reported. In the disclosure of [29], machine learning approach was used to achieve the optimizing outcome prediction in patients with DLBCL, which combined a number of predicted survival curves into one by means of a weighted average. The weights were selected so that the cross-validated integrated Brier score (IBS) is minimized. Different models were selected for catching survival curves, such as Cox proportional hazard (CPH) model, penalized CPH models, and accelerated failure time (AFT) model etc. Ref [28] reported the concordance index as 0.756 for Danish cohort and 0.744 for Swedish cohort. No medical images were involved in the disclosures of [28, 29]. Additionally, there are no reports disclosing the use of deep learning tools to predict treatment response in lymphoma based on medical images for patients with DLBCL or FL.

Although a relatively small number of patients were assessed in this study, a large number of individual lymphoma lesions were available for evaluation in these patients and data augmentation techniques were also utilized, enabling the successful implementation and evaluation of a DL methodology to perform lesion-level response prediction. In this study analysis was focused on lymph nodal lesions only, given the small numbers of extranodal lesions and splenic and Waldeyer's ring nodal tissue lesion sites present in the patient cohort analyzed. Yet, the results described herein demonstrate a high diagnostic performance of the current approach. In future studies, the current approach can be extended to include other non-lymph nodal lesion inputs into the DL system to determine if this would improve diagnostic performance.

Even though patient-level treatment response prediction using machine learning approaches were not performed due to the relatively small number of patients, the results described herein show a high diagnostic performance of patient-level prediction using a rule-based reasoning approach by utilizing the lesion-level results from DL.

In summary, the Example disclosed herein demonstrates the feasibility of a novel deep learning image analysis methodology to accurately predict lesion-level response of patients with lymphoma to chimeric antigen receptor (CAR) T-cell therapy based on pre-treatment CT and PET lCT images, as well as the feasibility of a rule-based reasoning approach to accurately predict patient-level response assessment. The results are promising, and as such, these approaches can be clinically impactful by providing new information regarding which patients will or will not respond to treatment in advance of initiating therapy. Such knowledge would be useful, e.g., to help prevent unnecessary exposures to therapy with potential associated toxicities and high costs in those in whom the treatment will likely not work, while simultaneously enabling treatment planning using alternative potentially efficacious treatments.

### DOCUMENTS CITED IN THIS EXAMPLE

[1] Schuster S J, Bishop M R, Tam C S, Waller E K, Borchmann P, McGuirk J P, Jager U, Jaglowski S, Andreadis C, Westin J R, Fleury I, Bachanova V, Foley S R, Ho P J, Mielke S, Magenau J M, Holte H, Pantano S, Pacaud L B, Awasthi R, Chu J, Anak O, Salles G, Maziarz R T, Investigators J. Tisagenlecleucel in Adult Relapsed or Refractory Diffuse Large B-Cell Lymphoma. N Engl J Med. 2019; 380(1):45-56. doi: 10.1056/NEJMoa1804980. PubMed PMID: 30501490.
[2] Schuster S J, Svoboda J, Chong E A, Nasta S D, Mato A R, Anak O, et al., Chimeric Antigen Receptor T Cells in Refractory B-Cell Lymphomas. N Engl J Med. 2017; 377(26):2545-54.
[3] Mato A R, Thompson M C, Nabhan C, Svoboda J, Schuster S J. Chimeric Antigen Receptor T-Cell Therapy for Chronic Lymphocytic Leukemia: A Narrative Review. Clin Lymphoma Myeloma Leuk. 2017; 17(12):852-6.
[4] Maude, S. L. et al. Tisagenlecleucel in children and young adults with B cell lymphoblastic leukemia. N. Engl. J. Med. 378, 439-448 (2018).
[5] Fraietta J A, Lacey S F, Orlando E J, Pruteanu-Malinici I, Gohil M, Lundh S, Boesteanu A C, et al., Melenhorst J J. Determinants of response and resistance to CD19 chimeric antigen receptor (CAR) T cell therapy of chronic lymphocytic leukemia. Nat Med. 2018; 24(5):563-71.
[6] Fraietta J A, Nobles C L, Sammons M A, Lundh S, Carty S A, Reich T J, et al., Melenhorst J J. Disruption of TET2 promotes the therapeutic efficacy of CD19-targeted T cells. Nature. 2018.
[7] LeCun Y, Bengio Y, Hinton G. Deep learning. Nature. 2015; 521(7553):436-44.
[8] Mnih V, Kavukcuoglu K, Silver D, Rusu A A, Veness J, Bellemare M G, et al., Human-level control through deep reinforcement learning. Nature. 2015; 518(7540):529-33.
[9] Naito T, Nagashima Y, Taira K, Uchio N, Tsuji S, Shimizu J. Identification and segmentation of myelinated nerve fibers in a cross-sectional optical microscopic image using a deep learning model. J Neurosci Methods. 2017; 291:141-9.
[10] Wand M, Schultz T. Pattern learning with deep neural networks in EMG-based speech recognition. Conf Proc IEEE Eng Med Biol Soc. 2014; 2014:4200-3.
[11] Nguyen M T, Nguyen B V, Kim K. Deep Feature Learning for Sudden Cardiac Arrest Detection in Automated External Defibrillators. Sci Rep. 2018; 8(1):17196. doi: 10.1038/s41598-018-33424-9. PubMed PMID: 30464177; PMCID: PMC6249221.
[12] Sari C T, Gunduz-Demir C. Unsupervised Feature Extraction via Deep Learning for Histopathological Classification of Colon Tissue Images. IEEE Trans Med Imaging. 2018. doi: 10.1109/TMI.2018.2879369. PubMed PMID: 30403624.
[13] Zhou T, Thung K H, Zhu X, Shen D. Effective feature learning and fusion of multimodality data using stage-wise deep neural network for dementia diagnosis. Hum Brain Mapp. 2019; 40(3):1001-16. doi: 10.1002/hbm.24428. PubMed PMID: 30381863.
[14] Vununu C, Moon K S, Lee S H, Kwon K R. A Deep Feature Learning Method for Drill Bits Monitoring Using the Spectral Analysis of the Acoustic Signals. Sensors (Basel). 2018; 18(8). doi: 10.3390/s18082634. PubMed PMID: 30103498; PMCID: PMC6111733.
[15] Hu G, Wang K, Peng Y, Qiu M, Shi J, Liu L. Deep Learning Methods for Underwater Target Feature Extraction and Recognition. Comput Intell Neurosci. 2018; 2018:1214301. doi: 10.1155/2018/1214301. PubMed PMID: 29780407; PMCID: PMC5892262.
[16] Liu J, Xu B, Zheng C, Gong Y, Garibaldi J, Soria D, Green A, Ellis 10, Zou W, Qiu G. An End-to-End Deep Learning Histochemical Scoring System for Breast Cancer TMA. IEEE Trans Med Imaging. 2019; 38(2):617-28. doi: 10.1109/TMI.2018.2868333. PubMed PMID: 30183623.
[17] Nishio M, Sugiyama O, Yakami M, Ueno S, Kubo T, Kuroda T, Togashi K. Computer-aided diagnosis of lung nodule classification between benign nodule, primary lung cancer, and metastatic lung cancer at different image size using deep convolutional neural network with transfer learning. PLoS One. 2018; 13(7):e0200721. doi: 10.1371/journal.pone.0200721. PubMed PMID: 30052644; PMCID: PMC6063408.
[18] Niazi M K K, Tavolara T E, Arole V, Hartman D J, Pantanowitz L, Gurcan M N. Identifying tumor in pancreatic neuroendocrine neoplasms from Ki67 images using transfer learning. PLoS One. 2018; 13(4):e0195621. doi: 10.1371/journal.pone.0195621. PubMed PMID: 29649302; PMCID: PMC5896941.
[19] A Krizhevsky, I Sutskever, G E Hinton, Imagenet classification with deep convolutional neural networks, Advances in neural information processing systems, 2012.
[20] International Non-Hodgkin's Lymphoma Prognostic Factors P. A predictive model for aggressive non-Hodgkin's lymphoma. N Engl J Med. 1993; 329(14):987-94. doi: 10.1056/NEJM199309303291402. PubMed PMID: 8141877.
[21] Federico M, Vitolo U, Zinzani P L, Chisesi T, Clo V, Bellesi G, Magagnoli M, Liberati M, Boccomini C, Niscola P, Pavone V, Cuneo A, Santini G, Brugiatelli M, Baldini L, Rigacci L, Resegotti L. Prognosis of follicular lymphoma: a predictive model based on a retrospective analysis of 987 cases. Intergruppo Italiano Linfomi. Blood. 2000; 95(3):783-9. PubMed PMID: 10648386.
[22] Grevera G, Udupa J, Odhner D, Zhuge Y, Souza A, Iwanaga T, Mishra S. CAVASS: a computer-assisted visualization and analysis software system. J Digit Imaging. 2007; 20 Suppl 1:101-18.
[23] Karen Simonyan, Andrew Zisserman. Very Deep Convolutional Networks for Large-Scale Image Recognition, arXiv:1409.1556 [cs.CV]
[24] Kaiming He, Xiangyu Zhang, Shaoqing Ren, Jian Sun. Deep Residual Learning for Image Recognition, CVPR 2015.
[25] Mei, Song. A mean field view of the landscape of two-layer neural networks. Proceedings of the National Academy of Sciences. 115 (33): E7665-E7671, 2018. doi:10.1073/pnas.1806579115. PMC 6099898. PMID 30054315.
[26] Hiroshi Inoue, Data Augmentation by Pairing Samples for Images Classification, arXiv:1801.02929.
[27] Jason Wang, Luis Perez, The Effectiveness of Data Augmentation in Image Classification using DeepLearning, 2017, http://cs231n.stanford.edu/reports/2017/pdfs/300.pdf.
[28] Galaznik A, Reich C, Klebanov G, Khoma Y, Allakhverdiiev E, Hather G, Shou Y. Predicting Outcomes in Patients With Diffuse Large B-Cell Lymphoma Treated With Standard of Care. Cancer Inform. 2019; 18:1176935119835538. doi: 10.1177/1176935119835538. PubMed PMID: 30906191; PMCID: PMC6421613.
[29] Biccler J L, Eloranta S, de Nully Brown P, Frederiksen H, Jerkeman M, Jorgensen J, Jakobsen L H, Smedby K E, Bogsted M, El-Galaly T C. Optimizing Outcome Prediction in Diffuse Large B-Cell Lymphoma by Use of Machine Learning and Nationwide Lymphoma Registries: A Nordic Lymphoma Group Study. JCO Clin Cancer Inform. 2018; 2:1-13. doi: 10.1200/CCI.18.00025. PubMed PMID: 30652603.

### EQUIVALENTS

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

What is claimed is:

1. A computer-implemented method for determining, e.g., predicting, a lesion-level treatment response to a therapy comprising a population of immune effector cells that expresses a Chimeric Antigen Receptor (CAR) that binds to CD19 (“CAR19 therapy”), said method comprising:
acquiring, e.g., receiving, an image of a lesion of a subject, e.g., a subject having or at risk of having a lymphoma (“acquired image”); and
processing the image with a neural network (“processed image”),
wherein the neural network outputs a classification result indicating the lesion-level treatment response to the CAR19 therapy.

2. A system for determining, e.g., predicting, a lesion-level treatment response to a therapy comprising a population of immune effector cells that expresses a Chimeric Antigen Receptor (CAR) that binds to CD19 (“CAR19 therapy”), said system comprising:
a processor; and
a storage device storing instructions that, when executed by the processor, cause the processor to:
acquire, e.g., receive, an image of a lesion of a subject, e.g., a subject having, or at risk of having, a lymphoma (“acquired image”); and
process the image with a neural network (“processed image”),
wherein the neural network outputs a classification result indicating the lesion-level treatment response to the CAR19 therapy.

3. A non-transitory computer-readable medium for determining, e.g., predicting, a lesion-level treatment response to a therapy comprising a population of immune effector cells that expresses a Chimeric Antigen Receptor (CAR) that binds to CD19 (“CAR19 therapy”), said medium comprising instructions that, when executed by a processor, cause the processor to:
acquire, e.g., receive, an image of a lesion of the subject, e.g., a subject having, or at risk of having, a lymphoma (“acquired image”); and
process the image with a neural network (“processed image”),
wherein the neural network outputs a classification result indicating the lesion-level treatment response to the CAR19 therapy.

4. A method for treating a subject having, or at risk of having, lymphoma, comprising: responsive to a determination, e.g., prediction, of a lesion-level treatment response to a therapy comprising a population of immune effector cells that expresses a Chimeric Antigen Receptor (CAR) that binds to CD19 (“CAR19 therapy”),
administering the CAR19 therapy to the subject, thereby treating the subject, wherein said determination, e.g., prediction, comprises:
acquiring, e.g., receiving, an image of a lesion of the subject (“acquired image”); and
processing the image with a neural network (“processed image”), wherein the neural network outputs a classification result indicating the lesion-level treatment response to the CAR19 therapy.

5. A method for evaluating, or predicting the responsiveness of, a subject having or at risk of having a lymphoma to a CAR19 therapy, said method comprising:
determining, e.g., predicting, of a lesion-level treatment response a therapy comprising a population of immune effector cells that expresses a Chimeric Antigen Receptor (CAR) that binds to CD19 (“CAR19 therapy”), with a neural network, wherein said determining comprises:
acquiring, e.g., receiving, an image of a lesion of the subject (“acquired image”); and
processing the image with the neural network (“processed image”), wherein the neural network outputs a classification result indicating the lesion-level treatment response to the CAR19 therapy; and
thereby evaluating the subject, or predicting the responsiveness of the subject, to the CAR19 therapy.

6. The method, system or medium of any one of claims 1-5, wherein the CAR19 therapy is a therapy comprising immune effector cells expressing an anti-CD19 binding domain, a transmembrane domain, and an intracellular signaling domain comprising a stimulatory domain.

7. The method, system or medium of any of claims 1-6, wherein the lymphoma is chosen from diffuse large B-cell lymphoma (DLBCL), follicular lymphoma (FL), mantle cell lymphoma (MCL), B cell prolymphocytic leukemia, blastic plasmacytoid dendritic cell neoplasm, Burkitt's lymphoma, hairy cell leukemia, small cell- or a large cell-follicular lymphoma, malignant lymphoproliferative conditions, MALT lymphoma, Marginal zone lymphoma, multiple myeloma, myelodysplasia and myelodysplastic syndrome, non-Hodgkin lymphoma, Hodgkin lymphoma, or plasmablastic lymphoma.

8. The method, system or medium of any of the preceding claims, wherein the lesion-level treatment response is indicative of responsiveness to the CAR19 therapy.

9. The method, system or medium of any of the preceding claims, wherein the lesion-level treatment response is evaluated using one, two, three or more (all) of the following parameters: 1) a change in lesion size; 2) a change in metabolic activity; 3) a change in lesion morphology; or 4) a change in lesion intensity. (e.g., lesion attenuation (on CT), lesion signal intensity (on MRI, whether T1-weighted, T2-weighted, or diffusion-weighted), or lesion contrast enhancement (on CT or MRI)); 5) a change in lesion morphology (e.g., a lesion size, a lesion volume, or a lesion shape); 6) a change in lesion radiotracer uptake (e.g., FDG uptake on PET imaging or DOTATE uptake on PET imaging); 7) a change in lesion texture (e.g., on CT, MRI, PET, or SPECT); and/or 8) a change in non-lesion tissue properties (e.g., on CT, MRI, PET, or SPECT in terms of tissue morphology, radiotracer activity, intensity, or texture).

10. The method, system or medium of claim 9, wherein a decrease in one, two, three or more (all) of 1-8 is indicative of a positive response to the CAR19 therapy.

11. The method, system or medium of claim 9 or 10, wherein an increase or lack of detectable change in one or more of 1-8 is indicative of a negative response to the CAR19 therapy.

12. The method, system or medium of any of the preceding claims, wherein the prediction of the lesion-level treatment response is followed by a rule-based reasoning method, to thereby determine a patient-level response prediction.

13. The method, system or medium of claim 12, wherein the patient-level response prediction comprises an All rule.

14. The method, system or medium of claim 12, wherein the patient-level response prediction comprises a Majority Rule.
